(19) United States
(12) Patent Application Publication
(10) Pub. No.: US 2024/0254488 A1
(43) Pub. Date: Aug. 1, 2024

(54) ANTISENSE OLIGOMERS FOR TREATMENT OF NON-SENSE MEDIATED RNA DECAY BASED CONDITIONS AND DISEASES

(71) Applicant: Stoke Therapeutics, Inc., Bedford, MA (US)

(72) Inventors: Isabel Aznarez, Jamaica Plain, MA (US); Jacob Albert Kach, Jamaica Plain, MA (US); Ana Corrionero Saiz, Cambridge, MA (US)

(21) Appl. No.: 18/545,753

(22) Filed: Dec. 19, 2023

Related U.S. Application Data
(63) Continuation of application No. PCT/US22/34344, filed on Jun. 21, 2022.
(60) Provisional application No. 63/212,981, filed on Jun. 21, 2021.

(51) Int. Cl. C12N 15/113

(52) U.S. Cl. CPC C12N 15/113; C12N 2310/11; C12N 2310/314; C12N 2310/321; C12N 2310/322; C12N 2310/3231; C12N 2310/3233

(57) ABSTRACT

Alternative splicing events in genes can lead to non-productive mRNA transcripts which in turn can lead to aberrant or reduced protein expression, and therapeutic agents which can target the alternative splicing events in genes can modulate the expression level of functional proteins in patients and/or inhibit aberrant protein expression. Such therapeutic agents can be used to treat a condition or disease caused by deficiency of the protein.

### CROSS REFERENCE

This application is a continuation of International Patent Application No. PCT/US2022/034344, filed Jun. 21, 2022, which claims benefit of U.S. Provisional Patent Application No. 63/212,981, filed Jun. 21, 2021, which is incorporated herein by reference in its entirety.

### SEQUENCE LISTING

The instant application contains a Sequence Listing which has been submitted electronically in XML format and is hereby incorporated by reference in its entirety. Said XML copy, created on Dec. 18, 2023, is named 47991_735_601_SL.xml and is 260,301 bytes in size.

### BACKGROUND

Alternative splicing events in genes can lead to non-productive mRNA transcripts which in turn can lead to aberrant or reduced protein expression, and therapeutic agents which can target the alternative splicing events in genes can modulate the expression level of functional proteins in patients and/or inhibit aberrant protein expression. Such therapeutic agents can be used to treat a condition or disease caused by the protein deficiency.

PHIP-related disorder, also known as Chung Jansen Syndrome (CHUJANS) is characterized by global developmental delay apparent from infancy, impaired intellectual development or learning difficulties, behavioral abnormalities, dysmorphic features, and obesity. The severity of the phenotype and additional features are variable. CHUJANS is a rare condition caused by a change in the pleckstrin homology domain-interacting protein (PHIP) gene. This gene plays an important role in several processes linked to brain and nervous system development. The PHIP gene is also involved in regulating insulin in nervous tissues. To date, PHIP-related disorder is not known to cause diabetes, however people with the disorder are at an increased risk for being overweight which is a risk factor for diabetes. The most common signs and symptoms, include mild to severe learning problems, behavior problems, and a tendency toward being overweight.

PHIP-related disorder is an autosomal dominant condition. Many people with PHIP-related disorder have mild to severe intellectual disability. People with the disorder who did not have intellectual disability, often have speech problems, global developmental delays in early childhood, and learning problems. Currently, there is no approved disease-modifying treatment for PHIP-related disorder patients and there is a need for such treatments.

### SUMMARY

Provided herein is a method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating a level of processed mRNA that is processed from the pre-mRNA, and modulating the expression of the target protein in the cell, and wherein the target gene is a PHIP gene.

In some aspects, the agent: (a) binds to a targeted portion of the pre-mRNA; (b) modulates binding of a factor involved in splicing of the NMD exon; or a combination of (a) and (b).

In some aspects, the agent interferes with binding of the factor involved in splicing of the NMD exon to a region of the targeted portion.

In some aspects, the targeted portion of the pre-mRNA is proximal to the NMD exon.

In some aspects, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon.

In some aspects, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon.

In some aspects, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon.

In some aspects, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon.

In some aspects, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 79004373.

In some aspects, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 79004373.

In some aspects, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 79004436.

In some aspects, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 79004436.

In some aspects, the targeted portion of the pre-mRNA is located in an intronic region between two canonical exonic regions of the pre-mRNA, and wherein the intronic region contains the NMD exon.

In some aspects, the targeted portion of the pre-mRNA at least partially overlaps with the NMD exon.

In some aspects, the targeted portion of the pre-mRNA at least partially overlaps with an intron upstream or downstream of the NMD exon.

In some aspects, the targeted portion of the pre-mRNA comprises 5′ NMD exon-intron junction or 3′ NMD exon-intron junction.

In some aspects, the targeted portion of the pre-mRNA is within the NMD exon.

In some aspects, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some aspects, the NMD exon is (a) within an intronic sequence with at least 80%, at least 90%, or 100% sequence identity to SEQ ID NO: 2 and/or (b) comprises a sequence with at least 80%, at least 90%, or 100% sequence identity to SEQ ID NO: 3.

In some aspects, the NMD exon comprises a sequence of SEQ ID NO: 3.

In some aspects, the targeted portion of the pre-mRNA is within the non-sense mediated RNA decay-inducing exon GRCh38/hg38: chr6 79004373 to 79004436.

In some aspects, the targeted portion of the pre-mRNA is upstream or downstream of the non-sense mediated RNA decay-inducing exon GRCh38/hg38: chr6 79004373 to 79004436.

In some aspects, the targeted portion of the pre-mRNA comprises an exon-intron junction of the non-sense mediated RNA decay-inducing exon GRCh38/hg38: chr6 79004373 to 79004436.

In some aspects, the target protein expressed from the processed mRNA is a full-length PHIP protein or a wild-type PHIP protein.

In some aspects, the target protein expressed from the processed mRNA is a functional PHIP protein.

In some aspects, the target protein expressed from the processed mRNA is at least partially functional as compared to a wild-type PHIP protein.

In some aspects, the target protein expressed from the processed mRNA is at least partially functional as compared to a full-length wild-type PHIP protein.

In some aspects, the target protein expressed from the processed mRNA is a PHIP protein that lacks an amino acid sequence encoded by the non-sense mediated RNA decay-inducing exon GRCh38/hg38: chr6 79004373 to 79004436.

In some aspects, the method promotes exclusion of the NMD exon from the pre-mRNA.

In some aspects, the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

In some aspects, the method results in an increase in the level of the processed mRNA in the cell.

In some aspects, the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

In some aspects, the method results in an increase in the expression of the target protein in the cell.

In some aspects, a level of the target protein expressed from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

In some aspects, the agent comprises an antisense oligomer with at least 80%, at least 90%, or 100% sequence identity to a sequence selected from the group consisting of SEQ ID NOs: 4-182.

In some aspects, the agent further comprises a gene editing molecule.

In some aspects, the gene editing molecule comprises CRISPR-Cas9.

In some aspects, the agent is an antisense oligomer (ASO) and wherein the antisense oligomer comprises a backbone modification comprising a phosphorothioate linkage or a phosphorodiamidate linkage.

In some aspects, the agent is an antisense oligomer (ASO) and wherein the antisense oligomer comprises a phosphorodiamidate morpholino, a locked nucleic acid, a peptide nucleic acid, a 2′-O-methyl moiety, a 2′-Fluoro moiety, a 2′-NMA moiety, or a 2′-O-methoxyethyl moiety.

In some aspects, the agent is an antisense oligomer (ASO) and wherein the antisense oligomer comprises at least one modified sugar moiety.

In some aspects, each sugar moiety is a modified sugar moiety.

In some aspects, the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some aspects, the method comprises contacting a vector encoding the agent to the cell, wherein the vector is a viral vector.

In some aspects, the viral vector comprises an adenoviral vector, adeno-associated viral (AAV) vector, lentiviral vector, Herpes Simplex Virus (HSV) viral vector, or retroviral vector.

In some aspects, the viral vector comprises an adeno-associated viral (AAV) vector.

In some aspects, the agent comprises a modified snRNA.

In some aspects, the modified human snRNA is a modified U1 snRNA.

In some aspects, the modified human snRNA is a modified U7 snRNA.

In some aspects, a portion of a single-stranded nucleotide sequence of the modified human snRNA comprises a sequence that binds to the targeted portion of the pre-mRNA.

In some aspects, the method comprises contacting a vector encoding the modified snRNA to the cell.

In some aspects, the method further comprises assessing mRNA level or expression level of the target protein.

In some aspects, the agent is a therapeutic agent.

Also provided herein is a pharmaceutical composition comprising a therapeutic agent described herein or a vector encoding a therapeutic agent described herein, and a pharmaceutically acceptable excipient.

Also provided herein is a pharmaceutical composition, comprising a therapeutic agent or a vector encoding a therapeutic agent, and a pharmaceutically acceptable excipient, wherein the therapeutic agent comprises an antisense oligomer with at least 80% sequence identity to a sequence selected from the group consisting of SEQ ID NOs: 4-182.

In some aspects, the therapeutic agent comprises an antisense oligomer with at least 80%, at least 90%, or 100% sequence identity to a sequence selected from the group consisting of SEQ ID NOs: 55-182.

In some aspects, the pharmaceutical composition is formulated for intracerebroventricular injection, intraperitoneal injection, intramuscular injection, intrathecal injection, subcutaneous injection, oral administration, synovial injection, intravitreal administration, subretinal injection, topical application, implantation, or intravenous injection.

In some aspects, the pharmaceutical composition is formulated for intrathecal injection or intracerebrospinal injection.

In some aspects, the pharmaceutical composition further comprises a second therapeutic agent.

In some aspects, the second therapeutic agent comprises a small molecule.

In some aspects, the second therapeutic agent comprises an antisense oligomer.

In some aspects, the second therapeutic agent corrects intron retention.

Also provided herein is a composition comprising an antisense oligomer with at least 80% sequence identity to a sequence selected from the group consisting of SEQ ID NOs: 4-182, wherein the antisense oligomer comprises a backbone modification, a sugar moiety modification, or a combination thereof.

Also provided herein is a composition comprising a viral vector encoding a polynucleotide comprising an antisense oligomer, wherein the antisense oligomer consists of a sequence selected from the group consisting of SEQ ID NOs: 4-182.

In some aspects, the polynucleotide further comprises a modified snRNA.

In some aspects, the modified human snRNA is a modified U1 snRNA.

In some aspects, the modified human snRNA is a modified U7 snRNA.

In some aspects, the antisense oligomer has at least 80%, at least 90%, or 100% sequence identity to a sequence selected from the group consisting of SEQ ID NOs: 55-182.

Also provided herein is a method of treating or reducing the likelihood of developing a disease or condition in a subject in need thereof by modulating expression of a target protein in a cell of the subject, comprising contacting to cells of the subject a pharmaceutical composition described herein.

In some aspects, the disease or condition is associated with a loss-of-function mutation in a PHIP gene.

In some aspects, the disease or condition is associated with haploinsufficiency of the PHIP gene, and wherein the subject has a first allele encoding a functional PHIP protein, and a second allele from which the PHIP protein is not produced or produced at a reduced level, or a second allele encoding a nonfunctional PHIP protein or a partially functional PHIP protein.

In some aspects, the disease or condition comprises an intellectual disability disease or condition.

In some aspects, the disease or condition comprises Chung-Jansen syndrome (CHUJANS), an autosomal dominant disorder, intellectual disability, speech delay, anxiety, autism spectrum disorders (ASD), Attention deficit hyperactivity disorder (ADHD), aggression, facial dysmorphism, café au lait spots, overweight syndrome caused by PHIP haploinsufficiency, developmental delay, obesity or dysmorphism.

In some aspects, the disease or condition comprises Chung-Jansen syndrome.

In some aspects, the disease or condition comprises an intellectual disability.

In some aspects, the disease or condition is associated with an autosomal recessive mutation of PHIP gene, wherein the subject has a first allele from which: (i) PHIP protein is not produced or produced at a reduced level compared to a wild-type allele; or (ii) the PHIP protein produced is nonfunctional or partially functional compared to a wild-type allele, and a second allele from which: (iii) the PHIP protein is produced at a reduced level compared to a wild-type allele and the PHIP protein produced is at least partially functional compared to a wild-type allele; or (iv) the PHIP protein produced is partially functional compared to a wild-type allele.

In some aspects, the subject is a human.

In some aspects, the subject is a non-human animal.

In some aspects, the subject is a fetus, an embryo, or a child.

In some aspects, the cells are ex vivo.

In some aspects, the pharmaceutical composition is administered by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

In some aspects, the pharmaceutical composition is administered by intrathecal injection or intracerebrospinal injection.

In some aspects, the method treats the disease or condition.

Also provided herein is a composition comprising an agent or a vector encoding the agent that modulates splicing of a non-sense mediated RNA decay-inducing exon (NMD exon) from a pre-mRNA that is transcribed from a target gene and that comprises the NMD exon, thereby modulating the level of a processed mRNA that is processed from the pre-mRNA, and modulating expression of a target protein in a cell having the pre-mRNA, and wherein the target gene is a PHIP gene.

Also provided herein is a composition comprising an agent or a vector encoding the agent that modulates splicing of a non-sense mediated RNA decay-inducing exon (NMD exon) from a pre-mRNA that is transcribed from a target gene and that comprises the NMD exon, thereby treating a disease or condition in a subject in need thereof by modulating the level of a processed mRNA that is processed from the pre-mRNA, and modulating expression of a target protein in a cell of the subject, and wherein the target gene is a PHIP gene.

Also provided herein is a pharmaceutical composition comprising a composition described herein; and a pharmaceutically acceptable excipient and/or a delivery vehicle.

In some aspects, the target protein is pleckstrin homology domain-interacting protein (PHIP).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B depict a schematic representation of a target mRNA that contains a non-sense mediated mRNA decay-inducing exon (NMD exon mRNA) and therapeutic agent-mediated exclusion of the nonsense-mediated mRNA decay-inducing exon to increase expression of the full-length target protein or functional RNA. FIG. 1A shows a cell divided into nuclear and cytoplasmic compartments. In the nucleus, a pre-mRNA transcript of a target gene undergoes splicing to generate mRNA, and this mRNA is exported to the cytoplasm and translated into target protein. For this target gene, some fraction of the mRNA contains a nonsense-mediated mRNA decay-inducing exon (NMD exon containing mature mRNA) that is degraded in the cytoplasm, thus leading to no target protein production. FIG. 1B shows an example of the same cell divided into nuclear and cytoplasmic compartments. Treatment with a therapeutic agent, such as an antisense oligomer (ASO), promotes the exclusion of the nonsense-mediated mRNA decay-inducing exon and results in an increase in productive mRNA, which is in turn translated into higher levels of target protein.

FIG. 2 shows an example schematic of a novel NMD exon inclusion event (Exon X) identified in the PHIP gene which leads to a non-productive mRNA transcript degraded by non-sense mediated decay (NMD).

FIG. 3 depicts the UCSC Genome Browser snapshot of a region in the PHIP gene (exons are rectangles and introns are lines with arrowheads) that contains an NMD-inducing exon inclusion event (GRCh38/hg38: chr6 79004373 to 79004436) depicted by the shaded area and black bar on the top. RNA sequencing traces from human neural progenitor cells (ReN) and human astrocytes treated with cycloheximide (CHX) or DMSO control.

FIG. 4A depicts confirmation of NMD-inducing exon via puromycin or cycloheximide treatment in HEK293, SK-N-AS and ReN VM cell lines. RT-PCR analysis using total RNA from water-treated (H), DMSO-treated (D), puromycin-treated (P), or cycloheximide-treated (C) cells confirmed the presence of a band corresponding to the NMD-inducing exon 15x (GRCh38/hg38: chr6 79004373 to 79004436) of PHIP gene.

FIG. 4B depicts confirmation of NMD-inducing exon via puromycin or cycloheximide treatment in a U87 cell line and astrocytes. RT-PCR analysis using total RNA from water-treated (H), DMSO-treated (D), puromycin-treated (P), or cycloheximide-treated (C) cells confirmed the presence of a band corresponding to the NMD-inducing exon 15x (GRCh38/hg38: chr6 79004373 to 79004436) of PHIP gene.

FIG. 4C depicts confirmation of NMD-inducing exon via puromycin or cycloheximide treatment in mouse embryonic fibroblast cells. RT-PCR analysis using total RNA from water-treated (H), DMSO-treated (D), puromycin-treated (P), or cycloheximide-treated (C) cells confirmed the presence of a band corresponding to the NMD-inducing exon 15x (GRCh38/hg38: chr6 79004373 to 79004436) of PHIP gene.

FIG. 5 depicts the conservation of the NMD exon event (15X) in non-human primates.

FIGS. 6A-C depict an exemplary ASO walk around PHIP exon 15x (GRCh38/hg38: chr6 79004373 to 79004436) region. A graphic representation of an ASO walk performed for around PHIP exon 15x (GRCh38/hg38: chr6 79004373 to 79004436) region targeting sequences upstream of the 3′ splice site, across the 3′splice site (FIG. 6A), exon 15x (FIG. 6B), across the 5′ splice site, and downstream of the 5′ splice site (FIG. 6C) is shown. ASOs were designed to cover these regions by shifting 5 nucleotides at a time or 3 nucleotides between the beginning or end of the exon and the adjacent ASOs across the splice site regions.

FIGS. 7A-B depict a PHIP exon15x (GRCh38/hg38: chr6 79004373 79004436) region ASO walk evaluated by Taqman RT-qPCR (for productive mRNA fold change) and RT-PCR (for non-productive mRNA fold change) in ReN VM cells nucleofected with 3 μM of the indicated ASOs in the presence of cycloheximide. Gel images and graphs of fold-change of the PHIP productive mRNA product relative to a control gene are plotted.

FIG. 8 depicts a PHIP exon15x (GRCh38/hg38: chr6 79004373 79004436) region ASO walk evaluated by Taqman RT-qPCR in ReN cells nucleofected with 3 μM of the indicated ASOs in the absence of cycloheximide. Gel images and graphs of the percent PHIP non-productive mRNA product, fold-change of the canonical PHIP mRNA and fold-change of the total PHIP mRNA relative to a control gene are plotted.

FIGS. 9A-B depict a PHIP exon15x (GRCh38/hg38: chr6 79004373 79004436) region ASO walk evaluated by Taqman RT-qPCR in HEK293 cells transfected with 120 nM of the indicated ASOs in the presence of cycloheximide. Gel images and graphs of fold-change of the PHIP productive mRNA product relative to a control gene are plotted.

FIGS. 10A-10D depict a PHIP exon15x (GRCh38/hg38: chr6 79004373 79004436) region ASO walk evaluated by Taqman RT-qPCR in HEK293 cells transfected with 120 nM of the indicated ASOs in the absence of cycloheximide. Graphs (FIGS. 10A-10C) and gel images (FIG. 10D) of the percent PHIP non-productive mRNA product, fold-change of the canonical PHIP mRNA and fold-change of the total PHIP mRNA relative to a control gene are plotted.

FIG. 11 depicts an exemplary ASO micro-walk around PHIP exon 15x (GRCh38/hg38: chr6 79004373 to 79004436) region.

FIG. 12 depicts a PHIP exon15x (GRCh38/hg38: chr6 79004373 to 79004436) region ASO microwalk evaluated by Taqman RT-qPCR in ReN cells nucleofected with 3 μM of the indicated ASOs in the presence of cycloheximide. Gel images and graphs of fold-change of the PHIP productive mRNA product relative to a control gene are plotted.

FIGS. 13A-B depict a PHIP exon15x (GRCh38/hg38: chr6 79004373 to 79004436) region ASO microwalk evaluated by Taqman RT-qPCR in HEK293 cells transfected with 120 nM of the indicated ASOs in the presence of cycloheximide. Gel images and graphs of fold-change of the PHIP productive mRNA product relative to a control gene are plotted.

FIG. 14A shows gel images that demonstrate modulation of splicing of exon 15x from PHIP pre-mRNAs by exemplary ASOs at different concentrations. FIG. 14B is a bar graph summarizing the quantification of the fold change in the amount of canonical PHIP mRNA and the change in the amount of non-productive PHIP mRNA in response to treatment with exemplary ASOs at different concentrations.

FIGS. 15A and 15B demonstrate that exemplary ASOs (IVS14X:-3, IVS14X-Ex15XX:7, Ex15X:19, and Ex15X:23) caused an increase in PHIP protein level. FIG. 15A shows Western blotting image of PHIP protein under different treatment conditions, and FIG. 15B is a bar graph summarizing the quantification of the change in PHIP protein level in response to the different treatments.

FIGS. 16A-16D demonstrate that treatment with exemplary ASOs via nucleofection led to decrease in the amount of non-productive PHIP transcripts with exon 15x (FIG. 16B) and increase in the amount of PHIP transcripts without exon 15x, as shown by Taqman qPCR using probes spanning exon 15 and exon 16 (canonical; FIG. 16C) and exon 35 and exon 36 (GE; FIG. 16D). FIG. 16A shows gel images of the RT-PCR amplicons under different treatment conditions.

FIGS. 17A-17D demonstrate that treatment with exemplary ASOs via free uptake led to decrease in the amount of non-productive PHIP transcripts with exon 15x (FIG. 17B) and increase in the amount of PHIP transcripts without exon 15x, as shown by Taqman qPCR using probes spanning exon 15 and exon 16 (canonical; FIG. 17C) and exon 35 and exon 36 (GE; FIG. 17D). FIG. 17A shows gel images of the RT-PCR amplicons under different treatment conditions.

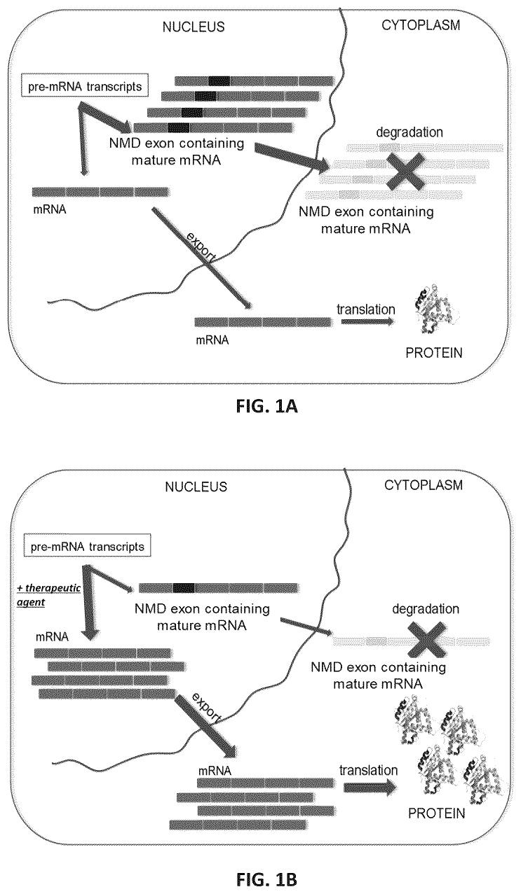

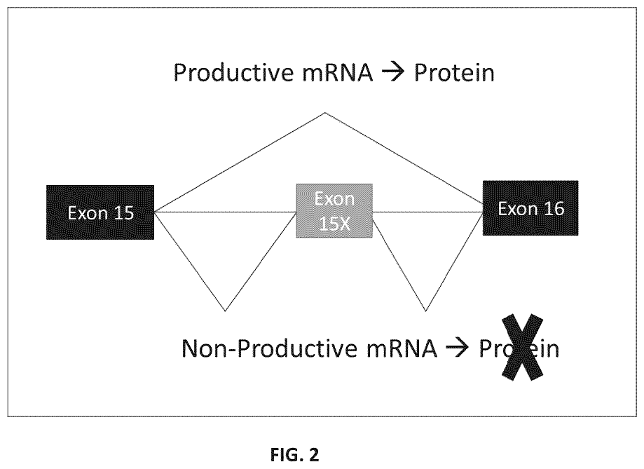

### DETAILED DESCRIPTION

Alternative splicing events in the (Pleckstrin homology interacting protein) PHIP gene can lead to non-productive mRNA transcripts which in turn can lead to aberrant or reduced protein expression, and therapeutic agents which can target the alternative splicing events in the PHIP gene can modulate the expression level of functional proteins in Chung Jansen Syndrome (CHUJANS) patients and/or inhibit aberrant protein expression. Such therapeutic agents can be used to treat a condition caused by PHIP protein deficiency.

One of the alternative splicing events that can lead to non-productive mRNA transcripts is the inclusion of an extra exon in the mRNA transcript that can induce non-sense mediated mRNA decay. The present disclosure provides compositions and methods for modulating alternative splicing of PHIP to increase the production of protein-coding mature mRNA, and thus, translated functional PHIP protein. These compositions and methods include antisense oligomers (ASOs) that can cause exon skipping, e.g., pseudoexon skipping, and promote constitutive splicing of PHIP pre-mRNA. In various embodiments, functional PHIP protein can be increased using the methods of the disclosure to treat a condition caused by PHIP protein deficiency.

mRNA Splicing

Intervening sequences in RNA sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

Alternative splicing is a regulated process during gene expression that can result in multiple isoforms of mature mRNA transcripts that are processed from a single primary mRNA transcript that is transcribed from a single gene, and the resultant multiple proteins that are translated from at least some of the multiple mature mRNA isoforms. In this process, particular exons of a gene may be included within or excluded from the final, processed mRNA produced from that gene. Consequently, the proteins translated from alternatively spliced mRNAs will contain differences in their amino acid sequence and, in some cases, in their biological functions.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

A NMD-inducing exon (“NIE” or “NMD exon”) is an exon or a pseudo-exon that is a region within an intron and can activate the NMD pathway if included in a mature RNA transcript. In constitutive splicing events, the intron containing an NMD exon is usually spliced out, but the intron or a portion thereof (e.g., NMD exon) may be retained during alternative or aberrant splicing events. Mature mRNA transcripts containing such an NMD exon may be non-productive due to frame shifts which induce the NMD pathway. Inclusion of a NMD exon in mature RNA transcripts may downregulate gene expression. mRNA transcripts containing an NMD exon may be referred to as “NIE-containing mRNA” or “NMD exon mRNA” in the current disclosure.

Cryptic (or pseudo-splice sites) have the same splicing recognition sequences as genuine splice sites but are not used in splicing reactions. They outnumber genuine splice sites in the human genome by an order of magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413.

The cryptic splice sites or splicing regulatory sequences may compete for RNA-binding proteins, such as U2AF, with a splice site of the NMD exon. In some embodiments, an agent may bind to a cryptic splice site or splicing regulatory sequence to prevent binding of RNA-binding proteins and thereby favor binding of RNA-binding proteins to the NMD exon splice sites.

In some embodiments, the cryptic splice site may not comprise the 5′ or 3′ splice site of the NMD exon. In some embodiments, the cryptic splice site may be at least 10 nucleotides, at least 20 nucleotides, at least 50 nucleotides, at least 100 nucleotides or at least 200 nucleotides upstream of the NMD exon 5′ splice site. In some embodiments, the cryptic splice site may be at least 10 nucleotides, at least 20 nucleotides, at least 50 nucleotides, at least 100 nucleotides, at least 200 nucleotides downstream of the NMD exon 3′ splice site.

### Target Transcripts

In some embodiments, the methods and compositions of the present disclosure exploit the presence of NMD exon in the pre-mRNA transcribed from the PHIP gene. Splicing of the identified PHIP NMD exon pre-mRNA species to produce functional mature PHIP mRNA may be induced using an agent such as an ASO that stimulates exon skipping of an NMD exon.

Induction of exon skipping may result in inhibition of an NMD pathway. The resulting mature PHIP mRNA can be translated normally without activating NMD pathway, thereby increasing the amount of PHIP protein in the patient's cells and alleviating symptoms of a condition or disease associated with PHIP deficiency, such as Chung-Jansen syndrome (CHUJANS), an autosomal dominant disorder, intellectual disability, speech delay, anxiety, autism spectrum disorders (ASD), Attention deficit hyperactivity disorder (ADHD), aggression, facial dysmorphism, café au lait spots, overweight syndrome caused by PHIP haploinsufficiency, developmental delay, obesity or dysmorphism.

In some embodiments, the diseases or conditions that can be treated or ameliorated using the method or composition disclosed herein are not directly associated with the target protein (gene) that the therapeutic agent targets. In some embodiments, a therapeutic agent provided herein can target a protein (gene) that is not directly associated with a disease or condition, but the modulation of expression of the target protein (gene) can treat or ameliorate the disease or condition.

In various embodiments, the present disclosure provides a therapeutic agent which can target PHIP pre-mRNA transcripts to modulate splicing or protein expression level. The therapeutic agent can be a small molecule, polynucleotide, or polypeptide. In some embodiments, the therapeutic agent is an ASO. Various regions or sequences on the PHIP pre-mRNA can be targeted by a therapeutic agent, such as an ASO. In some embodiments, the ASO targets a PHIP pre-mRNA transcript containing an NMD exon. In some embodiments, the ASO targets a sequence within an NMD exon of a PHIP pre-mRNA transcript. In some embodiments, the ASO targets a sequence upstream (or 5′) from the 5′ end of an NMD exon (3′ss) of a PHIP pre-mRNA transcript. In some embodiments, the ASO targets a sequence downstream (or 3′) from the 3′ end of an NMD exon (5′ss) of a PHIP pre-mRNA transcript. In some embodiments, the ASO targets a sequence that is within an intron flanking on the 5′ end of the NMD exon of a PHIP pre-mRNA transcript. In some embodiments, the ASO targets a sequence that is within an intron flanking the 3′ end of the NMD exon of a PHIP pre-mRNA transcript. In some embodiments, the ASO targets a sequence comprising an NMD exon-intron boundary of a PHIP pre-mRNA transcript. An NMD exon-intron boundary can refer to the junction of an intron sequence and an NMD exon region. The intron sequence can flank the 5′ end of the NMD exon, or the 3′ end of the NMD exon. In some embodiments, the ASO targets a sequence within an exon of a PHIP pre-mRNA transcript. In some embodiments, the ASO targets a sequence within an intron of a PHIP pre-mRNA transcript. In some embodiments, the ASO targets a sequence comprising both a portion of an intron and a portion of an exon of a PHIP pre-mRNA transcript.

In some embodiments, the ASO targets a sequence about 4 to about 300 nucleotides upstream (or 5′) from the 5′ end of the NMD exon. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the NMD exon region. In some embodiments, the ASO may target a sequence more than 300 nucleotides upstream from the 5′ end of the NMD exon. In some embodiments, the ASO targets a sequence about 4 to about 300 nucleotides downstream (or 3′) from the 3′ end of the NMD exon. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the NMD exon. In some embodiments, the ASO targets a sequence more than 300 nucleotides downstream from the 3′ end of the NMD exon.

In some embodiments, the PHIP NMD exon-containing pre-mRNA transcript is encoded by a genetic sequence with at least about 80%, 85%, 90%, 95%, 96%, 97%, 98%, 99% or 100% sequence identity to SEQ ID NO. 1. In some embodiments, the PHIP NMD exon pre-mRNA transcript comprises a sequence with at least about 80%, 85%, 90%, 95%, 96%, 97%, 98%, 99% or 100% sequence identity to SEQ ID NO: 3.

In some embodiments, the PHIP NMD exon-containing pre-mRNA transcript (or NMD exon mRNA) comprises a sequence with at least about 80%, 85%, 90%, 95%, 97%, or 100% sequence identity to SEQ ID NO: 3. In some embodiments, PHIP NMD exon-containing pre-mRNA transcript (or NMD exon mRNA) is encoded by a sequence with at least about 80%, 85%, 90%, 95%, 97%, or 100% sequence identity to SEQ ID NO: 3. In some embodiments, the targeted portion of the NMD exon mRNA comprises a sequence with at least 80%, 85%, 90%, 95%, 97%, or 100% sequence identity to a region comprising at least 8 contiguous nucleic acids of SEQ ID NO: 3.

In some embodiments, the ASO targets exon 15x of a PHIP NMD exon-containing pre-mRNA comprising NIE exon 15 (exon 15x) of a PHIP NMD exon-containing pre-mRNA comprising NIE exon 15. In some embodiments, the ASO targets exon (GRCh38/hg38: chr6 79004373 to 79004436) of PHIP.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 15x of PHIP, exon 15x of PHIP. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr6 79004373 of PHIP.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 15x of PHIP. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr6 79004373 of PHIP.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 15x of PHIP. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr6 79004436 of PHIP.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 15x of PHIP. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr6 79004436 of PHIP.

In some embodiments, the ASO has a sequence complementary to the targeted portion of the NMD exon mRNA according to SEQ ID NO: 3.

In some embodiments, the ASO targets a sequence upstream from the 5′ end of an NMD exon. For example, ASOs targeting a sequence upstream from the 5′ end of an NMD exon (e.g., exon (GRCh38/hg38: chr6 79004373 to 79004436) of PHIP) can comprise a sequence with at least 80%, 85%, 90%, 95%, 97%, or 100% sequence identity to SEQ ID NO: 3.

In some embodiments, the ASOs target a sequence containing an exon-intron boundary (or junction). For example, ASOs targeting a sequence containing an exon-intron boundary can comprise a sequence that is at least about 80%, 85%, 90%, 95%, 97%, or 100% complimentary to at least 8 contiguous nucleic acids of SEQ ID NO: 3. In some embodiments, the ASOs target a sequence downstream from the 3′ end of an NMD exon. For example, ASOs targeting a sequence downstream from the 3′ end of an NMD exon (e.g., exon 15x of PHIP) can comprise a sequence with at least 80%, 85%, 90%, 95%, 97%, or 100% sequence identity to SEQ ID NO: 3, or at least 8 contiguous nucleic acids of SEQ ID NO: 3. For example, ASOs targeting a sequence downstream from the 3′ end of an NMD exon (e.g., exon (GRCh38/hg38: chr6 79004373 to 79004436) of PHIP) can comprise a sequence with at least 80%, 85%, 90%, 95%, 97%, or 100% sequence identity to SEQ ID NO: 3, or at least 8 contiguous nucleic acids of SEQ ID NO: 3. In some embodiments, ASOs target a sequence within an NMD exon.

In some embodiments, the ASO targets exon 15x of a PHIP NMD exon-containing pre-mRNA comprising exon 15, NIE exon 15x of a PHIP NMD exon-containing pre-mRNA comprising NIE exon 16. In some embodiments, the ASO targets a sequence downstream (or 3′) from the 5′ end of exon 15x of a PHIP pre-mRNA. In some embodiments, the ASO targets a sequence upstream (or 5′) from the 3′ end of exon 15x of a PHIP pre-mRNA.

In some embodiments, the targeted portion of the PHIP NMD exon-containing pre-mRNA is in intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. In some embodiments, hybridization of an ASO to the targeted portion of the NMD exon pre-mRNA results in exon skipping of at least one of NMD exon within intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39, and subsequently increases PHIP protein production. In some embodiments, the targeted portion of the PHIP NMD exon-containing pre-mRNA is in intron 15 of PHIP. In some embodiments, the targeted portion of the PHIP NMD exon-containing pre-mRNA is intron (GRCh38/hg38: chr6 79003858 to 79015082) of PHIP.

In some embodiments, the methods and compositions of the present disclosure are used to increase the expression of PHIP by inducing exon skipping of a pseudo-exon of a PHIP NMD exon-containing pre-mRNA. In some embodiments, the pseudo-exon is a sequence within any of introns 1-39. In some embodiments, the pseudo-exon is a sequence within any of introns 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. In some embodiments, the pseudo-exon can be a PHIP intron or a portion thereof. In some embodiments, the pseudo-exon is within intron 15 of PHIP. In some embodiments, the pseudo-exon is within intron (GRCh38/hg38: chr6 79003858 79015082) of PHIP.

In some embodiments, the PHIP pre-mRNA transcript is encoded by a genetic sequence with at least about 80%, 85%, 90%, 95%, 96%, 97%, 98%, 99% or 100% sequence identity to SEQ ID NO. 1. In some embodiments, the PHIP pre-mRNA transcript comprises a sequence with at least about 80%, 85%, 90%, 95%, 96%, 97%, 98%, 99% or 100% sequence identity to SEQ ID NO: 3.

In some embodiments, the PHIP pre-mRNA transcript (or NMD exon mRNA) comprises a sequence with at least about 80%, 85%, 90%, 95%, 97%, or 100% sequence identity to SEQ ID NO: 3. In some embodiments, PHIP pre-mRNA transcript (or NMD exon mRNA) is encoded by a sequence with at least about 80%, 85%, 90%, 95%, 97%, or 100% sequence identity to SEQ ID NO: 3. In some embodiments, the targeted portion of the PHIP pre-mRNA comprises a sequence with at least 80%, 85%, 90%, 95%, 97%, or 100% sequence identity to a region comprising at least 8 contiguous nucleic acids of SEQ ID NO: 3.

In some embodiments, the ASO targets exon 15 of a PHIP pre-mRNA. In some embodiments, the ASO targets exon (GRCh38/hg38: chr6 79015081 79015216) of PHIP pre-mRNA. In some embodiments, the ASO targets exon 16 of a PHIP pre-mRNA, the ASO targets exon (GRCh38/hg38: chr6 79003729 79003858) of PHIP pre-mRNA.

In some embodiments, the ASO has a sequence complementary to the targeted portion of the NMD exon mRNA according to SEQ ID NO: 3.

### Protein Expression

In some embodiments, the methods described herein are used to increase the production of a functional PHIP protein or RNA. As used herein, the term “functional” refers to the amount of activity or function of a PHIP protein or RNA that is necessary to eliminate any one or more symptoms of a treated condition or disease, e.g., Chung-Jansen syndrome. In some embodiments, the methods are used to increase the production of a partially functional PHIP protein or RNA. As used herein, the term “partially functional” refers to any amount of activity or function of the PHIP protein or RNA that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the PHIP, protein by cells of a subject having a PHIP pre-mRNA, wherein the subject has a disease or condition, e.g., Chung-Jansen syndrome, caused by a deficient amount of activity of PHIP protein, and wherein the deficient amount of the PHIP protein is caused by haploinsufficiency of the PHIP protein. In such an embodiment, the subject has a first allele encoding a functional PHIP protein, and a second allele from which the PHIP protein is not produced. In another such embodiment, the subject has a first allele encoding a functional PHIP protein, and a second allele encoding a nonfunctional PHIP protein. In another such embodiment, the subject has a first allele encoding a functional PHIP protein, and a second allele encoding a partially functional PHIP protein. In any of these embodiments, the antisense oligomer binds to a targeted portion of the PHIP pre-mRNA transcribed from the second allele, thereby inducing exon skipping of the pseudo-exon from the pre-mRNA, and causing an increase in the level of mature mRNA encoding functional PHIP protein, and an increase in the expression of the PHIP protein in the cells of the subject.

In some embodiments, the method is a method of increasing the expression of the PHIP protein by cells of a subject having a PHIP pre-mRNA, wherein the subject has a disease or condition caused by a deficient amount of activity of PHIP protein, and wherein the deficient amount of the PHIP protein is caused by autosomal recessive inheritance.

In some embodiments, the method is a method of increasing the expression of the PHIP protein by cells of a subject having a PHIP pre-mRNA, wherein the subject has a disease or condition, e.g., Chung-Jansen syndrome, caused by a deficient amount of activity of PHIP, protein, and wherein the deficient amount of the PHIP protein is caused by autosomal dominant inheritance.

In related embodiments, the method is a method of using an ASO to increase the expression of a protein or functional RNA. In some embodiments, an ASO may be used to increase the expression of PHIP protein in cells of a subject having a PHIP pre-mRNA, wherein the subject has a deficiency, e.g., Chung-Jansen syndrome; in the amount or function of a PHIP protein.

In some embodiments, the pre-mRNA transcript that encodes the protein that is causative of the disease or condition is targeted by the agent, e.g., the oligonucleotides, described herein. In some cases, it is the NMD exon-containing pre-mRNA transcript targeted by the agent, e.g., the oligonucleotides, described herein. In some embodiments, a NMD exon-containing pre-mRNA transcript that encodes a protein that is not causative of the disease is targeted by the ASOs. For example, a disease that is the result of a mutation or deficiency of a first protein in a particular pathway may be ameliorated by targeting a pre-mRNA that encodes a second protein, thereby increasing production of the second protein. In some embodiments, the function of the second protein is able to compensate for the mutation or deficiency of the first protein (which is causative of the disease or condition).

In some embodiments, the subject has: (a) a first mutant allele from which (i) the PHIP protein is produced at a reduced level compared to production from a wild-type allele, (ii) the PHIP protein is produced in a form having reduced function compared to an equivalent wild-type protein, or (iii) the PHIP protein or functional RNA is not produced; and (b) a second mutant allele from which (i) the PHIP protein is produced at a reduced level compared to production from a wild-type allele, (ii) the PHIP protein is produced in a form having reduced function compared to an equivalent wild-type protein, or (iii) the PHIP protein is not produced, and wherein the NMD exon-containing pre-mRNA is transcribed from the first allele and/or the second allele. In these embodiments, the ASO binds to a targeted portion of the NMD exon-containing pre-mRNA transcribed from the first allele or the second allele, thereby inducing exon skipping of the pseudo-exon from the NMD exon-containing pre-mRNA, and causing an increase in the level of mRNA encoding PHIP protein and an increase in the expression of the target protein or functional RNA in the cells of the subject. In these embodiments, the target protein or functional RNA having an increase in expression level resulting from the exon skipping of the pseudo-exon from the NMD exon-containing pre-mRNA may be either in a form having reduced function compared to the equivalent wild-type protein (partially-functional), or having full function compared to the equivalent wild-type protein (fully-functional).

In some embodiments, the level of mRNA encoding PHIP protein is increased 1.1 to 10-fold, when compared to the amount of mRNA encoding PHIP protein that is produced in a control cell, e.g., one that is not treated with the antisense oligomer or one that is treated with an antisense oligomer that does not bind to the targeted portion of the PHIP pre-mRNA.

In some embodiments, a subject treated using the methods of the present disclosure expresses a partially functional PHIP protein from one allele, wherein the partially functional PHIP protein may be caused by a frameshift mutation, a nonsense mutation, a missense mutation, or a partial gene deletion. In some embodiments, a subject treated using the methods of the disclosure expresses a nonfunctional PHIP protein from one allele, wherein the nonfunctional PHIP protein may be caused by a frameshift mutation, a nonsense mutation, a missense mutation, a partial gene deletion, in one allele. In some embodiments, a subject treated using the methods of the disclosure has a PHIP whole gene deletion, in one allele.

### Exon Inclusion

As used herein, a “NMD exon-containing pre-mRNA” is a pre-mRNA transcript that contains at least one pseudo-exon. Alternative or aberrant splicing can result in inclusion of the at least one pseudo-exon in the mature mRNA transcripts. The terms “mature mRNA,” and “fully-spliced mRNA,” are used interchangeably herein to describe a fully processed mRNA. Inclusion of the at least one pseudo-exon can be non-productive mRNA and lead to NMD of the mature mRNA. NMD exon-containing mature mRNA may sometimes lead to aberrant protein expression.

In some embodiments, the included pseudo-exon is the most abundant pseudo-exon in a population of NMD exon-containing pre-mRNAs transcribed from the gene encoding the target protein in a cell. In some embodiments, the included pseudo-exon is the most abundant pseudo-exon in a population of NMD exon-containing pre-mRNAs transcribed from the gene encoding the target protein in a cell, wherein the population of NMD exon-containing pre-mRNAs comprises two or more included pseudo-exons. In some embodiments, an antisense oligomer targeted to the most abundant pseudo-exon in the population of NMD exon-containing pre-mRNAs encoding the target protein induces exon skipping of one or two or more pseudo-exons in the population, including the pseudo-exon to which the antisense oligomer is targeted or binds. In some embodiments, the targeted region is in a pseudo-exon that is the most abundant pseudo-exon in a NMD exon-containing pre-mRNA encoding the PHIP protein.

The degree of exon inclusion can be expressed as percent exon inclusion, e.g., the percentage of transcripts in which a given pseudo-exon is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

In some embodiments, an included pseudo-exon is an exon that is identified as an included pseudo-exon based on a determination of at least about 5%, at least about 10%, at least about 15%, at least about 20%, at least about 25%, at least about 30%, at least about 35%, at least about 40%, at least about 45%, or at least about 50%, inclusion. In embodiments, a included pseudo-exon is an exon that is identified as a included pseudo-exon based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, inclusion. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22(9):1616-25) can be used to aid in identifying exon inclusion.

In some embodiments, contacting cells with an ASO that is complementary to a targeted portion of a PHIP pre-mRNA transcript results in an increase in the amount of PHIP protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of PHIP protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of PHIP protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a PHIP pre-mRNA transcript results in an increase in the amount of mRNA encoding PHIP, including the mature mRNA encoding the target protein. In some embodiments, the amount of mRNA encoding PHIP protein, or the mature mRNA encoding the PHIP protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding PHIP protein, or the mature mRNA encoding PHIP protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding PHIP protein, or the mature mRNA encoding PHIP protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the PHIP NMD exon-containing pre-mRNA.

The NMD exon can be in any length. In some embodiments, the NMD exon comprises a full sequence of an intron, in which case, it can be referred to as intron retention. In some embodiments, the NMD exon can be a portion of the intron. In some embodiments, the NMD exon can be a 5′ end portion of an intron including a 5′ss sequence. In some embodiments, the NMD exon can be a 3′ end portion of an intron including a 3′ss sequence. In some embodiments, the NMD exon can be a portion within an intron without inclusion of a 5′ss sequence. In some embodiments, the NMD exon can be a portion within an intron without inclusion of a 3′ss sequence. In some embodiments, the NMD exon can be a portion within an intron without inclusion of either a 5′ss or a 3′ss sequence. In some embodiments, the NMD exon can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the NMD exon can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the NMD exon can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the NMD exon may be longer than 1,000 nucleotides in length.

Inclusion of a pseudo-exon can lead to a frameshift and the introduction of a premature termination codon (PIC) in the mature mRNA transcript rendering the transcript a target of NMD.

Mature mRNA transcript containing NMD exon can be non-productive mRNA transcript which does not lead to protein expression. The PIC can be present in any position downstream of an NMD exon. In some embodiments, the PIC can be present in any exon downstream of an NMD exon. In some embodiments, the PIC can be present within the NMD exon. For example, inclusion of exon 15x of PHIP in an mRNA transcript encoded by the PHIP gene can induce a PIC in the mRNA transcript. For example, inclusion of exon (GRCh38/hg38: chr6 79004373 to 79004436) of PHIP in an mRNA transcript encoded by the PHIP.

### Therapeutic Agents

In various embodiments of the present disclosure, compositions and methods comprising a therapeutic agent are provided to modulate protein expression level of PHIP. In some embodiments, provided herein are compositions and methods to modulate alternative splicing of PHIP pre-mRNA. In some embodiments, provided herein are compositions and methods to induce exon skipping in the splicing of PHIP pre-mRNA, e.g., to induce skipping of a pseudo-exon during splicing of PHIP pre-mRNA. In other embodiments, therapeutic agents may be used to induce the inclusion of an exon in order to decrease the protein expression level.

A therapeutic agent disclosed herein can be a NIE repressor agent. A therapeutic agent may comprise a polynucleic acid polymer.

According to one aspect of the present disclosure, provided herein is a method of treatment or prevention of a condition or disease associated with a functional PHIP protein deficiency, comprising administering a NIE repressor agent to a subject to increase levels of functional PHIP protein, wherein the agent binds to a region of the pre-mRNA transcript to decrease inclusion of the NMD exon in the mature transcript. For example, provided herein is a method of treatment or prevention of a condition associated with a functional PHIP protein deficiency, comprising administering a NIE repressor agent to a subject to increase levels of functional PHIP protein, wherein the agent binds to a region of an intron containing an NMD exon (e.g., exon 15x of PHIP) of the pre-mRNA transcript or to a NMD exon-activating regulatory sequence in the same intron. For example, provided herein is a method of treatment or prevention of a condition associated with a functional PHIP protein deficiency, comprising administering a NIE repressor agent to a subject to increase levels of functional PHIP protein, wherein the agent binds to a region of an intron containing an NMD exon (e.g., exon (GRCh38/hg38: chr6 79004373 to 79004436) of PHIP) of the pre-mRNA transcript or to a NMD exon-activating regulatory sequence in the same intron.

Where reference is made to reducing NMD exon inclusion in the mature mRNA, the reduction may be complete, e.g., 100%, or may be partial. The reduction may be clinically significant. The reduction/correction may be relative to the level of NMD exon inclusion in the subject without treatment, or relative to the amount of NMD exon inclusion in a population of similar subjects. The reduction/correction may be at least 10% less NMD exon inclusion relative to the average subject, or the subject prior to treatment. The reduction may be at least 20% less NMD exon inclusion relative to an average subject, or the subject prior to treatment. The reduction may be at least 40% less NMD exon inclusion relative to an average subject, or the subject prior to treatment. The reduction may be at least 50% less NMD exon inclusion relative to an average subject, or the subject prior to treatment. The reduction may be at least 60% less NMD exon inclusion relative to an average subject, or the subject prior to treatment. The reduction may be at least 80% less NMD exon inclusion relative to an average subject, or the subject prior to treatment. The reduction may be at least 90% less NMD exon inclusion relative to an average subject, or the subject prior to treatment.

Where reference is made to increasing active PHIP protein levels, the increase may be clinically significant. The increase may be relative to the level of active PHIP protein in the subject without treatment, or relative to the amount of active PHIP protein in a population of similar subjects. The increase may be at least 10% more active PHIP protein relative to the average subject, or the subject prior to treatment. The increase may be at least 20% more active PHIP protein relative to the average subject, or the subject prior to treatment. The increase may be at least 40% more active PHIP protein relative to the average subject, or the subject prior to treatment. The increase may be at least 50% more active PHIP protein relative to the average subject, or the subject prior to treatment. The increase may be at least 80% more active PHIP protein relative to the average subject, or the subject prior to treatment. The increase may be at least 100% more active PHIP protein relative to the average subject, or the subject prior to treatment. The increase may be at least 200% more active PHIP protein relative to the average subject, or the subject prior to treatment. The increase may be at least 500% more active PHIP protein relative to the average subject, or the subject prior to treatment.

In embodiments wherein the NIE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer comprising a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 2-5. The polynucleic acid polymer may comprise a sequence with 100% sequence identity to SEQ ID NO: 3.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence.

References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that induces exon skipping by binding to a targeted portion of a PHIP pre-mRNA, e.g., a PHIP NMD exon-containing pre-mRNA. As used herein, the terms “ASO” and “antisense oligomer” are used interchangeably and refer to an oligomer such as a polynucleotide, comprising nucleobases that hybridizes to a target nucleic acid (e.g., a PHIP pre-mRNA, e.g., a PHIP NMD exon-containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art (for example, in PCT Application No. PCT/US2014/054151, published as WO 2015/035091, titled “Reducing Nonsense-Mediated mRNA Decay,” incorporated by reference herein), can be used to practice the methods described herein.

In some embodiments, ASOs “specifically hybridize” to or are “specific” to a target nucleic acid or a targeted portion of a PHIP pre-mRNA, e.g., a NMD exon-containing pre-mRNA.

Typically such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of a PHIP pre-mRNA, e.g., a NMD exon-containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphorothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the disclosure, including, but not limited to, any of the ASOs set forth herein in Tables 5 and 6, comprises an ASO having phosphorus internucleotide linkages that are not random. In some embodiments, a composition used in the methods of the disclosure comprises a pure diastereomeric ASO. In some embodiments, a composition used in the methods of the disclosure comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second generation antisense oligonucleotides containing chiral phosphorothioate linkages,” Nucleic Acids Research 42(22): 13456-13468, incorporated herein by reference). In some embodiments, an ASO used in the methods of the disclosure, including, but not limited to, any of the ASOs set forth herein in SEQ ID NO: 3, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NO: 3, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of SEQ ID NO: 3, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of SEQ ID NO: 3, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; 2′-NMA moiety; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. As used herein, “2′-NMA” can mean a —O—CH₂—C(═O)—NH—CH₃ group in place of the 2′—OH group of a ribosyl sugar moiety. A “2′-NMA sugar moiety” or “2′-NMA moiety” is a sugar moiety with a 2′-O—CH₂—C(═O)—NH—CH₃ group in place of the 2′-OH group of a ribosyl sugar moiety. Unless otherwise indicated, a 2′-NMA sugar moiety is in the β-D configuration. “NMA” can mean O—N-methyl acetamide. In some embodiments, the sugar moiety modification is selected from 2′-O-Me, 2′F, 2′-NMA, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a PHIP pre-mRNA, e.g., a PHIP NMD exon-containing pre-mRNA, that is downstream (in the 3′ direction) of the 5′ splice site (or 3′ end of the NMD exon) of the included exon in a PHIP pre-mRNA (e.g., the direction designated by positive numbers relative to the 5′ splice site). In some embodiments, the ASOs are complementary to a targeted portion of the PHIP pre-mRNA, e.g., the PHIP NMD exon-containing pre-mRNA that is within the region about +1 to about +500 relative to the 5′ splice site (or 3′ end) of the included exon. In some embodiments, the ASOs may be complementary to a targeted portion of a PHIP pre-mRNA, e.g., a PHIP NMD exon-containing pre-mRNA, that is within the region between nucleotides +6 and +40,000 relative to the 5′ splice site (or 3′ end) of the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the included exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the included exon.

In some embodiments, the ASOs are complementary to (and bind to) a targeted portion of a PHIP pre-mRNA, e.g., a PHIP NMD exon-containing pre-mRNA, that is upstream (in the 5′ direction) of the 5′ splice site (or 3′ end) of the included exon in a PHIP pre-mRNA, e.g., a PHIP NMD exon-containing pre-mRNA (e.g., the direction designated by negative numbers relative to the 5′ splice site). In some embodiments, the ASOs are complementary to a targeted portion of the PHIP pre-mRNA, e.g., the PHIP NMD exon-containing pre-mRNA, that is within the region about −4 to about −270 relative to the 5′ splice site (or 3′end) of the included exon. In some embodiments, the ASOs may be complementary to a targeted portion of a PHIP pre-mRNA, e.g., a PHIP NMD exon-containing pre-mRNA, that is within the region between nucleotides −1 and −40,000 relative to the 5′ splice site (or 3′ end) of the included exon. In some aspects, the ASOs are complementary to a targeted portion that is within the region about −1 to about −40,000, about −1 to about −30,000, about −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the included exon.

In some embodiments, the ASOs are complementary to a targeted region of a PHIP pre-mRNA, e.g., a PHIP NMD exon-containing pre-mRNA, that is upstream (in the 5′ direction) of the 3′ splice site (or 5′ end) of the included exon in a PHIP pre-mRNA (e.g., in the direction designated by negative numbers). In some embodiments, the ASOs are complementary to a targeted portion of the PHIP pre-mRNA, e.g., the PHIP NMD exon-containing pre-mRNA, that is within the region about −1 to about −500 relative to the 3′ splice site (or 5′ end) of the included exon. In some embodiments, the ASOs are complementary to a targeted portion of the PHIP pre-mRNA that is within the region −1 to −40,000 relative to the 3′ splice site of the included exon. In some aspects, the ASOs are complementary to a targeted portion that is within the region about −1 to about −40,000, about −1 to about −30,000, −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 3′ splice site of the included exon. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the included exon.

In some embodiments, the ASOs are complementary to a targeted region of a PHIP pre-mRNA, e.g., a PHIP NMD exon-containing pre-mRNA, that is downstream (in the 3′ direction) of the 3′ splice site (5′ end) of the included exon in a PHIP pre-mRNA, e.g., a PHIP NMD exon-containing pre-mRNA (e.g., in the direction designated by positive numbers). In some embodiments, the ASOs are complementary to a targeted portion of the PHIP pre-mRNA that is within the region of about +1 to about +40,000 relative to the 3′ splice site of the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20, or about +1 to about +10 relative to 3′ splice site of the included exon.

In some embodiments, the targeted portion of the PHIP pre-mRNA, e.g., the PHIP NMD exon-containing pre-mRNA, is within the region +100 relative to the 5′ splice site (3′ end) of the included exon to −100 relative to the 3′ splice site (5′ end) of the included exon. In some embodiments, the targeted portion of the PHIP NMD exon-containing pre-mRNA is within the NMD exon. In some embodiments, the target portion of the PHIP NMD exon-containing pre-mRNA comprises a pseudo-exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length.

In some embodiments, the ASOs are 25 nucleotides in length.

In some embodiments, two or more ASOs with different chemistries but complementary to the same targeted portion of the pre-mRNA, e.g., NMD exon-containing pre-mRNA, are used. In some embodiments, two or more ASOs that are complementary to different targeted portions of the pre-mRNA, e.g., the NMD exon-containing pre-mRNA, are used.

In some embodiments, the antisense oligonucleotides of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N—Ac-Glucosamine (GluNAc), or mannose (e.g., mannose-6-phosphate), a lipid, or a polyhydrocarbon compound.

Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a PHIP pre-mRNA, e.g., NMD exon-containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line.

In some embodiments, the cell is in vitro (e.g., in cell culture).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to affect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In embodiments, the antisense oligonucleotide is administered in combination with another drug or therapeutic agent.

### Combination Therapies

In some embodiments, the ASOs disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents comprise an ASO that can be used to correct intron retention.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

In some cases, the subject pharmaceutical composition and method are applicable for treatment of a condition or disease associated with PHIP deficiency. In some cases, the subject pharmaceutical composition and method are applicable for treatment of Chung-Jansen syndrome (CHUJANS), an autosomal dominant disorder, intellectual disability, speech delay, anxiety, autism spectrum disorders (ASD), Attention deficit hyperactivity disorder (ADHD), aggression, facial dysmorphism, café au lait spots, overweight syndrome caused by PHIP haploinsufficiency, developmental delay, obesity or dysmorphism.

In some cases, a therapeutic agent comprises an oligonucleotide. In some cases, a therapeutic agent comprises a vector, e.g., a viral vector, expressing a oligonucleotide that binds to the targeted region of a pre-mRNA the encodes the target peptide sequence. The methods provided herein can be adapted to contacting a vector that encodes an agent, e.g., an oligonucleotide, to a cell, so that the agent binds to a pre-mRNA in the cell and modulates the processing of the pre-mRNA. In some cases, the viral vector comprises an adenoviral vector, adeno-associated viral (AAV) vector, lentiviral vector, Herpes Simplex Virus (HSV) viral vector, retroviral vector, or any applicable viral vector. In some cases, a therapeutic agent comprises a gene editing tool that is configured to modify a gene encoding the target peptide sequence such that a gene region that encodes the inefficient translation region is deleted. In some cases, a gene editing tool comprises vector, e.g., viral vector, for gene editing based on CRISPR-Cas9, TALEN, Zinc Finger, or other applicable technologies.

Suitable routes for administration of ASOs of the present disclosure may vary depending on cell type to which delivery of the ASOs is desired. The ASOs of the present disclosure may be administered to patients parenterally, for example, by intravitreal injection, intrathecal injection, intracerebroventricular injection, intraperitoneal injection, intramuscular injection, subcutaneous injection, or intravenous injection.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some cases, a therapeutic agent comprises a modified snRNA, such as a modified human snRNA. In some cases, a therapeutic agent comprises a vector, such as a viral vector, that encodes a modified snRNA. In some embodiments, the modified snRNA is a modified U1 snRNA (see, e.g., Alanis et al., Human Molecular Genetics, 2012, Vol. 21, No. 11 2389-2398). In some embodiments, the modified snRNA is a modified U7 snRNA (see, e.g., Gadgil et al., J Gene Med. 2021; 23:e3321). In some embodiments, the modified snRNA has been modified to comprise a single-stranded nucleotide sequence that hybridizes to a PHIP NMD exon-containing pre-mRNA.

In some embodiments, the modified snRNA has been modified to comprise a single-stranded nucleotide sequence that comprises one or two or more sequences of the ASOs disclosed herein.

In some embodiments, the modified snRNA has been modified to comprise a single-stranded nucleotide sequence that hybridizes to sequence of a PHIP NMD exon-containing pre-mRNA with a mutation. In some embodiments, the modified snRNA has been modified to comprise a single-stranded nucleotide sequence that comprises two or more sequences that hybridize to two or more target regions of a PHIP NMD exon-containing pre-mRNA. For example, a modified snRNA can be modified to comprise a single-stranded nucleotide sequence that hybridizes to at least 8 contiguous nucleic acids of a PHIP NMD exon-containing pre-mRNA. In some embodiments, the modified snRNA has been modified to comprise a single-stranded nucleotide sequence that hybridizes to any of the target regions of a PHIP NMD exon-containing pre-mRNA disclosed herein. In some embodiments, the modified snRNA has been modified to comprise a single-stranded nucleotide sequence that comprises two or more sequences that hybridize to two or more target regions of a PHIP NMD exon-containing pre-mRNA. For example, a modified snRNA can be modified to comprise a single-stranded nucleotide sequence that hybridizes to one or two or more sequences of an intron containing an NMD exon (e.g., exon 15x of PHIP (e.g., exon (GRCh38/hg38: chr6 79004373 to 79004436) of PHIP)) of the pre-mRNA transcript or to a NMD exon-activating regulatory sequence in the same intron. For example, a modified snRNA can be modified to comprise a single-stranded nucleotide sequence that hybridizes to a region within an NMD exon or upstream or downstream of an NMD exon (e.g., exon 15x of PHIP (e.g., exon (GRCh38/hg38: chr6 79004373 to 79004436) of PHIP)). In some embodiments, the modified snRNA has a 5′ region that has been modified to comprise a single-stranded nucleotide sequence that hybridizes to a PHIP NMD exon-containing pre-mRNA.

For example, a modified snRNA can be modified to comprise a single-stranded nucleotide sequence that hybridizes to a region that overlaps with an NMD exon and an intron upstream of the NMD exon (e.g., exon 15x of PHIP (e.g., exon (GRCh38/hg38: chr6 79004373 to 79004436) of PHIP)). For example, a modified snRNA can be modified to comprise a single-stranded nucleotide sequence that hybridizes to a region that overlaps with an NMD exon and an intron downstream of the NMD exon (e.g., exon 15x of PHIP (e.g., exon (GRCh38/hg38: chr6 79004373 to 79004436) of PHIP)).

For example, a modified snRNA can be modified to comprise a single-stranded nucleotide sequence that is complementary to an intron sequence that is downstream of an NMD exon (e.g., exon 15x of PHIP (e.g., exon (GRCh38/hg38: chr6 79004373 to 79004436) of PHIP)). For example, a modified snRNA can be modified to comprise a single-stranded nucleotide sequence that is complementary to a 3′ splice site of an intron sequence that is upstream or downstream of an NMD exon (e.g., exon 15x of PHIP (e.g., exon (GRCh38/hg38: chr6 79004373 to 79004436) of PHIP)). For example, a modified snRNA can be modified to comprise a single-stranded nucleotide sequence that is complementary to a 5′ splice site of an intron sequence that is downstream of an NMD exon (e.g., exon 15x of PHIP (e.g., exon (GRCh38/hg38: chr6 79004373 to 79004436) of PHIP)).

For example, a modified snRNA can be modified to comprise a single-stranded nucleotide sequence that is complementary to an intron sequence that is upstream of an NMD exon (e.g., exon 15x of PHIP (e.g., exon (GRCh38/hg38: chr6 79004373 to 79004436) of PHIP)). For example, a modified snRNA can be modified to comprise a single-stranded nucleotide sequence that is complementary to a splice site of an intron sequence that is upstream of an NMD exon (e.g., exon 15x of PHIP (e.g., exon (GRCh38/hg38: chr6 79004373 to 79004436) of PHIP)). For example, a modified snRNA can be modified to comprise a single-stranded nucleotide sequence that is complementary to a 3′ splice site of an intron sequence that is upstream of an NMD exon (e.g., exon 15x of PHIP (e.g., exon (GRCh38/hg38: chr6 79004373 to 79004436) of PHIP)). For example, a modified snRNA can be modified to comprise a single-stranded nucleotide sequence that is complementary to a 5′ splice site of an intron sequence that is upstream of an NMD exon (e.g., exon 15x of PHIP (e.g., exon (GRCh38/hg38: chr6 79004373 to 79004436) of PHIP)).

In some embodiments, subjects treated using the methods and compositions are evaluated for improvement in condition using any methods known and described in the art.

Methods of Identifying Additional ASOs that Induce Exon Skipping

Also within the scope of the present disclosure are methods for identifying or determining ASOs that induce exon skipping of a PHIP NMD exon-containing pre-mRNA. For example, a method can comprise identifying or determining ASOs that induce pseudo-exon skipping of a PHIP NMD exon-containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., pseudo-exon skipping, protein or functional RNA production). These methods also can be used for identifying ASOs that induce exon skipping of the included exon by binding to a targeted region in an intron flanking the included exon, or in a non-included exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the included exon (e.g., a portion of sequence of the exon located upstream of the target/included exon) to approximately 100 nucleotides downstream of the 3′ splice site of the target/included exon and/or from approximately 100 nucleotides upstream of the 5′ splice site of the included exon to approximately 100 nucleotides downstream of the 5′ splice site of the target/included exon (e.g., a portion of sequence of the exon located downstream of the target/included exon). For example, a first ASO of 15 nucleotides in length may be designed to specifically hybridize to nucleotides +6 to +20 relative to the 3′ splice site of the target/included exon. A second ASO may be designed to specifically hybridize to nucleotides +11 to +25 relative to the 3′ splice site of the target/included exon. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 1,160 nucleotides upstream of the 3′ splice site, to about 500 nucleotides downstream of the 5′ splice site. In some embodiments, the ASOs can be tiled from about 500 nucleotides upstream of the 3′ splice site, to about 1,920 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NMD exon-containing pre-mRNA described herein). The exon skipping effects of each of the ASOs may be assessed by any method known in the art, for example by reverse transcriptase (RT)-PCR using primers that span the splice junction, as described in Example 2. A reduction or absence of a longer RT-PCR product produced using the primers spanning the region containing the included exon (e.g., including the flanking exons of the NMD exon) in ASO-treated cells as compared to in control ASO-treated cells indicates that splicing of the target NMD exon has been enhanced. In some embodiments, the exon skipping efficiency (or the splicing efficiency to splice the intron containing the NMD exon), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping (or enhanced splicing of NMD exon).

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NMD exon, as described herein (see, e.g., Example 2). A reduction or absence of a longer RT-PCR product produced using the primers spanning the NMD exon in ASO-treated cells as compared to in control ASO-treated cells indicates that exon skipping (or splicing of the target intron containing an NMD exon) has been enhanced. In some embodiments, the exon skipping efficiency (or the splicing efficiency to splice the intron containing the NMD exon), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in exon skipping (or enhanced splicing of the intron containing a NMD exon) and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intravitreal injection, intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (e.g., efficiency, rate, extent) and protein production by methods known in the art and described herein. The animal models may also be any phenotypic or behavioral indication of the disease or disease severity.

Also within the scope of the present disclosure is a method to identify or validate an NMD-inducing exon in the presence of an NMD inhibitor, for example, cycloheximide. An exemplary method is provided in Example 3.

### SPECIFIC EMBODIMENTS

Embodiment 1. A method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating a level of processed mRNA that is processed from the pre-mRNA, and modulating the expression of the target protein in the cell, and wherein the target gene is a PHIP gene.
Embodiment 2. The method of Embodiment 1, wherein the agent:
   a. binds to a targeted portion of the pre-mRNA;
   b. modulates binding of a factor involved in splicing of the NMD exon; or
   c. a combination of (a) and (b).
Embodiment 3. The method of Embodiment 2, wherein the agent interferes with binding of the factor involved in splicing of the NMD exon to a region of the targeted portion.
Embodiment 4. The method of Embodiment 2, wherein the targeted portion of the pre-mRNA is proximal to the NMD exon.
Embodiment 5. The method of Embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon.
Embodiment 6. The method of Embodiment 2, wherein the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon.
Embodiment 7. The method of Embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon.
Embodiment 8. The method of Embodiment 2, wherein the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon.
Embodiment 9. The method of Embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 79004373.
Embodiment 10. The method of Embodiment 2, wherein the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 79004373.
Embodiment 11. The method of Embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 79004436.
Embodiment 12. The method of Embodiment 2, wherein the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 79004436.
Embodiment 13. The method of Embodiment 2, wherein the targeted portion of the pre-mRNA is located in an intronic region between two canonical exonic regions of the pre-mRNA, and wherein the intronic region contains the NMD exon.
Embodiment 14. The method of Embodiment 2, wherein the targeted portion of the pre-mRNA at least partially overlaps with the NMD exon.
Embodiment 15. The method of Embodiment 2, wherein the targeted portion of the pre-mRNA at least partially overlaps with an intron upstream or downstream of the NMD exon.
Embodiment 16. The method of Embodiment 2, wherein the targeted portion of the pre-mRNA comprises 5′ NMD exon-intron junction or 3′ NMD exon-intron junction.
Embodiment 17. The method of Embodiment 2, wherein the targeted portion of the pre-mRNA is within the NMD exon.
Embodiment 18. The method of Embodiment 2, wherein the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.
Embodiment 19. The method of any one of Embodiments 1 to 18, wherein the NMD exon is (a) within an intronic sequence with at least 80%, at least 90%, or 100% sequence identity to SEQ ID NO: 2 and/or (b) comprises a sequence with at least 80%, at least 90%, or 100% sequence identity to SEQ ID NO: 3.
Embodiment 20. The method of any one of Embodiments 1 to 18, wherein the NMD exon comprises a sequence of SEQ ID NO: 3.
Embodiment 21. The method of Embodiment 2, wherein the targeted portion of the pre-mRNA is within the non-sense mediated RNA decay-inducing exon GRCh38/hg38: chr6 79004373 to 79004436.
Embodiment 22. The method of Embodiment 2, wherein the targeted portion of the pre-mRNA is upstream or downstream of the non-sense mediated RNA decay-inducing exon GRCh38/hg38: chr6 79004373 to 79004436.
Embodiment 23. The method of Embodiment 2, wherein the targeted portion of the pre-mRNA comprises an exon-intron junction of the non-sense mediated RNA decay-inducing exon GRCh38/hg38: chr6 79004373 to 79004436.
Embodiment 24. The method of any one of Embodiments 1 to 23, wherein the target protein expressed from the processed mRNA is a full-length PHIP protein or a wild-type PHIP protein.
Embodiment 25. The method of any one of Embodiments 1 to 23, wherein the target protein expressed from the processed mRNA is a functional PHIP protein.
Embodiment 26. The method of any one of Embodiments 1 to 23, wherein the target protein expressed from the processed mRNA is at least partially functional as compared to a wild-type PHIP protein.
Embodiment 27. The method of any one of Embodiments 1 to 23, wherein the target protein expressed from the processed mRNA is at least partially functional as compared to a full-length wild-type PHIP protein.
Embodiment 28. The method of any one of Embodiments 1 to 23, or 25 to 27, wherein the target protein expressed from the processed mRNA is a PHIP protein that lacks an amino acid sequence encoded by the non-sense mediated RNA decay-inducing exon GRCh38/hg38: chr6 79004373 to 79004436.
Embodiment 29. The method of any one of Embodiments 1 to 28, wherein the method promotes exclusion of the NMD exon from the pre-mRNA.
Embodiment 30. The method of Embodiment 29, wherein the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.
Embodiment 31. The method of any one of Embodiments 1 to 30, wherein the method results in an increase in the level of the processed mRNA in the cell.
Embodiment 32. The method of Embodiment 31, wherein the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.
Embodiment 33. The method of any one of Embodiments 1 to 32, wherein the method results in an increase in the expression of the target protein in the cell.
Embodiment 34. The method of Embodiment 33, wherein a level of the target protein expressed from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.
Embodiment 35. The method of any one of Embodiments 1 to 34, wherein the agent comprises an antisense oligomer with at least 80%, at least 90%, or 100% sequence identity to a sequence selected from the group consisting of SEQ ID NOs: 4-182.
Embodiment 36. The method of any one of Embodiments 1 to 34, wherein the agent further comprises a gene editing molecule.

Embodiment 37. The method of Embodiment 36, wherein the gene editing molecule comprises CRISPR-Cas9.
Embodiment 38. The method of any one of Embodiments 1 to 37, wherein the agent is an antisense oligomer (ASO) and wherein the antisense oligomer comprises a backbone modification comprising a phosphorothioate linkage or a phosphorodiamidate linkage.
Embodiment 39. The method of any one of Embodiments 1 to 38, wherein the agent is an antisense oligomer (ASO) and wherein the antisense oligomer comprises a phosphorodiamidate morpholino, a locked nucleic acid, a peptide nucleic acid, a 2′-O-methyl moiety, a 2′-Fluoro moiety, a 2′-NMA moiety, or a 2′-O-methoxyethyl moiety.
Embodiment 40. The method of any one of Embodiments 1 to 39, wherein the agent is an antisense oligomer (ASO) and wherein the antisense oligomer comprises at least one modified sugar moiety.
Embodiment 41. The method of Embodiment 40, wherein each sugar moiety is a modified sugar moiety.
Embodiment 42. The method of any one of Embodiments 1 to 41, wherein the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.
Embodiment 43. The method of Embodiment 1, wherein method comprises contacting a vector encoding the agent to the cell, wherein the vector is a viral vector.
Embodiment 44. The method of Embodiment 43, wherein the viral vector comprises an adenoviral vector, adeno-associated viral (AAV) vector, lentiviral vector, Herpes Simplex Virus (HSV) viral vector, or retroviral vector.
Embodiment 45. The method of Embodiment 43, wherein the viral vector comprises an adeno-associated viral (AAV) vector.
Embodiment 46. The method of Embodiment 1, wherein the agent comprises a modified snRNA.
Embodiment 47. The method of Embodiment 46, wherein the modified human snRNA is a modified U1 snRNA.
Embodiment 48. The method of Embodiment 46, wherein the modified human snRNA is a modified U7 snRNA.
Embodiment 49. The method of Embodiment 46, wherein a portion of a single-stranded nucleotide sequence of the modified human snRNA comprises a sequence that binds to the targeted portion of the pre-mRNA.
Embodiment 50. The method of any one of Embodiments 46 to 49, wherein the method comprises contacting a vector encoding the modified snRNA to the cell.
Embodiment 51. The method of any one of Embodiments 1 to 50, wherein the method further comprises assessing mRNA level or expression level of the target protein.
Embodiment 52. The method of any one of Embodiments 1 to 51, wherein the agent is a therapeutic agent.
Embodiment 53. A pharmaceutical composition comprising the therapeutic agent of Embodiment 52 or a vector encoding the therapeutic agent of Embodiment 52, and a pharmaceutically acceptable excipient.
Embodiment 54. A pharmaceutical composition, comprising a therapeutic agent or a vector encoding a therapeutic agent, and a pharmaceutically acceptable excipient, wherein the therapeutic agent comprises an antisense oligomer with at least 80% sequence identity to a sequence selected from the group consisting of SEQ ID NOs: 4-182.
Embodiment 55. The pharmaceutical composition of Embodiment 54, wherein the therapeutic agent comprises an antisense oligomer with at least 80%, at least 90%, or 100% sequence identity to a sequence selected from the group consisting of SEQ ID NOs: 55-182.
Embodiment 56. The pharmaceutical composition of any one of Embodiments 53 to 55, wherein the pharmaceutical composition is formulated for intracerebroventricular injection, intraperitoneal injection, intramuscular injection, intrathecal injection, subcutaneous injection, oral administration, synovial injection, intravitreal administration, subretinal injection, topical application, implantation, or intravenous injection.
Embodiment 57. The pharmaceutical composition of any one of Embodiments 53 to 55, wherein the pharmaceutical composition is formulated for intrathecal injection or intracerebrospinal injection.
Embodiment 58. The pharmaceutical composition of any one of Embodiments 53 to 57, wherein the pharmaceutical composition further comprises a second therapeutic agent.
Embodiment 59. The pharmaceutical composition of Embodiment 58, wherein the second therapeutic agent comprises a small molecule.
Embodiment 60. The pharmaceutical composition of Embodiment 58, wherein the second therapeutic agent comprises an antisense oligomer.
Embodiment 61. The pharmaceutical composition of Embodiment 58, wherein the second therapeutic agent corrects intron retention.
Embodiment 62. A composition comprising an antisense oligomer with at least 80% sequence identity to a sequence selected from the group consisting of SEQ ID NOs: 4-182, wherein the antisense oligomer comprises a backbone modification, a sugar moiety modification, or a combination thereof.
Embodiment 63. A composition comprising a viral vector encoding a polynucleotide comprising an antisense oligomer, wherein the antisense oligomer consists of a sequence selected from the group consisting of SEQ ID NOs: 4-182.
Embodiment 64. The composition of Embodiment 63, wherein the polynucleotide further comprises a modified snRNA.
Embodiment 65. The composition of Embodiment 64, wherein the modified human snRNA is a modified U1 snRNA.
Embodiment 66. The composition of Embodiment 64, wherein the modified human snRNA is a modified U7 snRNA.
Embodiment 67. The composition of Embodiment 62 or 63, wherein the antisense oligomer has at least 80%, at least 90%, or 100% sequence identity to a sequence selected from the group consisting of SEQ ID NOs: 55-182.
Embodiment 68. A method of treating or reducing the likelihood of developing a disease or condition in a subject in need thereof by modulating expression of a target protein in a cell of the subject, comprising contacting to cells of the subject the pharmaceutical composition of any one of Embodiments 53 to 61.
Embodiment 69. The method of Embodiment 68, wherein the disease or condition is associated with a loss-of-function mutation in a PHIP gene.
Embodiment 70. The method of Embodiment 68 or 69, wherein the disease or condition is associated with haploinsufficiency of the PHIP gene, and wherein the subject has a first allele encoding a functional PHIP protein, and a second allele from which the PHIP protein is not produced or produced at a reduced level, or a second allele encoding a nonfunctional PHIP protein or a partially functional PHIP protein.
Embodiment 71. The method of any one of Embodiments 68 to 70, wherein the disease or condition comprises an intellectual disability disease or condition.
Embodiment 72. The method of any one of Embodiments 68 to 70, wherein the disease or condition comprises Chung-Jansen syndrome (CHUJANS), an autosomal dominant disorder, intellectual disability, speech delay, anxiety, autism spectrum disorders (ASD), attention deficit hyperactivity disorder (ADHD), aggression, facial dysmorphism, café au lait spots, overweight syndrome caused by PHIP haploinsufficiency, developmental delay, obesity or dysmorphism.
Embodiment 73. The method of any one of Embodiments 68 to 70, wherein the disease or condition comprises Chung-Jansen syndrome.
Embodiment 74. The method of any one of Embodiments 68 to 70, wherein the disease or condition comprises an intellectual disability.
Embodiment 75. The method of Embodiment 68 or 69, wherein the disease or condition is associated with an autosomal recessive mutation of PHIP gene, wherein the subject has a first allele from which:
   (i) PHIP protein is not produced or produced at a reduced level compared to a wild-type allele; or
   (ii) the PHIP protein produced is nonfunctional or partially functional compared to a wild-type allele, and
Embodiment 76. a second allele from which:
Embodiment 77. (iii) the PHIP protein is produced at a reduced level compared to a wild-type allele and the PHIP protein produced is at least partially functional compared to a wild-type allele; or
Embodiment 78. (iv) the PHIP protein produced is partially functional compared to a wild-type allele.
Embodiment 79. The method of any one of Embodiments 68 to 76, wherein the subject is a human.
Embodiment 80. The method of any one of Embodiments 68 to 76, wherein the subject is a non-human animal.
Embodiment 81. The method of any one of Embodiments 68 to 76, wherein the subject is a fetus, an embryo, or a child.
Embodiment 82. The method of any one of Embodiments 68 to 76, wherein the cells are ex vivo.
Embodiment 83. The method of any one of Embodiments 68 to 76, wherein the pharmaceutical composition is administered by intracerebroventricular injection, intraperitoneal injection, intramuscular injection, intrathecal injection, subcutaneous injection, oral administration, synovial injection, intravitreal administration, subretinal injection, topical application, implantation, or intravenous injection.
Embodiment 84. The method of any one of Embodiments 68 to 76, wherein the pharmaceutical composition is administered by intrathecal injection or intracerebrospinal injection.
Embodiment 85. The method of any one of Embodiments 68 to 76, wherein the method treats the disease or condition.
Embodiment 86. A composition comprising an agent or a vector encoding the agent that modulates splicing of a non-sense mediated RNA decay-inducing exon (NMD exon) from a pre-mRNA that is transcribed from a target gene and that comprises the NMD exon, thereby modulating the level of a processed mRNA that is processed from the pre-mRNA, and modulating expression of a target protein in a cell having the pre-mRNA, and wherein the target gene is a PHIP gene.
Embodiment 87. A composition comprising an agent or a vector encoding the agent that modulates splicing of a non-sense mediated RNA decay-inducing exon (NMD exon) from a pre-mRNA that is transcribed from a target gene and that comprises the NMD exon, thereby treating a disease or condition in a subject in need thereof by modulating the level of a processed mRNA that is processed from the pre-mRNA, and modulating expression of a target protein in a cell of the subject, and wherein the target gene is a PHIP gene Embodiment 88. A pharmaceutical composition comprising the composition of Embodiment 84 or 85; and a pharmaceutically acceptable excipient and/or a delivery vehicle.
Embodiment 89. The method or composition or pharmaceutical composition of any one of previous Embodiments, wherein the target protein is pleckstrin homology domain-interacting protein (PHIP).

### EXAMPLES

The present disclosure will be more specifically illustrated by the following Examples. However, it should be understood that the present disclosure is not limited by these examples in any manner.

### Example 1: Identification of NMD-Inducing Exon Inclusion Events in Transcripts by RNAseq Using Sequencing

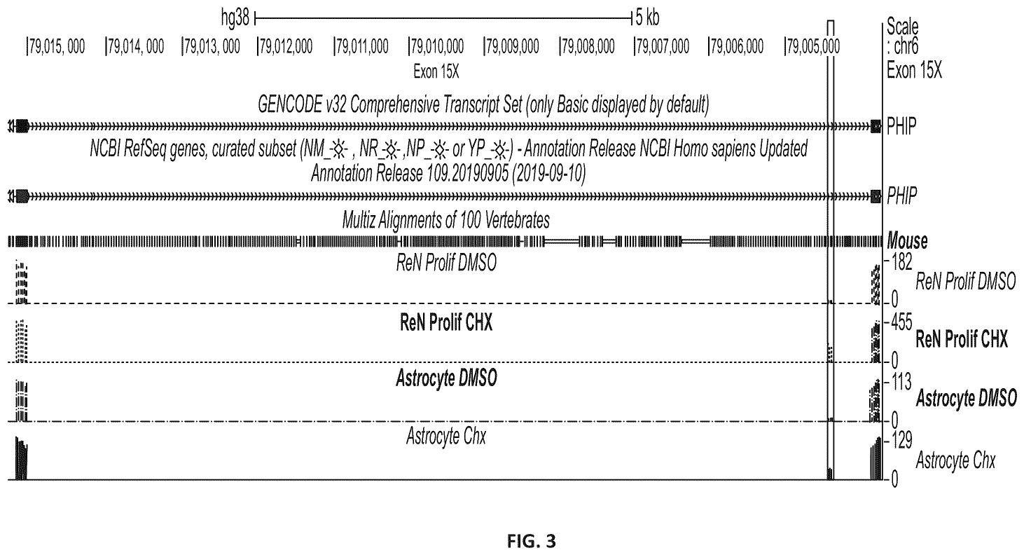

Whole transcriptome shotgun sequencing was carried out using RNA sequencing to reveal a snapshot of transcripts produced by the genes described herein to identify NMD exon inclusion events. For this purpose, polyA+ RNA from nuclear and cytoplasmic fractions of human neural progenitor cells (ReN) and human astrocytes treated with cycloheximide (CHX) or DMSO control was isolated and cDNA libraries are constructed using Illumina's TruSeq Stranded mRNA library Prep Kit. The libraries are pair-end sequenced resulting in 100-nucleotide reads that are mapped to the human genome (December 2017, GRCh38/hg38 assembly). FIG. 3 depicts identification of an exemplary nonsense-mediated mRNA decay (NMD)-inducing exon in the PHIP gene.

Exemplary genes and intron sequences are summarized in Table 1 (SEQ ID NOs indicate the corresponding nucleotide sequences represented by the Gene ID Nos). The sequence an exemplary intron is summarized in Table 2. The PHIP protein sequence is provided in Table 3. Tables 4, 5 and 6 list sequences of PHIP antisense oligomers of this disclosure.

TABLE 1
List of exemplary target gene sequences.

| | Gene | SEQ | | | | |
|---|---|---|---|---|---|---|
| Gene | ID | ID | | | | |
| Symbol | No. | NO. | Disease | OMIM | Genetics | Transcripts |
| PHIP | 55023 | 1 | Chung-Jansen | 612870 | Haploinsufficient | ENST00000275034 |
| | | | syndrome; | | | |
| | | | intellectual | | | |
| | | | disability | | | |

TABLE 2
Sequences of exemplary target introns/NMD exons
in pre-mRNA transcripts.

| | SEQ ID | |
|---|---|---|
| Gene | NO. | Intron |
| PHIP | 2 | gtaattatcattctcttcattttatacctactaaagatttgggagctttttgac |
| Intron | | aaaggtactctgcatgtcacaaaggaaatccatttaaaaattggttcattattt |
| 15 | | tgactttaaattcccatcaataaagaatgatttattccacacttttattgacat |
| sequence | | gctgaatttaaaagaaaattaattgtgcatattacagtgcctgtacattgtgat |
| | | aactgatacagttttatcagttttagcagtgaaactttggaaattcacaagtat |
| | | gaagatacaatatagagaagaaaaaatacttggaaaaccatgtgaagaaattat |
| | | aaataaatactcgtattattgtacttgtttaatagtcttctagcacaatagaat |
| | | tttactcaagtacactgattttaaaattaaggccaaagaatcaagcatttcttg |
| | | cttttaatcaattggtagtatattaaatatttgagtttatactcttctcttcac |
| | | attttgtttcttagatgttaacataattggccaagtatttgaattacaatgttt |
| | | taatatatcacatacaacttaccgagcagtgtttctatttactgtatgaagtat |
| | | taaggtctcctcaactactgcagtttgtgtagtcatattttttaaatgtctact |
| | | ttctgtatattagagatacctttcaacaaagaacaaaagaaaaaacagtttcca |
| | | ctatttcagcatgtttaaatttttttaaaaaaccaattatagaaatctttagta |
| | | atgaaccataaaacaatacaattagtacatgtggtcagaattattttgccaaat |
| | | aaattatttcacaaaacagttgtgaaacatggtttgtgaaaaaattacacgaat |
| | | gtgatgctatgcttgattgataatcagagtaaagttatttctatgtcgtgagta |
| | | agcagatataatagcagattttttagtgagggaaatagctttagctatctttaa |
| | | ggctgtgccttagaatacattaggtaagaaataaggtttgtaaaaccctctatt |
| | | tctagaaaacagtgttaattatttggtgtgcacttgttagtgatggatggattt |
| | | cctttgtgattgtattgagatttttttaaaaagccacctgtaaatatgctagta |
| | | aaaaatattttctgcttttctatccttgtgtttctgatttgctcctttttggaa |
| | | tatctgttgaataaggaaattagtcatttctcaatctgtctttatagcttaaaa |
| | | aaaataatatgtgagctatatatatactagtggcttcatttagtagttttgtat |
| | | ttccaaaggacattattatattggtagagtgtttggagggaaacttttctccca |
| | | taattgaataccgctttaacttttcagttatataaaggtgaattttcccttatc |
| | | aggtaagttggtcacactaatattctaccttttcagtagataacaaaaactgca |
| | | gcaccttgagagttaagtgatttgctcttagaccattaaaaattagtaatgtaa |
| | | ccaggagtagaagtcagccataatatattgtatatttttaaaaattagctgagg |
| | | gattggttacattactgccttaaaaattgtttattgttaaataagagtgttaga |
| | | tgaggatatttttctggttctttttatggataagagataggaaaactctaaaga |
| | | gcagaagaaacaaatacatgtaataagttgaattggatagtattcatagaattg |
| | | cagacagtgagcaaaacataaaccaaaaaagctaaaaaacagaaagtgagccac |
| | | ccttcatctcattaaaataaagatactacctttaattttttgtatgatgtttaa |
| | | tggtttccaaaatgctcccacatattttacctcctttgggtctcatgaaaacct |
| | | tgtaagatgatcaagatagattttattattcttggtcttaggaatggagaaact |
| | | gtacattacagagataaagtgctttgccccaatttactgagttcacacagctat |
| | | tatgcgggcacttggagcataaacccatgactctggttgtactcaaatcttgtc |
| | | atggttggctttttgagcttttgtcaatgttactgcatagctaacttttaacaa |
| | | tactcagttagtgatatgtctatgaagaaatgttttcataatggtaataaatct |
| | | gagtggatcttttgacatcttactctttaacttgattttttttcctgtctgtct |
| | | tttatgagttatctaattgccctaattctttttgctcttcactcttttgttctt |
| | | ttcattgcttgttttatttatgggcaaatggaagataaagcatccagcattgct |
| | | attaagaaaataaagagaaacaattcgagggagggcctgcgttctcactgcttg |
| | | ctatcatgaatgttgagttttggctataattatagctatttggctctctgtagc |
| | | taatcaataagtttttcagttactaaaataccacgatacttgattgcatacaaa |
| | | tttaatccctaatgttaagttgtagtagtaatcacaagttcagtttagtaaata |
| | | ttttttgagcaccatgtgaagggtctagtcatatatagtgctttggtctattag |
| | | aggatgaagaattctggtggaatactccttttttgactagaaatggtgaaagtt |
| | | atgtgaagtatttttaagattaaataatatttcatgtgactgttaaaactgaag |
| | | tttataaaacagataagtaaactcaccattccacctacagctagaaaataagga |
| | | atgtagactatacctttagaataacagttttagtacaaatttcttaaatactgg |
| | | tttttatcaagtatattttaagaatactttaatatttctcctgctttttgaata |
| | | ataattgaattttccattggaaatttatacctttgcaaaagataatggaaatca |
| | | tgtaacatataatttagtattttgtattcatcagcattttactgcattttttgt |
| | | gtaattatggaaatgtaaattctttttttgaaattttagaaatagccaaaaatc |
| | | ctgttatgttaaaaagcctttcaagtgaaaaggtgaactttcaaatgcctgttt |
| | | gatacatagtaattacattttaataatttttaaatataagattataaaacatta |
| | | atattttaatattgcactgatagtcatcattgtaataagtattctaattttttt |
| | | ctgtatcaagccatgacttatacttttaaaaatttcttgtgctttattttggga |
| | | ccatctaaatttgtcagcaaattaaagagtttgagattgggaattgagataaag |
| | | ctatttagttcttttatgtttaaataatttacttcattctgaaatcttataaat |
| | | ggattctcaactttcaagtagtattctccagagaaactgaggggtttatttttc |
| | | ttgtttttactgctaactttcctcctaatagaggatctcaatgataaaagcagt |
| | | tggggagtgggtgctgggtttgtttttgttacttataagaagtatatgacacgt |
| | | gagatgatgttttaaaagaggctaacatagggcatgaaatggaatagacagtat |
| | | atgtagcttgtactccccctaccctttctgttgtctcatctttaactaaaaact |
| | | tttaaaagcacttattgggaaagcctttgtggttctttttgttgttgttgtttt |
| | | tcttttttaagtgaacaggagaagaaaccaaagagaattttgtagaaaatatta |
| | | tgagacaactttattctttaaaaacaaacaaactacattctaggccttcagtac |
| | | ttttatttctttacgaatcaactgaacagtccttacataacttgagtattcaga |
| | | ggaaatgtagtatatatttggatattttactgaagcaagtagtgtatattaaga |
| | | atattccaaactataatagttgggtataacttagtaagcccacatttgtttggc |
| | | attggcaggaggtaaacatcagaatagctttgttttgatgagagttgttctaaa |
| | | ctctaaaggctctatactcccattaaatgctaaaaggcagataaataattccca |
| | | gaaatagcttgaaaatgtgtgcaactctcaggactgaagctgagggccttctgt |
| | | gaaagaggtcgccaggtttagaagttaattataaccttaaatgttcctaaatat |
| | | aaattgttttatggtcttttattccccattaaattagcaccggacagctaagtt |
| | | ttaactacgagtttaactaaatgaatattaaattaatttgaaaagatagagact |
| | | tttcacatttcttagtactaggccatttgaatattaaatagtaaataagagatg |
| | | cttggagttgaatttgtagaccccatgtttttagtagctttcctcatgtttctg |
| | | gcggttttgctttatttctcttactctttcttttctctatagccctagctgtgt |
| | | ctctatctctctttccatatctttctatttctgtcattactcaaagctgtagtt |
| | | tgttgcctcctgtctggttttcattttaatcaaagagtatagtatgtctcttaa |
| | | tagaaagcagttttactgaatttgatttttgctttactttgttgtttcatgggt |
| | | aaattccaaataagtttttatttgattaaagttttccttatttttataattgtt |
| | | ctcttaacctttcttttacttaagagatttttttaaaaaaaaaaagttccagtg |
| | | tccactaatccagaacaatatatttctccttgcaacgttccaaacgagcagagg |
| | | ctgccaatctaccacccagaatttttgtacatgcatacttcttctgatgttaac |
| | | atgttctcagtgtgacctgtactttagttttagataattgtagctgtatgtatc |
| | | taggaaagggagaaagagaagctcaaaataggtacagctcatattgtgttatct |
| | | cttattaatcaggagaagagtctctctctttctctttcttttcttttctccctc |
| | | acccaaccctccctttctccctccctcccttcctcccttctttccttccttctt |
| | | tcttcctatcatcatctaacaagataatgtcctatatttatactctctgtgtta |
| | | aaaggactctctgatgcttccagtctatgtttcagcttctttgggtaaggaagt |
| | | actttcggtcttcttaccatacactttagtaattatcactttcaggttttaaga |
| | | ttctttagattatatctctcttatatatatatatcttggtacaaaaggcatgtt |
| | | aatctgttgccatgaatgtgagtttgtctctccagcttctcacactgcattata |
| | | tgtggtaaattacttttatgtagtaattctttgacatagtccatgacttgctat |
| | | cacaagggtatttgagatttgatagttatatgagaaggtatatgaaattataca |
| | | gaatttaaacatgcagatgaaaacattttaacaaatatttgagtgttttctttt |
| | | gtgttaagcacaaaatatgaggaccacaagggagaataagacgtggtcttttta |
| | | gtcagcctacagttaagttggggagacaaaatatacaaaatataccttaaggga |
| | | taaatattgataaataatttcaaacagtagcatgacaaatgagaaaagacagtt |
| | | gctgtatcttaagatgtagagatcattgtcagggtggacgattggagacgtttt |
| | | gttgaggagaaggaattgaaactatgccttgaaagataaataggaatcgagtca |
| | | gtaagaaagagaatgaattttataaacaagaggaacattataaaaagctaagtg |
| | | tcaagacatgttcatagaaatttagatatttaggaagcttgattgcagtagatt |
| | | ttggaaataatgacgaaaaacaaattgagatgaactgtcagtgacttcaaatga |
| | | tctcataggcataaatacctatgaaccttgtgggggctttagtaaattatttta |
| | | taggatggtcgagaagctaagagaaaaatagtttggttgccagtagtacaattt |
| | | caggtatgaaattgaataagtgtgaggacaactactttaattagtcatgaaata |
| | | ttataggggtagttggtaggaaatttgtaaagaaaaaaatacataaatgacttg |
| | | gtgttttgtgactatattaggatttgggctataggatgacagggaggaaagatt |
| | | taataaatgggtttatagagtgttaagcccgggtgaccttgagaacaatggttt |
| | | tattagtggaaatatgaaagacataagggagaactgacttctttattcagttca |
| | | tttgtaataatgtttattgcgttgaactacctatgtggaaatggccagctggca |
| | | cttggagatagactacttagaggggctaggagtaaaaagaagaaagtgttgaag |
| | | tgtttcttatagtatatagtttactttctcgtggagacaggagaaggaagtgca |
| | | gaaggcttagaagtaaactttgggaaatactcatatatttagcagaatggaaaa |
| | | ggggtcacaatccttaagagggtgtgtcaaggaagacacaccatgctaatatgc |
| | | caaaaagaccaaaagaaaataagcctcaaggaaggcctaatcaatagtattgct |
| | | ctccccagagaaatcataatatgggtacaaaatgatgttaaatttgaagatatt |
| | | tattttggtaatatttttcttttgatgaaaatctagagctgatttttcagaatc |
| | | acttttatataataatttttttttcattttatgtaataatctgatctactgatt |
| | | tttatcaagtcaggtccagcagtttccttttttatgttggcataagttaaagaa |
| | | ttttcaagattaaaacagccatgcaataactattttaaagtaatttgataacac |
| | | ctgaactgttggaatttggatttttttttttttttagacggagtcttgctctgt |
| | | caccaggctggagtgcagtggcacaatctcagctcactgcaacctccgcctect |
| | | ggcacaatctcatctcactgcaacctccgcctcccgggttcaggtgatttcctg |
| | | cctgcctcagcctcccaagtagctgggactacatgcgagegccaccacacccag |
| | | ctaatttttgtgtttttagtagagacagggcttcaccatgttgaccaggatcca |
| | | acacttatttcaaatatacatctgagtcttttttgtatctagtttacatatttt |
| | | tccctaggttggcaagctctaatctaaaagtttttttaatacataaggaaggac |
| | | ttcttaagggttattcttagcataaattccatttatcacctcttttcattcttg |
| | | gtatatgagtccatttcataagattattttagggtattttaaaagttgcataca |
| | | taatttaaaattttggagtatttcctaggtaagatgctttcttctaatgaacgg |
| | | tgtgcttaaaaaaaaaaaaaaaaaaaaaagaacaagacatactgaccctgctaa |
| | | tagaattgaaataactctagaagaggatggttctgcctaagatagcccatattt |
| | | tttaaccttcctactttgtgttaaggttttctagaagcatatacttctcatatt |
| | | cactctctgtatattcttcatgattgatataattgagatcctgaattttagtat |
| | | gcattctgcttttaatatacttcatctccagatttgtccagtttttttcaacat |
| | | acctttcagaacctcagatggtaggattttagaactggattctagtgatcattt |
| | | atctccatcattccacagagattaggtgatttacctattactgatggagagatg |
| | | tcttgggtcttcgacctcattcctctgtctaatcattcatctttctgcttcagg |
| | | aaaattatgttagttgtgtatagtaccttccctcaaaaaaggtattagtattct |
| | | ttccttaatgagtcctcatcagagctcttagctcataagtttttgtatcattta |
| | | atttctttaaagtttagtataaatctctaaatgcctaacaagtttatactactc |
| | | atttattggtagtatttatagaattctttagtcagtatgagaattttcaaataa |
| | | gggagtcattattttaacacatctgatcctcccatctcctccttttaaaaataa |
| | | aacaaaatacaataattgctgaatatacttttattttctttttactcctcttat |
| | | tctttttgaagttttcacagttactggtaaatactgtctcatagattatctcag |
| | | ctaaaccttattgatatttaatgtctatgagctttatgtggtctttatttctat |
| | | atatccaaaagaatgcataacaagcatacttgattttattagaggaatagttca |
| | | tctaaaatgcacttattttgataatttctcattacttattagtttcactctatt |
| | | tctaattattttactacaactgcagtaagtgctagctagcataagctatatgta |
| | | gtattaaattttatgttgacacattttcttgatacgcagtgcatttaattttat |
| | | gataggcttatatacctttcatttggtgagagagggtattttcttttcgttttt |
| | | gaagataaaccttactgctttctagtagatgtgaatattcaaatagtgccttca |
| | | tttaaaattttgtctttgagaattcttactcataaatactttgacccttgttta |
| | | aaaagataaaattcttttcttttgattggcttacttaatcgtaccatacatttt |
| | | aaaaccagttaggctatttttagttaatatagagaaagtaaaatattttcctgt |
| | | tttatatcatcaactgtttggaagcctggtctggaaaacctagtctgcagttta |
| | | tagatatgggtgtttattgtctgaggatctattaataaatagagagttggtctt |
| | | ttaattgatgaagttagaggcaccttttctaactggttcatgagtaaagcagat |
| | | tcttttaatgcctcctttagtaaacaaatggttaatccaaataatatccttttc |
| | | ttttttctgatatgattgaaatatactttcattttctccatataaaagtcactt |
| | | tggagttaaacagatgccttataccaagaaaattaatactgtcttgccatattc |
| | | acaatgaaatgtgttatataggacacacaggcatcttaaatatgattgctattt |
| | | tttctttgctcttttagagctttataagcttcaactgtccccaagattatttca |
| | | gagatctactaactgtaaatgtggcaacattgataaataatagggtcaagggtt |
| | | gatcagtcctacttgccccgcaaccctcacccccaaaaagaaaaaatgtttgtt |
| | | taaacaacacatgaaggataccatgcttaggaacatttatcagatttcctttga |
| | | aattgctaaattggactactttcaagtctccactctacttcaaattgtaatctt |
| | | agacttgtaattatacttctggaaacatttattagagtagatttataaagtagt |
| | | tgcttttttgttctctctcttggcttctgtgaaattttgagtgaactgagttaa |
| | | aaaaaaatcatttaacaacacatggctaaggcagtttcaatcccctaattttta |
| | | gtttttgatataaaagtctatttagaggtgtactttgttcatgtggttgcttat |
| | | ttataacagttcatgtaacttttttttttatttgcctaggaaaacactgaaaaa |
| | | taaagacttgaatcctgaatttgtacttatagtctttcatttttggcaaatgcc |
| | | tagctgtatggctcctggtgcccttatggtaatctggagaaatagttatattct |
| | | ttcttgtttttgaattgttttaaagtagacttggaatcagttaccttcttctcc |
| | | tatcttatcatcctccaactttcttcacactcgagatataatcctgtttcactt |
| | | atgattcatttcctttcttttctaagtttttgtgtatattaaacattagataat |
| | | tttcctagttttatctaatatggttacattttttaaaaagttattctttctttt |
| | | ctattattgtaaccatccaaaagagcattaataagcactgttgcacacatttct |
| | | tctttttagcaagccaaaggaaaaagctagctttctttttgaggcagtcccatt |
| | | tgtggtattgtattaacttcttaggtaaagtcctttcttttatagaaatatttt |
| | | ctctttttaccaaaatgcaaaccttccttgagaaatcagttattcttcttctga |
| | | attttaacttctgcatttggtgaactgtacatccatccttatgccacattttta |
| | | atactaaagaaagaaagttactacctttctcttttccaagcatttcctgggata |
| | | ttactaataaagctcaatttgttctagatcagtggataccggaacagaaaggag |
| | | gtatttcttacctgataatgtgtgtgtgttccgattctgctaatccagaaatcc |
| | | aacagcatttgttgaaaatcatccttagttgttatttgttagggagtcaactcc |
| | | ctattttagttgtttctgaaattcagtccctagatgtgctttagatagggtaga |
| | | atttttacctctttcagaacactgctaaagaagttttcaactggaacatagtct |
| | | gaagaagcactgagagcaaaaaattcagagtggtgattggccacctctgtttga |
| | | ttgacagGTTGCAGGGAGGAAACCCATTCGTTCTGGATTAATGGAACTGGAAAA |
| | | CAGAAAACAATTATCAGgtaaaaaaaaccttttctttaacctttgaaattcacc |
| | | atttaaaagacgagtcagactaccagggaatttattaccaaaaataataaaatc |
| | | tagtgtcatttgagcaatgatctctggctactacttccaagaattaccaaaaaa |
| | | tccgtacagttgctacatgtcagcttttgcagaaagggcatgattgaacggaga |
| | | tagatggagtgtaagacataattatgtgttaaatctgagtttaaaacatgaagc |
| | | agaattctatgggtgggggagttggtattcttttatttcctgaattgtgtttgg |
| | | tctgtggatagaatactttatttttcaaggctattaatctggcacttttcatga |
| | | atattcataaattttattactgtacaatgcttgttttacttaatcttcacttca |
| | | ttaggatgtatgcttttatcttaaaattattgtaataatagtgaccaattcttt |
| | | tcattttaatggtagttttcttactgttggttaaatatatttcag |
| PHIP | 3 | GTTGCAGGGAGGAAACCCATTCGTTCTGGATTAATGGAACTGGAAAACAGAAAA |
| NMD Exon | | CAATTATCAG |
| sequence | | |

TABLE 3
PHIP Protein sequence

| Gene | SEQ ID NO. | Protein Sequence |
|---|---|---|
| PHIP | 162 | MSCERKGLSELRSELYFLIARFLEDGPCQQAAQVLIREVAEKELLPRRTDWTGK |
| | | EHPRTYQNLVKYYRHLAPDHLLQICHRLGPLLEQEIPQSVPGVQTLLGAGRQSL |
| | | LRTNKSCKHVVWKGSALAALHCGRPPESPVNYGSPPSIADTLFSRKLNGKYRLE |
| | | RLVPTAVYQHMKMHKRILGHLSSVYCVTFDRTGRRIFTGSDDCLVKIWATDDGR |
| | | LLATLRGHAAEISDMAVNYENTMIAAGSCDKMIRVWCLRTCAPLAVLQGHSASI |
| | | TSLQFSPLCSGSKRYLSSTGADGTICFWLWDAGTLKINPRPAKFTERPRPGVQM |
| | | ICSSFSAGGMFLATGSTDHIIRVYFFGSGQPEKISELEFHTDKVDSIQFSNTSN |
| | | RFVSGSRDGTARIWQFKRREWKSILLDMATRPAGQNLQGIEDKITKMKVTMVAW |
| | | DRHDNTVITAVNNMTLKVWNSYTGQLIHVLMGHEDEVFVLEPHPFDPRVLESAG |
| | | HDGNVIVWDLARGVKIRSYFNMIEGQGHGAVEDCKCSPDGQHFACTDSHGHLLI |
| | | FGFGSSSKYDKIADQMFFHSDYRPLIRDANNFVLDEQTQQAPHLMPPPFLVDVD |
| | | GNPHPSRYQRLVPGRENCREEQLIPQMGVTSSGLNQVLSQQANQEISPLDSMIQ |
| | | RLQQEQDLRRSGEAVISNTSRLSRGSISSTSEVHSPPNVGLRRSGQIEGVRQMH |
| | | SNAPRSEIATERDLVAWSRRVVVPELSAGVASRQEEWRTAKGEEEIKTYRSEEK |
| | | RKHLTVPKENKIPTVSKNHAHEHELDLGESKKQQTNQHNYRTRSALEETPRPSE |
| | | EIENGSSSSDEGEVVAVSGGTSEEEERAWHSDGSSSDYSSDYSDWTADAGINLQ |
| | | PPKKVPKNKTKKAESSSDEEEESEKQKQKQIKKEKKKVNEEKDGPISPKKKKPK |
| | | ERKQKRLAVGELTENGLTLEEWLPSTWITDTIPRRCPFVPQMGDEVYYFRQGHE |
| | | AYVEMARKNKIYSINPKKQPWHKMELREQELMKIVGIKYEVGLPTLCCLKLAFL |
| | | DPDTGKLTGGSFTMKYHDMPDVIDFLVLRQQFDDAKYRRWNIGDRERSVIDDAW |
| | | WEGTIESQEPLQLEYPDSLFQCYNVCWDNGDTEKMSPWDMELIPNNAVFPEELG |
| | | TSVPLTDGECRSLIYKPLDGEWGTNPRDEECERIVAGINQLMTLDIASAFVAPV |
| | | DLQAYPMYCTVVAYPTDLSTIKQRLENRFYRRVSSLMWEVRYIEHNTRTFNEPG |
| | | SPIVKSAKFVTDLLLHFIKDQTCYNIIPLYNSMKKKVLSDSEDEEKDADVPGTS |
| | | TRKRKDHQPRRRLRNRAQSYDIQAWKKQCEELLNLIFQCEDSEPFRQPVDLLEY |
| | | PDYRDIIDTPMDFATVRETLEAGNYESPMELCKDVRLIFSNSKAYTPSKRSRIY |
| | | SMSLRLSAFFEEHISSVLSDYKSALRFHKRNTITKRRKKRNRSSSVSSSAASSP |
| | | ERKKRILKPQLKSESSTSAFSTPTRSIPPRHNAAQINGKTESSSVVRTRSNRVV |
| | | VDPVVTEQPSTSSAAKTFITKANASAIPGKTILENSVKHSKALNTLSSPGQSSF |
| | | SHGTRNNSAKENMEKEKPVKRKMKSSVLPKASTLSKSSAVIEQGDCKNNALVPG |
| | | TIQVNGHGGQPSKLVKRGPGRKPKVEVNTNSGEIIHKKRGRKPKKLQYAKPEDL |
| | | EQNNVHPIRDEVLPSSTCNFLSETNNVKEDLLQKKNRGGRKPKRKMKTQKLDAD |
| | | LLVPASVKVLRRSNRKKIDDPIDEEEEFEELKGSEPHMRTRNQGRRTAFYNEDD |
| | | SEEEQRQLLFEDTSLTFGTSSRGRVRKLTEKAKANLIGW |

### Example 2: Confirmation of NMD Exon Via Cycloheximide/Puromycin/DMSO Treatment

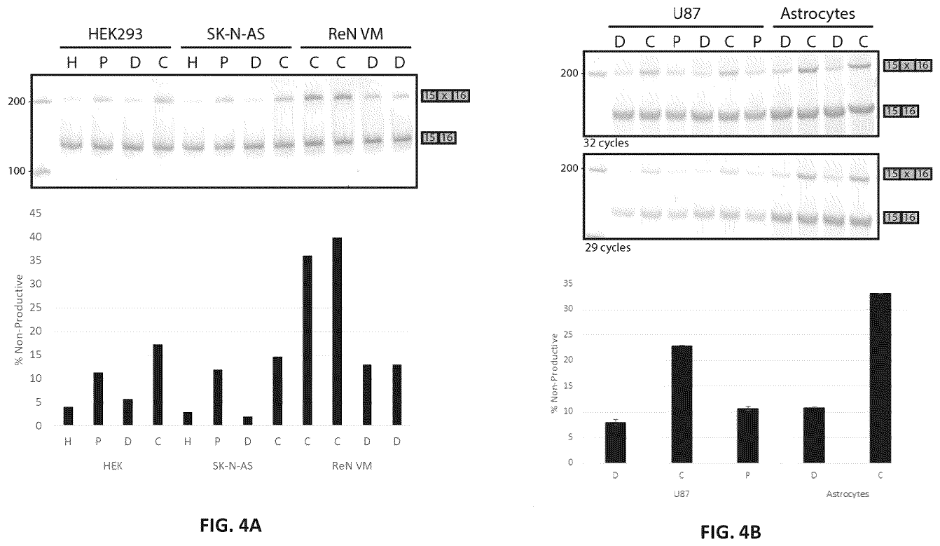

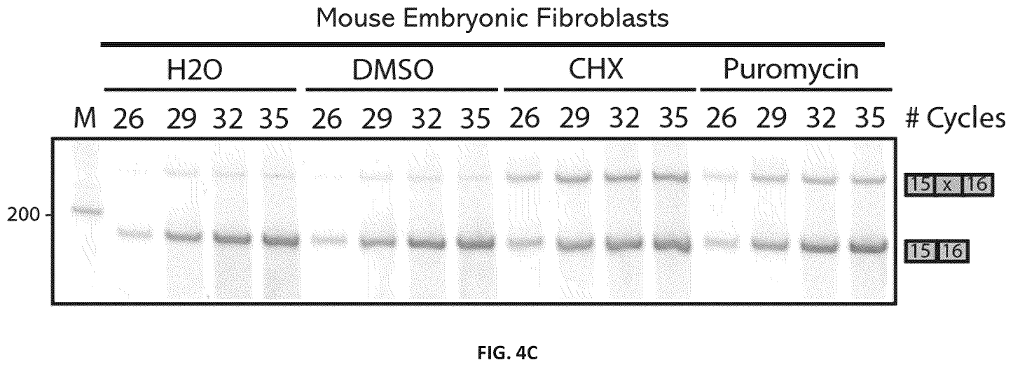

RT-PCR analysis using total RNA from water (H) DMSO-treated (D) or puromycin (P) or cycloheximide-treated (C) human embryonic kidney cells (HEK293), human neuroblasts (SK-N-AS), and human neural progenitor cells (ReN) cells and primers in exon 15 and 16 of the PHIP gene were used to confirm the presence of a band corresponding to an NMD-inducing exon. Densitometry analysis of the bands was performed to calculate percent NMD exon inclusion of total transcript. Treatment of cells with cycloheximide or puromycin to inhibit NMD can lead to an increase of the product corresponding to the NMD-inducing exon. FIG. 4A depicts confirmation of exemplary NMD exons in PHIP gene transcripts using DMSO, cycloheximide or puromycin treatment, respectively in HEK, SK-N-AS and ReN VM cells. U87 cells and Astrocytes were similarly treated with cycloheximide (C), puromycin (P) or DMSO (D). Primers in exon 15 and 16 of the PHIP gene were used to confirm the presence of a band corresponding to an NMD-inducing exon. Results for U87 cells and astrocytes are shown in FIG. 4B.

Similar results were found in mouse embryonic fibroblast (MEF) cells treated with water (H2O), DMSO, cycloheximide (CHX) and puromycin. Four different amplification cycles (26, 29, 32 and 35) are shown. NMD exon 15x inclusion was found to be conserved in MEF cells as is shown in FIG. 4C.

### Example 3: PHIP NMD Event is Conserved in Non-Human Primates

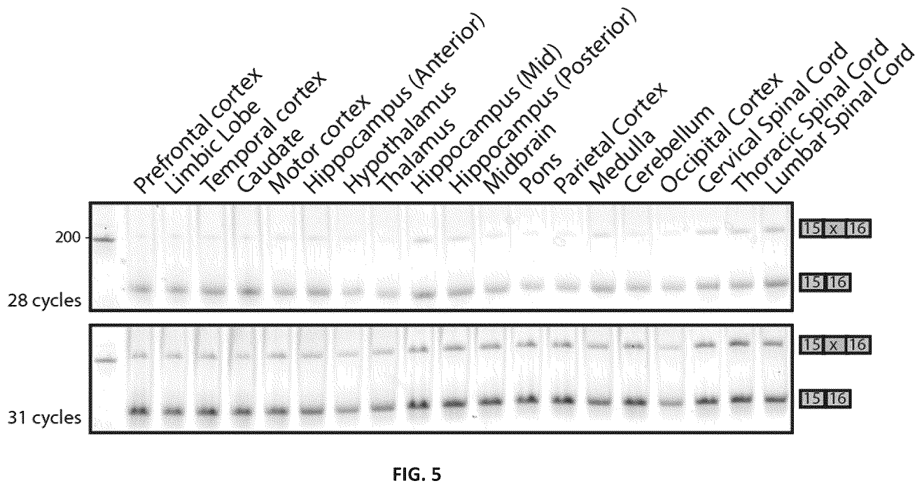

RNA was extracted from the indicated brain regions of a cynomolgus monkey (Macaca fascicularis). cDNA was generated and the products of interest were amplified by PCR using the forward primer in exon 15 and the reverse primer in exon 16. Two different amplification cycles (28 and 31) are shown in FIG. 5.

### Example 4: NMD Exon Region ASO Walk

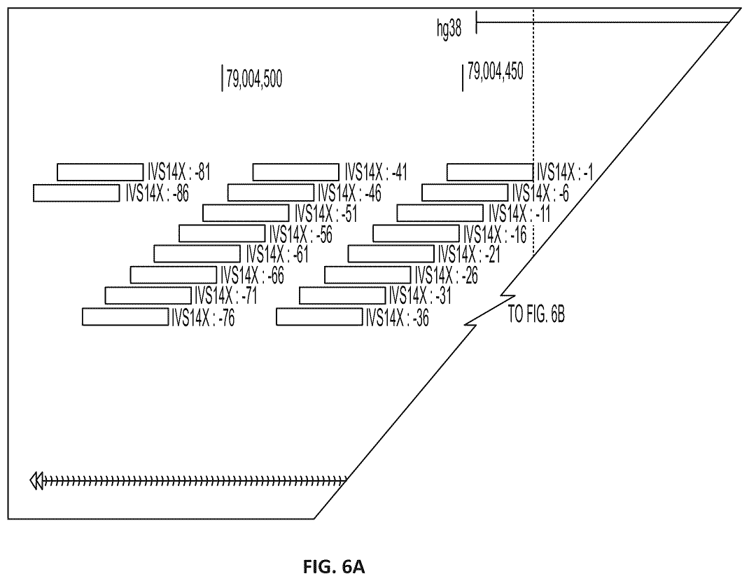

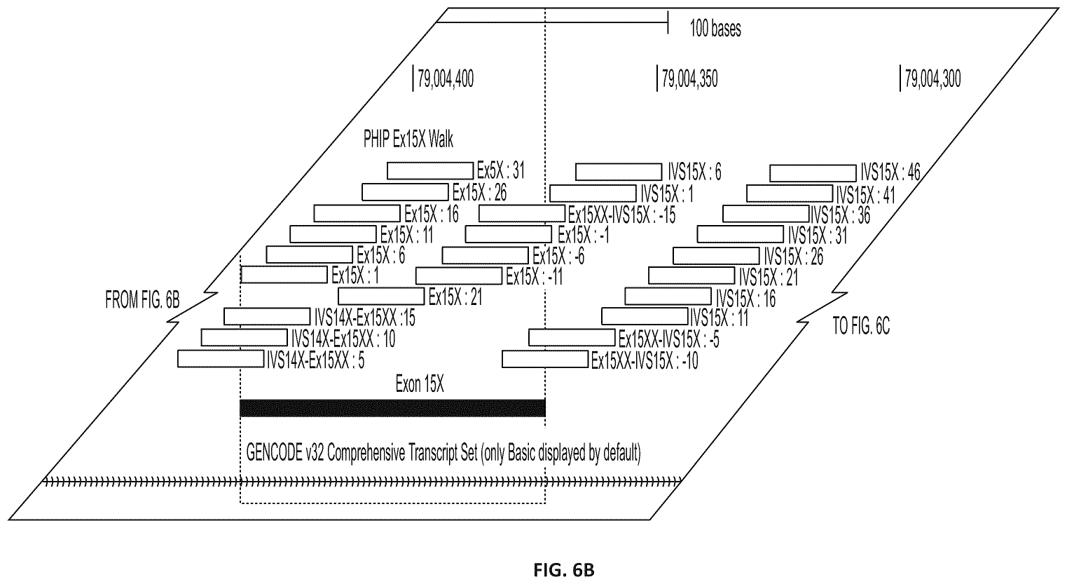

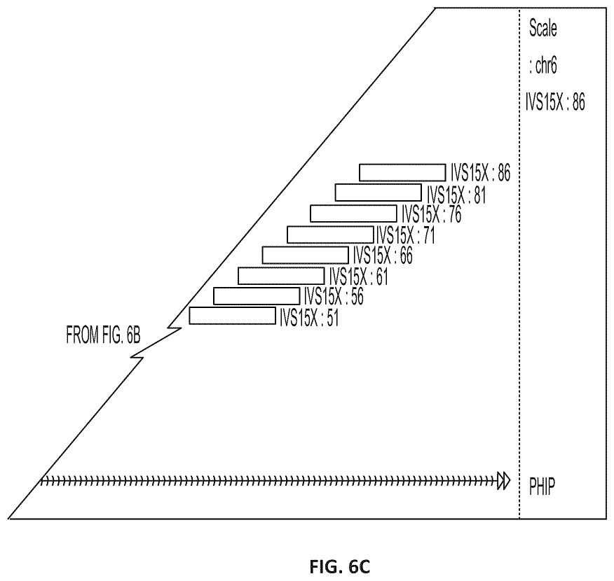

An ASO walk was performed for NMD exon region targeting sequences immediately upstream of the 3′ splice site, across the 3′splice site, the NMD exon, across the 5′ splice site, and downstream of the 5′ splice site using 2′-MOE ASOs, PS backbone. ASOs were designed to cover these regions by shifting 5 nucleotides at a time, except for the last 3′ splice site ASO and the first exon ASO, and the last exon ASO and the first 5′ splice site ASO, which were 3 nucleotides apart. FIGS. 6A-C depict an ASO walk for an exemplary PHIP NMD exon region.

The labeling of ASOs can be explained as per the following: IVS14X=intronic sequence portion of the canonical intron (canonical intron 15) that is after the canonical exon (canonical exon 15) immediately preceding the NMD exon. Ex15X=NMD exon (within canonical intron 15). IVS15X=intronic sequence portion of the canonical intron (canonical intron 15) that is immediately after the NMD exon and before the canonical exon (canonical exon 16) immediately after the NMD exon. For the ASO walk, see nomenclature above. Note that ASOs spanning IVS14X and Ex15X or spanning Ex15X and IVS15X, are labeled as “XX”. ASOs that are entirely within Ex15X are not labeled as “XX”.

### Example 5: NMD Exon Region ASO Walk Evaluated by RT-PCR

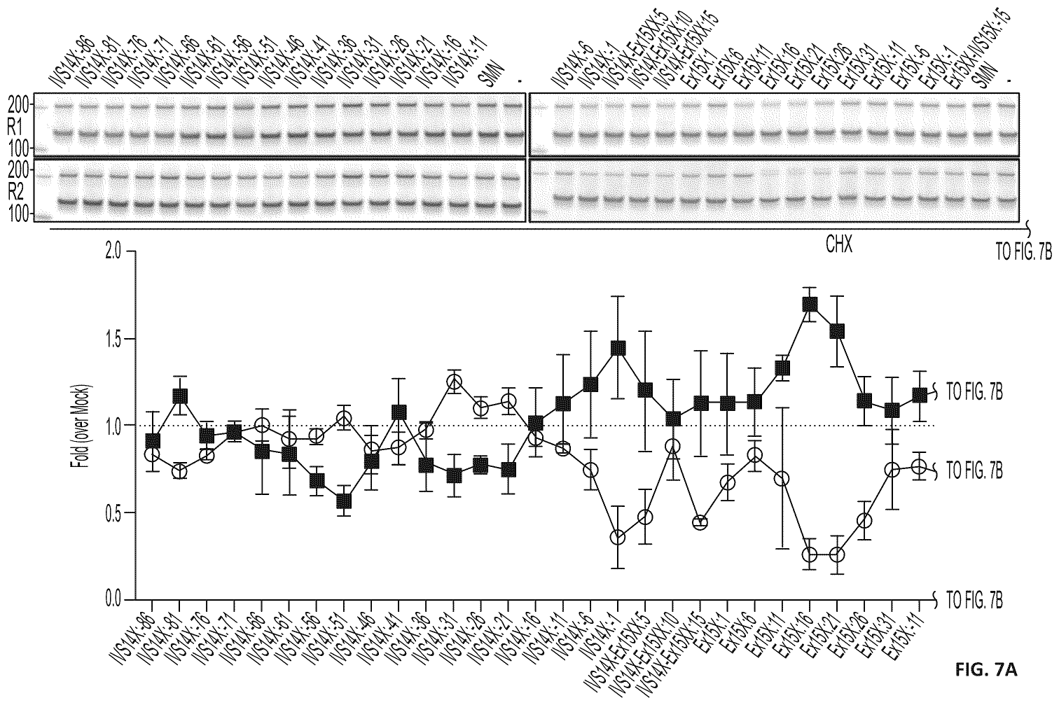

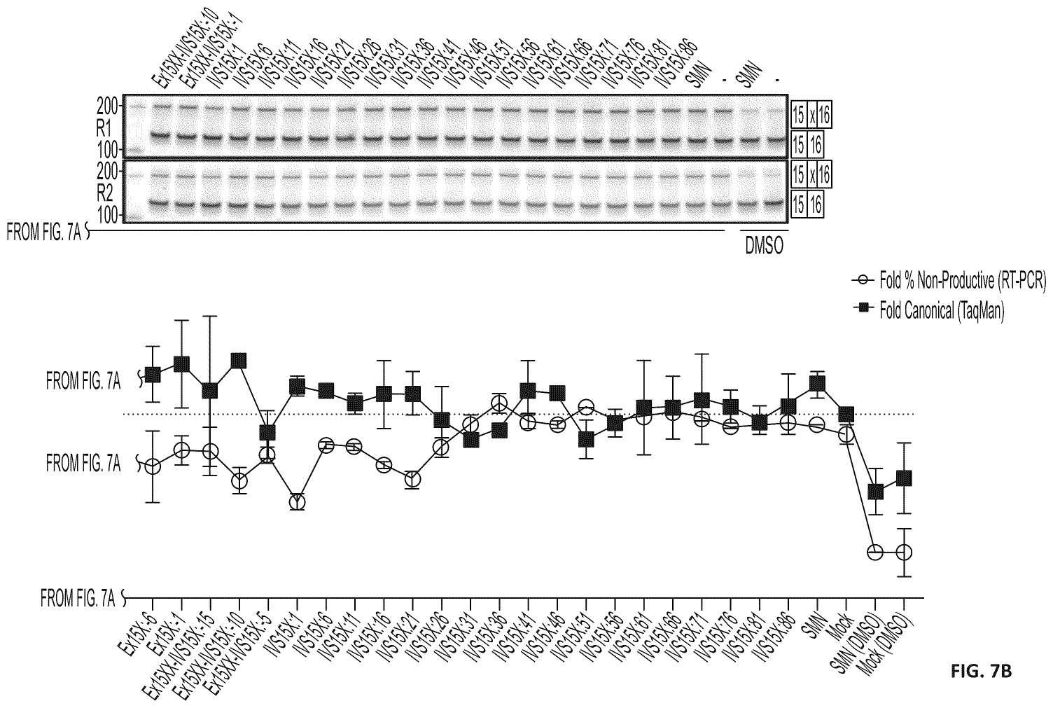

ASO walk sequences were evaluated by RT-PCR and TaqMan qPCR. HEK293 cells were transfected using Lipofectamine RNAiMax or ReN cells were nucleofected with control ASO treated (“Mock” or “-”), or with a 2′-MOE ASO targeting the PHIP NMD exon regions as shown in Table 4 below. Products corresponding to PHIP non-productive mRNA (RT-PCR) and productive mRNA (TaqMan) were quantified and normalized to internal control, and fold-change relative to control was plotted. FIGS. 7A-B depicts evaluation via RT-PCR and TaqMan qPCR of various exemplary ASO walk along exemplary NMD exon regions in ReN cells. The measurement of the amount of PHIP mRNAs was carried out with the cells 24 hours after treatment with 3 uM of exemplary ASOs after a 3 hour cycloheximide treatment, by Taqman qPCR using probes spanning exon 15 and exon 16 and RT-PCR using primers in exon 15 and exon 16.

TABLE 4
Exemplary ASOs

| SEQ ID | | | | | |
|---|---|---|---|---|---|
| NO: | Chr | Start | End | ASO Name | Sequence |
| 4 | chr6 | 79004521 | 79004539 | IVS14X:-86 | CTTTAGCAGTGTTCTGAA |
| 5 | chr6 | 79004516 | 79004534 | IVS14X:-81 | AACTTCTTTAGCAGTGTT |
| 6 | chr6 | 79004511 | 79004529 | IVS14X:-76 | TTGAAAACTTCTTTAGCA |
| 7 | chr6 | 79004506 | 79004524 | IVS14X:-71 | TCCAGTTGAAAACTTCTT |
| 8 | chr6 | 79004501 | 79004519 | IVS14X:-66 | TATGTTCCAGTTGAAAAC |
| 9 | chr6 | 79004496 | 79004514 | IVS14X:-61 | CAGACTATGTTCCAGTTG |
| 10 | chr6 | 79004491 | 79004509 | IVS14X:-56 | TTCTTCAGACTATGTTCC |
| 11 | chr6 | 79004486 | 79004504 | IVS14X:-51 | AGTGCTTCTTCAGACTAT |
| 12 | chr6 | 79004481 | 79004499 | IVS14X:-46 | CTCTCAGTGCTTCTTCAG |
| 13 | chr6 | 79004476 | 79004494 | IVS14X:-41 | TTTTGCTCTCAGTGCTTC |
| 14 | chr6 | 79004471 | 79004489 | IVS14X:-36 | GAATTTTTTGCTCTCAGT |
| 15 | chr6 | 79004466 | 79004484 | IVS14X:-31 | ACTCTGAATTTTTTGCTC |
| 16 | chr6 | 79004461 | 79004479 | IVS14X:-26 | TCACCACTCTGAATTTTT |
| 17 | chr6 | 79004456 | 79004474 | IVS14X:-21 | GCCAATCACCACTCTGAA |
| 18 | chr6 | 79004451 | 79004469 | IVS14X:-16 | AGGTGGCCAATCACCACT |
| 19 | chr6 | 79004446 | 79004464 | IVS14X:-11 | AACAGAGGTGGCCAATCA |
| 20 | chr6 | 79004441 | 79004459 | IVS14X:-6 | AATCAAACAGAGGTGGCC |
| 21 | chr6 | 79004436 | 79004454 | IVS14X:-1 | CTGTCAATCAAACAGAGG |
| 22 | chr6 | 79004431 | 79004449 | IVS14X-Ex15XX:5 | GCAACCTGTCAATCAAAC |
| 23 | chr6 | 79004426 | 79004444 | IVS14X-Ex15XX:10 | TCCCTGCAACCTGTCAAT |
| 24 | chr6 | 79004421 | 79004439 | IVS14X-Ex15XX:15 | TTTCCTCCCTGCAACCTG |
| 25 | chr6 | 79004418 | 79004436 | Ex15X:1 | GGGTTTCCTCCCTGCAAC |
| 26 | chr6 | 79004413 | 79004431 | Ex15X:6 | CGAATGGGTTTCCTCCCT |
| 27 | chr6 | 79004408 | 79004426 | Ex15X:11 | CAGAACGAATGGGTTTCC |
| 28 | chr6 | 79004403 | 79004421 | Ex15X:16 | TAATCCAGAACGAATGGG |
| 29 | chr6 | 79004398 | 79004416 | Ex15X:21 | TCCATTAATCCAGAACGA |
| 30 | chr6 | 79004393 | 79004411 | Ex15X:31 | GTTTTCCAGTTCCATTAA |
| 31 | chr6 | 79004388 | 79004406 | Ex15X:-11 | TTTTCTGTTTTCCAGTTC |
| 32 | chr6 | 79004382 | 79004400 | Ex15X:-6 | AATTGTTTTCTGTTTTCC |
| 33 | chr6 | 79004377 | 79004395 | Ex15X:-1 | CTGATAATTGTTTTCTGT |
| 34 | chr6 | 79004372 | 79004390 | Ex15XX-IVS15X:-15 | TACCTGATAATTGTTTTC |
| 35 | chr6 | 79004369 | 79004387 | Ex15XX-IVS15X:-10 | TTTTTTACCTGATAATTG |
| 36 | chr6 | 79004364 | 79004382 | Ex15XX-IVS15X:-5 | AGGTTTTTTTTACCTGAT |
| 37 | chr6 | 79004359 | 79004377 | IVS15X:1 | AGAAAAGGTTTTTTTTAC |
| 38 | chr6 | 79004354 | 79004372 | IVS15X:6 | GTTAAAGAAAAGGTTTTT |
| 39 | chr6 | 79004349 | 79004367 | IVS15X:11 | CAAAGGTTAAAGAAAAGG |
| 40 | chr6 | 79004344 | 79004362 | IVS15X:16 | AATTTCAAAGGTTAAAGA |
| 41 | chr6 | 79004339 | 79004357 | IVS15X:21 | TGGTGAATTTCAAAGGTT |
| 42 | chr6 | 79004334 | 79004352 | IVS15X:26 | TTAAATGGTGAATTTCAA |
| 43 | chr6 | 79004329 | 79004347 | IVS15X:31 | GTCTTTTAAATGGTGAAT |
| 44 | chr6 | 79004324 | 79004342 | IVS15X:36 | GACTCGTCTTTTAAATGG |
| 45 | chr6 | 79004319 | 79004337 | IVS15X:41 | AGTCTGACTCGTCTTTTA |
| 46 | chr6 | 79004314 | 79004332 | IVS15X:46 | CTGGTAGTCTGACTCGTC |
| 47 | chr6 | 79004309 | 79004327 | IVS15X:51 | ATTCCCTGGTAGTCTGAC |
| 48 | chr6 | 79004304 | 79004322 | IVS15X:56 | AATAAATTCCCTGGTAGT |
| 49 | chr6 | 79004299 | 79004317 | IVS15X:61 | TTGGTAATAAATTCCCTG |
| 50 | chr6 | 79004294 | 79004312 | IVS15X:66 | TATTTTTGGTAATAAATT |
| 51 | chr6 | 79004289 | 79004307 | IVS15X:71 | TTTATTATTTTTGGTAAT |
| 52 | chr6 | 79004284 | 79004302 | IVS15X:76 | TAGATTTTATTATTTTTG |
| 53 | chr6 | 79004279 | 79004297 | IVS15X:81 | GACACTAGATTTTATTAT |
| 54 | chr6 | 79004274 | 79004292 | IVS15X:86 | CAAATGACACTAGATTTT |

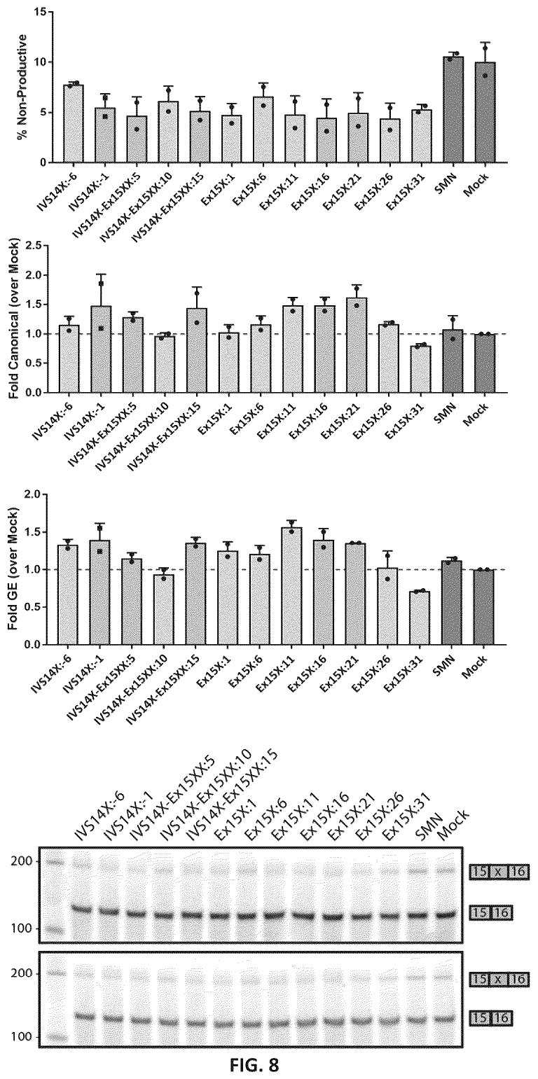

FIG. 8 depicts the measurement of the amount of PHIP mRNA. The analysis was carried out with ReN cells 24 hours after treatment with 3 uM of a few exemplary ASOs in the absence of cycloheximide treatment, by Taqman qPCR using probes spanning exon 15 and exon 16 (canonical) and exon 35 and exon 36 (GE). Various ASOs increased the production of canonical and total (GE) PHIP mRNA and decreased the amount of non-productive mRNA produced.

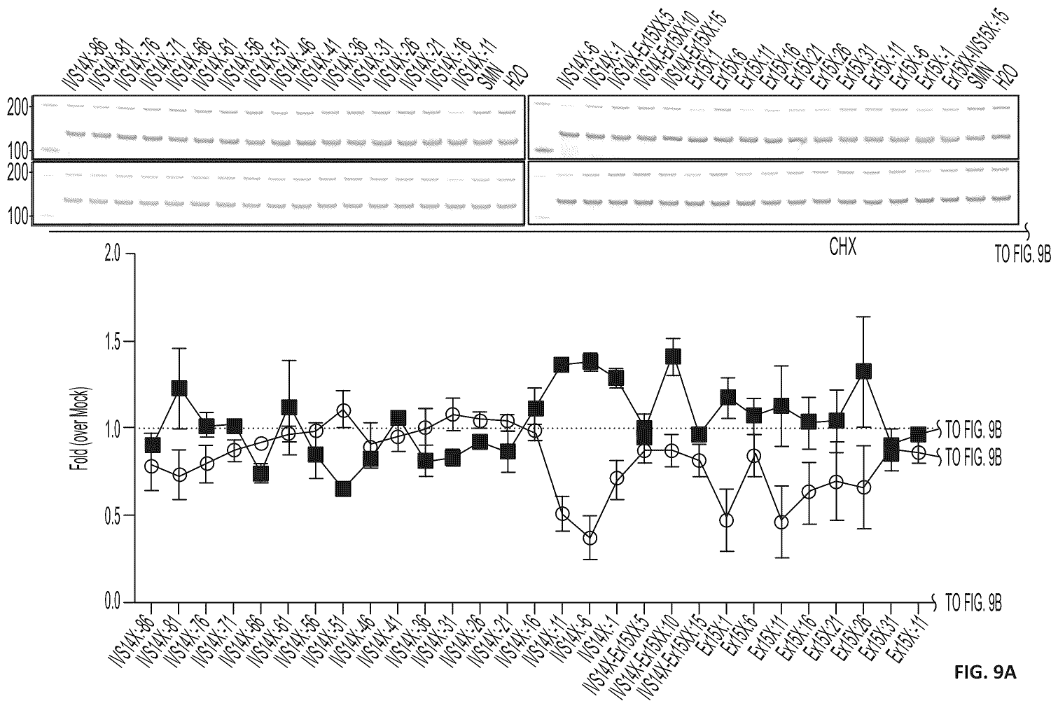

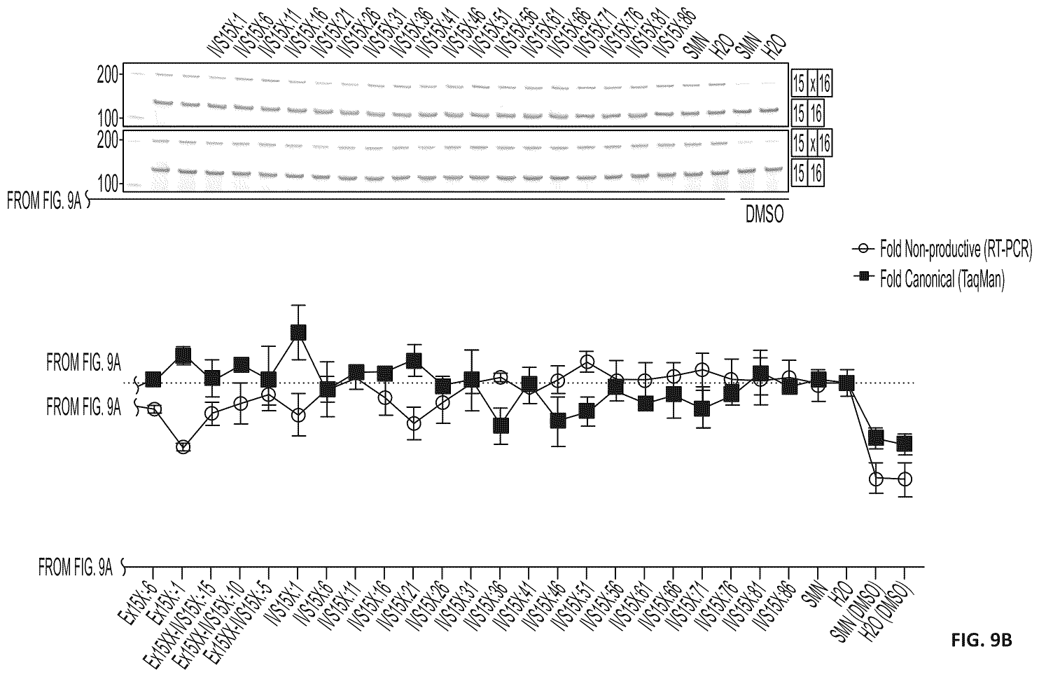

FIGS. 9A-B depict evaluation via RT-PCR and TaqMan qPCR of various exemplary ASO walk along exemplary NMD exon regions in HEK293 cells. The measurement of the amount of PHIP mRNA was carried out with the cells 24 hours after treatment with 120 nM of exemplary ASOs after a 3 hour cycloheximide treatment, by Taqman qPCR using probes spanning exon 15 and exon 16 and by RT-PCR with primers in exon 15 and exon 16.

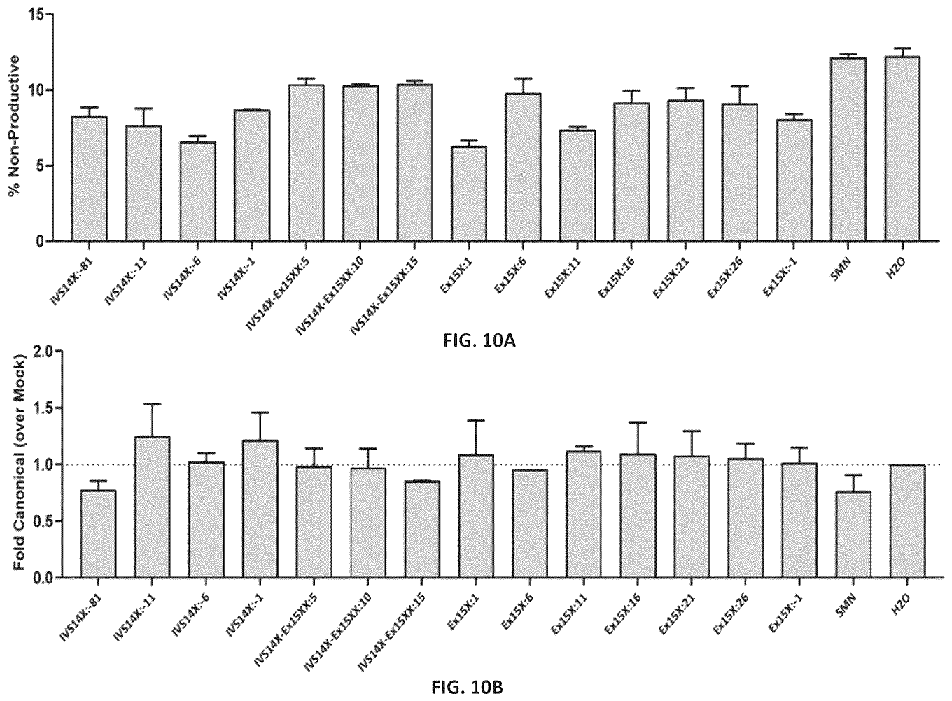

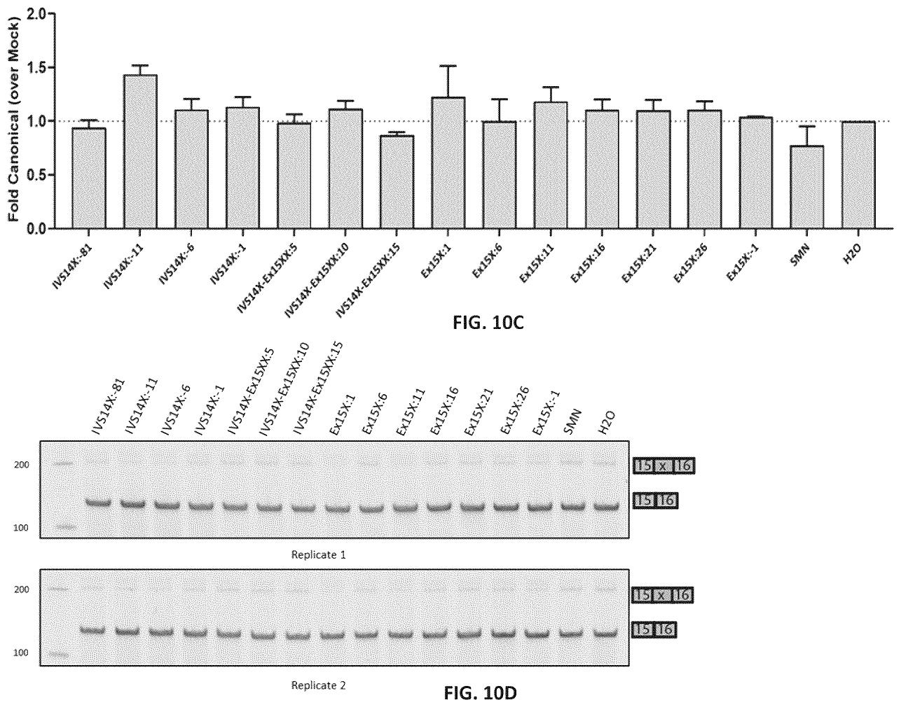

FIGS. 10A-10D depict evaluation of a few exemplary ASOs via RT-PCR and TaqMan qPCR along exemplary NMD exon region 15x. The measurement of the amount of PHIP mRNA was carried out with the cells 24 hours after treatment with 80 nM of an exemplary ASO in the absence of cycloheximide. As shown in the figure, various ASOs increased the production of canonical PHIP mRNA and of total PHIP mRNA, and decreased the amount of non-productive mRNA produced.

### Example 6: NMD Exon Region ASO Microwalk Evaluated by RT-PCR and RT-qPCR

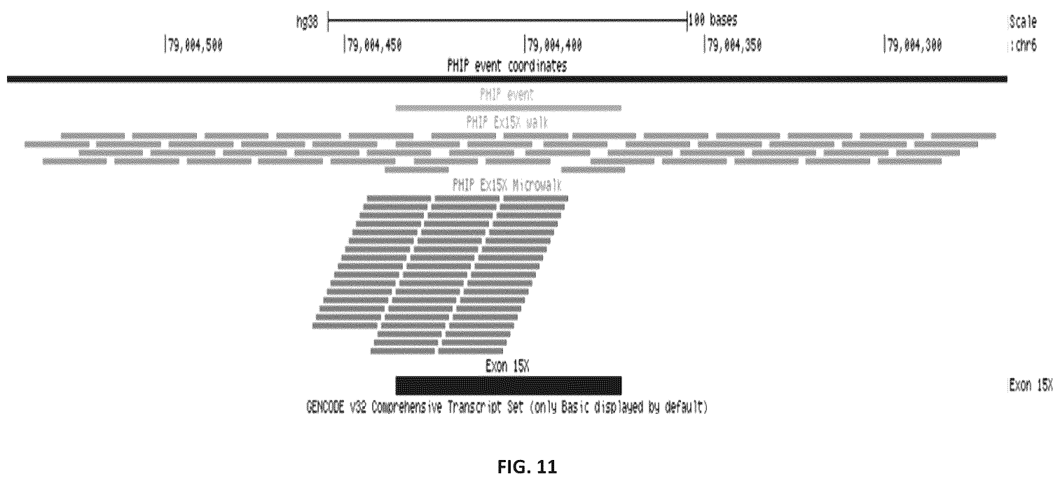

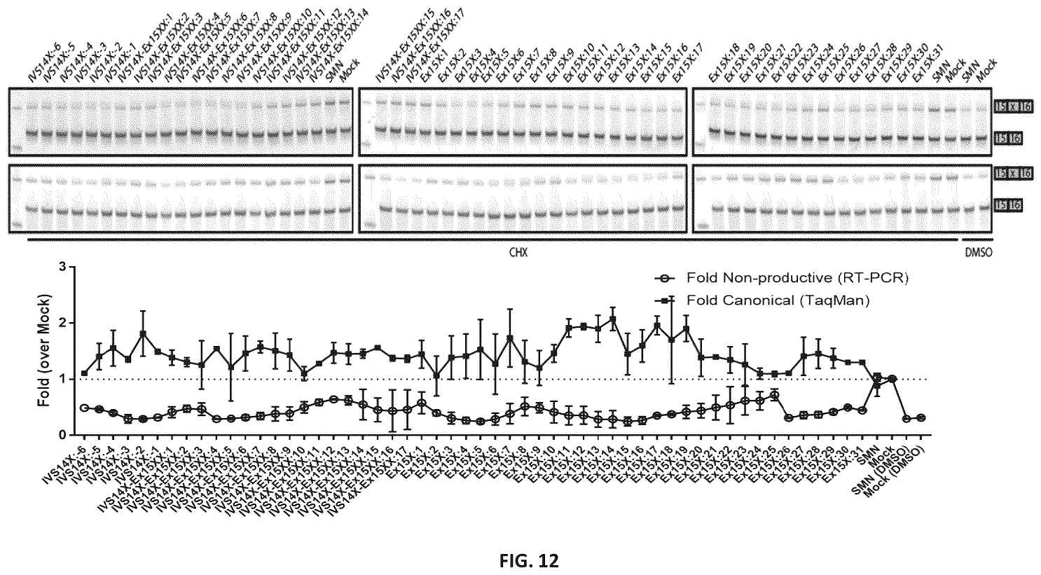

FIG. 11 depicts an ASO microwalk for an exemplary PHIP NMD exon region. ASO microwalk sequences (across exon 15x) were evaluated by RT-PCR. Cells were transfected with control ASO treated (SMN), or with a 2′-MOE ASO targeting the PHIP NMD exon regions as described in Table 5 below. Products corresponding to NMD exon inclusion and full-length were quantified and percent NMD exon inclusion was plotted. FIG. 12 depicts evaluation of various exemplary ASO walk along exemplary NMD exon regions in ReN cells. The measurement of the amount of PHIP mRNA was carried out with ReN cells 24 hours after transfection with 3 uM of an exemplary ASO after a 3-hour cycloheximide treatment, by Taqman qPCR using probes spanning exon 15 and exon 16 (FIG. 12).

TABLE 5
Exemplary ASO sequences

| SEQ ID | | | | | |
|---|---|---|---|---|---|
| NO: | Chr | Start | End | ASO Name | Sequence |
| 55 | chr6 | 79004441 | 79004459 | IVS14X:-6 | AATCAAACAGAGGTGGCC |
| 56 | chr6 | 79004440 | 79004458 | IVS14X:-5 | CAATCAAACAGAGGTGGC |
| 57 | chr6 | 79004439 | 79004457 | IVS14X:-4 | TCAATCAAACAGAGGTGG |
| 58 | chr6 | 79004438 | 79004456 | IVS14X:-3 | GTCAATCAAACAGAGGTG |
| 59 | chr6 | 79004437 | 79004455 | IVS14X:-2 | TGTCAATCAAACAGAGGT |
| 60 | chr6 | 79004436 | 79004454 | IVS14X:-1 | CTGTCAATCAAACAGAGG |
| 61 | chr6 | 79004435 | 79004453 | IVS14X-Ex15XX:1 | CCTGTCAATCAAACAGAG |
| 62 | chr6 | 79004434 | 79004452 | IVS14X-Ex15XX:2 | ACCTGTCAATCAAACAGA |
| 63 | chr6 | 79004433 | 79004451 | IVS14X-Ex15XX:3 | AACCTGTCAATCAAACAG |
| 64 | chr6 | 79004432 | 79004450 | IVS14X-Ex15XX:4 | CAACCTGTCAATCAAACA |
| 65 | chr6 | 79004431 | 79004449 | IVS14X-Ex15XX:5 | GCAACCTGTCAATCAAAC |
| 66 | chr6 | 79004430 | 79004448 | IVS14X-Ex15XX:6 | TGCAACCTGTCAATCAAA |
| 67 | chr6 | 79004429 | 79004447 | IVS14X-Ex15XX:7 | CTGCAACCTGTCAATCAA |
| 68 | chr6 | 79004428 | 79004446 | IVS14X-Ex15XX:8 | CCTGCAACCTGTCAATCA |
| 69 | chr6 | 79004427 | 79004445 | IVS14X-Ex15XX:9 | CCCTGCAACCTGTCAATC |
| 70 | chr6 | 79004426 | 79004444 | IVS14X-Ex15XX:10 | TCCCTGCAACCTGTCAAT |
| 71 | chr6 | 79004425 | 79004443 | IVS14X-Ex15XX:11 | CTCCCTGCAACCTGTCAA |
| 72 | chr6 | 79004424 | 79004442 | IVS14X-Ex15XX:12 | CCTCCCTGCAACCTGTCA |
| 73 | chr6 | 79004423 | 79004441 | IVS14X-Ex15XX:13 | TCCTCCCTGCAACCTGTC |
| 74 | chr6 | 79004422 | 79004440 | IVS14X-Ex15XX:14 | TTCCTCCCTGCAACCTGT |
| 75 | chr6 | 79004421 | 79004439 | IVS14X-Ex15XX:15 | TTTCCTCCCTGCAACCTG |
| 76 | chr6 | 79004420 | 79004438 | IVS14X-Ex15XX:16 | GTTTCCTCCCTGCAACCT |
| 77 | chr6 | 79004419 | 79004437 | IVS14X-Ex15XX:17 | GGTTTCCTCCCTGCAACC |
| 78 | chr6 | 79004418 | 79004436 | Ex15X:1 | GGGTTTCCTCCCTGCAAC |
| 79 | chr6 | 79004417 | 79004435 | Ex15X:2 | TGGGTTTCCTCCCTGCAA |
| 80 | chr6 | 79004416 | 79004434 | Ex15X:3 | ATGGGTTTCCTCCCTGCA |
| 81 | chr6 | 79004415 | 79004433 | Ex15X:5 | GAATGGGTTTCCTCCCTG |
| 82 | chr6 | 79004414 | 79004432 | Ex15X:6 | CGAATGGGTTTCCTCCCT |
| 83 | chr6 | 79004413 | 79004431 | Ex15X:7 | ACGAATGGGTTTCCTCCC |
| 84 | chr6 | 79004412 | 79004430 | Ex15X:8 | AACGAATGGGTTTCCTCC |
| 85 | chr6 | 79004411 | 79004429 | Ex15X:9 | GAACGAATGGGTTTCCTC |
| 86 | chr6 | 79004410 | 79004428 | Ex15X:10 | AGAACGAATGGGTTTCCT |
| 87 | chr6 | 79004409 | 79004427 | Ex15X:11 | CAGAACGAATGGGTTTCC |
| 88 | chr6 | 79004408 | 79004426 | Ex15X:12 | CCAGAACGAATGGGTTTC |
| 89 | chr6 | 79004407 | 79004425 | Ex15X:13 | TCCAGAACGAATGGGTTT |
| 90 | chr6 | 79004406 | 79004424 | Ex15X:14 | ATCCAGAACGAATGGGTT |
| 91 | chr6 | 79004405 | 79004423 | Ex15X:15 | AATCCAGAACGAATGGGT |
| 92 | chr6 | 79004404 | 79004422 | Ex15X:16 | TAATCCAGAACGAATGGG |
| 93 | chr6 | 79004403 | 79004421 | Ex15X:17 | TTAATCCAGAACGAATGG |
| 94 | chr6 | 79004402 | 79004420 | Ex15X:18 | ATTAATCCAGAACGAATG |
| 95 | chr6 | 79004401 | 79004419 | Ex15X:19 | CATTAATCCAGAACGAAT |
| 96 | chr6 | 79004400 | 79004418 | Ex15X:20 | CCATTAATCCAGAACGAA |
| 97 | chr6 | 79004399 | 79004417 | Ex15X:21 | TCCATTAATCCAGAACGA |
| 98 | chr6 | 79004398 | 79004416 | Ex15X:22 | TTCCATTAATCCAGAACG |
| 99 | chr6 | 79004397 | 79004415 | Ex15X:23 | GTTCCATTAATCCAGAAC |
| 100 | chr6 | 79004396 | 79004414 | Ex15X:24 | AGTTCCATTAATCCAGAA |
| 101 | chr6 | 79004395 | 79004413 | Ex15X:25 | CAGTTCCATTAATCCAGA |
| 102 | chr6 | 79004394 | 79004412 | Ex15X:26 | CCAGTTCCATTAATCCAG |
| 103 | chr6 | 79004393 | 79004411 | Ex15X:27 | TCCAGTTCCATTAATCCA |
| 104 | chr6 | 79004392 | 79004410 | Ex15X:28 | TTCCAGTTCCATTAATCC |
| 105 | chr6 | 79004391 | 79004409 | Ex15X:29 | TTTCCAGTTCCATTAATC |
| 106 | chr6 | 79004390 | 79004408 | Ex15X:30 | TTTTCCAGTTCCATTAAT |
| 107 | chr6 | 79004389 | 79004407 | Ex15X:31 | GTTTTCCAGTTCCATTAA |

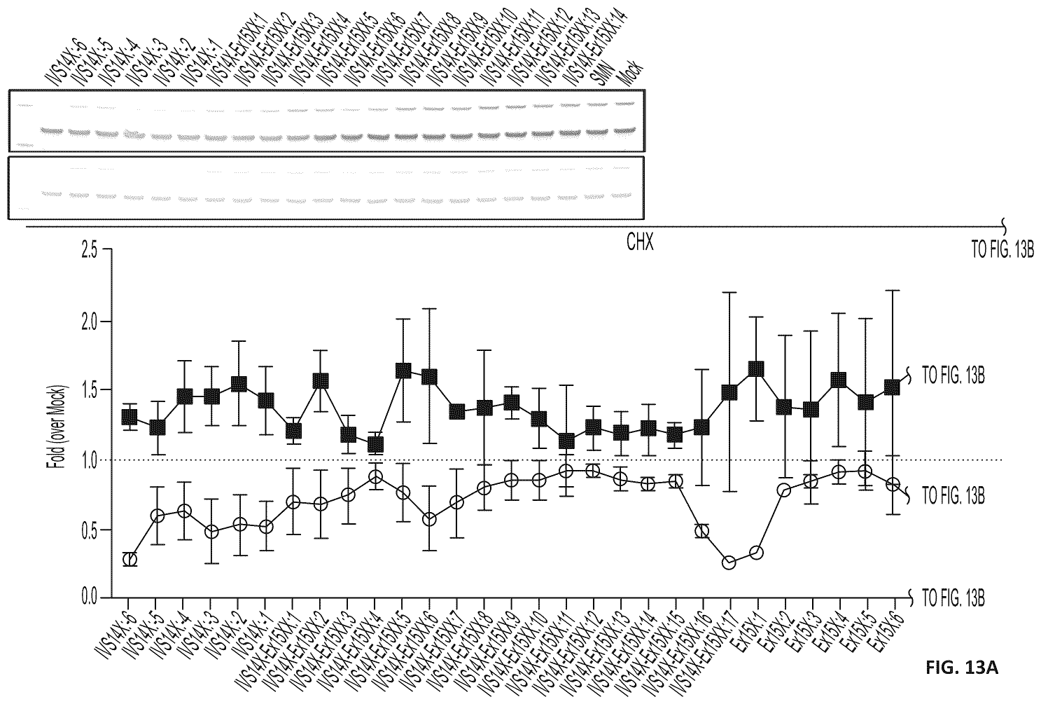

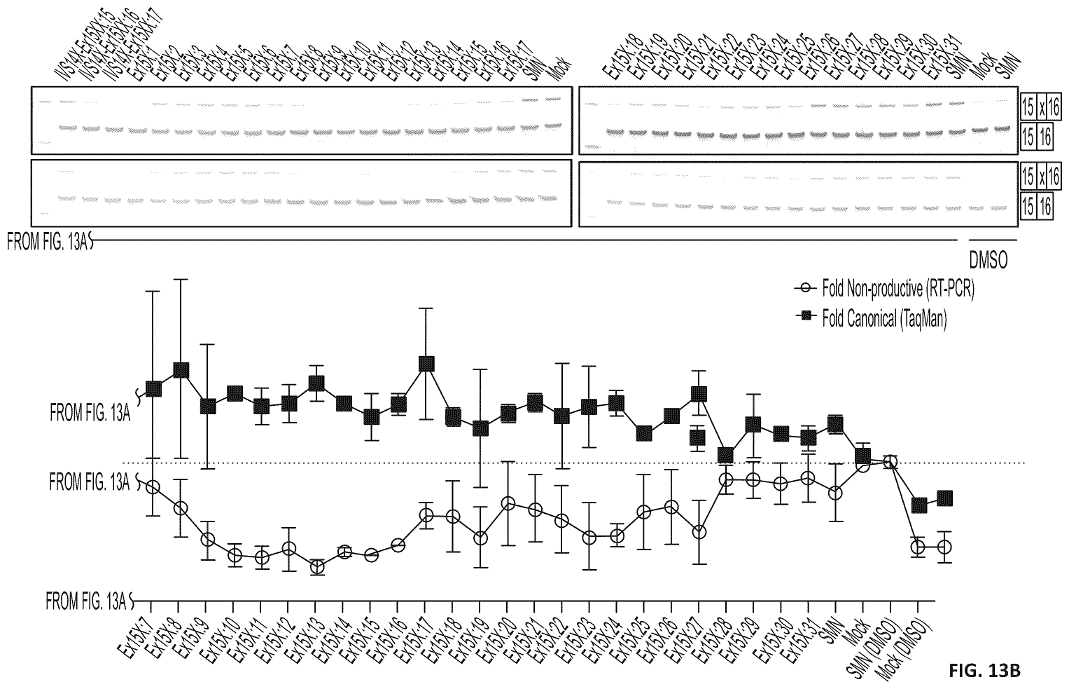

FIGS. 13A-B depicts evaluation of various exemplary ASO walk along exemplary NMD exon regions in HEK293 cells. The measurement of the amount of PHIP mRNA was carried out with HEK293 cells 24 hours after transfection with 120 nM of an exemplary ASO after a 3-hour treatment with cycloheximide, by Taqman qPCR using probes spanning exon 15 and exon 16, and by RT-PCR using primers in exon 15 and exon 16 (FIG. 10).

Table 6 below provides additional exemplary PHIP NMD exon region ASO microwalk sequences (across exon 15x).

TABLE 6
Exemplary ASO sequences

| SEQ ID | | | | | |
|---|---|---|---|---|---|
| NO: | Chr | Start | End | Name | ASO Sequence |
| 108 | chr6 | 79004441 | 79004459 | IVS14X:-6 | AATCAAACAGAGGTGGCC |
| 109 | chr6 | 79004440 | 79004458 | IVS14X:-5 | CAATCAAACAGAGGTGGC |
| 110 | chr6 | 79004439 | 79004457 | IVS14X:-4 | TCAATCAAACAGAGGTGG |
| 111 | chr6 | 79004438 | 79004456 | IVS14X:-3 | GTCAATCAAACAGAGGTG |
| 112 | chr6 | 79004437 | 79004455 | IVS14X:-2 | TGTCAATCAAACAGAGGT |
| 113 | chr6 | 79004436 | 79004454 | IVS14X:-1 | CTGTCAATCAAACAGAGG |
| 114 | chr6 | 79004435 | 79004453 | IVS14X-Ex15XX:1 | CCTGTCAATCAAACAGAG |
| 115 | chr6 | 79004434 | 79004452 | IVS14X-Ex15XX:2 | ACCTGTCAATCAAACAGA |
| 116 | chr6 | 79004433 | 79004451 | IVS14X-Ex15XX:3 | AACCTGTCAATCAAACAG |
| 117 | chr6 | 79004432 | 79004450 | IVS14X-Ex15XX:4 | CAACCTGTCAATCAAACA |
| 118 | chr6 | 79004431 | 79004449 | IVS14X-Ex15XX:5 | GCAACCTGTCAATCAAAC |
| 119 | chr6 | 79004430 | 79004448 | IVS14X-Ex15XX:6 | TGCAACCTGTCAATCAAA |
| 120 | chr6 | 79004429 | 79004447 | IVS14X-Ex15XX:7 | CTGCAACCTGTCAATCAA |
| 121 | chr6 | 79004428 | 79004446 | IVS14X-Ex15XX:8 | CCTGCAACCTGTCAATCA |
| 122 | chr6 | 79004427 | 79004445 | IVS14X-Ex15XX:9 | CCCTGCAACCTGTCAATC |
| 123 | chr6 | 79004426 | 79004444 | IVS14X-Ex15XX:10 | TCCCTGCAACCTGTCAAT |
| 124 | chr6 | 79004425 | 79004443 | IVS14X-Ex15XX:11 | CTCCCTGCAACCTGTCAA |
| 125 | chr6 | 79004424 | 79004442 | IVS14X-Ex15XX:12 | CCTCCCTGCAACCTGTCA |
| 126 | chr6 | 79004423 | 79004441 | IVS14X-Ex15XX:13 | TCCTCCCTGCAACCTGTC |
| 127 | chr6 | 79004422 | 79004440 | IVS14X-Ex15XX:14 | TTCCTCCCTGCAACCTGT |
| 128 | chr6 | 79004421 | 79004439 | IVS14X-Ex15XX:15 | TTTCCTCCCTGCAACCTG |
| 129 | chr6 | 79004420 | 79004438 | IVS14X-Ex15XX:16 | GTTTCCTCCCTGCAACCT |
| 130 | chr6 | 79004419 | 79004437 | IVS14X-Ex15XX:17 | GGTTTCCTCCCTGCAACC |
| 131 | chr6 | 79004418 | 79004436 | Ex15X:1 | GGGTTTCCTCCCTGCAAC |
| 132 | chr6 | 79004417 | 79004435 | Ex15X:2 | TGGGTTTCCTCCCTGCAA |
| 133 | chr6 | 79004416 | 79004434 | Ex15X:3 | ATGGGTTTCCTCCCTGCA |
| 134 | chr6 | 79004415 | 79004433 | Ex15X:4 | AATGGGTTTCCTCCCTGC |
| 135 | chr6 | 79004414 | 79004432 | Ex15X:5 | GAATGGGTTTCCTCCCTG |
| 136 | chr6 | 79004413 | 79004431 | Ex15X:6 | CGAATGGGTTTCCTCCCT |
| 137 | chr6 | 79004412 | 79004430 | Ex15X:7 | ACGAATGGGTTTCCTCCC |
| 138 | chr6 | 79004411 | 79004429 | Ex15X:8 | AACGAATGGGTTTCCTCC |
| 139 | chr6 | 79004410 | 79004428 | Ex15X:9 | GAACGAATGGGTTTCCTC |
| 140 | chr6 | 79004409 | 79004427 | Ex15X:10 | AGAACGAATGGGTTTCCT |
| 141 | chr6 | 79004408 | 79004426 | Ex15X:11 | CAGAACGAATGGGTTTCC |
| 142 | chr6 | 79004407 | 79004425 | Ex15X:12 | CCAGAACGAATGGGTTTC |
| 143 | chr6 | 79004406 | 79004424 | Ex15X:13 | TCCAGAACGAATGGGTTT |
| 144 | chr6 | 79004405 | 79004423 | Ex15X:14 | ATCCAGAACGAATGGGTT |
| 145 | chr6 | 79004404 | 79004422 | Ex15X:15 | AATCCAGAACGAATGGGT |
| 146 | chr6 | 79004403 | 79004421 | Ex15X:16 | TAATCCAGAACGAATGGG |
| 147 | chr6 | 79004402 | 79004420 | Ex15X:17 | TTAATCCAGAACGAATGG |
| 148 | chr6 | 79004401 | 79004419 | Ex15X:18 | ATTAATCCAGAACGAATG |
| 149 | chr6 | 79004400 | 79004418 | Ex15X:19 | CATTAATCCAGAACGAAT |
| 150 | chr6 | 79004399 | 79004417 | Ex15X:20 | CCATTAATCCAGAACGAA |
| 151 | chr6 | 79004398 | 79004416 | Ex15X:21 | TCCATTAATCCAGAACGA |
| 152 | chr6 | 79004397 | 79004415 | Ex15X:22 | TTCCATTAATCCAGAACG |
| 153 | chr6 | 79004396 | 79004414 | Ex15X:23 | GTTCCATTAATCCAGAAC |
| 154 | chr6 | 79004395 | 79004413 | Ex15X:24 | AGTTCCATTAATCCAGAA |
| 155 | chr6 | 79004394 | 79004412 | Ex15X:25 | CAGTTCCATTAATCCAGA |
| 156 | chr6 | 79004393 | 79004411 | Ex15X:26 | CCAGTTCCATTAATCCAG |
| 157 | chr6 | 79004392 | 79004410 | Ex15X:27 | TCCAGTTCCATTAATCCA |
| 158 | chr6 | 79004391 | 79004409 | Ex15X:28 | TTCCAGTTCCATTAATCC |
| 159 | chr6 | 79004390 | 79004408 | Ex15X:29 | TTTCCAGTTCCATTAATC |
| 160 | chr6 | 79004389 | 79004407 | Ex15X:30 | TTTTCCAGTTCCATTAAT |
| 161 | chr6 | 79004388 | 79004406 | Ex15X:31 | GTTTTCCAGTTCCATTAA |

### Example 7: Dose Response Evaluation

Experiments were performed to examine dose response relationship of a number of exemplary ASOs according to some embodiments of the present disclosure.

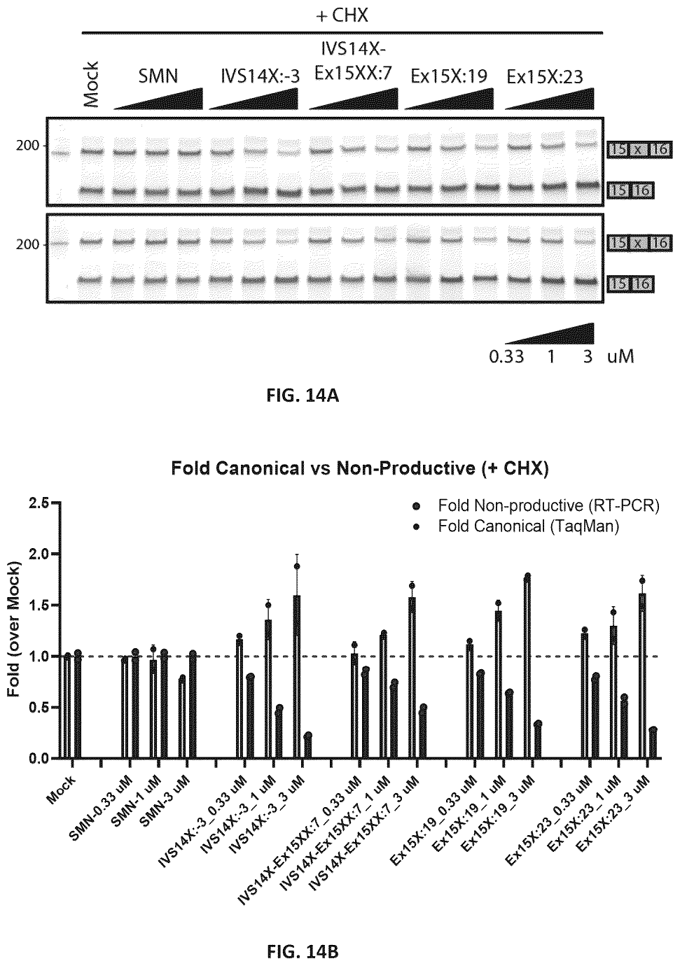

In one experiment, ReN VM cells were nucleofected with ASOs at different concentrations, and then cultured for 24 hours in the presence of cycloheximide before being collected for RT-PCR analyses that examined the splicing of NMD exon (15x) in the cells. As depicted in FIGS. 14A and 14B, the cells were treated with control ASO (“Mock”), an ASO targeting SMN mRNA (“SMN”), or exemplary ASOs such as “IVS14X:-3” (SEQ ID NO: 58), “IVS14X-Ex15XX: 7” (SEQ ID NO: 67), “Ex15X:19” (SEQ ID NO: 95), or “Ex15X:23” (SEQ ID NO: 99), each at 0.33 μM, 1 μM, or 3 μM. 24 hours later, the cells were collected for RT-PCR analyses. Primers that target exon 15 and exon 16 were used for the RT-PCR reaction, and the amplification results were visualized as shown in FIG. 14A, with the lower band being from mRNA transcripts without Exon 15x (“canonical PHIP mRNA”), and the upper band from mRNA transcripts with Exon 15x (“non-productive PHIP mRNA”). Quantification of the change in the amount of canonical PHIP mRNA and the change in the amount of non-productive PHIP mRNA in response to different treatments is summarized in FIG. 14B (normalized by their respective amount under “mock” condition). For all tested exemplary PHIP ASOs, the higher the concentration they were applied at, the more canonical PHIP transcripts and the less non-productive PHIP transcripts were observed.

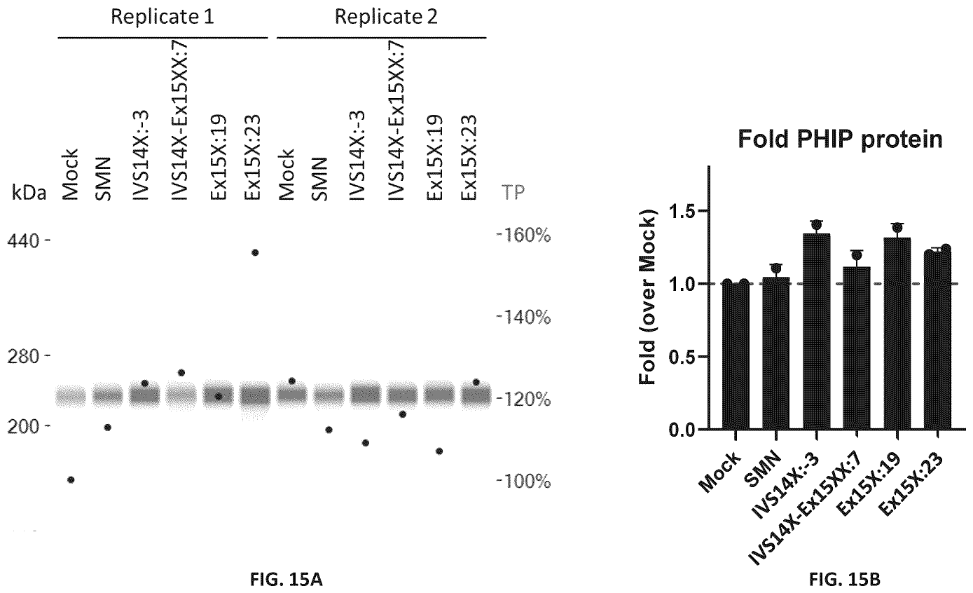

In another experiment, ReN VM cells were nucleofected with various ASOs at 3 μM, and cultured for 72 hours before being collected for JESS assay that examined PHIP protein level in the cells. FIG. 15A shows western blotting images of two replicates, and FIG. 15B shows a plot demonstrating that all tested exemplary ASOs (IVS14X:-3, IVS14X-Ex15XX:7, Ex15X:19, and Ex15X:23) caused an increase in PHIP protein level as compared to mock condition, while SMN control did not.

### Example 8: Modulation of Spicing by Exemplary ASOs

This example illustrates further experiments that demonstrated the modulation of splicing NMD exon 15x from PHIP pre-mRNA by exemplary ASOs according to some embodiments of the present disclosure.

TABLE 7
Exemplary ASO Sequences

| SEQ ID | | | |
|---|---|---|---|
| NO: | ASO ID | Length | ASO Sequence |
| 58 | IVS14X:-3 | 18 | GTCAATCAAACAGAGGTG |
| 181 | Compound 1 | 17 | TCAATCAAACAGAGGTG |
| 182 | Compound 2 | 17 | GTCAATCAAACAGAGGT |
| 163 | Compound 3 | 16 | CAATCAAACAGAGGTG |
| 164 | Compound 4 | 16 | GTCAATCAAACAGAGG |
| 165 | Compound 5 | 16 | TCAATCAAACAGAGGT |
| 67 | IVS14X- | 18 | CTGCAACCTGTCAATCAA |
| | Ex15XX:7 | | |
| 166 | Compound 6 | 17 | TGCAACCTGTCAATCAA |
| 167 | Compound 7 | 17 | CTGCAACCTGTCAATCA |
| 168 | Compound 8 | 16 | GCAACCTGTCAATCAA |
| 169 | Compound 9 | 16 | CTGCAACCTGTCAATC |
| 170 | Compound 10 | 16 | TGCAACCTGTCAATCA |
| 95 | Ex15X:19 | 18 | CATTAATCCAGAACGAAT |
| 171 | Compound 11 | 17 | ATTAATCCAGAACGAAT |
| 172 | Compound 12 | 17 | CATTAATCCAGAACGAA |
| 173 | Compound 13 | 16 | TTAATCCAGAACGAAT |
| 174 | Compound 14 | 16 | CATTAATCCAGAACGA |
| 175 | Compound 15 | 16 | ATTAATCCAGAACGAA |
| 99 | Ex15X:23 | 18 | GTTCCATTAATCCAGAAC |
| 176 | Compound 16 | 17 | TTCCATTAATCCAGAAC |
| 177 | Compound 17 | 17 | GTTCCATTAATCCAGAA |
| 178 | Compound 18 | 16 | TCCATTAATCCAGAAC |
| 179 | Compound 19 | 16 | GTTCCATTAATCCAGA |
| 180 | Compound 20 | 16 | TTCCATTAATCCAGAA |

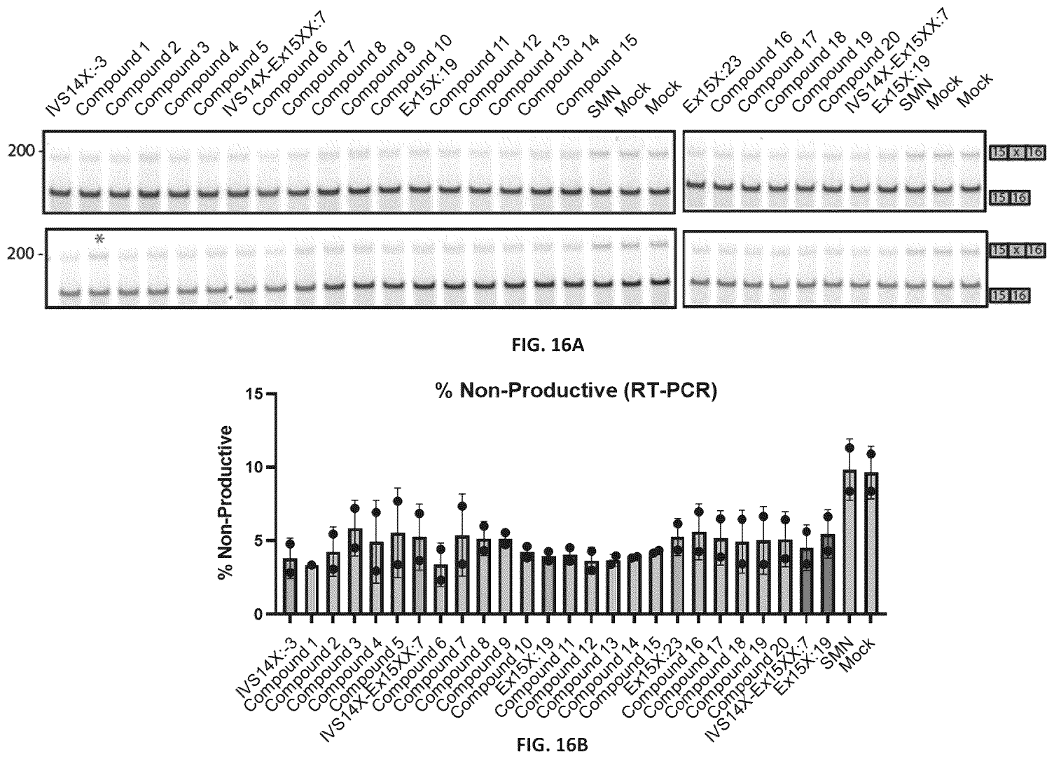

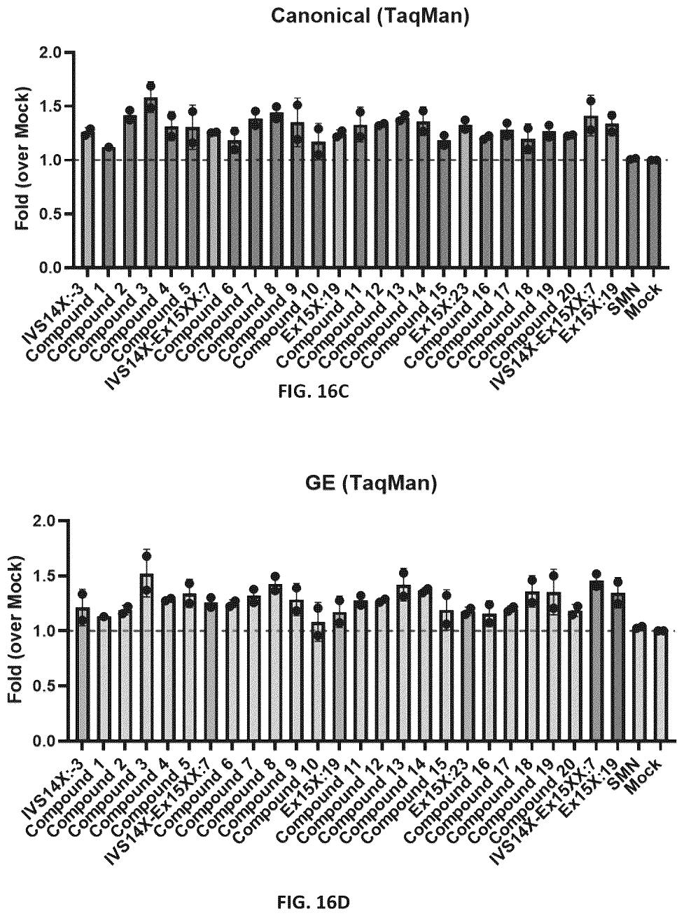

In one experiment, ReN VM cells were nucleofected with ASOs at 3 μM, and cultured for 24 hours in the absence of cycloheximide before being collected for RT-PCR analyses. Specifically, the cells were nucleofected with one of the exemplary ASOs listed in Table 7, or control ASO (“mock”) or SMN ASO, all at 3 μM. 24 hours later, the cells were collected for RT-PCR (FIGS. 16A-16B) or RT-qPCR (FIGS. 16C-16D). As shown in FIGS. 16B-16D, the exemplary ASOs led to decrease in the amount of non-productive PHIP transcripts with exon 15x (FIG. 16B) and increase in the amount of PHIP transcripts without exon 15x, as shown by Taqman qPCR using probes spanning exon 15 and exon 16 (canonical; FIG. 16C) and exon 35 and exon 36 (GE; FIG. 16D).

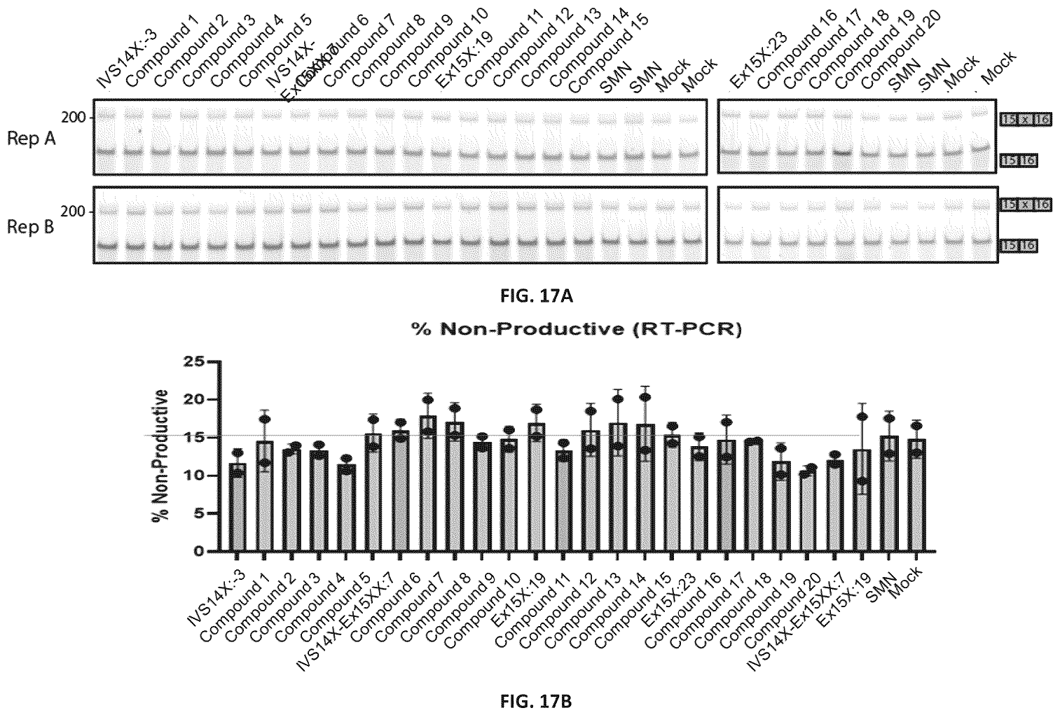

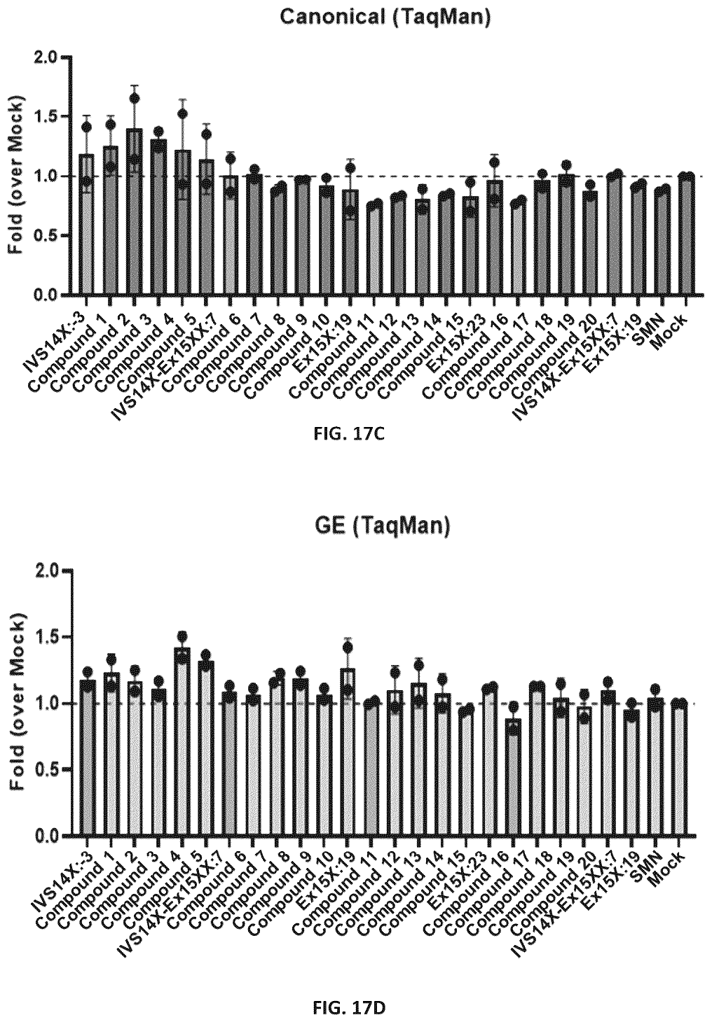

In another experiment, ASOs were added into culture medium of ReN VM cells at 60 μM for free uptake, and the cells were then cultured for 3 days in 3D condition and in the absence of cycloheximide before being collected for RT-PCR analyses. Specifically, the cells were treated with one of the exemplary ASOs listed in Table 7, or control ASO (“mock”) or SMN ASO, all at 3 μM. 24 hours later, the cells were collected for RT-PCR (FIG. 17A) or RT-qPCR (FIGS. 17B-17D). As shown in FIGS. 17B-17D, the exemplary ASOs led to decrease in the amount of non-productive PHIP transcripts with exon 15x (FIG. 17B) and increase in the amount of PHIP transcripts without exon 15x, as shown by Taqman qPCR using probes spanning exon 15 and exon 16 (canonical; FIG. 17C) and exon 35 and exon 36 (GE; FIG. 17D).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1-86. (canceled)

87. A composition comprising an agent or a vector encoding the agent, wherein the agent (a) binds to a targeted portion of a pre-mRNA that is transcribed from a target gene and that comprises modulates splicing of a non-sense mediated RNA decay-inducing exon (NMD exon) and (b)_modulates splicing of the NMD exon from the pre-mRNA, thereby modulating a level of a processed mRNA that is processed from the pre-mRNA, and modulating expression of a target protein in a cell having the pre-mRNA, wherein the target protein is encoded by the target gene, and wherein the target gene is a PHIP gene.

88. The composition of claim 87, wherein the target protein is a PHIP protein that lacks an amino acid sequence encoded by the non-sense mediated RNA decay-inducing exon GRCh38/hg38: chr6 79004373 to 79004436.

89. The composition of claim 87, wherein the non-sense mediated RNA decay-inducing exon is defined by genomic sites GRCh38/hg38: chr6 79004373 to 79004436.

90. The composition of claim 89, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 79004373.

91. The composition of claim 89, wherein the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 79004373.

92. The composition of claim 89, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 79004436.

93. The composition of claim 89, wherein the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 79004436.

94. The composition of claim 87, wherein the targeted portion of the pre-mRNA is located in a region between two canonical exonic regions of the pre-mRNA, and wherein the region contains the NMD exon.

95. The composition of claim 87, wherein the targeted portion of the pre-mRNA
a. at least partially overlaps with the NMD exon;
b. at least partially overlaps with a region upstream or downstream of the NMD exon;
c. is within the NMD exon; or
d. comprises the 5′ NMD exon-intron junction or 3′ NMD exon-intron junction.

96. The composition of claim 87, wherein the agent comprises an antisense oligomer with at least 80%, at least 85%, at least 90%, at least 95%, or at least 98%, or 100% sequence identity to a sequence selected from the group consisting of SEQ ID NOs: 4-182.

97. The composition of claim 96, wherein the antisense oligomer comprises at least one nucleotide with a backbone modification, at least one modified sugar moiety, or a combination thereof.

98. The composition of claim 97, wherein each sugar moiety of the antisense oligomer is a modified sugar moiety.

99. The composition of claim 97, wherein the backbone modification comprises a phosphorothioate linkage or a phosphorodiamidate linkage.

100. The composition of claim 97, wherein the antisense oligomer comprises a phosphorodiamidate morpholino, a locked nucleic acid, a peptide nucleic acid, a 2′-O-methyl moiety, a 2′-Fluoro moiety, a 2′-NMA moiety, or a 2′-O-methoxyethyl moiety.

101. The composition of claim 96, wherein the antisense oligomer is from 8 to 50 nucleobases in length.

102. The composition of claim 87, wherein the agent further comprises a gene editing molecule.

103. The composition of claim 87, wherein the composition comprises a vector encoding the agent, and wherein the vector is a viral vector.

104. A pharmaceutical composition comprising the composition of claim 87; and a pharmaceutically acceptable excipient and/or a delivery vehicle.

105. A method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating a level of a processed mRNA that is processed from the pre-mRNA, and modulating the expression of the target protein in the cell, wherein the agent binds to a target sequence of the pre-mRNA, wherein the target protein is encoded by the target gene, and wherein the target gene is a PHIP gene.

106. A method of treating or reducing the likelihood of developing a disease or condition in a subject in need thereof by modulating expression of a target protein in a cell of the subject, the method comprising: contacting an agent or a vector encoding the agent to the cell of the subject, whereby the agent modulates splicing of a non-sense mediated mRNA decay-inducing exon (NMD exon) from a pre-mRNA that is transcribed from a target gene and that comprises the NMD exon, thereby modulating the level of a processed mRNA that is processed from the pre-mRNA, and modulating the expression of the target protein in the cell of the subject, wherein the agent binds to a target sequence of the pre-mRNA, wherein the target protein encoded by the target gene, and wherein the target gene is a PHIP gene.


SEQUENCE LISTING (182 sequences)

SEQ ID NO: 1 - DNA, 143836 bp, Homo sapiens
  source 1..143836: mol_type = genomic DNA
agtcgctgcctgagagttgttgtttcctcctcctcctcctccgcctccgccgccgttgcttgaatggtggagccgaagctcggctcgtgaacacacactgacagctatagggcaggcggcggcaccgtccccgcttcccctcggcggcggggtgtcccgtcggcggccctgaagtgacccataaacatgtcttgtgagaggaaaggcctctcggagctgcgatcgggtaagtcggggggctgccggggccgctggcaccgggcgattccgcccgcggtcccccgccccccgccgcccgaccgctcagcctgtgtgtctccccgtccctctctcccgcgcagagctctacttcctcatcgcccggttcctggaagatggaccctgtcagcaggcggctcaggtacggcggcgcgcggcgcgggccgggccggcgctcgccgctcgcgggcgtgggggagggaggcggcggcggcggggccgcggcccggggcgcggggctcagctctccccgctttgttgttgcaggtgctgatccgcgaggtggccgagaaggaggtaagggcggcggcggcccggcgcgtcccgccgccgcgcggccgcctctgggccgctgcgccgccgctgccgccgccggcagggcgcggggcgcggggctcggggtccccgccggggcgcggggcgggcgggggaggggagcgggcgggtaggggccgggggccgggctcacggggtgtcctctccccgcagctgctgccccggcgcaccgactggaccgggaaggagcatcccaggacctaccagaatctggtgagacattcacgtcgcagagcaaagaaacttttccctgagtcgaataaaaattgaatttcccgcaattttttacagagacaaaaacttggccctagaacgtgaaagctccaaaggccatggggagggttgggaggccggcgcggggtctgcgggggccagtggccgggggtactcgtacttcggggccgcaggggctgcggacaatttatttgtgatattttgttaatgtgctagaaataaagatacgtcatgagtgttgtgcagtgcaaggccggaggaggaggggccggcgcgcgcgtccgtgcgggcgcgggggcgcgcgagagtcggcggcggcgggagcggcgcgaagagggagagcgcgctcgctcgttcgcttgctcgctctgcacgcgcggccggctcggctttctcctgccctacacaatagctggtgactaactgctccgaagtggcattagcatttcacaagcaggctatccagccgctgcggtaccgtggcaagacattattttttccccctctgtcgtttattttgttttccttaaattcagtcctgggttttttgttggaaggattctgatgtgtgatgatagttggtcagtgcttcgtcttgcatttactaaaacacatctttgcagaagtgtaaagcctgtaattccctaaaaccagaagtttagtaggctttttttaattcttatggtatgcctctcgtttaattaaaaattattattggagtacactgaagagttttgatttttgtccacatatttactgtgggatggaacttggcctatgaaagttgtgtttatattgggacatattttgtttaatagcttaaaaaccttaattttgtgggttgtcaaaatgagtattttaggaatctttaaaaattgcttttcagaagtggatttatcacttcagtattgtgattggaagttactttaaaggagctatattttctgtaacactttctcctaaactgatacaggttcgttgtgtgaatatgtgtgtggggcagttaaagcagtcgcaccggacccaccaagcctttgaaatttatgtagtgtgttgattttatgaggcttttgtaactgttttgagatagttatgattactaatgttacttcatcagaacttctctaatgactaggaagtttcagcgagcttagaatgagtgtcttttgtgaggtgaaagcaggacatgattataatggcgctttttttggtatgtagtgtatttcctaacttttgtgtataaatctcttgtggtttaatataagatgttgataaccgctcaaggctgttgaaataagcaagggtactgaggtttaatacatctaatggagatttaacagtgtattagtgtattcattttcacactgaacgtgtgttaattttcaaggagtgatttgggttactttcattcaattcttggccaaagtgaatggagctggaagtaaactccaagatttactaatttgaaattaatttttaggtttacaaatacttgatagaagtcttcagtgaatttacatgtcttccttgatatttgaaaaaattatcgcaaagattataatactccatttttaagatggggcaggttttacaaaactcgtctttgcatctgcttttacatctgtgcatctcttaaaaacccacaaaccttaagtcactgaggaatgctgcattttcttaatcttttggtaatgttacattttttaaatgaagcagcttaatgttatctggctaaattatggtaatggctaatcctcgtaggaatatcattttgtttttttgttttttttttcagtaaaaatgagctatcaattttgattttgagttaatttaatttttggggggagggtggggggaggaattattagtaaagtgaatttctatccctagtctgcatcgcagatttaagatacagtaagcattctttctatctatgtattatgctcaaaattcacctattttgaatggaagtggtttttcttgcttggagttttctccttactagaacaggaacgttggcatactcatcagacagtttagaaatcttattagaacttggagttctttgactcgttttctcttttcattcattcagcatattgcttactctatatctactggatgcaaggtaccatttgaggtgctttctcatccatcaaactgtatatctaggtgtgtgagttaactttattttacctttttagttgagatttagttaggtctaaaaacatttggaacactggatttttttgttcaatagttgatctcgctgtttaatatctaagtcagcacatgcaaagtctcttgtacagtattttctattttaattattctgacttaaaaagttttacgttgttgtatatcgagttgaatttatttaacattgatttgaaaattacttgttgagtcattagtttctactctacctggaaatgatttgttgtattaactaataaactgagcaatattttaaatgggagaactcatatatcacatcgcattgaaatttctaggaaacctcaaattattgctgcttaaaatggggtgtaatatttgtagatgtgaatttaaaaatagtcactgatcatctgtagttagtctagttacaatcgcaatggttgaattttaatcaagaggaaagtagaattttaagagcagttttaaaagtttgcaggttaaaattgatgcgttcatttcaagtgtgattgagaaaggaaatggtcgtatcttatggttgtttctgtctctggtataaaacatgactgattatcaccatctaccttaagatttaaaacatttttaacaaagtaaatggaattggataggaagctaaaagttgtttttggtactatggtatacgtttaattagattttacattttttaaaatatgagatttattttaaagaaaaggggcactttttccaagtatcagaattcttaaactgtagaataaaaaactttttttggacgaaaggaaagctttgtcatttgtttatttcttagtttgaatagtgaatctagttagtttattaagtctgctttctagttgtcacttaatgatgattgcatatttacgagatctagaggcagaattttcaagcattttaccgaatttggcactctcttgattacattttttcttgtaagacttctcttttttccccccaagaactagttcatgttttgccaatctgttaaaatgatttaattttttccttgtaaaatagtgatacttgcctttcttaaaattattccactttattagaaatcttttttgtttgtttgcttgtttgttttaagtgaaaggattgggagtcaggagatgtggattgtggttttaagccccgctgacagttaaactgtaggtaatcttgggcaagtcatggaatttctctttgtcccagttatctcatttgaaaaataagtggattgaaattttttcaatctgaaaatctgtgattttgaactagaaatagcagtacattgtatttaagaactaagctttatttcattcttagagcttagttttccaaaacacagaaaagtagaaatcacataggttacaaaattatataaatgcttttcatagtgccattctttggtggtaatatcatttcttgttgcaaagatgatttgagacactaactacgttgtaaaatgccccaaaattaccatgatttccatcatagtttaagtactagttttcattattgttggtctcaaattcagagatgaataggaatgatggataggatttatttaagtatatatcttaggtatacatttatttagtgtgtgctgattaatgtgaaagttaaggtataaaacctagagacaactttcagggaaaaaaaaaagatatcatattaaatgttttagaagtagggattcccattctatattgaagataacatagtttcaacacttgattattataattttttggggttgggtgaacatgtaataaagtaaatgtgtgtagttgtagtagagttactatttccttttttagtagagtatcaatgaaatagaagctttgcgtggtagtctctaaaacagctttaatctcatatggtcgttataatgatgaataaaatatgttaggcgtgaagggttatatgatgaataaaatgttaggtgtgaatatgttgctgagaggctgaaggagcattcttcattctttcttacatatagaattattctattgatgcttataccattttttaaaatttgaggcatttacaaactttttaaatgcaatacttactatattgttaatattcttaaatagttgtaaagcggcatgagcataaagcgggagctggaatgtccctttcccaaaccataatggtttttatttgattgttgtgaggcagaatcgtattgagggagtctgttttccattctgtaaaatgggctaataatagcttctactttgtgtggtctgtgagtaataactggcatgaatcaagtactatgtaagggttttggtttgctattatcagaactttagtagtggtagtaaaggttgttctgcttagagctttataataaagttttctttaagaaaagcatttacatttttattccaggcacaagtatgtatatactttctgaacatagaatctgcagtataaaaaggccacattaaaacttcagaagagtccaggtgtggtggctcatgcctgtaatcccagtactttgggaggccatgagttggagaccagcctggacaaaatggaaaaccccatctctacaaaaactaaaaaaaaaaattagccaggtgtagtggcgcatgcctgtagtcccaattaccccgaaggctgaggtgagaggatcgcctgagcccagggaggttgaagctgcaataagctgtgccccatgccactgccctccagcgtgggtgacacagtgataccctgtctcaagaaaagaaaaaaaaaatcttcggaagatactataattactttatagactcaaggatgaaacaggtctaaaaaataaaggaaaaatgtttaataccaaaaataaatcatggttaaggcattgtggatagacctctggaaaaattgtgctactgatttctactgtgacttgtttttatggaacactgaacattaagaaataatttgggtggttgacatatcctgaatggattagagaatatgcaaagctttttttctttgggtaatcatcaaatgacatttaggaatatatgaagagaatttggctatggcgtctgcctccttctatacagttatttgtgagatttacaaccatcacaatagtttgtattctttttggaaactgtcagagaagagatgtcttgacttctgttttgtcaatctgtttatatatattttaaacagaattcaagaaagactttagctttaggtaaaataaatcctaaccgatttcagttttttagttgattcagaatttctttgtaagttagtttttggttttctttttttttttcataatctggaaagttgtggatgaataataagaaaataataataataatagtctaatgcagcttcagcttaagttaaaaccaattgacagtcattcttccctttctcaagttttgtgggtatttaccatgtcactacagaattgtaacaaataaaatgtatcttgtacaacttactctgaacaagtaatactaaataggttagtttcattcttactagtagtttccttgtataaatttcagaattcatagaaaaatatctgtaatactaagctaagtagcctttagttgccagagaataccaaacatacttatttaaaataacttcctattttaatgcacttttacttagaaaaaaatttttaacagctaattttaggatatttaagttgcttttgtggttatatctaaaagtggcaaaggctattataggaatcaaatttaaaattttagaaagacattttaaatgtgaacattctcagtttctattattttttgagaagctatgtcaatgaaattatgaggaaattgtgagttaataaattactttgcagtgaataatgttattggacctatggaatatagaggagtgtaagtattttataacaaattggtgaaaatgataacatgttttttcttgatggaatttgataaagcagggtactgaaaaatcacaaactgttatcttattgaatagtgatgaatgtaagttgacaagttactacagagaaggcaatcatcagttgtgaggcaggtcctcctttatttaccttacacactcaaaaacagccgtatttgagcttaggcatcacaggtaagaaaaggatggttggatgaggagtttttgataagaaaatttaggtggcaacatagtcgaatgcagcttcttcttatattaaaactaaattgtaaggagaatgagatgtgtggtataggtaagccctaggttatccatgacagtacagatggctatggcaggtttttttgcagaagagacacaggaagaattcatgtacagcaggccttccttatctgcgggtgtcacatctgtgaattcaactgtagatcaaaatagtttagaaaaaaatacataaaaaatttttaaaatacattgtaacaattatttacataacatttacgttgtattagatattataagtaatctagagatgatttaaagtgtgcaggaggagtgtaaaggttatacgaaaatactgtgccagtttatatgaggaattagagcatcctggattttgattttggtgtggggtttcctggaaccattcccccatgaagattggaggatgactgtgttctggactccagctgacccatggtcatgtgtcatggtctatttggtctctgtcatgaatagaaaaaggcatgtaacttgtcttgggaaaggttatagtataatgcaagaaatacataccttgtgtttgactctggtttagtttttgggtgtagagacactaaattctaggcaacttagtgaaacatccgttgtctgaaacaactttgctgtcattgtataagaattgtgttattttgagtactgtgcttgaaactgaaacattaaaaatgaaactggtcattcaagattatttacattggccttagtataagcagttaaaggatgtttcgtccttaaattaatacacagtaacttttcatcagattcctagatttgagtttaacgttttactaataatgctttaaagagaattttaaaattacacaaattagcacatatgacgacttaaacatcaaaacttgaaaatttgttattttaagcagttaaatgttgtcattttgttgtattttaaattattttttatggagcatccctactttggtttatggaaacctcaattctgaatagagtctcttttttttagcaagatgtaattggtttatgatgaataaaatttggaagcgtagtattgcattttctttttttgtttgtttttcttatagtattgcattttctttaaatgacaaatttgtgttggtatttgtattgataatgtaaactgaattattatctaaatgaattcttaacagttaagtaaattctgaatatgacatattctttgaagcttgtttattcacaattacttgagcaaatagaccatcaagatttttacacttaataaggttctatattaattttattataattataaagaaataataggcatctaagggtccttctgagaatagttacccaggttagaggacttgacttgtactgacttatatgggttctattaagtttttaatcaaatctacgtgtcgatggggagaaaaactaaagttctaataaggaagctgtaacataataaaaatatcagtaaacttggaatcaggagacctgggtttgattactatttctcctattttccaaatgtgtgccctcagacaaactgctgcctttgaacttcagttttctttatgtatgaaatagggagagtagttaagcttatgtgcttatgaagataaataaattttgggaacactcagcacaaagtaagtactctgtttttctgtggagaagctattacagttaaaaaatcagaaaatgcattctttctttaaagattatagatataatttaacccacactgttagtaacagatttataattgtaagcttggatgtttttagaattttaggggctgggattttaatataggccttacacagtaattatcacatagtatttctataaatttatatttatatattgtatattatatattataaataataaaatataattaatgtatatacaatatattgtatttctataattttgtttatatgctacagtagtgtacaaataagaactatattccaataaactggccttttagaactcaaaaacataatagagagcagaagtgtaactagccttttatatctgacaaaactgtgagagaatagatttgagaagatctgaaaaactgacttttttagaaccaattaggtgttcagaacataaggatgaagtgaaagcctagttagacaaatgatactggtgccaaacagcagggtcttattttcataataattatatactagaggaaagaagggcacttctaggtcttcacagctaaatcaaataattggaatgaatatattgatctaacatcactttgaaaaggttgttaaaaacatgctcaaatttttttcctatttagatttacaaagattttgttcaacacgaagggtcaatatgtaatatagaatgtaattaaagtaacagccaactgttaggtacagtactaactgcttcatatggtttaatttaataatttatgactactttccaaggtggtgactgttgtgataccagtcatacagatgaggacactgaggcttagaaagagtaaatgtttttcccaccctcagccttagcaactttaaacccaggctgtgtgacttgagatttcgtggggttttttgtttatttcgctttgtttttgagacaaagtcttgccctgtcactcaggctggtgtgcagtggcatgatcttggctcactgcaacctctgtctccagggttcaggcaattctcctgcttcagcctcctgagtatctgggactacaggcacgtgccaccacgtccggctaatttttgtatttttggtagagacggggtttcaccatgctggcagggctggtcttgaactcctgacctcaagtgatccatcagcctcagcctcccaaagtgctgggattacaggcatgagctaccatgcccagtgagatttcgcattttaaactactgaccatttctttaaaaactgagtagagttcttagtgactgtaagtctggtagaatgcagcagcttccagatccattgtattttcttcctactgataacacttgccttttaacaataccttccactgagataatggcaaaattttttggctgggaaggatggctgtttatgtgaagtagcatactatcagaaaatgttgttttagccaaaaatgcgggataataaacctctcaaagagctcagtttcttgtttcttccatactgtaattcacattctcagaagatgacccttttgttcaaattgcaatggataattttagaaatactttatatatgtgatctggaaaagggatgatacagatttatgacttgggccaagatggacaaggaatgagcagagttgctgctgtttcacagaattcttttgttcttgctgatggaaacgttgctagaaccatgaatcattctttcgatcagggggtggtaaactaatggctgctgtgggtaaattgtgcaggagatctgtttttggaaggccattaagctaagcattgttattgcatttttaaagaattgtttttaaaaaataagaacatgcaatggagatagtatattattcttaaagcctcaaatatttaccatatgatggttacaaatgtttgctgacttgcactctaaataatataacatgatattttaaaaaaaggatacctttggctaattcaaattttaactatgaaaaagagttaaaatggatggacagttagccaaagattgttgttcccatagcaagcaactcaactgagaacggaggtggaagggacataagtttgaaaaagctctgactagcctcctggtttagccagtagaggtgtgaatctcaatgggatggcatcaggatttgcatccatagggcccattcttgcatcttattggaggagagaaaatgttttgttcatcaaaaagggatgagagctaggcatggtggctcatacatgtaatcctagctacttgagaggctgaggcaggaggatcacttgaggccaggagttccagactagcctgggcagcatagagagatgctgtatctaaaaaaataaaataaaaattaggtgtgatgtaacgcacctatagtctcagctgctctggaggctgaggtcggaggatcgtttgagcccaggaatttgaggctgcaatgagctgtgactgtgccccctagccttggtaacagaacgagatccccctgcccccttaaaaaacaaacaaaaacaaaaccagggtggatgagtttaaaaagttttgagcactactgatacatgggcattcttggggaaatttaaaaaatagtaacaaatttggtattaactctttaatctgtttttactttctaattttaaattaagggtttagagcagtataatcttataaatattgtagttttatcttctacttttacccttagaattttttaaagtgttcccttttacctataggaatttatgcttttatgctagtttccttagtagtgcaagtgaactgtgaaatgtttctctttttctggctcagtttaatgtctgttgaaaactattcaaaatgtctgttgtatgacttgcttatatcttgcacattgatatatacacatatttgaggtaataggtcatttgtgtgatagtatgaattgtagggtccagagttagattgctatggtttgactctcttatgtgctgtgtttgaacttgggaaggtctcaatatctttttaaataaattctataatttttttatagaatgggaataatgatagtatatacctctcagagttatgagtagttccatgtaaaacttaccacacttaccatagggcctggcattcagtgtgcagccaatatgtgttagctatattagtataatttatcacactgactttataatctggtcatactttttatgtcttgaatgttaagaatttgaaagcacttagggtcacagaaagtaactcgtatatcagaaattggggatcttgaggggttatttttcccctgaaataatattgtatggcagattactgagaaaccacatatatacaatatagtaagagattttattattatattattttaaatgtgtcttttaattatttaccagtgaaataaaagctaatttaaattaaaagcttaagtttatattaaagatatggggttcttactaagttgaaaactgaaaatcacatatgtatatctgtaaataatggattaatatttatgaagaatatgaatatgattaatatgtttataaagaatgtttagaaaaagtagaaaagattgttttattttaaaaaatatttttgtttattctgtgtgtgtgtgtgtgtgtgtgtgtgtgtgtgtgtgtgtgtggtatagttaagagcaaaaagattcatggcccctgctcttttgtagttaatggtgaagtagaaaaacattaaattacacttttaaatgtataatcacaagccaatatattgagttaataaaggaaagtcacaggaagtgtgcaaccaagaaaacagacttggattgtgtagagtatagaaaaacaaaggaagttacattagagggaagatctgagacacatggaagagttgtctgggcaaggaagtgtggggaaatgcagagtgtatgctatatgataaagtctgacaggagagagagattatgcgtaaataacctgaaagaaggccatgtgtttgaaacgtgaaaagtgagagttggggaaagggactgggaatgtgccaggtgaagctagaaaaataagaagtgaccatacatgcatttctgggctgatagaccaagttaaggatttggatctttatccaaagagtgaggaaagcatcagaagtgttttaagtaggggtgacttgatccaatatgtattttggaaagaacattctaattgcagtatagagaatagatttgaatggggcatgagtgcatatgggaaaggtatgcatttattgcaaagattatttggactgttatggagatacaggtagatggatttaaaaataggaaggaaaaattaagttgaacttggtgactgcttggatattgggaatatgatgggaaggaggaaggaatgaactgtgtgcaaaatgcagctccttggtttctggcttccaaggttggataaatggggttgacatccacccaggtagggaacactttagtggggaatgttttcatgaggatccatcaattcagttttgcatatgttgagtttcagatgctttggggctctagagagctgttttaagaaagtttgctatgcaagtctatgctataaatagaaatttggcaatcattgttataacaattgtccaaagagagagtatatagtgagaagaaaagaggatctggaactggactctggtacctaggatcttaaggaacttcactatttaatggctatatagaggagtataagcctaaaagaaaaatgagaaggaatggttggagaggaagaaagcaagccaaggagtgtcacaaaagttaagagaagatagtttgcaaatgaggaagtgttcaacagttaggtgaaatattatgggacattaaataaggtatagatttaaaaatgcttattggagttattgataattgaaggtgaacttttgataagagctattttttgtaaagtcatgagtgtgggagctagtgtggagtagattgaggagaggatgggatacgaaattagatggactatggaccattcttttaaaaacatgtttccggagaggaagagaacgggaaaggggtagccaggatatttgggtggggaggtgttggataatagcagttaggcaggatttttgttttttcctaaagtggatgcaaaatgagcatgtttacatgtagatgggaaggagttggttgagaggaagttagtgtacatactagaatgtggaggtctaatttatagtgtaggatttccgtgcagtcagagaagttaggattcaaagcataagtagagggattggatcttaaatatttattaagagggacagttgtgttaataagagacagaattggtacaggtgtagataggtttacagatttgatagggaagaaacaaagcttcagctttaaaaaaggttctcctgtaaagtaggttaggtcatctgctgagaaaatgaggggtggggaagaggattcttgggaaagagaggataatagcattgtttgttagtctgtttccactgttctttccagagaccctttcatggtggtacctcagggactgctaagaggaagcaagtagagagtcaagcattgagatatgtgttccctgatgccacttaagtaagatactttctgagtacctttactttaatctgttttgtctaaaatttgtttgaagacatgttctaaggctttctttaaaaagtgttttaaaactgttagaattggccggacgctgtggctcatgcctgtaatcccagcacttgggaggcctagggggcgaattacttgaggtcagaagttcgagaccagcctggtcaacatggcaaaaccccgtctctactaaaaatacaaaaattagccaggttaggtggtggggcgcctgtaattccagctactcgggaggcgaggcaggagaatcacttgaacctgggaggcggaggttgcagtgagccgagatcccgccattgtactccgtcctgggtgacagagggagactccgtctcaaaacaaacaaactcaccccaaaactattagaataaagaattataccatgtaaaaaaattttactgcatctatttttatgtgctgggataggtgctaacaaaatgttttgggttttttgagagtggggaagtcagtcgtttctgcatattcttaaatcttcatatatccaactagaactttattaagcacttaactataagtactgcattaggtattgggtagacagaggtaaacatggcccaatctctccttatgcaggacacagacaagtaaactggcaattttagtatagtgcatgagtcttatgaacccccaaaacagggcacataatggtagggcttgtgtatttttgcagggaagaagatcgtagctgtcattagattcttaaaagtctaagtatcaaaatatattaagaacaattaatatggtaagtactacacagtgctagagggatgaagggacattcgaggagtataatgtgctgtgggaacacaaagtgtccaaaaaggggcacaggtggtgtaaataggtcatggaaggcagtgagtgagacatagactgagacatgaaggagaatgaggtgtagaggtgagcaggtaagacaagattgccagcagaggcatcagcttgtataaagtctcaaaagccagagaaagcacaaggaatttaagaactggaagtatctcttctaactttaaaatgaaagttgattttgaaaaattcaaacattctaaatcaccaaagaaagcttgagtcagagtggattcataatattttattgaaagatagaatattacagtaaggattttcagtttcactacctgaaaatatattttgccagaggatactaaaacaggatcatcacacatctgaagttaataagtaatgaacttccatcttctttaaagtaaaatttttaaaaggaattttgatcaattaagtcacattaaaataatcatttgcaagcttagaactggaagcaaccttacaagtattctagacttctaaattttatttttgagtaaattattttggggaagattaaagaaaagtatacgattttaaaaactcattttggtaagttttcttttaaatgctttttaatttagctaatcatactagtgtttcctaaagtatgttctgagtgaatatgatagatattattagggaaaaaagggattctgtagctaaatgagtttgggaaataatctcaggatttttcagagacttcaatgtcagtgtgctttttgactgcaaaacaaggaggtaatatgtataattcatatgatacaaactgtattagaactcctttttccaaaaatactcattttactggcttatcacctactgtatgccaaactgctgttttaaataatcttaccaaagattatttggtggcagtatggctccatgttatctttgtgaaagaaggagtgtattttgataaagagtaaggtaggtttctcctattggcttcagtttagatgggtattagctacctttaaaaacatatcattcatggatttctgcagttaagagtatagcagatattgcaagatggaatgagatgatttaaatcctcacctccccccacatcagtcttatcaaaaggaattcatgttaaagttgccattacttggtaaacttgaggaacaattttcaaatttcttggtcttaggacccctttacacttactaaaatgtattgaagttcctttatttgtgtagcttataatctattggtatttaccatattagaaacttagcatttaacatattggagacctattaaataatactaataaactaatcttattttaatataagtaacatttttataataattattcccaaacaaaacaaatttgtgagaagcgtggcattgttttacatttctgtgaatctttaatatctggcttaatagaggagacattttcctatctgcttctgcattcagtctgttaagacatcacatgttatgtgtcctgtggaaacctccaccgtgcattcatgagaaaaagtgaaaaacaactaatatagtgttattcataagttttatctgagggacctccaggggttcccaaaacatactttgagaatagctgaactagatactactgtggaaatttttttctaacttgatagctaattatgaagtaatacaatagtatttttttccaatataattttatttagatagtgaatattgtttatttactgaaagactaataaaaatattagcatttcatgtcatggtttcaaatgccggtataaattaagcagtattttgaagttgttccaaaagtgctcaaactttttatagtgggtagctttattttatgattgaaagatatttaaagcagcagttggaattttgcctttctctctgtaaggtttttggtaaattatgaaattcagcatagatgttaagtgttcttaaattttttaataacaaactatcagttcttaattcttaattattctggagatttactttacatcaaagacgcaaaacaaaatattttatacttggatgaatttttttattgttgctcaaagattttgaaaattaaaatgataaactgaaacctactatatttgtcttttacataataggtgaagtattacagacacttagcacctgatcacttgctgcaaatatgtcatcgactaggacctcttcttgaacaagaaattcctcaaagtgttcctggagtacaaactttattaggagctggaagacagtctttactacgcacaaataaaagtatgaaaattttctcataggatttagacattatttatctcacttttgtttttgtgttctttgtatgcataaaaatacatgtgtttaataggctgcaagcatgttgtgtggaaaggatctgctctggctgcgttgcactgtggaagaccacctgagtcaccagttaactatggtagcccacccagcattggtgagttgtttgagttcactagtagttaatagcctcttggtaaagagcttttgaaaatgaaaaggttttttagttactgtggtattagagaacttcatattatgtacatctgcattcaaaaagaaaaaaatagattttttttcttagtagtagttttaatagtgaaaaagtcatgtataatgagggattgtgcacattgcaccttatgtaggccaattagctcattggaattttatttttacgaaagttccatattaaggtatatattattaggtcgagttagtagaatcaatatataattcagattagttataatggtaggagacaaaagtagttatctttttaagagcacagtacttttttttttttttctttttttggctaaagtttcaaagagggagtactgatgcaagtatgtcttcttgagatgcctcctaggttcatgcctttcacatctgctactaccaggcccttgccttacccggattaatacagcatcatccaaactaatcttcttgcctatggtgtcttctatctctctctccctcccagtatagtgtgtaacactgtaatagtaaccttattcagttctacctttgttgtgtcactcttctgctgaagatagtataaagaggttaagatattttaatagtgttgtactttctctaagtgtactcctaattttcagagaccttcaaagttcaatcatagcttcttttccactctcatgttcctgcctagccaggaatgttactcttctttcttccacctgttcaaatcttacttaattcttccttttctggatacttattatacagatcgtaagtttatatgacttttgtttggcatttttatatatatatatatatatatatatatatatataattttgctttcaactttttgtttccttttatttcttgtcttttaaaaatataaaagtggcttttaaaaatacaatatgataatctgtgttttatctggaacatttagtcatttacatataatcattgatgtacttgggtttaaatctactatctaacttgctactttgaaggcaatccctttttctctaattgtttttaagattttttctctttctggttttctgcagtttgagtatgatatgtcttgatgttaattttttaaaaatttgtcttgcttagattctggaaatgtcttttattgacattgaaaaattctcagtcattatcttaaaataacctctgtgttcctttctctctcctttcctatgggacttaaacactttatctgcttcgttatcctttcttctatgttttacatgtttttgactctctgatgtattctgaattgtttgctctgacttacttttcagagataacgtatgtgatatctgcgtggtgggtaatcatctatgattttttttaaaacataagattgtcctgtttaaataatttcaatatataatcttgctataataaacaaccattaaaacaacaaaaaaatttgagagacgagggtatatttgtacttagaaaagtattctaaggtggtataataggattcctgcaagaagggaaagataaaatattataggagttttcccatttaggaaaagtttatcctgtctttgaaaaactatagatccacataataaggagaacacgttatgcttgttctttcagtacattgtcagtcaggcctgtggtaggtaatgaaagtgtaagaatgattgatggtaaataacatttgctttgtgagtaattcctgtatgccagacactgttctaagcattttctatttatttactcttttaaaaccttaaatgttctataggtaggaactattatgcttattttacagaggagactgaagcatggaaagattttggtaacttgaccaagttcacacatgcagtgagaagtgaagccaggattcaacccagactgtttgactccattatgctatgctttattcccagtaaactgaatttattaataataacacttggctactcaagaaaattggtgttagtcattttttgtaaaagggaaccaaatatttataaaatcaaatataatattttaggccacgtcgttgatggtcagattagtaaatactgctgacaaggagagaggaacatttatcttcccttattcacaggagatacattctgagatccgtagtggatgtcggagacaacagatggtacagaaccctatatatacgtatggcaaaaataccttgggttttgcatccaagcatagaaatgtaattgattcatttaggtaacagaattaattgccaagcaggtgattgatgtacctttctgaaagactattttatttttagagtttaagataatttttttctctctctcttccattgtgattttcaggaaaatctttgctttattttaagaaacacctgtagtggattgcccattttgcctacttttaaaaattgtatttacatatattttttgaaagcactgcagagaagctagactgaccaaaatgactcaacttgcatagtcataggttagtgtctgtgttttgctagagttcaccatggtatatattttgttttattatcgctataaagtaatgaacaggatggtgttggggagaaataattcatcagtcttcttgccagaatagttgcctgcatagtggtacaatgcattatttcaagctgttacagtatggttcagaagattgaaaaaaatatatatgaatgtttgtagtactgacttatttcttcttttgttgagtctttgccattttctgtctggctacctttaaaagttttctttgctctttccttggtcagtgtggtattaccaagtatcagtggaggtaagaaaaaattcttgatattttagcttttcttaagatacgtacgtagatttaaacgtttaatatagatcccaaatattaaaattttgacacatttggggtagtagtttttctttaaataaacttttaattttggagtaagaaattatttactgagaagttacacagatggtttaaagagctcctgtatacccctcatgcagtttccccaaatgttatacttttgcattacctggtctatttctcagaactactaagccaatattggtgcattactgttaactaaacagcagaccttattcatgtctcactaagttttcccactaattacgttttcttattccaggatccagtccaggataccgtacttgacatagggtttgttaattttttattggactccttactgtagttcagaggataattttaggttaagcttgtggcaaaattggattttcttagatatttcaaaccatatggaaactgttggacctcttgaatatctcacattcatctcaaacttaaaaatacctaaaatgtaatttttgacactctctgaaccttgttactccaagccagaagcctggagataattttattttagatttactgttcatttcttatgtccgtttaataaataatacctgtttattctactttcttgaatttgttctccattatcattgccacagccacatttcaggccttggtacttcttgcttggagttctacattattcattcactcgctgagtaaatattgtatatctctaattatatctggcactgggctagggtctggagttgcggataagcaagaacaagacctctgctctcccagaacttaattgctacttgagcccctgcccctagtctattcctgtatgtatcctaggagtcagattctaaccagggttttttaaccttggcactgttgagacattttgggccagataattcctggatgtaggcagctttcctgtgcattgttggatgtttattagcatccctggcctctgcttgctaattgccagtagcatccccactacccccttgtcacaatttaaaaagtctccagacattgccaaatatctcctgggcaaaattatcactgctctaaactttttgaagtgcacatattaccatgtcatgcctcctcctttcccccattgagagtgactgaatgatagtgtcaaccattttaatgtgtcacacaagactgaccttttcccacttttcctgccatgtcctcacctgcactttgtgctctagcttagtacaactctttggcagttcttagaatcagccatgttggttttcatccttttgtctcctcacatgttttatttctccctggaatgcccctctgttttctgtacgttgtacaaatatctgtttatcctttaaaactcagatgttaaactgggcagaattaagtggttttacttatctctactattgattttatcacatggttatggctatttatttacctttgtttcctctactatactgttaggtcctggaggacagacattatgttttattgctctttagagacccaactcttcagcattagtaaatattaaatgaaaagtagtaggtagttgatgaaattcatacactgggatgggacaagtaaagttcaaaggtgggtaaggtattacaaaaatggaatgaaggttggtagaaagaagagaagcttgatccaagaatagctttgtggtaagagaagataaaatcaccaaaccagaaactgatttttaagaaattaaggtttatgagtagactgagttatcagttcattacacttggaatataaaatacatacctatacaatttgagaagccacttgagcttgtctcagtgttttcttatcctgttttattttattatgtgcttagaattcccaactactttgaaccttttggtctttgatgtaagttgtgaaaattgaactagaactcagtctcagcaagagacccaaagtggttcacctgccttccagtgctctagaacctcctttaatactgttttcaaaagcaaagacaaaagatcccactgatctgttgttattactagcttgtgattcttttattgtggaagagagatgtgagtgtgtacttggcatagcagcatttacttccaaaggtattagcgaatctttagtattgtcctttctcttacctccatacccacattatgcctgttttcagatatagtttattctatatttgcttatacttcagtaataaatgtgcatgagtaaggggagataggaaagaagagacctaaatacattaaaggggtttttaagattaaatttggaagttggtttaatgcatgtttatcttgtgaaaaagattgtaatacattaaattaaagcaatttctgctgattgctttgtttgcatttttcttacatttgacatgttagcaaagtaaactttttatctctaggtctttcatttgtcatcgtttttggtgttttgcctaagtatttccatatttttattttacttgtctgtggaggtttgctattaagttctgcagttttgtgtgaggataaggagttttgagaatcctgattcttaaacatgtttgagggtaaggaaaaaactttgccttgaaaagtagcttcactacccagacatccaattaatgctaagaagcctttgcatttaaaaaaaaaaaaagtcttaaatcttgtctacatttataattttgtgattctctgagaagactagtttgaataaagacagcaaacagtcacttaaagctagatgcaaatctttgtagagaagactcttctttctcaccttgtgctatattttgccatatttccaatattgtgtttgaattttctgtagctggtaatttcttaaatttattcaatcaaggtttttttttcttagactcttaggtttatatttttcctcatatatttcttacataattattagaatttttttatattacaaatatatgagattacataattttaatatataagttaacagttacatataattcagcaaattataggatataatatatggggtttttttcctgttcacttatctttttctaatatgctatattcagtgttttttctttccattttcttactcaatccatactttattcctagttagatatctactcatcacttatttaaatatatttattttaacctattgtttctcagtaaccatttttacttttagagctcattttctgtttttctagtcataattttagtcttataaaggttttatacttcgcttttttttttttggtttaatgattgttgactcagttatattcttttttgtcttggtctttgttttttagtgttttccctgaatgatcttcatatttctatttgacacaggttttgttgttttattaactattttctccctagcccctcccctgcctttctatttcctcccttttttttgcctcttaaaaacaggttagctgtgagagacttgcaaggtgtattagcattttttttttttttagggaaataatgttcattatgccaaagtatcagatgtacaaaatatatatttatttattttttcctctgcttaaatttcttttgaaattaatggttgacaaaaaaaagggggcaaaactttgaagaaatgaaactcccttaggtgagtttaaattgttgcttttgtagctagctgtgttttaatttcttctgtaactaataggataacatttgtttttaggttatagatgtgatgagggatatcccctccacttcatataaaccatctggcaagcaattccagtagtaattaggaatcactagccattttcccttttagtttgcatcacatatttttccagagtgattattgggtttgattttgttgaatactcatatctgtgtaggtgaagcaaattatgatattattttctcataaggtcatgactttttattttgaatttcttcagggatcctttaaagttacgtattcagtgttttcttagacatcttttggacctgggaagtccatgatcagatcaggctgaatagatatttttataaacaaaggaataatttgttcagctccataaggaagcctcggggaaaattgggctccaacaaaagtcttttaaaaagtaaaagaaaactatgttcagttaaattaacttttatggcacatgaagatttgattgtttagggacagtccatatcaatgttagtataaaatacctttgaatttgcaaaacattttgtctcgtctgttttctatagcaactctttaagataaccctacatttttaatttaattttattcttttgagattgagtctcgctctgttgcccaggctgaagtgcagtggcgcaatgtcggctcactccaacttgctcctcccaggttcaagccattctcctgcctcagcccctcaagtagctgagactgcaggcatgcaccaccacacccggctaatttttgtatttttagtagagacggggtttcaccatgttggccaggctggtctcgaactcgtgacctcaagtgatccacctcggtctcccaaagtgctaggattacaggtgtgagccaccacactcagctgatcttactttattaatgaggaaactgaagcttagaaaagcagtaaccaatgacattatatctctgtttttgtgtgtatatgtatgcatgtacctcagatcttatgcttttaactttatgtcacatttatatagcacctttttgccttctctgtgaaattctttatgtgttaatgtgccagctctgtccatttgttgatttggtaatagcaggtgtttgaattttgcctcagtatttgaactctagaaacaatgaaacatggtgtgttcttttcaagtttttatatagtgccagatacttatgagatcaggtatttaattccttgggaaagtgttgataattttggtgtcaagctgattaaattttttctaaaatgaaattttgttttcagaaccctttgatttttttgcaaagttagcttttgcttttacttatatcctttccaattcattctcattgtggtcaagagaaaactgcaatgagttatcccaaagtaaatcttactatgtatattactccattcttttctcccccaaaacttcaacccgttattactggaatttagtccaaaatacctaacctagcttagaaagcactctatatagtccgctcccctaccttctcagtctcttctcttgacacttgccctttctctactctgtgtgcaagtcaatcttgacttcttcatgttgcttgaatgctatgctctttccagcctcaggctattgcacatgctgtttcctcaacctggaatatgctgcgctcacatcagcattctgtggctgttcatttttcaggtcttagtttaaatgtcagtttttcagagaagttttcccagactcccagatatccacccctcagccccaacgtgtttagaacccttttgctatatatttcaagatttccctataccttctatttgataacacttaacattgttgtaagtccttgtttcatgtctttcttccctgctagactgcaatctccatgagggtaagtaccatgtcttttctttttccctcttattctctagcccatatttggctcatataggtgctctataaatgtttgttgactcaaaattgagtgattaaaatgtttgacattaaaaaaaaccacctaacttaaaaaatgtttatttaaaagaaaaataaattgttattaaaacaactgtaaacattttaaaatatttaaaactgaaaacattatgtcattgtgtgttttctacttgaactagcatcagactggtttccaggaaataaaataggaggaagattaaatactattcctaataattatcatatctaccctttttattagtgctcttaatgtagttggttcatgaaattttttgcttacttcttggaatcatcattttctgtattttatttcagtttagtatttgggttttccttttcagacattatgcatctttatggtatctgctaaattttaaaaacatagtatgtttcaaacaggaggtgcaactacacaccaaaattgaatatctagaaatctggaaagtagctgctgtgcatactgattattttagttatagtattaaagcgaagagagaagctatcatgggtgctatgttgctcaggtgactatttccccttactcttctattcccaatacctgccctcctttttttgtagacaaaaaggtgactacaagattatctttttatattgcaacccagttctttgtactttaactgaaattgggactatcattcattatagttactattacggtgatttatttgtcctgtttctatgttggttggttgctacttgctggtacagtatgatgtagagaccagatggtactgtaggagtaaaccaacctgtgttgaataatttcccagattaattctggccctcgaattatgagtatttggttgtcattacttccttgatacccgtgttaataacaggtctttccattgtggctctatagtgaattaaacattcctccttatatgattccctccgaaacaaaattgaacctgaactatctttgacatgtttctctttttgtttaaaaattcagtgggaaagattacacaaacaaatcttacaacaaagttgatggcctgctttgtagtattatcttacagctttcaaatcaaaactgatgtgaattatttctttatagcatatcattttttcagtccattttcaaatacgcaattgtcattctctatagcataatatacattcatacactgtaatatgcttaggcattagtgtttcttttttatgattaaataaatttaaattccttgcatctttatctctaggtttatttttgtcagtctttgaatttaagctagtctctttagatattttagagttttcctactgcttttttgaattccttgaggtagattgagttataaaaaattagaatgaacgtatgctctatagagaaagtttggaaaacagaacagctcaaaaattaatcagtctcactccttaactgctgtacatactttggcatgttttctttcagtctttcttttctaatattgatctgcataggcagcatataatagttaagggtacagtctttggcaccagactgcctgagtttaaacctgggcttcacctctagtaaattacattttatcctttggtttccttactggtaaggaatatctgcttattgggaataatggaagtatccacctcaaaggattagggaggattaaatgagaatatatgtaaaatatttagaatagtgcctggcatgtagtaaaacactcagtaaatactagctgttattctctaaatttgctacacgtcgtaagagtgttctgtagcctttttcctctccttgttatgtattgtatggtgacatttatacccacattattaattgcagcagataagactttttttttttaatcgtcagattcttgaagtcctagtaaaaacctctcataactctaaaccatggttgctttcctcttcctttatagccattttctcaagggtttgatgcctggctttaacgtaacttctgttagaaaattatgattagcaatttttttttaccctcatatttccttttcattaaaacacttttgtggatctctaaaatgagcaacatttcaagtaattttatgccttattaaatactttatgccacacaatacatttatctgatagaaattctgaaatcaccaacaccatatatagacaactatttggacttgtaatgaactaatgaacttggagggtcatttttatagacattactctctgatgtctagtcatgaatatagcattattttcttggaaaatctaattatcatctaaaattggagatggcacatatgaggggtaaatcagtttggaatttgttttcatgtactcttaagttactcaattttagtgtcatggagttccaaactgttgttttacagtgatagttattaatcgtatttgtagaaagccaaagcctttattaatacagatggtggagattaaaatgaaacctgttactgattatttagaagttactcccttttatattttaatttaggaatcatttctgtagttgttaattataaattataattacttttgcattttatttacagaaaacctgggagctttccttccaagtgttttctttaattagaatcacagaataaaactagtcaagaccagccatggtggctcacgcctgtaatcccagcactttgggaagttaaggcaggcagatcacttgaggctgggagttcgagaccagcctgaccaacatggtgaaaccccgtctctactaaaaatacaagaaaaattaggcatggtggtgcacacctgtaatcccagctactcaggaggctgaggcatgagaatcgcttgaacccaggaggcggaggctgcagagattgtgccagtatactccagcctgtacgacagagtgagactctgtctcaaaaaaaaaaaaagtaaaactagtcaaaagaggataaaaaatgaattaagtaaaattaatgaaaaccttctcataaaatactgatgcgtcttgtctgagtacccccaattctgcagcagaaaaggcccaaattttactgagataccttccgagttacatattcttgtgcctcctcacttaattcttctactgtataaaaattggttctccaggttttagaatctacagctacatgcttttccttgcatttcattaggctgccagcagggctctgctttcatgagttggcagaagttgtaagcactgtttttgtggtcctccatttctgattaagagggaacagctttcctacactggagtaggtattatatagcatattatattgttattaacagttagaaaacaataataacaaaaaacactaacctttgtattttgttaattcatagtcattttcatttttttccaagtcatcttttttaaccatggttaattttgatcttgggtaatttattgcagctttatcatgtgtgcttgttcattttacaaagatctttgcaaaaggagaaaaaagtctaaacattttgtagggaggtaatggtatttctgctatagtacttatttgcagggttttttttatcatgtatttttctagtgccttcaaaaatgtatttactaaggttattacagatactcttttatggcagctacctttttctttctttgtgaacttggttcaaatgatgtagtgagagaaaatgaggtaactttattatgtttatctttttaaaaaatcatctgcattgaaaatttgggcccaacaacaggaagaaagcaccatgttctagatagaaagatgtcagtttggtagtaaagttaaatttacatttccaacaaaattttgtaactttctgtttattttcaaaattaacaaataataaagatacaacatttttacgtggacttatgagaggaatacccaaataaatattaaaacatcattgattgtaagattaaaattttggtattggcatataaatagactgtcagtcatgtcagtggcccagcacagaaaaaaaaaaaatacatatgtatatataaccatatatacaataatgtaatcaaaatttaatagaaaagaattatttagtaaatggaaaggctggaacaaatagttacttgaggaagaaaaaataatctttagagacctcataatgagtattataattgtagatggattaaagagttaagtgagaaatctaaactacaaaaaagtaaaagaaaatgtaagtagatgtttattgtactgttgggctgagtaggagaacttcatattaaagcagtagaagatattaatataattgactgtgtaaaatttgtgaaattgtggggtgtcaaagaacagcataatatgaaagccaggtatattgtggtaaatggaaatgtttctctaataatgtaactggagaaattaaagtttagaacgagtaacttgcttaaagtctccatagtgacaaatttgaacacaggtctgtcagacttaaaacctcttatttcattaaggaatacaacatagctgttaaaatgctgtttttgaggtaaacctttgcgaaattttaaaagtgattgctatagacaatagaattatgggtttttctttcatttatgttcttttctaaattgaatatgcattagtattatgattggataagtaaatcagttctcgaaaaataactaggtgtgaagtaagaagtgacttatgggggaatttgacttattatcctagcacctaaagaagtaatggatgagaacccaagggctgaatatgcgcatagtcacatctaatgtttaagaagttgtgggccttttgttgatctctactaattggctttgtagacttgagcacatatactttatttcaaaggctgttctgaatgatttgctgcaaatgacatgcttcagtattttgctccttttttacttcagactttattcaaatggataacagtgatttctggaatttttatttcatttatcctctatattcagaaaattaagtgaatacgttctccccttatttcccaattaaattcatttagcagggcagttaaaatgaagcactatttagagatattttgaatattttaatctgtgagataaatcttcctttttttttttttttttaattcttaaacctaatttgttgttgtttgagatggagtcttgctttgttccccaggctggagtgcagtggcatgatctcaacttatggcaacctccgtctccggggttcaagctgttcttctgcctcagcctcccacatagctgggattacaggcacctaccgccacaccagctaatttttgtattgttagtagagatggggtttcaccatgttggccaggctggtctcgaactcctgacctcaagtaatccacccacctcagcctcccaaagtgctgggattacaggtgtgagtccctgtacccactaaacctaattatttttgttaatatgtaatttatttgttaattcaccaacctgtgtcagctcttaggctaatcagagaacaaaataaaatcctgcccatgtgaagtttgttatcttgtagtaggagcaggcaggaaataagcaatgaatataataaataattaaataggtctggtaataagtgatagatgaagagcaaatataaagctgaataaaaggggatcagaagttatgggtgatggcaggggctgtgttgctattttcagtgggctggttaggttaggctttattcagaaggtaacagacatctgagaaaagacttgaaaaggtataagggaattagccatatgtggaaaaagacttcatgaagagggaacagccagtgcaaagaccctaagtttggagcacacctggtgtgttggaagaacaggagattggagtggctggagcaggtcaaggcgagaagtagcaggtaaggttagagataatgggagataagtttctgcagggccgtcttaggctgatgtatcaggaagttggtgtttattctgaatgaaatgaagagcattgcagttttgaacagaggagcagtgttgtagtgcaaccagttaggatgcttttgaaatcttcctagaagaggttgcatttgaactgatcctagaggataaaaaagaatttgccaagtgaaaaaagaacagatttgctttgggaagctttatttaagaatagtttctatcactatacttatgaaaataaatgtacttttaaatgaaagtcatatatgcttcttgatttaaggttaaattataatgtcatataaagaatttatccttttgtgtccccttatctgtttaaccatgctcattttaaaaattgatgttctagcttaataaaatttaaggacttccaataatccaagagacatttcctttttactcaaattaattggtctcttattaatttctttagaaagtgaataattgccatttgcccttctgccataaaactataaattaaattgaaccatcttgtatatcttattattaaggaagatttacttatatacttttttaatttaaaaggctctatactcaagtttgagaaatataagagaatttttgtctttaaggtacttaagatctgataggagagatagacattttccaaatggtattataaaaaaatgggagttcatcgaatgtgagtcctagctcatccttctatctttataaaaattatctacctatttgatatggtcaagccttcctcaaattcaacctcttcagttgtacaactactgaactatacttacattttctcctttaaatcctatagttagaaagtttgaaccaccaccttattacctgggaatctacgtatgttctttctgtatgtcttattttttagtgctgttaacacaagtttgtcaatgtttgagagccaggactctgctaagtaccttttctttcagaatatagtcatagtatagatagccaatagttaattggtagctaagcattacaaagggtagtgtggaatatagttttagctataaatatgtttcacctaatgtgattctggaaaatgttgcttgaaaatttgactgtaaagtaactttgaaaaggttattcttttttttcaattggcttccaaaatgaaaatagatttaatcccagtttaggtcaaagattcagtaaaacctgtgtttaagaagcagtaacattttcttttaagagaagtagtttttgcttataaaatactttatcatctccgatttttagatggtctttataacaaatttatcttaaaaattcttacctatatactaatgcttaatcatcgagaaatgggtcttgatatattatttccaacaatgatattttcacacctgaacactgttagtttcctaaaattcgacattttggaaattttggatagagggaattaaagtacaccttggtaatactatcaatgtttttgtggcatgtcatcacttattttcttacaacagacagctgagtctgagatgtgagtgatccttggtgtttggataagatgattttaattctcatttggcaggaaaattgatgtctatggtttttgtgtgattcttgcattaaataagtttggagccaaagcattggaggatgtcttttaccaggccattcctgggcgtgaagaaaatgtgtctggggtctaagctatgattgaaacttattgagtctctgttaaattggtatataaccaacttaagcctaaatatgctaattgacaactgtcaaaccttgttttagaaattcccaagtgttggggtagattgttgcatagaacttacatgctaacagcaagagaaatactgtatcagaatataaaattattgctgtgatgaaagaacatataagcatagtgtaattattctttgaaatattactgcttaggaacctgtaaaggctatatatctcctcaccaggcccaaagattttttgggggtttggggtgggggcagcgggagtgtggagtggttggaaatatagtgtgagccaaacatttattggatgtgaataatataagcaatactgagattcttcataatacaaccaatatatttatgttttatcaactttagggcataattactgggctctgtaccttggctaagtagtattttgtatatatatttagttttctttagggctaaattcgaccgagaaaccctagtgctaattacccaatgtagatggcagtcactgttagaaaaaaaaaaaaaagcctgtattagaagatcatctgtaggttaataatgtgcctgcagatcagtcaacatatatttattgagcaccttctttgtccataagttaccagtatagaaaataagtacagtagtaatttagatggaagaggacattttcagctggagtcaatgtatcaagctttgggacagaagataaacttcagtcagatcttgaataatgattagaatgaatgagttgaaagggagaactaagggagagctaatagattccagagaaggaatcattatacaggtgtttggcaagattcccagtttaattattattttttaataatgcaaatgacgtattcctttatcttcacttgttataaagatgtttttaaatatcattctgcatgtcagatttcgagtgttcaagtgttactgatttatgaaaagtacactgggttccagttttaagtgcctttgtaagaaaaactatttcatagacagcttttaaggaaataacatataaatgctttaggtttttaaagagcactagccagagagtaaataaatctgagctttaatcctagcttcatgccttactttgtgatttttggacaggtcatttaacttacaaattaagttataaagtgagaatgatagtgcctgacttatttcaggtgaaacaatgtttttattcattactttaaagacttttaactagaagccatatatatcagaaagcttctttatttttacctgcacatgacaaattggagtatgtgaattcttaaaaggtgatcatgtaagaaaattaatttccagattacatttattttccttctttttcttggtagcggatactctgttttcaaggaagctgaatgggaaatacagacttgagcgacttgttccaactgcagtgtatcagcacatgaaaatgcataaacgaattcttggacacttgtcatctgtgtactgtgtaacttttgatcgaactggcagacggatatttactgtaagtaattgctaaagtattatttatattcatatatgtaattgaggaaatattgcttataggtgaacttgaagtaaaaccttgcttttttttcttttaataggaaaccggaaattcaggtggtttcacttactctaacagaagagtttagttaaatttggactttatccaaattcagaaagtttctaaatgacatgttgcagcacagtcacagttattatatggttgtacatcatgaataagtatggttctcaacatatatcaaatacacagtttgacattttgcaaaatggcataaaataattttaattttgaactaaggacatctaagtatgttgagagattattaaatgtgcctttttataattgtatgagattattgaatatacctttttatattatctgtagaaaatcacttgcttgtctatgagaaatttcccttaaaatagtgattttaaaaaagctaaaaaattctgagcatatctaatactcttattacctgttattcttaacagttcattcttcaaaatatctgatgttttgtttatagagcctctttgggtttgtttatttgttattgtttcttgtaacctgttgaatagagaaaagcaaagcttttgagctttattttaagtgagaaaattacttttggaaatatttgattttcaggtgccttaacatctacattcattaagcataatactacactattatgatgatatttattattaaacactaccctgtgggctgaaggtatgcattcattcacaaggtttttaatttcttttgaaataagaaaatttgaagtggaagttaaatggaaattttcctgtctttgaagagagcagctgtgaattttacgtagttatgacaattatagattcctctttttagtaaatttgcttgtaaactgagacctatttatacctactacttctcctctatccatacacccagtcatctgtaaaacagctaaatagcaggatgtaggaatgcatttttatacttttcttttcccttggctctaatctacctcaggcatttgcacatcacttcttatatcagtattttccctttttacgttctcttgtatcaagtattattttcagagtggaaaataggcatctctatatttgctgaagtgcctgacatacctacagtagtaatttgctctcaaatagtaatcgaaaattaatttagccattcaagtttaatatgggtatattcaaattcacctctcaaaaaccttaagtaaatttgatttttctaatgtttgaaagaattctttcaaaaacagtaataaattaaaataatgaaaaaggagcactatattgtgccagtaggtcagtataaagttatgccattgtaaagaaccttgtaggaattctttctttaaaaatgatagggattgtaagcatttgtttatcatattttatgaagcattttacatgttttgatattaattagacatataatctcctggaatttttttagggagaatgcagtgaaatagtgtgctttttgctatctcatgagtgtggtaggtacagacttggaagagctttttgaaccagggttcagcatgctgctgcctgcgtctcaaacccagtttgtcacttgttcttataaatgcaattttattgaaacacaggcatgttcagttgtttatatattgtctatggttgcctgtgcactacaacagcagagttgagtagttttgactgagaacatatggcctgcaaagcctaaaatatttatccagctctgtacagaaaaaaattgctatccttttctagacatttgattctttcttttaagggcatgtgatttagagaacaagaataatatgcgaagaggaagctgcttgttggttatttctttagttgaaagcacatcaattaagagaagaaaagtagcactgtactattttatatggtcttaccaaagtctaaagatgtttattttttaagaatgtgatagaatttgttttgctaaattcagtctcatttgtaattttttactggtggtttggagttacagatttcagaatgttaccttacaataagaaataacgaagatatgagggcactatttttttgaaaaattcttgtatttttattattttcacttcaagtggtggtagggtacacatatgttcactattcatattgctcattaataaagtgggtgctgagtgttttttgcatttcactaggcacagactagacgctaactagtcctatggaaatcagtcttgaaaatagatagaatcagcctttaagatagtagatcatgattgaccagtgaaacactcctgtgagagaacatactgttattatttttggaggtaacaatttggtaatggatcaaccagtatttcctttttatataattggtattttaagaccttgcagatttatttctaagatatttgtgtatatatatttgaaagctttttccaagggattctcttgagtcctttctgaacaggcacagctggaggcatacagtactgtggttaaaagcatcagctttgacatcagagggcttttagtcacagctttaccacttctactagtgaaactttgacagattacctcatctctttaatctccagttttctcatcacctaaagagagaggggagtagatgataatagtaccaaccacataggatttttgtgggattacatgaaataatgcatataattagtactgaacaaaatagaaagcagtcattaggtttttcctactgctacttatagatttaagtaaattttatcttttattttttatccaaataaaatcttatttataaatggtatacaagaattaaaaaggaaatcattgtaattgataatgaaattctgtattaggaaaatgtatcattattttgtatgacagtattttaagaatgggtgctttcccaattattttaatgagaaaaacccaaggagggaaatagcataattgccaagtcagattttgttcactaattctcttttagccttttaatgttttttacttggttttaaaatcatttccttaagtgacatttaacggagaaaagcagagttaatgaattttcacaactaaggcactcaaaagtacatggaacgccatataaaaagcagagttttccccccttttcagaaacataaattgtctctctgattctaattaatatggtataataaagaaaatgagtttatgcattcctttaatgaacttcagagttttatgaaccagccactgttttaagcagttgggatatagaattgaaagtagtatttgttcttggagagttcacagatggtaaacaaacaatattgatataatgtgataaaaccttgacagaaggtgtacaaggtactgagataccataggagataaagatacctaatccaaccttgtggtaggagatggtaagggaaatttactagagaagtaacagcttggtaaatttaggagtcgaattggatattgcagacttagggaaaggcaattacagactttgggagaatgtaacatgcaattgtgtattaatggatatggcatagaataaatatggctcatcataaagaactagcgatattggtgtggtacaaaatcaagatacttttgtgacctgctaaatagtgggtttcttttctgaaggcaatggagagctaatgattgatgtggttagaagagtgaggagaatttttcttttcattgaacccttttgcattattgtagaagatggattcaaactaactctatatacagagaaattggcaaaaagtttctggcagtgatctataaaagaaatcagggattaaacagtcacagtgggatggaattgacagattaaaaataggatttgtaagactcgagagactgatttgtatgtagggagtgagatggaagagtataggatgacatattttttcacttctgtgatttagaaagtcctgtcatttactgtggtaggaatttaaggggagaagtttgtgaaggtgaaacgtattgactttgagatatctatgtggaaagtcaagtagaaatgcccagtaggcagtgggaactcagatttaggtgagaagatttagtttaggaatcattcagcttggaaaatggaatacattatcttgtatgtatctaatgtaagaaggctgaaacttcagcttttaaaaaagcagacaaaagaagagtcaataattgagacagagaagctgcctgagaggctaggggaaaaaaaagaagagtggtgtcttagaagccaggagatgagaatttcatgaaaggcagaaacctaaagtaagattagtactgaaaaacatcctttgggtttgtcaaggtaattgatgactgctatggtctgaatgtttctgtcccctcaaaattaatatgttgaaacctaattaatccccaatgtggtagcattaagaggtgagggctttaggagatgaggcctcaagaatggagcccttgtgaatgggattagtgcccttacaaaagaggctcaggggagccatgtaaagatacagctagaaggcactatctttgacacacaaagagagcctttaccagacaccacatctactggcactttggccttggccttcccagcctccagaactgtgagtaataactttctgtttacaaattaacaagtgtctaaggtattttgttatagcagctccaacagactaagaccaaaaaatgttctgttttagcattggggataggaatgaaattgcagtgcagtgaagagggaatgggaattgaaaaactgaatacacttaatataaactaaactttcaaaaacttgacaattaaaagatatgatagtctgtattctgaatgtggagtactgccgcttaactttgtgcttctaatacattgtattttcctattacattgccatgacagatgtctgttttcctattaagttgtgagctttgtctttgcctttgttgttattttttagatctgtagtgccttgcagagtatctgatgtatacaggaggcataccacaaatgatgaatgaatcaagcaatttgagatataaaaatgactaatgtccattcttgcagaaaacttagcatctgttaggggaaatacatacaaatatctgtaagataagttcctctctgctactgtacttcggttacactgatctcttgtacttcagacttggaaagcccatttgtggcttgaagtctttgtattaggactttgcctgggatccccttctccaaagtattcatgtggctgattattattattattttttaattagcagataaagaccagtagaagatcttagtttgaagatcttgcaatatgtcccccaaagatactaatcaattaaagagcaaacattgcaaatttatggggaaaaaccttggaaatactaatgtaaccatgttaacttcactagctctgggatgtgttgacgttgtgttctcagaagtgatgcgctgagaaaaacacagcttcttgtttgtgggattactgtaaaagataatatgacctggatctggtcatgaagaaacattgacagacctaggttttgggtcatcctgcagattgtaaggcctgtattctttataacctgctgaggacatgaaagagaaagcctgagaaagtattccaaattgaaggagcctgatgagacatgtcagttaaatatagtgtatatgggtttgtgggccaaaaaggaaaagagaacattgttgacaaaacagttgacaaaatttgaataggccctgtagatttggatgttaccagtattgttattggtagtgttatcagtattgatttcccaacttacatagggaaatatccttgttttagaaaaatatacactgcgatatttagaggtaatggggtatcaagtcagcagccttgctttttttttttttttttttttttttgagacggagtcttgctctgtcacctaggctggagtgcagtggcaccatctcaactcacttgcaagctccacctcccaggttcatgccattcttctgcctcagcctcccgagtagctggcattacaggcacccaccaccacgcctggctaattttttgtatttttagtagagacagggtttcaccatgttagccaggatggtctcgatctcctgacctcatgatctacccgcctcggcctccaaagtgctgggattactggcgtgagccaccgcgcctggcccagcgttgcttttaaatggttcagaaaaaagattaatggtaatggagttatgtgtgtatgttatatggagagagagggagggaagaaaggtggtaatggagatatgggaaacatttgtactgttcttgaaacttttgtacgtttgaaattcaaaattattttaagtgcataggaggttgtttagaagcaatagtgagcatttagcatgtttgataataatggaaaagaggcagagtatggtcatttagaggacagtaaattgtaggaaaactggattgagaggaagaaactactaagatagcaaatttgaagatacgagaggagattattgatatgtaatgatatttaaggaggtgggatgtatacaatagcagtgtggactttttagaaaggtagttatttggtccacaattaacagtttctacttttcaatatgaaatgttctgtctggcgagagtcttgtttatggattctagaaattactgataaagcttggcaatagaagagtattgaatagagacaggaaaaatgattgctgataatgcttagcctacccagtaaataggagattcatgaattcgtagtgttgtggaattagtatgcttatattccttaccgtaatgtttcttggcatcctggtagcagtatcagagcaagtgatggttagattgacaccgggttggtcattggtggtatagatgaataagggtcgagaatgaaatcagacatgtgttgaaatgtttgaccctgaggtttaggttgatttgtaagggaaatgaaacacaggtctgatagattggtaagaaagaaaataaatgacctagaaaaaaattgaaccaaatttaaattgtacgcaatttttagttttgctagaaaagcaggttactttttagcttgaataggaaaatgaaattgtagtaaatatgacaatacagacaacatttttggcattcatttctactgcttattctaatggcaataagagatgagatctctgactcaaaagctgtaaagtagcctcaagagaaattaaaattctccttgttgactctgccagataacagtaggttttcaaggaatgccaaattctctaagatatttttagttaaaaaatcacataatttcttttattttcatgtcaaagtagtaatctttgtataatttatgcctctcttttaggtacataggtcaatattttaattttaaggtttctcaagttatctgaacagaagtaaactgtttcccaatgtgtacagttttctgcttgtgttattgagcagagggggcttgctgcccagtgtactagaagccaatactatgacactgggtttttgagaaaagaaaagctttatattgaaagtcaactcccaagaagactcaggggtcaagctcaaatctgtctcctcatgctggtttcaaggcagtatttttattacaggttcagtaggggtgaattctgagattagtaggtgtttggtggaaggaaaggggaggtctggaatgtccctgggcatgcacagttaactcattatgatacctcatgggtcacatgtgcaaattctagggtagttagtatgaaatgtggtagatagtcaggctatgatgtcagcaagcttgttctgtgcagactccagttggccatcttggttccaactgatttcagccagttctatctcataagtagagggagtttcagtgtttggacaagttgtttcttagttgccatcccgcacactcaaagaatattagtcattcagtgttaatcaatgttagtcgttgatttctttaactctttggggcatgatttcacttagagaattcaaccattccttgtcatttatgctgcttcctgtaaattactttattactgaacagaattgtcctctctaattgtctacttcatagttttgcaacatctgggcctaaattgacttcttggccttgcatagaaaggcctgtatttcatttatgtagtcagagtatctgtaaataagccataccagaaagagctctatcaagttgttctgaaacttagcttttctttaaataaataaatagttcaattaggccttttgaagcttaacctctggaaactcattaaattattaaaatttgggggcatagactttaggtgctgtttggtttctgcatcgaattgaagtgattcatggccctgtgtctgaagttgtttgaagttttcatggcagtacgtaggccatttttttctggattttgaggataattttaataccaaagtgcctaacagtagaaagccacgtcataaagacctgtggaacttttcttcatgagaacattaaagtcaaatgtgttactagacactgcctaggctttatgaaaaatgctaatacagacgtaccctgtttcattgtgcctttattgcattttgcaaatactgtattttttacaaattgtatatttgtgaaaaccctgagtaaagcaagtctgtcagcatcatttttctaacaacatatgctcaagtcttgtctctttcacatttcgatacttctcacaatatttcaagctttttcattattattataactgttatggtgatctataatctctgatgttcctattgtaagtctttttagggtgccatgacctgcacccttatggcgaacttaatgaatgttgcatgtgttctgattgctccaccaactggcccttccccatttttctctccttgggcctgcctattgaaattaatccaattaataaccctacaatggcctcttagtgttcaagtgagaggaagagtctcaggtctctcactttaattcaaaagttagaaaagattaaggcgagtgaggaatgcatgttaaaagagttagccaagttgtgaatgtaaagcaaaagttcttgaaggaaattaaacgtgctactccagtgagcacacaaatgatgagaaagttaaagagctcattgctgatatagagaaagtttaagtggtctctataggagatcaaaactagccacaatactcttttaagccaaaggctaatccaaagcaagaccctgactcttaattctgtgaaggctgagagatgaggaagctacataagaaaagttggagctagcagaggttggcttacgaggtttaaggaaaaaagccatctccataacataaaagtgcaacgtgaagcaacaagtgctaatgaagtagttgccgcaaattatccaaaagacctagctaagatcattgaagaaagttgctacactaagcaatagattctcaattacaggtgaaacagctgtgtattggaaaaatacatcatctaggactttcataattagagagaagtcaatgcctggcttcaaagcttcaaaaagcaggctgactcttttgttcagggctgatgcagctagtgacttgaagttgaaaccagtgtccatttaccattttttaaattctaggaccataagaattatgctaaactactttgcttgtgctctgtaaagggaactacaaagtctggatgacagcatatctttttatagcgtggtttactgaatattttaagcccactgttgatacctactgctcagaaaaaaaaaaagattcctttcaagatattactgttcattaacaagatgcctggtcacccaggaaccctgatggaaatatacaaggtggttagtgttgtttttatgcttgctgacaatacatttattctgcagcctgtggatcaaggagtaattctgattttcaagtcttatttaagaaatacatttcatatggctgtagctgtcatggatagtaattcatttgatggatctgggcaaagtacatcgaaaacctcctgtttcacctgtaattgagaatcgattattatcgccattctagatgccattaagaacattcgtgattcttgggaggaggtcagaatgtcaacattaacaggagtttggaagaagttgattgcatcccctgtggatgactttcaggggttcaagacttcagtggaggaagttaactgcaatagcaagataactaaaattagtagtggagcctgaaggtctgactgaattgctgcaatctcatgattaaacttaaatgggggaggagttgcttcttatggatgagcaaagaaagtagtttcttgagatggaatttactcttggtcaagatgctgtgaacattgttgaaatgaaaacaaaggatgtggactattacatgaactcagttggtaaagcagtggcttgatttgagagggctgactccaattttgaaataagttctactgtgggtaaaatgctgtcagatagcattccatgctgcagagaaatctcttgtgaaaggaagagtccaatggatgcagcagacttcattgttgtcttattttaagaagttgtcacagctaccccaaccttcagcaactccaccctgatcagccaacagctgtcagcatcaaggcaaagccctccaccagcaaaaatattactacttgctgaagtttcagataattagcattttttagcaataaagtatttttaagttaatgtatgcccattttttaaagacataatactattgcacacttaatacactgcagcataatgtaaacgtaactcttacatgcacttggaaaccaaaaaatttgtgtggtttgcttcgttggaatacttgcttaattgcagtggtctggaactgagcctgcggtatctaggaagtatgactatatttgtttttatttgagtcttgcctctgaaactttttattttgaagaatgttgaagtttttaaagggatttacaaaatcagaactttttttggatatacagaaaaaaatactaattgcttatagtaagaaatccagctgcatgctgaaaggaaaagtaattggtaccattacctccatactgtaaaagtactagaagaaataacagtggttacaaagtaaatatctgatgctgttttcttccttcatcctaaagtgcagaagtgtgttgactgtttttaaggcacatttctttatcataatatgtgtgtttttttcaggtgtggcagtagactaagctaattttagagtacttgaagccaaattgttaggattggaaaaatgtaaattttgtttaattcccaccctttctcaacacactttctcataaataatatctatgctgttgtgttttcaagtatagagatgtactcttttagcttccttggcttcatcctttgatgacaagattgtcttgacttaaaaagaaaccctgtaaatttggtgtctcattttgttttgggatttaaataagctaagggctatacagatttgcttgagatttttccagcttatgctaagtttgatataaacaaatgataatattaatgagcactctttcgcagtaattataaccattttacattattgaaaaatgaggcttggtgtgatggctcatgcctataatcccagcactttaggaggctgaggcagatggatcacttgaggtcaggagtttgaaaccagcctgaccaacatggtgaaaccctgcctctactaaaaatataaaaattagctgggtgtggtggcggacgcctgtaattccagctattctggaggctgaggcaggagaatctcttgaacctgggctgcggaggttgcagtgagccgagatcgcactacaatactccagcctgggcgacagaggaagacttcgtctcaaaacaaacaaacaaacaaacaaaaaaaaacaagaaagaaaaatgaaacaagtgatttactactctcttatctttgtgattaaacaatagcacaataatttgattttaaaatgaattataaaaaagattttaaattttgtacccaccccatggatttcttaaaacctgttcatagttttccagataaaaatctacactaagtggtctctcttcgtaatctcatctgctatcattgttttgactactatggtataaactgataacacccagatctctatctctaacctaaacctcttcttgtagcttcagatctattactcacataaacattagcaggatctggctaaaatactatagtgaccatgtcttgtctgcttacaaattgtctagtgactacctcagcaggttaaatacaaactcttaacaggcatttaaaactcaattatcattcttaggtaaaacatttccagcataatcacttattttcacttacactttgtactttcaagttttcttactttgccaattcctagcccgagcagcaatccccagcaacattgagatgttactgaaaagaaatgactacttagttttcccttgagtattggagctttgctgaaaaaactatgtcagttgaggtatttctaacatcatgctatttaaatttagcaggtctttaattgttgcttgcagaatttttccacctttcttactgcttttattccactgcatttccaaactttcttcacttttttagttcattcgcttgttgttctctccctctcacttttaatcctcagattttgttttgttttgtttttgctgaggttacatgatctctccctacttccttttctttcatacaccttagaagaggtatatcttatgtcattgccccttttcaccatttacgtaacattatttttaatatccataacttggttttaacattttatttttctttggtcaaaagactataatatcttactctttggcttattgatcttagtaacattcgtggaactagaggtaggcttatggaaaagagagtgtatccgttcaaattatgttagacattttggtcttttttaaaataatcatatttatcgatttttaattcattcttcctcatttatttaaaacacatttatagcctgtaatcccagcacgttgggaggccaaggtgggtggattacttgaggccaggagttcgagaccagcctggtcaccagggcgaaaccccatctctagtaaagatacagaaattaactgggtgtagtggtgcacatctgtaatcccagctactcaggaggctgaggcatgagaaccacttgaacctgggaggtggaggtcacagtgagccatgatcacgccactgtactccagccagggtaacagagcaagactgtctcaaagtaaataaggaaatgtataaataataaaataaaacacatttatagtgctttaaacattttttcccaatacatttaatttttcctctgcagttcttctagtcaattatttttgagtacttgtatattgtatagtcatcttaaaagagtttcattgaattcatgctcagattatcttttgtttcaaatttccataaagttaaattaattatcacaagcgatgatatatgagatagaaattgcttaagaatgcttgcagttctgttggaaaatcttccgggagtcctgaggggtagaattaggggtaggggcagcaaacttctgtgggtcatggaagaggataatggagagatttttgtgaaaggatggtcaaaaatttctctctctggcaatttgatggaagaaaagaaaggttaattgagataatatgcactcagagggaagcatatgcatttttgtttgttttcagcacttgtctaaaaagagcaaatttaatcaagcaatactaaagtggtaagataactaaatgataaacccagtaggaatatagtaggacttcctgttacattttaattggtgaaagtcactggaggttaattttaggtcacagattaccattgctcctttcattaaaactgacattttggtcaaaagactggtgtttttatttgttaacatgaaaaatatgtgaagcatctttatagtggaatgtaaaacacactcttattcaaagattcataagaatgatatgaattgtatcagtattgtgaagcataacatgatgtcatgattttacttttgttatatggtctacctactgtagtgtacttaaaattctttgtgtactgaatgcaggtgagaaccaaaattgcttcagttttgggaagataaccaaatgtagtgaatatgttgattcaatttgtgaaatgtagagaacttaatttgaatgaatgccctaaagcattttttttactcagcaaatccctaatgtgtttccgatgtgcgtatagaccagctttctgggagtcatagtctgctacagctcttccttcacttccaacagataatcagtgatgtgctccttatcccttttattccccaaacagctgttataaccatgtctttgattattcttttttgctataataatttgtgttaccacttattctctcctgaccatactattttaacaactttcagctggtctcctttctgtcttagcctaattcacactgatgccaggatgatctttagaaaacacaagtatgatcctgatactcacctgtttgatatcttcagtgaccctcattacacaccacttttcttagtaaaccgtaaaaggcctttcacaaattttttttactttttcatccttatgccttcacctcacactctaaattaccttgcaggctgcttgcagttctatgaactactcacattgtttcacattttcatgcttttgaccatactttatagtcacattatggaatgctctttttaaaataataataatgatattattaaaataagaatatgaatactgtcaccagcaaaagcagtgttagaagataaagctgtaaccatgtgtcagacactgtctcatctcttcaccctcatttagttttgtgacaatcctatgaggtacgcataggtgagaaatctgaggcacaagaagtgtatgtaacatgtccaaagtcatgcatttgaccctgaaggctgtcctctaagctacattacattgcctcttcttgcgtgagactctattcaagcatcaccacttctgggttcctttccaccccgtacctctgcatctctcagaaaaagtagactgtttccttgtttgaatctctgctgattaacagtggttaaacacttaggacatataagtgtttatatgtcctatataatacataggacgttatcacatttacattcttctgtcttgcttcacaaggacaagaatactgtgtctttatatctcattcttatatctttagtggttgtcacatagtcacaagacaaagggataaatgaatgcatgaatcgcttaattttaaaggtttagggaactttaaaggccatccattttaacctccgtattttatagatgaagagcctgaggtgaggttaagggtatgtgataagaatactagacaacaatatggatagttgaccaaagttaagttttactggtctttatagtggttagtaacccatttattttactccaaagcttaatcagaggaactggttttatcgttaattggggggtgttttaggtatcattggcagagtctgatttaagatacagcatttaacatactctgtttggaaacattaatatgttatatttattgtcagtatgatgatttatatgttatatttattgtcactatgatgcagaaagttgtctgaattatatgcctccataggtcctctgagttctttctaatgtcagatatgaaggtgtttctttaagaattagattggaaggtcgttaagcattctaacatgaacccttagtaaaactgtatcctgtaaaatattcagtaattttgcaaaagtgtgtgttttctcttttaaccttgtgttaaagactagaaaatgtggcaaaaatattaagttttgaatctgggaagtagatatatggggattaacgatactattctttctactttcttatatatttaaaattattataataaaaaataaaaatagagtctgaatgttctatctctattttggctatataaaatgggaatgttcatttgtaaatatagatattcataggaaactccatatcctttacaatttctttctggacttacactactatggcagagtaaacaagtaattatgtatttttattgacttcatacagatgagattaatttttttctgataactggctttagataatgtcatgtttttagctgaagatcagtgataggaaactcacagcttcctgagagaaaaagccccaaacaagcttacttttagttttgtattacaaaacaaaatagataatattagtcaccattgtagcttgcatttgttttgcagctatgatttttttttttttatttcagaagttcaatttcttttacagaaatccacattgagggtactttgatagtagatagggaaccaaaggtaaatattaattatcttatcccacttgtcttatttgctggtgagtacacaagtgctctttttaagtggtttaataaagagctttgtgtaagctaacacaagtatttaacactttttggtatttaaaaatatacttgttcagaagtggacagaacaggtagatttatcagtgttaagattttgaattaaaatttatggggttaattctattaatatccattcttactttaagggttctgatgactgtcttgtgaaaatatgggcaacagatgatgggaggttgttagctaccttaagaggacatgctgctgaaatatcagacatggctgtaaactatgagaataccatgatagcagctggaagttgtgataaaatgatccgagtctggtgtcttcgaacctgtgcacctttggctgttcttcagggccatagtgcatctattacatcactacaggtaattgtcttaatccctatacattgttgttgttgttgttgttataatgtaaagtaaatttaatgaagtcacttttggttattaaattttaagaagttacttttataatcacctttataatccagtatataaggccaactaaaatgaagacaggagtatgtctgctttctttataaatatatggactttaaataacataaccttggtaaattcttgccattttctaactgttgttgttagttgctatttgtatggcaaaaagtagattttattttgactgtgtcactcttgtaaagattttttttctgtgattgtcttttcagttctcaccattgtgcagtggctcaaagagatatctatcttctactggggcagatggcactatttgtttttggctctgggatgctggaacccttaaaataaagtcagtttctattttatttcatagattaaatgtgtttagtttaatgagtgaaattgcaaaagtataaagtagtcgacaataggaatacatttactttttctccttgagtaaataatagaaatcctcttttggctctttactaacctgttttatttttattcctcccctttttttatgtttcaaagaaagtttctcatgttcacattttttttctctttatcctaatcctagctctttccttcagcatttacctgttttattaccactcctttctatgtgctgcatagttttaatagctgcttttgatgctcagaacttaatcttaagaatttctaatgctgttctttagaataattttaattattgatgctactgaatctataaactttagattttattttagatctaggaaaatgtagggagacagaatcattgaagtttcactaaactgcatcaaggtagatagatgtattcccctccctccccataataacattttgcatttgtgcggtcgcccatattaatacttcggtttatcaaaacaagttggctggatagtattatctttggattaagggcactgaaatggttaaatgactttcccatagttaaaactatgtttaaaaattggcaaggccaggactagagcacttctctgccctttgatgctttctataccatatcttttatttatttatttatttatttatttttgagacggagtctcactgtgtcgcccaggctggattgcagtgtcgcgatctcagctcactgcaagctctgcctcccgggttcacaccattctcctgcctcagcctcccgagtagctgggactacaggcgcccgccaccactcccgtataagttttttgtatttttagtagagacagggtttcaccgtgttagccaggatggtctccatctcctgacctcgtgatccgcccacctcggcctcccaaagtgttgggattacaggcatgagccaccgtgcccagcctctataccatatcttttaagtggatgtagtggggagaggaaatagtgtatttgctccatggatagtctttgaacaagaaagtagtgtattcaattgattattcagttgatttcacagtgttgaaatggccatttctgccactcggggtggaaagtcattcagattagaaggtatatatcagtaattatctctgactgctatgtataaattagcaacccaagttgagttatttaataaaataatatatgtatgcctttgttattttaaaaacagtagtgtttagataattgtttgtaatgcaacacatgcttctttaacttcatgtatatgaacattcttgtagagtggttcttatggcagcttgggggccatggtagctcgagtaacaaaactacttcatttgaaaaaagattgtgatttaaaacatggttctttggacaaagcaagttgacttcacttcagttgcatccctcgtatttaaagtctttctgtagatagataaaagttgaaatattttactgatgtatatggacagtagcaatagttaattgttaattgttgctactgttttttaacctcctcatggacctgatggggatacctttctcttttcctgtcttattgttttagagaagaaaaagtgattagcaatgttcaaattgggataggaacagaaaatgttttacagaattaaactcacaccttatttgtgcttcttgaggattttattgctaagtacaataacatttcatgtttctggatgcttgagaagtaagcttgtccatataaatgattttttcagaacctttatgtagaatcacatataaaccactaccctaatacagtattttttctccatgaatgtttttatatagatataattaggaatatatatataattttgttttcttaatattttaccatagatatttatatgtattttaatagcttgcattatgttcaatagagttgttatatacaacttagtaatttttttatttttggacttcaaagttttttctaggtatttataagtaatattctttcctattcctatattctttcctattcttgtcccttaaaatttttaaattcagagttcaggcataagaatgagtactctggctgggcacagtggctcattcttatctaatcccaggactttgggaggtcaaggaaggaggatcacttgagttcagaactttgagaccagcctaagtaacagagcaagatcttgtctctacagaaattcaaacaaacaaacaacaacaacaacaaacgccagacatggtggtacacacttatagtcccagctactcaggggtctgaggtgagaggcttgcttgagcccaggagttaaaggctgaagtgagctgtgatctcaccactgtactgtagcctctgcgacagggccaagatcctgtcttattaaaaacacaaaaacaagaacaatccattgctctttagatataagccttggaacacctattttctttatgtgtttacacatagttgaaaactgccaaaaatttggcaggatatagatttatactgtttatgtcacataggatttaattaataatcaaaggcaaaaattgtatttagctataatgcttttgtatattctgctgggggaaatgactttagaaatcctgcatctgcattagttgaatgaatcaattcaactttgggaactcaaatatctttataaattcacttgtatgtgtgtattaccaactcacgagcaaagatgctgtttcttaaatgggtagtctaagtggctaagtgaaccataagctagctccagaggtctctgggtaggaggaagctgtccaagtatatttagacttaggtgtaaagagactaggaatcctaaaatatgcatataatatgatgattacaaatttttatttgtgattattaggcgcttagtctgccaagctagtactaagcatttcacatttgttatctcaatccctataacaaccctttgagaaaggtacagttattatctctggggcagataagggaaaaacctggggcacagcaggttacattgttagcaggtggtactggagccaggatttaaatactactagaggtataatgtcagaaaccccttttaaccactgtgccaaactaagtggcagttagtcattgaaggattggctagaacttgttgaacctaataaaaatactaatatttattgagcatattggacattgtactaagcactttacatgtatcacactcatttaattctcccagcaactctgaaataggttccgttttacccctgttttacagatgaggatactgaggtacagaaatgttaacttggcctaaggtcgcactcttgataagtgatagacctagaaactgaactcggcccaaggccctttttaaaaatgtttcctcaaataatgcatctaaaaacaaattatgggtagctacttgttttttccctttaaaaatataaaatctttttattttttaattttagtttttgtgggtatgtagtaggtgtatatatttatggggtacatgagatcttttgatacagacatgcaatgcataataatcacatggtgttaaagggggtctccatccctttaagcatttatcctttgtattacaaattatccaattacactcctttagttatttaaaaatgtataactactattgattatagttaccctgttgtgctatcaaatactaggccttattcattctttctattagttttgtactcattaaccatccccacctccctcctcttcctcccactacctttcccagcctataatagccatcctctactatctcatgaattcaactgttttgatttttagatccccaaaataagtgagaacatgtgatgtttgtctttctgtgcctgccttatttcacttagcataataacctccagttccatctgtgttgttagaaatgacaggatttcattctttttttatggctgaatagtactccattgtgtataagtaccacattttctttatccgttcatctgttgttggacactgtggttccttccaaatcttcactattgtggcggggcgtgttggctcacgcctgtgatcccagcactttgggaggccaaggcaggcagatgacctgaggtcaggagtttgagaccagcctggccaacatggtaaaactcctgtctctactaaaaatataagaattagccgggcatggtggtgcacacctgtaatcccagctacttgggaggctgaggcatgagaatcacttgaacctgggaggcagaggttgcagtgagctgagatagcgtcactgcatttcaccctgggtgacagagtgagcctctgtctccaataaaaaagtaacttgtagtgctttattaaatttattggttaatcaaaataattcaccagtgtgtaaagagaagtgtctaaaaataagtaacattagcaatgacaaagactcaaatgatggtccatcaggttccaaagcatctatgtgtgtgtgttttccattaaactaaccttactgatttgctttaaacctaaacaagtaaacaaacacataattacttacattgtcacactaatttaatactttaaaatataatttcattgcttttgtcatttggctgtaatttaacttataaaattttaagttgaggcaggagaatcacttgaacctgggaggcagaggttgcagtgagctgagattgtgccattgcactctagcctgggccacagaacgagactccgtctcaaaaaccaaaaaacccacaaatcttcgctattgtgaacagtgttgtagcgaacatgggagtgcagatatcccttcaatatactgatttcctttctaaaataaaaaccttttaaaaatggtagtttataggactatttgcatatttttgtaatgagtttcaggtaaatggttgcttggaacttatgtgagcagatgtaccaaaaattaaaaaaaatctagagctataatccataaggtagtaatgaaagcaatgcatttagttgccttcataattcagattaatttacttaaattacagttaattttgctctgaataaatgtttaggttttaagaatgttctaatatctgtctctctaaatgagatttgttctgctcatctgttacattagagcctgtgaatattaatagaatttgctgtaaatactaatgtaaaactatttacaaacttattttttaaattcttaacattttgttgcattctgtatcagtaaaaaagataattctctttgctcagaatatatattaggttggtgcaaaaataattgcagtttttgccattattttaatggtaaaaagcttttttttaaaaaaaaggcaaaaccgcaattatttttgcaccaacctatagctcagaaatattttatgtatatacttttttccagtaaatgatcacaaaactaaaactgctctaaatttgttatatattgtgaaagttatataggcttaagctctaattatttttgcatttcatcaaatagtgtagaagaactatgccaaaatgtacagtaagtagggcttcaaatataatcagtatattggaacattacatacacattgttaaatgtattagagctggtgtcatgataaaaatcttcattttctctaattggttgactgtaacattaaacatttaatatcagtagtattgatataaaatatgtaatctgtaatacagttaggcaattaatgtaaatacagctgtgtgtaacctgttttctgtagactgctattttgaatgagcattcattgtagttgacgtaagagtcaaagtatcttcagattttcactaggttaaatgtaatatccctactttttggggtcttaaaagacatgttttatcacattagtaggtcaaagaagggaaaagcttaattaatgtaaaatactgaaaaagttcagttattgatggtatacataaggcacagacagcacctattttgctaatgtattcatgtttgcgaatagcttagttccttatatgtttgtacgttgctattctatatgaaaaactgagaacatgttaaatttgattagaagtaagtgtaactagtaactgcatgtctggtgtgacagtgaatttggaattgttccatgtacgtattgttgtgtaataaggagctcctacaaaatctatatctagaatttctacaagatcttctgatttatgacacataatgatttctaagttgtttttaggaagggagccatctgttgcttcttaattttgtccttctgggattattttttgaaacagattatttctgctgctactggttcctttaggatatttttaattctttattttttgtattacagcccaagacctgcaaaatttacagagcgccctcggcctggagttcaaatgatctgttcttcttttagtgctggtaagtttccctcattctaattttcgacttaaagttgttcatttagaagccttatagtggagtgtgtaatatttaggaaaatatttactgttgtattatcaatcatgttcttatacatatgccagagctaataagtaactgagaattattataaactaacttagaaatatttcatttatgcaagagtttagtttatataatcacatgactgtttttctttaagaagactataatgttctagtaggagagaataaatagtttcaaaatcgaccctgttaaagaatgaatgtttaaatagttgtttcattgtaagaatggtattttaaatctggtttatgttttcatggattttgaaggtattctatagttaagtaaaataaatttgattcatatttgtacatctttatcagttaattcgctacatattgttcttatttgcactttaaaaagtgattgtatagcagtgctaagattccaattcagatttgtgtttatgcagcagcattttgaatggatgttagggtatttttaaacaagttataaaaaaaatctggttatttgtagtaaattgataagaaaattaaatgaaacatgagtagatccaaatagtgtctaatacctgttcctcctacctttattttcagttttactttttccgctcattttataattgtggataggagtattacctgagataagggatagcaaactaatgaagtgagtctgtctttgccatactaatatccaaacatgttcattttctaacttctagatgttcagtgccactgccatggattcagtattccttgtagtagtcatacaaatataggcatcatacatgcatccagaatactctgaatttttactcctttttcaaggacaatttgatttttttaaggggttagtgggtagatcatttagcaaaagtacttaaatattttattatatacaggtattgttttcaatactgtggaagagtgttactcttgagtacactgcatatacaaataactgtaatgcaaggtagaatgaatatgctacaaaacaaagtcttaggggagttctgaagagagcagcctcatttaattgcaaagtttgtaaagaaaagttactgagggagttgggccttaaaagtttgataagattttgacacatgggaaatgagacaacagaaaaatttcagcagaattgcgagagaaatattttctagtgaaggtgactgataattttaacatggtatggttatatattcacttagtgaaattattttttgagcaaagctttacttctataaatattgaaaaattcattctagtcagatatattcataaacttctttcttaaattccagttagatgtgttatatatttaggataaatgtgaatggttttggagtaaaatattaattcagtatatactgtaattttgcttacattttattatctaattttcagagattttgaagttatgttttacttcttaacaattgctgttattcttcataggtggaatgtttctggcgacgggaagcacagatcatattattcgggtttatttttttggatcaggtcagccagagaaaatatcagaattggagtttcatactgtaagtattggtgaaaagaattagatgagtataatttatgtgagtccaagagtttatattcagcacccacttttttgtttatttgtttatatataggacaaagttgacagtatccagttttccaacactagtaacaggtaaaatatttgtctaatttaattctaattttaaaattatgaaataaagacaattttagtcatcagattttatttgaagctgtataggaaataatcacctacctaaaagatcttgaccagttatacttaaaagtgaatatagccttcacatgcataggcacgtctttctcttatttgtaataatgttagtatacttggacacttttagtaattactgctttttaaatcaccctatatattccagatatgtttcaaaatgtagagaagcttttggtatagattgaaaatgtatcacagtataatatttaaggcagacctgtttattgatgatattgtataaattttcccatgttcttgttataaaaggaaataaagctacttctaataccttgagtagaataagtcagattctttaatattgagaagaaaatcattgttttttccttggaaaatgtcgattttatttctagggtagtacatagtgaccttgaatcacttttgaaaatgttattttgtgacctagccatgaaaattggaatgtttgcatataaaattctaaaaagcccataaagtttcagaagttattagaatgctgcataaataaagatgaatgctgtctttttaaacaagatctctgggaaaagtgcattttggtaaagcatgtatgatttttctttgggggatcggattccttttaatacaggtttgtaagtggcagtcgtgatgggacagcacgtatttggcaatttaaacgaagagagtggaagagcattttgttggatatggctactcgtccagcagggtaagaggtcaaatttgaaatattatgtatatattgattttttaaagtatgttatagtgcaggttggtgactctcagcctataaaattatatatcatgttaaaattactacttttctgcgataactagaggtacaattggagatgagcagtgttgatttttatgaaatgattcattggcatatttcaggacattgcattaatttttttaaggagatgataaaagtactgtaagaaaagttttaactttaaatatttttttcagaaaccagaacatattatgtttcgtttatcacagagggacaatattttatgtccaatggtttcttcaagccaagataaattaaactgcgtgttgtagctgttattttggttttaatatttttctccacttgtatttgtgtgtagtcatctcagatggatgggaaagcagaacagtcagcaggggatgacagcaacagtaaaagatggagagtgagcagcatggcacagaattcctggatctagatagagttatagcagtgatatttatgcaagatcatcttactaatggcttagaagtgctagaattctggaaatcctggatttcctgactcagtgcagggatttttaccatcattccatgttgattcaaacttctcagttactgttaagtgatagttattatgttagtgaatataaatactaattatagattatatgttttggtttttgttgtgtcagtattttaatagtcagtgtaaaacacttcctcactttcagccaaaaccttcaaggaatagaagataaaatcacaaaaatgaaggttactatggtagcttgggatcgacatgacaatacagttataactgcagttaataacatgactctgaaagtttggaattcttacactggtcaactaattcatgtcctgatggtgagaaaaaattctaatctttatttgatataactgaagctgactatagaatgaaacattctcattgaggaataaagtcttttatgttagctaaaaactcttgcttgctggggtaacggctttgtgagtgattgggaataattttaacagtgcagttaaacaggtttggcttggaaatatatagtagatatttgctgtttttttaatatgaaaagatccatccaccaagccctggacaatgatattagctcagtttcattattatatatacatgacaaatttgaagatttttgccaatttaatacttcttaaaactccatttctagaaatagtgccatattggtatttcattatgtttgtttttctttcttttttaatatctatgattctttgacacttcaaaaatatttcagggtcatgaagatgaggtatttgttcttgaaccacacccgttcgatcctagagttctcttttctgctggtcatgatggaaacgtgatagtgtgggatctggcaagaggagtcaaaatacgatcttatttcaatatggtaattatcattctcttcattttatacctactaaagatttgggagctttttgacaaaggtactctgcatgtcacaaaggaaatccatttaaaaattggttcattattttgactttaaattcccatcaataaagaatgatttattccacacttttattgacatgctgaatttaaaagaaaattaattgtgcatattacagtgcctgtacattgtgataactgatacagttttatcagttttagcagtgaaactttggaaattcacaagtatgaagatacaatatagagaagaaaaaatacttggaaaaccatgtgaagaaattataaataaatactcgtattattgtacttgtttaatagtcttctagcacaatagaattttactcaagtacactgattttaaaattaaggccaaagaatcaagcatttcttgcttttaatcaattggtagtatattaaatatttgagtttatactcttctcttcacattttgtttcttagatgttaacataattggccaagtatttgaattacaatgttttaatatatcacatacaacttaccgagcagtgtttctatttactgtatgaagtattaaggtctcctcaactactgcagtttgtgtagtcatattttttaaatgtctactttctgtatattagagatacctttcaacaaagaacaaaagaaaaaacagtttccactatttcagcatgtttaaatttttttaaaaaaccaattatagaaatctttagtaatgaaccataaaacaatacaattagtacatgtggtcagaattattttgccaaataaattatttcacaaaacagttgtgaaacatggtttgtgaaaaaattacacgaatgtgatgctatgcttgattgataatcagagtaaagttatttctatgtcgtgagtaagcagatataatagcagattttttagtgagggaaatagctttagctatctttaaggctgtgccttagaatacattaggtaagaaataaggtttgtaaaaccctctatttctagaaaacagtgttaattatttggtgtgcacttgttagtgatggatggatttcctttgtgattgtattgagatttttttaaaaagccacctgtaaatatgctagtaaaaaatattttctgcttttctatccttgtgtttctgatttgctcctttttggaatatctgttgaataaggaaattagtcatttctcaatctgtctttatagcttaaaaaaaataatatgtgagctatatatatactagtggcttcatttagtagttttgtatttccaaaggacattattatattggtagagtgtttggagggaaacttttctcccataattgaataccgctttaacttttcagttatataaaggtgaattttcccttatcaggtaagttggtcacactaatattctaccttttcagtagataacaaaaactgcagcaccttgagagttaagtgatttgctcttagaccattaaaaattagtaatgtaaccaggagtagaagtcagccataatatattgtatatttttaaaaattagctgagggattggttacattactgccttaaaaattgtttattgttaaataagagtgttagatgaggatatttttctggttctttttatggataagagataggaaaactctaaagagcagaagaaacaaatacatgtaataagttgaattggatagtattcatagaattgcagacagtgagcaaaacataaaccaaaaaagctaaaaaacagaaagtgagccacccttcatctcattaaaataaagatactacctttaattttttgtatgatgtttaatggtttccaaaatgctcccacatattttacctcctttgggtctcatgaaaaccttgtaagatgatcaagatagattttattattcttggtcttaggaatggagaaactgtacattacagagataaagtgctttgccccaatttactgagttcacacagctattatgcgggcacttggagcataaacccatgactctggttgtactcaaatcttgtcatggttggctttttgagcttttgtcaatgttactgcatagctaacttttaacaatactcagttagtgatatgtctatgaagaaatgttttcataatggtaataaatctgagtggatcttttgacatcttactctttaacttgattttttttcctgtctgtcttttatgagttatctaattgccctaattctttttgctcttcactcttttgttcttttcattgcttgttttatttatgggcaaatggaagataaagcatccagcattgctattaagaaaataaagagaaacaattcgagggagggcctgcgttctcactgcttgctatcatgaatgttgagttttggctataattatagctatttggctctctgtagctaatcaataagtttttcagttactaaaataccacgatacttgattgcatacaaatttaatccctaatgttaagttgtagtagtaatcacaagttcagtttagtaaatattttttgagcaccatgtgaagggtctagtcatatatagtgctttggtctattagaggatgaagaattctggtggaatactccttttttgactagaaatggtgaaagttatgtgaagtatttttaagattaaataatatttcatgtgactgttaaaactgaagtttataaaacagataagtaaactcaccattccacctacagctagaaaataaggaatgtagactatacctttagaataacagttttagtacaaatttcttaaatactggtttttatcaagtatattttaagaatactttaatatttctcctgctttttgaataataattgaattttccattggaaatttatacctttgcaaaagataatggaaatcatgtaacatataatttagtattttgtattcatcagcattttactgcattttttgtgtaattatggaaatgtaaattctttttttgaaattttagaaatagccaaaaatcctgttatgttaaaaagcctttcaagtgaaaaggtgaactttcaaatgcctgtttgatacatagtaattacattttaataatttttaaatataagattataaaacattaatattttaatattgcactgatagtcatcattgtaataagtattctaatttttttctgtatcaagccatgacttatacttttaaaaatttcttgtgctttattttgggaccatctaaatttgtcagcaaattaaagagtttgagattgggaattgagataaagctatttagttcttttatgtttaaataatttacttcattctgaaatcttataaatggattctcaactttcaagtagtattctccagagaaactgaggggtttatttttcttgtttttactgctaactttcctcctaatagaggatctcaatgataaaagcagttggggagtgggtgctgggtttgtttttgttacttataagaagtatatgacacgtgagatgatgttttaaaagaggctaacatagggcatgaaatggaatagacagtatatgtagcttgtactccccctaccctttctgttgtctcatctttaactaaaaacttttaaaagcacttattgggaaagcctttgtggttctttttgttgttgttgtttttcttttttaagtgaacaggagaagaaaccaaagagaattttgtagaaaatattatgagacaactttattctttaaaaacaaacaaactacattctaggccttcagtacttttatttctttacgaatcaactgaacagtccttacataacttgagtattcagaggaaatgtagtatatatttggatattttactgaagcaagtagtgtatattaagaatattccaaactataatagttgggtataacttagtaagcccacatttgtttggcattggcaggaggtaaacatcagaatagctttgttttgatgagagttgttctaaactctaaaggctctatactcccattaaatgctaaaaggcagataaataattcccagaaatagcttgaaaatgtgtgcaactctcaggactgaagctgagggccttctgtgaaagaggtcgccaggtttagaagttaattataaccttaaatgttcctaaatataaattgttttatggtcttttattccccattaaattagcaccggacagctaagttttaactacgagtttaactaaatgaatattaaattaatttgaaaagatagagacttttcacatttcttagtactaggccatttgaatattaaatagtaaataagagatgcttggagttgaatttgtagaccccatgtttttagtagctttcctcatgtttctggcggttttgctttatttctcttactctttcttttctctatagccctagctgtgtctctatctctctttccatatctttctatttctgtcattactcaaagctgtagtttgttgcctcctgtctggttttcattttaatcaaagagtatagtatgtctcttaatagaaagcagttttactgaatttgatttttgctttactttgttgtttcatgggtaaattccaaataagtttttatttgattaaagttttccttatttttataattgttctcttaacctttcttttacttaagagatttttttaaaaaaaaaaagttccagtgtccactaatccagaacaatatatttctccttgcaacgttccaaacgagcagaggctgccaatctaccacccagaatttttgtacatgcatacttcttctgatgttaacatgttctcagtgtgacctgtactttagttttagataattgtagctgtatgtatctaggaaagggagaaagagaagctcaaaataggtacagctcatattgtgttatctcttattaatcaggagaagagtctctctctttctctttcttttcttttctccctcacccaaccctccctttctccctccctcccttcctcccttctttccttccttctttcttcctatcatcatctaacaagataatgtcctatatttatactctctgtgttaaaaggactctctgatgcttccagtctatgtttcagcttctttgggtaaggaagtactttcggtcttcttaccatacactttagtaattatcactttcaggttttaagattctttagattatatctctcttatatatatatatcttggtacaaaaggcatgttaatctgttgccatgaatgtgagtttgtctctccagcttctcacactgcattatatgtggtaaattacttttatgtagtaattctttgacatagtccatgacttgctatcacaagggtatttgagatttgatagttatatgagaaggtatatgaaattatacagaatttaaacatgcagatgaaaacattttaacaaatatttgagtgttttcttttgtgttaagcacaaaatatgaggaccacaagggagaataagacgtggtctttttagtcagcctacagttaagttggggagacaaaatatacaaaatataccttaagggataaatattgataaataatttcaaacagtagcatgacaaatgagaaaagacagttgctgtatcttaagatgtagagatcattgtcagggtggacgattggagacgttttgttgaggagaaggaattgaaactatgccttgaaagataaataggaatcgagtcagtaagaaagagaatgaattttataaacaagaggaacattataaaaagctaagtgtcaagacatgttcatagaaatttagatatttaggaagcttgattgcagtagattttggaaataatgacgaaaaacaaattgagatgaactgtcagtgacttcaaatgatctcataggcataaatacctatgaaccttgtgggggctttagtaaattattttataggatggtcgagaagctaagagaaaaatagtttggttgccagtagtacaatttcaggtatgaaattgaataagtgtgaggacaactactttaattagtcatgaaatattataggggtagttggtaggaaatttgtaaagaaaaaaatacataaatgacttggtgttttgtgactatattaggatttgggctataggatgacagggaggaaagatttaataaatgggtttatagagtgttaagcccgggtgaccttgagaacaatggttttattagtggaaatatgaaagacataagggagaactgacttctttattcagttcatttgtaataatgtttattgcgttgaactacctatgtggaaatggccagctggcacttggagatagactacttagaggggctaggagtaaaaagaagaaagtgttgaagtgtttcttatagtatatagtttactttctcgtggagacaggagaaggaagtgcagaaggcttagaagtaaactttgggaaatactcatatatttagcagaatggaaaaggggtcacaatccttaagagggtgtgtcaaggaagacacaccatgctaatatgccaaaaagaccaaaagaaaataagcctcaaggaaggcctaatcaatagtattgctctccccagagaaatcataatatgggtacaaaatgatgttaaatttgaagatatttattttggtaatatttttcttttgatgaaaatctagagctgatttttcagaatcacttttatataataatttttttttcattttatgtaataatctgatctactgatttttatcaagtcaggtccagcagtttccttttttatgttggcataagttaaagaattttcaagattaaaacagccatgcaataactattttaaagtaatttgataacacctgaactgttggaatttggatttttttttttttttagacggagtcttgctctgtcaccaggctggagtgcagtggcacaatctcagctcactgcaacctccgcctcctggcacaatctcatctcactgcaacctccgcctcccgggttcaggtgatttcctgcctgcctcagcctcccaagtagctgggactacatgcgagcgccaccacacccagctaatttttgtgtttttagtagagacagggcttcaccatgttgaccaggatccaacacttatttcaaatatacatctgagtcttttttgtatctagtttacatatttttccctaggttggcaagctctaatctaaaagtttttttaatacataaggaaggacttcttaagggttattcttagcataaattccatttatcacctcttttcattcttggtatatgagtccatttcataagattattttagggtattttaaaagttgcatacataatttaaaattttggagtatttcctaggtaagatgctttcttctaatgaacggtgtgcttaaaaaaaaaaaaaaaaaaaaaagaacaagacatactgaccctgctaatagaattgaaataactctagaagaggatggttctgcctaagatagcccatattttttaaccttcctactttgtgttaaggttttctagaagcatatacttctcatattcactctctgtatattcttcatgattgatataattgagatcctgaattttagtatgcattctgcttttaatatacttcatctccagatttgtccagtttttttcaacatacctttcagaacctcagatggtaggattttagaactggattctagtgatcatttatctccatcattccacagagattaggtgatttacctattactgatggagagatgtcttgggtcttcgacctcattcctctgtctaatcattcatctttctgcttcaggaaaattatgttagttgtgtatagtaccttccctcaaaaaaggtattagtattctttccttaatgagtcctcatcagagctcttagctcataagtttttgtatcatttaatttctttaaagtttagtataaatctctaaatgcctaacaagtttatactactcatttattggtagtatttatagaattctttagtcagtatgagaattttcaaataagggagtcattattttaacacatctgatcctcccatctcctccttttaaaaataaaacaaaatacaataattgctgaatatacttttattttctttttactcctcttattctttttgaagttttcacagttactggtaaatactgtctcatagattatctcagctaaaccttattgatatttaatgtctatgagctttatgtggtctttatttctatatatccaaaagaatgcataacaagcatacttgattttattagaggaatagttcatctaaaatgcacttattttgataatttctcattacttattagtttcactctatttctaattattttactacaactgcagtaagtgctagctagcataagctatatgtagtattaaattttatgttgacacattttcttgatacgcagtgcatttaattttatgataggcttatatacctttcatttggtgagagagggtattttcttttcgtttttgaagataaaccttactgctttctagtagatgtgaatattcaaatagtgccttcatttaaaattttgtctttgagaattcttactcataaatactttgacccttgtttaaaaagataaaattcttttcttttgattggcttacttaatcgtaccatacattttaaaaccagttaggctatttttagttaatatagagaaagtaaaatattttcctgttttatatcatcaactgtttggaagcctggtctggaaaacctagtctgcagtttatagatatgggtgtttattgtctgaggatctattaataaatagagagttggtcttttaattgatgaagttagaggcaccttttctaactggttcatgagtaaagcagattcttttaatgcctcctttagtaaacaaatggttaatccaaataatatccttttcttttttctgatatgattgaaatatactttcattttctccatataaaagtcactttggagttaaacagatgccttataccaagaaaattaatactgtcttgccatattcacaatgaaatgtgttatataggacacacaggcatcttaaatatgattgctattttttctttgctcttttagagctttataagcttcaactgtccccaagattatttcagagatctactaactgtaaatgtggcaacattgataaataatagggtcaagggttgatcagtcctacttgccccgcaaccctcacccccaaaaagaaaaaatgtttgtttaaacaacacatgaaggataccatgcttaggaacatttatcagatttcctttgaaattgctaaattggactactttcaagtctccactctacttcaaattgtaatcttagacttgtaattatacttctggaaacatttattagagtagatttataaagtagttgcttttttgttctctctcttggcttctgtgaaattttgagtgaactgagttaaaaaaaaatcatttaacaacacatggctaaggcagtttcaatcccctaatttttagtttttgatataaaagtctatttagaggtgtactttgttcatgtggttgcttatttataacagttcatgtaacttttttttttatttgcctaggaaaacactgaaaaataaagacttgaatcctgaatttgtacttatagtctttcatttttggcaaatgcctagctgtatggctcctggtgcccttatggtaatctggagaaatagttatattctttcttgtttttgaattgttttaaagtagacttggaatcagttaccttcttctcctatcttatcatcctccaactttcttcacactcgagatataatcctgtttcacttatgattcatttcctttcttttctaagtttttgtgtatattaaacattagataattttcctagttttatctaatatggttacattttttaaaaagttattctttcttttctattattgtaaccatccaaaagagcattaataagcactgttgcacacatttcttctttttagcaagccaaaggaaaaagctagctttctttttgaggcagtcccatttgtggtattgtattaacttcttaggtaaagtcctttcttttatagaaatattttctctttttaccaaaatgcaaaccttccttgagaaatcagttattcttcttctgaattttaacttctgcatttggtgaactgtacatccatccttatgccacatttttaatactaaagaaagaaagttactacctttctcttttccaagcatttcctgggatattactaataaagctcaatttgttctagatcagtggataccggaacagaaaggaggtatttcttacctgataatgtgtgtgtgttccgattctgctaatccagaaatccaacagcatttgttgaaaatcatccttagttgttatttgttagggagtcaactccctattttagttgtttctgaaattcagtccctagatgtgctttagatagggtagaatttttacctctttcagaacactgctaaagaagttttcaactggaacatagtctgaagaagcactgagagcaaaaaattcagagtggtgattggccacctctgtttgattgacaggttgcagggaggaaacccattcgttctggattaatggaactggaaaacagaaaacaattatcaggtaaaaaaaaccttttctttaacctttgaaattcaccatttaaaagacgagtcagactaccagggaatttattaccaaaaataataaaatctagtgtcatttgagcaatgatctctggctactacttccaagaattaccaaaaaatccgtacagttgctacatgtcagcttttgcagaaagggcatgattgaacggagatagatggagtgtaagacataattatgtgttaaatctgagtttaaaacatgaagcagaattctatgggtgggggagttggtattcttttatttcctgaattgtgtttggtctgtggatagaatactttatttttcaaggctattaatctggcacttttcatgaatattcataaattttattactgtacaatgcttgttttacttaatcttcacttcattaggatgtatgcttttatcttaaaattattgtaataatagtgaccaattcttttcattttaatggtagttttcttactgttggttaaatatatttcagattgaaggccaaggacatggcgcagtatttgactgcaaatgctctcctgatggtcagcattttgcatgcacagactctcatggacatcttttaatttttggctttgggtccagtagcaaatatgacaaggtagagtatgatgactatatatcatgtccttatttttttttaatgcatgtttaggggttgtatgtttttggagtgggtgagaatcagcatctttacattcaggataaaattcgaagaatcttgtgttaagtagtctggagacttagtttcggaaaggaaggtgaagacagcagttaaatctcactttaatagggcttattatttgaaaatctgcatatttcaggaaaaaaataactcttagcctcctggggcaatctagattggaattcttaacttggggtatataaactggagatggaagatgttatccatagtgggactctgaaattatgtaaaatgtttttttaatatagataatacgtacttatctagggtacaaatctgtggctttcatgtggctcttaaaaatgtattatattacctgattgtagttaatgttttttattaaagatcctgtccagtttttggggggcttttttttacagagtaggggtgaaatgtctcactatgttgcctagactggctttgaattcgtgggctcaaacaatcctctcatttcagcctgctaaataactgcaggtggcactacaggcatatgccctgccactttgctcagcagttaaagaccccaactttaagattagtgactaaaagaattaaattgcttacttgtggtggaatggtataaaaggttatgatagtaaatattgagtagtttataatttagtttaaaaaattgcaaattaaccagtgaaattttatataataaaatttatgtcctggttgagtttatattaataattttatatcatctcaaaacatttagagaaaatgcttattaacccttagaaaatcttatgtgatttttatagtaatattacacaaatcatacttactgtatacctaaggatttaagataaagcatggtagctctgtggaagtaaaaatactgttgaatgatttatgactagatttgatgttaccatttaatttaaaattgaatattgaatagcttatttttagtggtagcttttctacacagtagcctcctcttatccacagggaatatgttccatgactccaaatggatgtctaaaaccttggaaagtaccaaaccctattcacactgtgtttttcctatacagtaatgggtgggtagcatatctagacaatgtggatatgctggacaaagggatgatttgtgtcccaagtgagatggcacgtgatttcattatgcattttggaacagtgcacagttaaacttatgagttgtttatttctggaacagcatttttggagtcaggttgaccacaggtaactgaaaccatggaaagcaaaattgcaaataagaggggactcctgtgtataatcccttacttgtttcagagaaaatttaaagcagctacttttctgatcataatacttaaggtagaaaattaaaatacctatttacaattaattcaggaaaataatattttaaaaattgttgcttaataccattattcgtattcaggtgtttgctttctattagaaatgatgtagactttttttctacaatgatacatttttatttttccagtcttttatggacttttctttttgcagatagcagatcagatgttctttcatagtgattatcggccacttattcgtgatgccaacaattttgtattagatgaacagactcagcaagcacctcatcttatgcctcccccttttttggttgatgttgatggtaaccctcatccatcaagatatcaaagattagttcctggccgtgaaaattgcagggaggagcaactcatccctcagatgggagtaacttcctcaggtaggtgctcattttcttagacaatcaaattttcttcttcccaccgaactgttaaaattgctaattgagtaaaactttgtttgcagttgttaagtggataagaagtaaaagtatgttttatacttttatacaggtctttgatcatgcttaaggattacaagttaattatggaacatgggcgatttacagtaataattttgatttattatccatttgtaacccctagggatcaataaggtagttcctgtatttggtgtgaaacaagtgaattaatttacttactcagcaacatttattgataatctcttctgtctctgacacctgctaagccatggctattcagtagtggattaaacaaacatgatccttgctttcatggaatgggttagtagattaataaacacagttcattagaataaacaaatacatagttcaaattgtgatgaatgttactaaagggaataatagcatgatgtgagagagattgtagattgcctgttcacattgaagatgttttaaaaagttaccaacttttaaaggtttataagaggattgacatggaaagtgttgagagctgttttagtgaagtataatggagtgttgaagaattaaatgagagttaaaatggagtcaaagtctacaaactgctttccctcttttgacaagaaatgcagcactagtgtattaagtaaaaataaggaaaggttctgtaaagaaggttaaaccagttttgtcattatgaatacttctactatatttttagactagaaaaatatttatgaaaatattttaagttctagacttcaatcgacaaaagcttctaagtagcttttgtgttgagtgacttttaaataatgaaattagattctgtaaggagcacagaaaaaatcaacctcaaacgtgcatcttatttttcttaaaggatttagcatgaagttgcaataaagatggacatactggatagaaggggctcctcttggaattagacacgtccacttagaatggagggtgagaagaagcattagaggaatcattcctgctgctactcctagagtagtgcagtccagtggacttatctggagtttgtgagattaatgtgtctcagtacgtggacacagcatgaactcaaagaataagtagtattcgagaagtatggacagatactcagactcagattagataagtttcagtattagagaagttctggtcataggactgaaattaagagaaatgtcttcagtctctcggagaggaattggcaaaatcagattgttcccaagcctcaaaatagatagctactttagggtttcagacacagggaaataaggaaaagactcatttattaaagtatcagtgctaattaattattattattactttttaaatccatttaccaatttataaagggctttcataaacaatatttaatcctgtatctttgatattccagtcctgcaggttgagagaattcaaaatttaatggtaatgtgtcatagaatcacagggactctaacctcagctctgattctcagtccattattctttcttccatataaaataggcataatcttaaaaatgaaatagataccagttgttggtatccataagagaatccatagcaataatactggctttgtaatgagtcccaaaatttttcagtgttctgattctcagaattcagagtaaaaatctttttttgtcatattttagacttattgagtctaaaattggttaatacaaaaacagaacaaaagcaaaattcttttcaaaaaaaaccttagctttttatttgcaaatccagataataaaatttattctgggaaatctctcagagttagggaaaaatagatttgagttattataataataaattagcttgttgtattggcaccatacaacatactttaaatgtgtttttttcagttgtaaacttgagctatatcagattattcatcagtattgcaacatggttttccaacagcaaaccaggaaagaagtataagacatttgaacttttacttctgtctgcttttactacttctactttggtgaccttaaacaagttattttatcctattgaattttagttttcttttataaatgaaattatgcatgtaatacatgtaatttctttttccttttacctttcttactactacttcatttcagaatattcttgctctcttttcttaccatttaagcccaccctaaaattctgagctgatcagatcttctcatatacttgtatggctgatactaattgctcccttctttatactcatggcaatatttttcatatattttataatataccttttaaaaatttattcagcaagagtgaatattttttaatgcctagcattgtactagatttagataaggtaagtgagacagttcatgcttttgaggaactaatgggtttggacttcatttatgttttatattggtctgaatacttagatatgtatgggttgtttaaggaagtgaaagagggtagcaggtaattctttcagaagtaattggaaagatttcacagaggaaatgtcatggaattgccattttagttggtgctggaatggtaagttaagagattgtcatgtttatctttatagtgggaatattgtgttcaaaagtttcagtgcatgaaggtgtttactttttagaagggcagaagtcaagtggtcctctatgacaagtaagaattaatgctggaaaacttggatggattaaaatacaagtttctccctgcttcacagcgtttgtacctcttgcgttctacctgaagtgttccttatctagatctttatgtggctatctaatttaaatctcagcgcaaatgctaccttgtccgagaggacttctctgatttttctgacccaccatcaccttctttatttcaacacctctctttcatactctgtattaactcctgggttattgtttactttccccatcagtatgtcagctccctaaagagtagggatcttcatgtcccttatttaccactagatctcagtacctggcgtagttcctaacatatagtagacagtcaataatgtattgaataaatgaggcagtaaacattttttttaagaaaatgggtaatcagatgtgtcattttaaaagaaaccatgatctaagtggaaaaccttactattgccacttattagatatgtgacttcgaacagttattttggttttctcatctataaagtggagttgtttatacatacctaatggagttaccatacggattatgagtatctgagtcatgataaacacttagtaaaagttagcaaccatcctcatcgttatttttgataaactaagatggacagatgttttctttcctttttttttttaactcccatcatttatagttgatcatttaagtcttttacccaagcaacatttatgtttaattctgaactcataagtgaggttggtgagtaaaatggtttgaatagtagctaaaatattcgtaatattttgtgtggtattgtataggactgaatcaagttttaagtcagcaagcaaaccaggagatcagcccactggacagcatgattcaaagactacaacaggagcaagacctgagacgttctggtgaagcagttatcagtaataccagccgtttaagtagaggtaagcaggtattctattttaagtgaaatatttaaaaatttgaaacagcctaaatctcccacagtgttaagtggtttaaaaaaattatggtacatccgtaaaatgaaatattaggttgccaaaatgatcatggttttgaagaatatttaatgacctaagaaaatggttcataatgagatttcaagttagagataatgagtttttaaatgtgtatgtcttccacattttctatagtaaacatgtagaatagtcaaagccacattcttgaacatctcatcacccttattgttgtatattattaaactttaagagtcaaaacaaatttatgaaccattaatactcttgggtttgtctagtttgcttcttatgaagttttctcaaaaatacttagcatttttttaaagtaatgattttgtatccttggggcttaatagtctatagttatgattcattgacttatctaatcaactgaataaccatgttttgttttcattaaaccacttttgcctgtggtttagttcctgatatggtaatttaattattttgccacaatatgaagtttcttatggaagatagtgttttatctcttttttctgttataaactaatggagaaatatctctaagtagaacttaatatagtaactttttcacttaggctccataagttctacctcagaggttcattcaccaccaaacgtaggactaagacgtagtggacaaattgaaggtgtacggcaaatgcacagcaacgcaccaagaagtgaaatagccacagagcgggatctggtagcttggagtcgaagggtggtagtacccgagctatcagctggtgtagccaggtaagggaagttgtgaatttttatgtaccatagttccttgggttcattttggagttaacagcattcacagctgttattttcagctctgtaattcctctgaaatttttctggaagggcagtataaactattcatgactgtagaaaaattaagtttctaaaatatatttatttctagaatgatttatgtaaatgataagaatgatacgtgttaaggtcaacaggaaaatcataaatttggattgaccaaataggaggttttattgagtagcatatttcagtcttggtttgtcctgtttaactatatcttaaaatacattttataaagtttgttgtaattatttaattttttaatggatttagccagccattcacatgatattcagatgcaaaaaaaaaataaattggaaaatacaaaaagattttatatatattaaaatatttatatttaaaagtatacatttatgactttaaaagtattgatttaataaaattctattctcttagaccatgccatgagggaagtttatatccagttccttttgatcttcagtcatgaaatactcatgaatgaggcctatatgaacttaagtaaggctgatttctgtgtatgaagtgtgttctaagtatgaaggtcaaaatgtgaatttgaatttaagattgtctagttcttcaactcagaagatagtaattacataattaaaattttggtcttaatgttcatatagattatgctttttgattggacatactttgaatttatgttaggatttagaattaaatacacaccaaaggtatacatttacactttcttatgacaaatatgcccagttatccgtcataaattaattaactcacatggcatatttttagttagattcttgtttactttttaattctaatgttaaggtttgaaaggggtcaaataaataatgcccattttttcaacaccaattctgaaataattgaaaaataacatggagaataagacatagtaaatcagcctatcactgtttgttactatcactttttatttcgtgaaataaaacccatatcagtaacaaaaagtaatattccacaaaaaccatatcagtaacaaaaagtaatattcaccagacacagatagcagtgggccacagtttttctccccttgtgggacaggacatgtggtttgcttctttaagatgcagggccaaatgtataattgcacacaatggcataaggctgggctgtgctaagctgggtttttcttccagagcatatcagttgactaatttactcctcctctggagaggaatgagtcaggagataaggttgctagttgtaggttgaggctcctgcacatggaagggaacctgaggagtgaggaagatttatttctcttcaaatattcttcatccttttattcctttatcattgggcttaactattcattggcctatggtcaagcaagactgtggccataactttaacagatattctttctttgggcttcctggagctttgtctctaaaactttgtactcatcctatgggaatcttcctaagaatctctccagtgggtagtctaaagtttccttttaaaaaggctctctgaaatgggtcagcctcatccattaattaaaatggaatccaatgacatacccaacagggggctaagtaacatagaaataattctcataactcaatagtaaacaacagaatccaaccggagaatgggcaaaagacatgaacagacatttaccaaagaggatctatagatgacaaataagcacatgaaaagataatcaatgtcattaaccactagggaaatacaagttgaaaccacagtgaaatatcactgcattcttctcagaatggttaaaataaaaaataagtgacagcaccaaatgctagagaggatgtagagaaaactgaattactcatgcattgctagtgggaatgtaaaatgatagagccattttgggaaacagttttgcactttcctaaaatcctaaacatggaactgcctgtcatatgacccagcagttgcactattgggaatttatcctaaagaaatgaaaactcatgttcacacaaaaacctgaacataaatgtatagcagctttatttgtaatacccaaaagccagaaagatacccagatgtattttagtgggtagatggttaaacaatggtatattcacaccatggactactactgtgcaataaaaaggaacagcatattgatacaatgggtatgaatcttgagggaattatgctgagtggagaaaaataatttcaaagtacatcctgtatgattccatttatgaaatattcttgaaatgacaagggtataggaatggagaacagatttaggttgctaagatacaggcatactttggggatattgcaggtttggtttcagaccagtgcagtaaagcagctcacatgattttgtttgtttgtttccaagtgcgtataaaagttaaggttatactattctataatctgttaattgaacaatatgattatgtctgaaaaaaactacatacctaatttgaaaattctttactgctgaaaaacgctaacaaccatctgaaccttcagtgattatactcttcttgctggtgaagggtcttgcctcgatgttgatggctgctggctgatcagagtggaggttgctgaagcttgtagtggctgtggcaatttcttaaaataagacaataataaagcttgccacattgatttgactcctctcatgaaatatttctgtgtggaacttatttcaaaactggggtcagtcctctcaaaccctgccattgctttattaggttcatgtaattttcttttcttttctttttgagacagagtcttactttgttgcccaggctggagtgcagtggcgtgatctcggctcaccgcaacatccacctcccagtttcaagcaattcccctgcctcagccttcccagtagctgggactacaggcgcatgccaccatgcctggataattttttgtagttttagtagagatggggtttcactgtgttagccaggatggtcttgatctcctgaccttgtgatctgcccacttcagcctcccaaagtgctgagattacaggcatgagccaccgcgcctggccaagttcatatagttttctacaatctttgttaacatttcagcaatgttcaaagcatattcactagagtagcctccctcaagaaacctctttgttcattcatgagccactcctcattcatttaaagttttattatgcaacttcagcaattcagtcacatctttagttgccgcttctaattatagttctcttgccgtttccattacatctacagtaacttcttccactgatgtcttcagccttcggaatcatctatgagggttggaatcagcttctttcaaactcctgttaacagtgatacttttcctcttcccatgaatcataaatgttcttaatggcatctacaatggtgaatcctttctagaaggttttcaatgtattttgcccagatctgttagcagaataactagtgcagctatagccttatgaaatgcaatttcttaaataaaaagacttgaaaatagaaattattccttgacccataggctgcagaatggatgttctgttagcaggcatgaagagaacactaatcaccttgtatatctgcatcagagctcttgggtgattaggggcattgtcaatgaacagtaatattttgaaagcaatcatgttttccgagtagtaggtctcaacagtgggctaaaaatattcagtaaaccatgctataaatagatgtgctgccatctagactttgttgtttcatttatggagcacaagatgagtatatttatcataattcttaagtgccctaggatttttggaatgataaatgagcattagcttcaacttgaagtcactagctgtattagcccctaagaaaagatttagactgccctttgaagcttggaagccaagcccttacttctctctagctgtgaaagtccagatgatatcttcttccatttagatggctgtttgatctgttactacattgaaagtctgttgtttaatgtagctactgtcttcagtgatcttagctagttattctggataaattgctgcagctacttcatcagcacttgctgcttcaccctacacttttatgttacacaggtagttcttaggcctcatgaaccaacctctgctagcttccaacttttcttctgcagctttttcccctcttccaaccttcatagaagagaatagagttagagccttgctctggattaggctttggcttaagggaatgttgtagctggtttaatcttctatccagaccgcaaaaactatttctgtatcggcaacaagcctgcttcactttcatatcattcctgtgttcactggagtagcatatttaacttccttcaagagcttttccttttcattgaaaacgtggttgttgggcacaagaggcatagctttcagcctgtctcagattttgacatgcctttatcacagagtttaatcgttgctacctttgatttaaagtgagagatgttttcactggatcagttagggtttttaattggcctaactttaatattgttctgtctcagggaatagtgaggcctgaggagagggagaagatggggcaatggctagttagtggagcagtcagaacacacgtaacatttgtcagttaagttcattgttctatataggtgaggttcatggtgcttcaaaacaactacaatagaagcataaaaaattactgacaacagatctccataatagatataatggtagtgaaaaagccagaaatattttaagaattaccaaaatgtgacacagagacacgaagttagcatatgtggttgaaaaatggcatttgctcattgcagggttccacaaacttgcagcttgtgaaaaaaaaaaatgcactatctgcaaagcacagtaaaacaatgtatgcctgtgtacacacacacacaaactaatagaaactaatagaagtaaaactggggaagtctcagtaagatcagtggattgtataaatgtcagcatcctccttctgatgttgtaccatgttttacaaaatgttaccatttgggggaattagataaaggatacaaagttttgcaaaatgttaccattttgggccggcgcggtggctcacacctgtaatcccagcactttgggaggccgaggtgggggggatcacgaggtcaggagatcgagaccatcctggctagcacagtggacccccgtctctactaaaaatacaaaaaattagccgggcgaggtggcggacgcatgtagtcccagctactcgggaggctgaggcaggagaatggtgtgaaccccggggggcagagcctgcagtgagccgagatcatgccactgcactccagcctgggcgacagcgagactccatctcaaaaaaaaaaaaaaaaagttactatttgggggaactggataaaggatacaaataatcgctgttatttcttacaactgtatgttaatctataattattgcagtaaaaagtttaatttaaaaaaaaaaaattaagaacaaaaaaattaaatccaagcctgttcttttcacaagatttaaatttgctcattttattttccattcagaattatggccagccacaatggagattgtgccgttaagtaacatgataacattaatatcatctagtaaagaaatatcaacagtatatagttgcactgtgttttagtattaactaccttcttattataaaatcttaagggagaattaaatttcaaatacccttgcatttcaaaaactttaaagttgtaagatattgaaataaaaaataaaattatcttctcatgagttcattttttgtattaaagtttaaaattagtgttcctcaatttgtgttgttcagtttcaccttgatggtttgagttttttcagtcggtgaaacctgacctccctgtctctggaaagagtgtagttctttcagggctttccgtgtaacctcagggtctgttgtattgaatttttaaagagattccagctatctatatggaacagaagattatcaaagcataggccctatcctaaaaaaaaaaaaaaaaatccaatttctcactcttggaaaaattaccctaggaatcaccacattcccagaatctaatacttggtttgccagagaccatagcaggaaccagtagtatcaagttggggtagacagagtttgtctttccattatttcagtcatttaatgccggatgtcctgagcccacaacctatgaaattaaacttagaaatctttatcacatgcttccatcttaaaataaaacatcaggagtagcgttcctttcctgatacctaacattccttttggaggaaagttatgtgtaaaattctagattaatgcatatggcttttgtctttcagtaggcaagaagaatggagaactgcaaagggagaagaagaaataaagacttacaggtcagaagagaaaagaaaacacttaactgttccaaaagagaataaaatacccactgtctcaaaggtacatattaattattttgatgtttaatatttgcttcttaagtatagtgaccattttaaggctccattttgtaatttaacatggtattataaacaggttaatgataaaaattaacatagtaaatcttggttatctctggattatctattgtttactatttaaaatttttaattccatttctgctttgtagcataccctttcccaaagtgccaattcttacatactcatgaaattctaataaattacactgaagcctcatcaagaactttctttagctcttcataatcttgtgtgccatctttgtacagtctaaaaatagcattttgttatgtttttctatgtgtttttgtggtgtcttgcatctattcattattctagataatcaagtcagctaatgctgctatagataatatttctcattttcttcattaagagaggtagattgtccctaatagttttgacttattggcttttctgtcatcctactctggaaaaactgttgcacccagtagatttcaattggtcatggcctaattgagagagatacgtacttcctgagtgtgaaggaatcacaaatatcgctttttcctttttttcttttcttttcttttattttggagaaaaacatcttagcttgaaagcttcagagccttagagtctgaaattgaggaagcctatgtgaaacttctttttaggtccaagtcactatctaaagcaattattgaaacaaaggaaaaataaataactggccttcttcaccagcttcgtgttccttcctttttttcatttgagttttttaaaactcctaaatcagtcttcttctctagaattcacttttctgttttattactcatctcttcaaaatactcaagtagtccttttttctttactgagtgtatcataccctgggatcagtaggacctcacaatgctagaattcctgttagtataataaaggaaaatttctttctgaaataatcttgttttttgcttgattgattggttggctttttcatggtaagtggaaagcgtaccttttttgcatcctttgctgagataaacttatataatgatgatgagagtttgtattgcttgctactacctttatggatggaatatgttaatatctattaaaattaaaagatgtgcttatctcttaattcagtagtactttgaggactttttaagaaataaaaggaatgatatgtatatgtatacatatatgtatgtgtgtgcatgcacactcacatatttttagcgtctgtggtcgcaaaaactggaataaccagttattatttatgtggtcagctggctatcatttatttttcccaaatttaattggttattttcgatttcaaggatgtctaattgagtctttgtttttttctaagacaagaccctgcatttatttcatggatgtgattctcttttttgtttggttggtggggagacagagtctcattgtgttacccaggccagagtgcgttgggacagtcctagctgactgcagccttggactgcctcaagtgatcctcttgccttatcatcccaaatagctgggactataggtttataccaccatgcctggctaactttaaattttattttttaactttgtagagacagggtattgccattgccctggccagtcttaaactcctgaccccaaagcaggattctcccacattggcctcccaaagtactgggattacaggcatgagctaccatggctggcctttgtattcacttttgagatagctttattaagttttttgtgttttttaggtataaattttaatgtttattgagtttgtcttcatcactgtgttgtactttatcagttttttagtgtttattggttgtattctcatctgcagaagacttctcacatttattccagcagaagaaaaataagccagccacatggcatcttatcttcatagtggcagggtagctttatgatccccattttggtttccaatttgcctctgcctattacatatgcctcatagcccattatcctttttcttacatcatttctataggtttgatgtagatacagaagagttcagtgtttggtttagtcttttatcatgaatccttatgtctgtcattaattagaaaaaaaaaaaacagcatttttgtaactaatcaatcagcaagtaaatatcagcataaccatgataagcctttaacttgatgaatgttaaaatatttgagttggtactataacctcatccatttaagtgatatcttaaatatgaacaaatgtttttattgttgttgtatgttaggataaattgtctcactttgtcacccaggctgtagtgcagtggtgcaatcatccctccctgcagccttgaactcctaggctcaagcaatcttcccaagtagctgggactataggcatgcgccaccatgcccagcttgaatttttttaattaaaaaattttaacttttaaactaaaaatcctgctctttaaacaatctgtgaataaatttaatttatttttttctagaatcatgctcatgagcatttcctggatcttggagaatccaaaaagcaacagacaaatcaacacaattatcgtacaagatctgcattggaagagactcctagaccctcagaagagatagaaaatggcagtagttcttcagatgtaagatatcagcacattcataaattagatgtcatttttacttatttttttaaatatgagtttcattcttcgcatttattgatatgtttgtagatgtagcttaaatatggcattttggggtcttatgatacagcttttattttctaaaaatctatttctaggcatggcacacatacacataatttcaaatccacaaaataaactcgtgaattaattattctcattttatagataaggattctgaggctcaaagatatttagactatacagtcacctattaagaggtagagcagggattaaatgcaagtttgttttacttgaaagccaagctttttaagtggtaccatgaagctgtagtgtagtgtaagtataacaacaaatcaataagtatttgacaatttttgttagtttttaatctctgtaccagtttacagctttctcagtgtaatttttaaatattcctcatgcgtcattatttcagtaaatattactgtccttactaaattatatattatttcagtgaaggcaatcaatttgcaatcataaaccaatcttattaagcagttaaaccatctccattcctcttttcttctcaacctttcccatcaccttccttctttacaggttagagatgtaatcattttagatattgactactatacacttttgaagccatgttgtgctttgaaatgctaggcatatatgagaattctctaaatatattctctttatccagggcaacattaactctcaaactgagagtattctaaccacaatttaagttagtctccaatagctttgggtcctcatatagccaggtcaaaatgatatattcattctggcaaaaaaagagaaatatactgattatatgaatttgacacatggtaatttggatagacatacaaaacataaaatacatgtgggctaattatttatcttataaaatgtctctttatttttgaaagagtttcctctaaatattaagtttctatataatatttaaaatattctgtttattagaaacttaagaactgtatttccagaaacacatctgttacaacaagtccttcaagtgtcttcaaactaatttatttatgctttaacatccttttagaaatttagaatatatttctagaagagtccattgagaacaaagtatcactttgggcacagtatactaattaattggaaagaattctgatttttttggttcagatgttttgtactttagtattttgtatttggaatgtaggaataattctgtttctttttcccccctttaaggaagtaacacacaaaatgtgggaaaaatcatatcacttgcttagatttgttatatcgagcagtgattaaatttgttaataaaatatgctttttgttaataaaatatgcaaatgtgattttttcatttttgcttagttttcataactgtggaatagacatataattcacattcttcattaaaacatcaaaatgggaaaacttttgtcccaagtatattattttatttaaatattaaaagaatccaccccattgtcctctttaaaagttatctgaatatgcccagcaagtaagtaacaccattcttacagaatgttgtccagggtttggactaaagtgacctgggcttaaatcaagttttgttgcttattagttgtgccgccttgggcaaaattctcaaatttttaaagctttagtttgtttatttaagtaactctctatcaccaatagtgcattgtcaagcccaaatggggtgatacatacaaagtgcttagaatagtttgtaacatacagtaaaagcttaataaatatgaggtgttattgttattggagcataaagtttatttaagtgaggaagacatgcaacatttttgccatcaaaaaacccaattttatgaagaaaatgctttcacagataatattctttgtttactcatgaatatttaccaaagagaaacagagtgtttaagtattttttaatcctttaccatttagataaaataagaccagaagtggaacatccttttttatatgggacagctgttttaataagtaaaacataaatgtgcctataataaataaaacataaatgtgccttctcaaagcacctatagaagagaaagcttagtgttttaaaaaggctcaagaagtaaaaattaatttcaaatgtaaatatggaatttgttatcattttatctagatacgttatgaggcttttacaatggtaatctgtaggattgctctgaatccaatattagtattatgtctttatatacatttacatattctcatttatttgtgaaaaaaaggtaaaagctgtattttgcaactaattttttaaagagaagcagctgactcttttattattttattattattatttttgagacagtctctggagtgtagtggtgcgatctcagctcacggcaacctccgcctcctgggttcaagcaattccgtgcctcagtctcccaagtagctgggattacaggcatctgccaccattcccagctaattttttgtgtgttttagtagagacagggtgtcgccacgttggccaggctggtctcgaacccctaaccttaagtgatctgcccaccttgacctctcaaagtgttgggattacaggcgtgagccacaacaccctttgcaactaatttaaatcaaattttaaatatcccaatatgactgatattataagatatcactgatattttctgtaatcataaaatgttattttataaaattatgcaataagacatgtattcaacatatcttaggaaggcgaagtagttgctgtcagtggtggaacatccgaagaagaagagagagcatggcacagtgatggcagttctaggtaaatgaatgctttttatttttccaaatgttatataaagtctttagaaaggtgtttttttttttaaccagcaacacgtttcaaagtctctgtaagttcaaagttcttcagagagtttttgaagttgtatgaaatgtagattttctgatatatgttctctatagaaaataattttttggtgattgcaactagatctgcaaaatgatgcttttaactttatcatcttagtattgtttcttctgaataccgttataatgttgaattgcaaaccagtttcattactggttttgaaaagtatgaagttaagaaagcaatgcaagcctcctgagttaactatcttatatagtggaatttatggaatactaatgataagaggaataagctctcctttttcttgttatgcctaccttatttgaccagaccagtgaggattccttcaactccttatactggcgattcacattgttgcagagcttatgaccttggctaactttgcaaggtcttaatagcaaaaagagagtatagcctttggggatggcaattatgattagctgctacacttatcccccaccttagtggctcttaaaatagacgagacaaagtatgaagtagaggctgtaagagttcctcagttgtacaatcaagggctctcctattcccacagtagtgatttctggtaatgatgtgcaaatgtgtgtggttatatctagtagttttctgtattcaaaacaggtcaatgaaggaaaaattgaatttcttttggctcgacagcagaatcagagtatttcaaaccctgagatgacataggagttcctgtattaaaagagtagtggtggttttagcagttgaggacggttgtaaagaatgctggactctggagtttgtaattatgtttatgctgacagcagaagatcttctgtaagagccaagtactccagagagtgataactgtgtgatttggcaggtaccatttacagtggccatggctggagctagtataaataaaatgctcataagttgaagacaacctattttagaatagcatagctgaaaatagtgtaagttcttaaaaatcattttaaatttcatagtgaaatcactaaatcatgctagagaacatcttaattatttgattcttatctgggatgattaggataatcagagatttaggtacgtgcttataaagaacatttggacagtggagaggggtagtggggagggttagtgacaatggaataaagctccagaaaaatctgctttttcagtgttaatgttaacagcagtatttttagaggcatgaggtcatatatcattaaatctgggaaagctaattattgcttttaacatagtataacttaggttcctttaagacatttatgttcataaaattgtacaaaataagtaaatatgctcacatactaaaattaatatagaagataatacaactagattctttattttgcaacatgattggttcagtaaatcgtttgtcaatgaaggtaaactaaaaagaaatgacaaacatgattggaagcagtatacaaaatatagtaaggcctgtatttttctgaatcatggcatgctttgaagcataagatacacctaacgtgtagggtgtaggtaggtagaataattttatctaaatgaaattttgttactttacatgcagagtaaagtttgcaaacacagtctttcccctgggtaggaacacttctgactctgccagttcaatatccagctaccctgagagcactagtactggaccataagagttcccctgtctggaactcccacctccccttaaaaaaggaatccccatatctccttaatgttgacaccttctgtgctctccagggttatctttttgctgtcaatagaactgagaaaagtgaccccctcttatttttatatgctcacagaatttgtaacatatgcaacactcagaagtcagtatttttcttaaaatcttggaaggtacttaaatttgttttttcacttaaagttgtgactttatgtatcttcaggttttgggggagtttttttgcgatatttaagtaaagcctatagaatttcaagaaacttttgaagatgagttgaaaattagggaagtttttttgtagtttaagtatttgttctgtcatttctctttattataatctttcttttcgtttttataaaacaagtcttaaaattttatcttgtgtgttatctaataatcccttctctccttctagtgactactccagtgattactctgactggacagcagatgcaggaattaatctgcagccaccaaagaaagttcctaagaataaaaccaagaaagcagaaagcagttcagatgaagaagaagaatctgaaaaacagaagcaaaaacagattaaaaaggaaaagaaaaaagtaaatgaagaaaaagatggaccaatatcaccaaagaaaaagaagcccaaagaaagaaaacaaaaggtttggtttttaacattacaaatttggtgttttctagagaaacagtttaaagcacacatcttgaagtacaattgatcacaaacattgttgaagtgtatcagcaaataataatagaaaaaactcagaaatacacagtagtaaaaaagtttacctaaatatatttagtttattcgagtttttagatatttaacatgtaaaagtatttacttacaaaattgccatattctaaacattgaacatttaaccttaaaaagatcagatttttcttgacagtttggtgggatttttttatgtgaaatacataatgatacaaaacatatagtgtattaatctatgaattgcctttatgtttaaccatccttattaattgttattgctgaaaataaacaaagtctgtgaggaacatcacacagagtagaaatgacacaacttgttacagttgaccccaaggcgaataaagtggggtaagatggtcttgtaacattccttcattaatctacagtgtgtgagctataagttttcagtcctttggtcagtagcagtattgatttgattaccacaattaggtctgtggacaatttgagagaaagcagagaacgtaagaaatattataggaagaatatctgtcagctcttatggagttgaaattctttaacataggaatgaaaatgagacctgtttaatttttgttctaaagtaaatacaacacgaggggcatgttttctgcttcccaaaagactgtaaatgggaagcagtatttggttaaaaggaagatttttttgtaacctctgccatttaagtataaattgtcgtttgtagaattaaatagtaggagaccgtagggtctttattcccacagagctttcaaaaagtataagaatatatctgttttaagtgtttattttatctacatttgttgagttcttgtttcagctttataattatgagttactttaatttggattccacagctgctgtttttcttgttgtctcctttgcttttgtttggggaagctttaccaaagagaatgccacacatttacccacacattttcttttctaccttgtcagtgatgataaatgtagtctgatttgttattttatgtatgtagtagacagattggttaaagaaaactgatttagccatgtgtctggctaattaggtaaatattctttttcatctcaggacagttttgatgaacttttgaggtttgcagtgataagttactgtgagaacagttccttcatcccaagagtacagtttgcttcatctggtccccttaatttgataattttcctcaaactaataccatgggctccttcatgcactggcacatatagatctattgttaacatttggcaggttttaatttcactgtgttgttgcaacaaagtattgtgataggatgactctgccaatagaattatgtgggtcactttaaatatggtaaggttaggatactggagggtattccctttagtttaaattttgttcgcatgcctagcagtgatgtaacagtagatggacttgttcttattccctagtcctctagttatctcaaataaacataaaagtatttccattcttaaacagggtcatcttatgttgggatttcagatgagatttcctagaggaaataagatttgagctgcgcctagaagcattcccatggttggaaaggaaagaaattatttatactttccatatttaaaaaaacctcaaatcatggagctagaaatgttaaagaatactcaatgcattcttaaccccttaatggttataattaataagtgacagaaatcttcctcaaccaaacattctaagttcctcaggaattattggggaataagatatgtcctcataatgatttttggtgatgcccaaagtctttcagtcatacatatgattattcttatttaatatacaacttacctgtcttgatgtaaaagttgttagcttccccattgaacatagataaattccttatggggagaaatccttgcctagcaattaatagtccatattgaggctagaattatgattttcaaacttttctgtctttcttttcctttttattagtcaaaggaaacctttttcaaacaaacttttagctcctcagtatacaaaactgatagcagctgctctgaataaagcacagtggaagttcctgggttgtgggatctctgtgatatactcagtgcataacataatggctactttatttgataactgtgtatatatcaaattactgtttggatatcattttactcctcacatccaacacctacaaaacttatttaccaagctttttcctcaaacccgctgtaccccagccctctttatctcagtaatatgacgactctcttagttgtttataccagaaaccctggagatatctttgattcctctttttctctcatattccacatccagtccatcagggaattaattatctaatggattataagtatattcagcactttactgtttttcaccactccactgccatcacattggtgtgaatcaccctgaactctcatttatattactgtggtagcatctcatgtgtcttcctgtttctccttgtttctccctagggtctattctttaaagcataggttgacagattttttctacagaaggccagctactctgccataatagcatttatagacaatacataaatgggtatggctgtgttccgataaaaatttatgaaaattgtggcaggccagacttgccctgagggtcatgatttgctgatccttgttttgatgcatccagagtgatccttttaaaatataagtgagatcgtactatgcttctgcctaaaattctgtaacgagcccatctcacgtacagtaaattccaaagacctttaccctaactctccagggtaatgctcttgatatgtttgactgagaaacactcagtgtggctgtggctaataggaaaaagaggaaaatgacaactactacagttgggaagtagtagtattacctattgcaagtagtgttctctgatatcatgttatggcattgcttcttttagtaaacaagtattattttatactgataattacactaccttgtttttaaaaaaacaagtaaaatgtagcagtcttagtgtacaatcatttttaatgttttacgtgtgattcacaaaagactttaatcaattgcataactttttttacttgtaaaaatattaggtttcaaatgttcttagaaagttcatttttttcctgagtctaaggtaacaacatcaagaccattgtggccacttgtgggtgaccagcaaagttaaggaactttctcagaggcatagtttctcatgctgtgtagaagtgtaatgtccagcttatggaaataattagctttctacatgttgcactactcaactcctatgagaataccttatttagttgtaacttactataactaggattaaaaaaaaactctaggctgtcttggtttgttatatggaatgtttataaaattgatagcattttgaaaggaaaggagaatacacagagcttgtactttggggtttgtggggatatcttgttaccttgttgtgagtgttggctatggatttttgggttttgcttctagttagttctctttgtagacagtgatttggagagattgaataactctgttgccactgctgccattttttctagaataagtgttttgttgcttaaaggatagtcatctctcagtagacacggaggattggtcccaagacccctgcagataacaaaatccagggatgttcaagtctcttataaaaaatggcatatagtatttgcatgtgacctacacacagtctcctatatactttaagtcatttctagattatttataatacctactacagtgcctacacatcattttatttgtgtggattcaacatagtattcagaatgtagcaaattcaagttttgcctgttggaactttgtggaattttttttccaaatattttcagtcaaggttggttgaatgcatggatgtggaacccatggatacacagggccaactgtatataatattgtacaatggtgcccttccccttttatttaattatctttatagttattttaaagagtagatttgtggattaatgcttttttgattattacttaacaattattacttaaattttgtcagagattggctgtgggagaactaactgaaaatggtttgacattagaagaatggttgccatcaacatggattacagataccattccccgaagatgtccatttgtgccacagatgggtgatgaggtactttaaaagttttaaattatccatttttcctcacatagttttaaacattttttaacagctcttgaaatagacttctggaaaatagaagcagttttgtaaagtatctcattctagatatttgtatctatgtatggagaaagtacaattaggagtgaaagtctaataatcttctttgaagctgctgtaaatctgaatattataagaatgaaaattttgttagtgttttaaaaagaaatgtatgttttagtaagaggtttttattgccagctttctttttctttttaaattaaaatatattcatttatcttaaggcttaatgtataacatgttaaaggaaaaatgaaatttagcaatttgttttcatttaattttatttcaaaaagtgttagtgttttgtttacccacagtgttcacagttcattagatctttaaaaaatctataatcttagaataagaaatactatttaatttttaagaaactgaggatgagaccattgttttagaatatgtacttgtgaatttagaatgaaaatgtaagaactggtctcaacattttttactcaaggttaatgtctccttgttttgtacacaaattttaagaggcagtgttatctggtgtccaacagattgaccataaataaattgggaattaaaatagaggcttctttagcaaagcacactaatttacagtataaagtggtaattgacactttaagaaaaaaaagaaaacaaaaactttttaatgctagagtgattctaaagaacagtctgaaagaatttatttaaaaatcctacttgcctcatacgacttgaagagcacagaacattaccacattgttgtgatatagtacagtgatagatcttaacatcctgtagcattaagatgtgaaattatatttaatacattatttcaacatggccagaaaagtttgtaattccttgttgaatgatttcctttcaaaatggttaggttgtctttttagctgctggtgcttgaaggtcttcattattcagcactattccatcatttaaagttccagacaactggagcaataatggtatttttttaatttaagttttagggtacatgtgtacattgtgcaggttagttacatacgtatacatgtgccatgctggtgtgctgcacccactaactcgtcatctagcattaggtatatctcccaatgctatccctcccccgtcccccaccccacaacagtccccagagtgtgatgttccccttcctgtgtccatgtgatctcattgttcaattcccacctatgagtgagaatatgcggtgtttggttttttgttcttgcaatagtttactgagaatgatggtttccaatttcatccatgtccctacaaaggacatgaactcatcattttttatagctgcatagtattccatggtgtatatgtgccacattttcttaatccagtctatcattgttggacatttgggttggttccaagtctttgctattgtgaataatgccacaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaattagtttacagtcccaccaacagtgtaaaagtgttcctatttctccacatcctctccagcacctgttgtttcctgactttttaatgattgccattctaactggtgtgagatggtatctcattgtggttttgatttgcatttctctgatgggcagtgatggtgagcattttttcatgtgttttttggctgcataaatgtcttcttttgagaagtgtctgttcatgtccttcgcccactttttgatgtggttgtttgtttttttcttgtaaatttgtttgagttcattgtagattctggatattagccctttgtcagatgagtagattgcgaaaattttctcccattttgtaggttgcctgttcactctgatggtagtttcttttgctgtgcagaagctctttagtttaattagatcccatttgtcaattttggctttggttgccattgcttttgttgttttagacatgaagtccttgcccatgcctatgtcctgaatggtaatgcctaggttttcttctagggtttttatggttttaggtctaacgtttaagtctttaatccatcttgaattgatttttgtataaggtgtaaggaagggatccagtttcagctttctacatatggctagccagttttcccagcaccatttattaaatagggaatccttttcccattgcttgtttttctcaggtttgtcaaagatcagatagttgtagatatgcggcgttatttctgagggctctgttctgttccattgatctatatctctgttttggtcccagtaccatgctgttttggttactgtagccttgtagtatagtttgaagtcaggtagtgtgatgcctccagctttgttcttttggcttaggattgacttggcaatgcgggcccttttttggttccatatgaactttaaagtagttttttccaattctgtgaagaaaggcattggtagcttgatggggatggcattgaatctgtaaattaccttgggcagtatggccattttcacaatattgattcttcctacccatgagcatggaatgttcttccatttgtttgtatcctcttttatttccttgagcagtggtttgtagttctccttgaagaggtccttcacatcccttgtaagttggattcctaggtattttattctctttgaagcaattgtgaatgggagttcactcatgatttggctctctgtttgtccgttgttggtgtataggaatgcttgtgatttttgcacattgattttgtatcctgagactttgctgaagttgcttatcagcttaaggagattttgggctgagacagtggggttttctagatacacaatcatgtcatctgcaaacagggacaatttgacttcctcttttcctaattgaataccctttctttccttctcctgcctaattgccctggccagaacttccatcactatgttgaataggagtggtgagagagagcatccctgtcttgtgccagttttcaaagggaatgcttccagtttttgcccattcagtatgatattggctgtgggtttgtcatagatagctctgattattttgaaatatgtcccatcaatacctaatttattgagagtttttagcatgaaaggttgttgatttttgtcaaaggccttttctgcatctattgagataatcatgtggtttttgtctttggctctgtttatatgctggattacatttattgatttgcgtatattgaaccagccttgcatcccagggatgaagcccacttgttcatggtggataagctttttgatgtgctgctggattcggtttgccagtattttattgaggatttttgcatcaatgttcatcaaggatattggtctaaaattatcttttttggttgtgtctctgcccagctttggtatcagaatgatgctggcctcataaaatgagttaggggggattccctctttttctattgattggaatagtttcagaaggaatggtaccagttcctccttgtacctctggtagaattcggctgtgaatccatctgatcctggactctttttggttggtaagctgttgattattgccacaatttcagatcttgttattggtctattcagagattcaacttcttcctggtttagtcttgggagagtgtatgtgtcaaggaatttatccatttcttctagattttctagtttatttgcgtagaagtgtttgtagtattctctgatggtagtttgtatttctgtgggatcggtggtgatatcccctttatcattttttattgcatctatttgattcttctttttttcttttattagtcttgctggcggtctatcgattttgttgatctttcaaaaaaccagctcctggattcattaattttttgaagggttttttgtgtctctatttccttcagttctgctctgatcttagttatttcttgccttctgctagcttttgaatgtgtttgctcttgcttttctagttcttttaattgtgatgttagggtgtcaattttggatctttcctgcttactcttgtgggcatttagtgctataaatttccctctacacactgctttgaatgcgtcccagagattctggtatgttgtgtctttgttctcgttggtttcaaagaacatctttatttctgccttcatttcattatgtacccagtagtcattcaggagcaggttgttcagtttccatgtagttgagcggttttgagtgagattcttaatcctgaattctagtttgattgctctgtggtctgagagatagtttgttataatttctgttcttttacatttgctgaggagagctttacttccaagtatgtggtcaattttggaataggtgtggtgtggtgctgaaaaaaatgtatattctgttgatttgaggtggagagttctgcagaagtctattaggtctgcttggtgcagagctgagttcaattcctgggtatccttgttaactttctgtctcgttgatctgtctaatgttgacagtggggtgttaaagtctcccattattaatgtgtgggagtctaagtctctttgtaggtcactcaagacttgctttatgaatctgggtgctcctgtattgggtgcatatatatttaggatagttagctcttcttgttgaattgacccctttaccattatgtaatggccttctttgtctcttttgatctttgttggtttaaagtctgttttatcagactaggattgcaacccctgtctttttttgttttccatttgcttggtacatcttcctccatccttttattttgagcctatgtgtgtctctgcaggtgagatgggtttcctgaatacagcacactgatgggtcttgactgtttatccaatttgccagtctgtgtcttttaattggagaatttagtccattgacatttaaagttaatagtgttatgtgtgaatttgatcctgtcattatgatgttagctggttattttgctcattagttgatgcagtttcttcctagtctcgatggtctttacattttggcatgattttgcagcggctggtaccggtttttcctttccatgtttagcgcttccttcaggagctcttgtagggcaggcctggtggtgacaaaatctctcagcatttgcttgtctgtaaagtattttatttctccttcacttatgaagcttactttggctggatatgaaattctgggttgaaaattcttttctttaagaatgttgaatattggcccccactctcttctggcttgtagagtttctgctgagagatccgctgttagtctaatgggcttccctttgagcgtaacccgacctttctctctggctgcccttaacattttttccttcatttcaactttggtgaatctgacaattatgtgtcttggagttgctcttctcgaggagtatctttatggcgttctctgtatttcctgaatctcaacgttggcctgccttgctagattggggaaattctcgtggataatatcctgcagtgttttccaacttggttccattctccccatcactttcaggtacaccaatcagacgtagatttggtcttttcacatagtcctatatttcttggaggctttgctcatttctttttattgtttttttctctaaacttcccttctcgcttcatttcattcatttcatcttccattgctgataccctttcttccagttcatcgcatcggctcctgaggcttctgcattcttcacatagttctcgagccttggttttcagctctatcagctcctttaagcacttctctgtattgattattctagttacacattcttctaaatttttttcaaagttttcaacttctttgcctttggtttgaatgtcctcctgtagctcagagtaatttgatcgtctgaagccttcttctctcagctcatcaaagtcattctgcgtccagctttgttccattgctggtgaggagctgtgttcctttggaggaggagaggtgctctgatttttagagtttccagtttttctgttctgttttttccccatctttgtggttttatctactttttgtctttgatgatggtgatgtacagatgggtttttgatgtggatgtcctttctgtttgttagttttccttctaacagacaggaccctcagctgcaggtctgttggagtaccctgcagtgtgaggtgtcagtgtgcccctgctggagggtgcctcccagttaggctgctcaggggtcaggggtcagggacccacttgaggaggcagtctgcccgttctcatatctccagctgcgtactgggagaaccactgctctcttcaaagctgtcagacagggacatttaagtctgcagaggttactgctgtctttttgtttgtctgtgccctgcccccagaggtggagcctacagaggcaggcaggcctccttgagctgtggtgggctccacccagttcgagcttcccggctgctttgtttacctaagcaagcctgggcaatggtgggcgcccctcccccagcctcgctgccgccttgcagtttgacctcagactgctgtgctagcaatcagcgagactccgtggggtaggaccctccgagccaggtgcgggatataatttcgtggtgcgccgttttttaagcccattggaaaagcgcagtattcgggtgggagtgacccgattttccaggtgccgtccgtcacccctttctttgattaggaaagggaactccctgactccctgtgcttcctgagtgaggcaatgcctcaccctacttcggctcgcacacggtgcgcgcacccactgacctgcgcccactgtctggcactccctagtgagatgaacccgttacctcagatggaaatgcagaaatcacccgtcttctgcatcgttcaggctgggagctgtagaccggagctgttcctattcggccatcttggcccctccccgcaataatggtattaatgaatagaatgaaagtatgggatagtggctatcccaaaatgttactaaaaacttagcattttaaaacacgttcttctactagtgggtgtcagactgctgtgttcaagtactagttctccaagacagtagtcaggagtcacttaaatttgatcttcatgaaatgtggataaatgtatgccctgctgtcttaccaggatttgtgtaaggattaaaatatatgtgaaagtacatttttcatttcaatgttttctgcatatgtacaaactacttgctaaaaaagttaacaacaaaagtggacaataaacttattagtcaacgttattatgcaacatagattaacttattttgattttaagaataaaatggtgaaaccagaaaataacttttcatcttaacttataagtgtgtattcaaagtaatgttatttagaacactttggttatgatttagtgatgttaagacaacttgagagaaaggaggagggagaaaatattagaatttctattagctgaaaaggcattagattttctctttgcaatttcagctgataccctgtatattggattaaaggaaacacatccaggttgtaagattatgactttattttttaggtttattatttccgacaaggacatgaagcctatgtcgaaatggcccggaaaaataaaatatatagtatcaatcccaaaaaacaaccatggcataaaatggagctacgggtatgacattgatttattatttaatagccccaaaaatacaattatggaatttggagaaaattgtatcaaaggttactattaacacaataagaaactgcatcatgaagataccttgtttaattgaagcagtcctagagagtaggaggtgtttaattttcatccagagaaacacaagatttgactgaatttatatactccaatgcagtttatgggttgggtacaaaatttagttatagtgaaaattaggtaataagtctaaagtaagctttgtattcgtaaatttgatgcgtatttctagatttgtcatcagtagttcagttaccaataggtctttggaatggtgatagaaaacttactttagtaactatactttagtaaagttctccttatttttttttccttttttacgttttaaaaatttcattgtaacttgcatactataaagtgcacaaatcttaatacagagctcagtgaatttttacatgtacacagtcatcaccatccagatcaagatttaaaccaagaagttttttctcaacaatgtttagcagtctattttgtggtttattttgtaaagatggttccttatattctctgtctctctcaggaacaagaacttatgaaaatagttggcataaagtatgaagtgggattacctaccctttgctgccttaaacttgcttttctagatcctgatactggtaaactgactggcggatcatttaccatgaagtaaggactgttgaaagaggcaatggttttcttagtattgtagattgtttttcaagattcagatacatttgaacatcttttaggtatttttaggtatgtgacctaatcaatttgtttctgtttgtaattagataccatgatatgcctgacgttatagatttcctagtcttgagacaacaatttgatgatgcaaaatacaggcgatggaatataggtgggtaatttatttagaagctatcgatgtttgatgtggtttttttttcttagtaagtgatttactatttttcataattgagaaaaagattatcagagaaatctttttgctatttgtaggaatgttaatagcaaagtaagatactatgtcagaggtattgttaagttttgtttaggaatgaaaacagtcgatgttgaaatttaaaacattttttctgttacatttgactttattttcatgttttacatgttaagttaaatttttgtataaaaatattaataacaatttaaatttgaagccaaaagaagcttaacaagatgtataagtttgtactgggactaaagctaaactgacatttttcttttattggttaatcctattataatctttaaaattagtatcataaacaagtctcaaagaaactaaaatctgtaaaagttttcaggcacttaaggtgcattaatttacatatagatgatgtgccacttatgagagtagttctcttaaaaatttcatataggaccagtgtgtgtcatggtatgatgtgtacacggtatgtatggtttgaaaaagcacatcaattgatataccagtctgtccttcagtgccactagaacgcattgacatggtggtttgtcctttgttaaaaacttgtatgtgctctgtaaggagaaattaggaaaatttggaagctatcaaaattatggacatggaagttagaaagaaaatttacatctcatgttccatttccctagaagttttgaaagatgtgtttcatcaaataagggagtaaccaagaaagggaaaagaatggaatcccctcaggaaacaggaagtccagaacaggaaaagttaaaaggaatcccaaataatattcagtagaagcctcaggatagcagctacctagcaggtccaaatctccttcaagaggaaagtcttcagggggaaaaaattggaacacataacttatctaacaagtttgtctttgtggaaaattacattgaagaatgttttttacagcttgagctgttagagattgtgggaagatttaattgtaaattcactgaaaaccaagcattaaaaaaatgaagcaattattaattttagggaaaacaaaattatataaggaaggaattatatgctgttttgcttaggagcaagcagtatttacacagtcatgattaaaaattgaatatggattaaatataatacacatactttttatgcacagttgtattgaagttatagttgtcccttagtatccacagggaattacttccaggacctccatggataccaaaatctttgcatacataggttccagaattgatcctacggaaccccccgatgtgaaaagttggcccttccatattcatggatttcacatcccactaatactgtattttcaatctgcatttggttgctgatgcagaatctgccaatacagagggccaaatctgtttattggaaaaatctgtgtataagtagacctggtcagttaaaacccatgtcttcaagaatcaactctgatacttactgtcaaaaattgatgagtgaagaaatctgtaagtcaagaagtagtaggtaaaatagatgaaaacatttttcttggttttggctatcagcctttggtacatctgattgtttaaatttttcacatacattacttgatatgataaattgtattttattttatttttgagatggagtctcgctctgtcacccaggctggagtgcagtgccgtgatcttggctcactgcaagttccacctcccaggttcacaccattctacctcagcctcccgattagctgggactacaggctcctgccaccacgcctggctaattttttttttgtatttttagtagagacagggtttcaccgcgttagccaggatggtcttgatctcctgaccttgtgatccgcccgcctcagcctcccaaagtgctaggattacaggcatgagccaccgcgcccggccgataaattttattttttaaggagttgcatttgccaataaattgcggaacaaataatagagatcaaagtgcagcaatattccagaagtgagctcataaccttggcaaagtaatgtggctaacattggacaaatcacagagaattaaaatgaatgagcatcataattttctcatttggatcttacaattttattctaatttatgggttaatatttctctaaagttagtatctaattaatcttttatatctgaggatatatgcattcacccaccgactgccatttaaaattacattagtaatttttacttcagcccctcctcggaaatgtattaaggcttactctcaatggtggacttcagtagtagtccaggttctttgcttaactttcttgttattctacagatattgttcgttatgattaggcacacattactcagtccttcctctttagcataataatcagtctgtgtagggtcataagatacagctctttgaaactttatgtttagtgtgaggtagtggtggtgtcaagaatgcttcttattaaactatatagtagctttttgttaagtgtcactagtatgcatctgaggaacaactgtcaacagcatgcagttgtgttctattaacttttctcctggctccaattttcttttttatcatattcttttaccttgtttttctaatttttaattcatttcatgctatatgtttttataaacttttaaatcctttttggtaagaaaatgagaacataactacttttctctttgagtttaaaaataaaactgattcagattaattgctaacttaattcattttaacttaactgtgtgtcaaaatgtggagtgctttaatagaactcattagtcaaaaactaccaggaggtagacatcaccatttctattttatagacaaggaaaatgaagttcaaaaataattaatatattcaaagttatacagatacgttatttgaacccaagtctttcaggctctagcctatgctagccacgagaaaactcaagtactgtgatagctgaggaacaggaaaacattgcataaaggaagtaagtgacttacagttttcttatgacacagaaggaacatgagaatatttagataagaaaaagccatagcatttatagcttgttcaggtctttgggaaaaagcaggttaagtaatacgacgacagcaattggataaggaaatagacacaaggagtgtcctctgttttaaaaagaatagttgttttatttttgtttcttttttgtttccttttacaggaaagggagagtgagtgagtattaagggtgattgagatgaaggaagacaggttggattttatgttgtttgttgtttttagcgtggagtcttgattgattgttggttggggaaacttgcttacagaaagtaagaaattaaaagactaagtaattgttgagtggaacttgccgcaattataattttatttcagtgaaaagtttgggtatagtgttaatgatgaatttcaaatgctagattggtaaaaatgcctaaaacaagtgaatttctgtataccttaatgaaatataaaaaatagaattgaaaactgttaagtcacacttcaagtatgcttactaaattccttttttctcttggtattgctgattgtacttacttatttttggagtttgtggttcactttttgtcttctgaaatcatacaggcagtttaggtacttaaaggttaacgttaggaaaaagcagtgacagcagccatgcatttcagaatgatgagttagtttttgttcttggccaggtgaccgcttcaggtctgtcatagatgatgcctggtggtttggaacaatcgaaagccaggaacctcttcaacttgagtaccctgatagtctgtttcaatgctacaatgtttggtaaggaaatatttgttgtattttagttcacctaggcttgctttgaatttttttttccatctattaagagataaagagacttcttttaaacacagaagcaattattttgataattgattttattaaaattgtgatacaataatttagattttttaatataatgatttttctttctttaagctgggacaatggagatacagaaaagatgagtccttgggatatggagcttatacctaataatggtaagtgtgtgttaggctttttgtttctccattatggagttaatttcttaagaaattatttaagatttgctaacatgctaccacttacagttgtgagtatcaaatagaaaacattcatttggcaaatatttattattgtacaccttgaagtaggggagattcagcagtgaagaagatagatataactcttgcctacttggaattgattgaaattaatttattacagcaaaacatgaaattgttttattaattactattcacccaagaaagtgtgcatttgggaggcagagacacttttgtaactttagcagagtgtttaaagtgtaacataatagttaagaggtcatgcttttgagtgagacggacatcagttcatatcctgacttgtcattttttatgtgatcttggcaagttccataaagcctcacaccccaactttttagtactcggccttagaaaccgtcaaagctaataccttccttatagggctactttgaagattaaatagcatcatgtgtgtaaagtgcttattgtagagcctgacacatataagagctcaatgaagatatagttgtaattaataatgataatataactgtcattatgcatatggcctgacacattcaggatttaataattagccttaggcagtgagtatgacatcttccttgcactgagaaaaggacgttgggaccaaagcctggagattatattttctttccacttcattgcccatttttgtttgtttgtaaaaaatacaacacacatgcacaattgcataagccaaaaagggattagaaggatattgtcgagattttaataatgattatttatagagtagcattataaagacttcataaattcttttacgttattctatattgtttgatttttaaaaaataatatattacagggcaggagtggtggctcacacctgtaatcccagcactttgagaggctgaggcaggtggatcacgaggtcaggtgttggagagcagcctgaccaacatggtgaaaccccgtctcaactaaaaatacaaaaattagcctggcgtggtggcgtgtgcctgtagtcccagctactcagaaggttgaggcaggaaaattacttgaacctgggaggcggaggtggcagtgagccgagattgcaccactgcgctccagcctgggcaacagagcgagactccatctcaaaaataataataatatattacatctcttagcagataaaaataaataagatctttaaaaaacaaaaaggaaaacatctaatttctaacatagtagaaataatcttttattaattgagttgctcagtagttattgtaatccaaagtcagagaaaaaataactgttggagaaaataacccagtctcgtagacctcagaaatgagaaaattgtcatactgattagctaagattagttctctgcaggaattatcactttgaaaatttaatgccaataatatatttttttttatctcactgctaaattagagctgtttaaaattataaaatatcttaaatcaattggtaaccatgttacatggcttatgtttttgttttgtttctctcatttattaaaaagaaagtttgaatttgaactcacattgaactgttgtgaatatgatagcaatgcagccagttggtgttataatgccttggatattttttaaatgaataagtgatactgattcactgttttgtttcctacctaaatatattgaagggtggttactgttgttgttctatttggatacacattcatacgaacctatagagtttaatcgtagtttgtgatgagaattttgattgtacacaaacagtggcgtattttctagagtgtatctgccaaaacttagtctaaaggaaaaattacctagcacgtttctagattttgggttaccgcaaccctctagtgattctctcagctaaaactgttaaagattcaagcatgcagaaagcataccttcataatttatgagtaataagcataagcatcatttgtcagttttcataattctctctgttcctacattcaaaaattgaagctttgattgaagtatttctagaaaatgtcactttagttcagttttaccttgtgtatactttatcaaagtaaacatggcaattatgaagtgattcagaaaactttgaaatttgcatattttatagaaatgatttctgcacatgtacttgattacagagcagattttcaggtataattttggagaagtaattatttagagaattacttatagtatcatttaaaaattaatttaaattttagtgacccttaaaacatactttcctttgttaaaataaagagtatatttacaaatctatacaaaatatactttaattttgtgactgttatttttacattctaaagctaacctgaatactaataagttaccatgtatttgcaatgatctgcttttctatttcagctgtatttcctgaagaactaggtaccagtgttcctttaactgatggtgagtgcagatcactaatctataaacctcttgatggagaatggggtaccaatcccagggatgaagaatgtgaaagaattgtggcaggaataaaccagttgatgacactaggtaagtaaagcaacacgcgagtatagaaaaaactggcagaacacaaagtaatggaaaaatactgttattctccaacaaaaattttttttcacaatatcctaagtcagtggtttcagacattttaagtggaatctctttttgaatgtggtcttaacacaggacttaactatttaaaacagttataaattaggttatgttagctgaattttagaataagggcctcctagaagtgccagaggcatctctgaaaaaactgggtcctccacaacccaatttaaaacacttgacatattcaggctgacatgatatgaattggaggaggaggattgaaagctgagttataattatatgggtagcttgtactggtctcccccaacttaatatacacacacaaaagaggatcttcttaaggggagaggagagggtatattaattgctctgccttgttaaaagaaaacagtgaccacaaaacttgattatctaaattctgggcttctgaagtaactagattatgtggtggtttgtagcaacccactctctcaataaagaatcagctgcaaagagtctagagagaaattaacatgagtaagaagagctggatggacacggagattgaggaatattcaaatctgaagtattttataaaatgccaacactttataggaatagccttataaatactaccttaaataattttagcaatattttaggatattaagtattatatgcgaatgagcaaaaaattgggagtgatcttaactagttatagtttttgctgtacaactttacggtgtgtattgggtcagtatcactgaatcaaaacttctgggtctgtttccttttctctacttgtctaatatttcacagaataatgtaaagttgagataaagggatacgtttgagaaagtcaaaggttagatttgaatgcagtatgttacagtgttaataatagaatggctttgccaaatatagattcaggaatggccaactgtcaattacggcaatgtatgtgtacttattaggtctgatatatgtatatataatttgtttctttctcccggtgaacctatgaataaaacaatacccctagaatttggcagtgtaaatgtagacataataaaaagtgagactctttatgtcagctacactatattgtttatgaaaagacagtggcagactaatgattatgtagattaggactttttaagtcttaattcaagcagaattcttcttgaattattttaggcatacaaaaataaatttacaaacttatctatttattttagatattgcctcagcatttgtggcccccgtggatctgcaagcctatcccatgtattgcacagtagtggcatatccaacggatctaagtacaattaaacaaagactggaaaacaggttttacaggttagaaccatttgatgtagctactatcaaaggaaagctggtgatctttccacccatttactgatctttagcagtgtttctcaactttttgtatataggaatgcacataagattatggggattatgttaaaatgtagaatcttattcagtagatttggagtgggacctgagattctgcatttctaacaagtcccagtaatgtcgatgctgctggtctttagacacttaaaatacaattcctagatcctagatacagtttttcctagaggctgacatgatttgatattcactgtctttatattgtattttataaagcaaggcaaggtatttcattttctcctagataacattaaaatatttgttcattgatttttaatataatatataataaaaattttgtattcgtattatgtattttgttcttattttgcctgtattcagcttatcattacttataacactgaaatttgaaaatactcagtttttcatagttgtagtttagcatgaaaaagaattagagttaaatggcccaggcataatttcaattaatttgcattatctaggtaattttgatgaatgtctttaatcgtcagtaggctttattattacctaaaatgttttgtcaaaaaatatgtttatagtttttaaataatggaatcctgtttttagttttaatttttatcatatgacctttgcttagcagtccaacaatttaatttgtgtaacattgcattaaaagtttttgtgaccagttatttattactgaagtcctttccgttattcttctttttaacatagagaagctttattcatattttgtattaatatttttctatataaaatgtgcctttggttctttttatgacttaaattttatttctgtatctcattgtgaggggctatcctgtgcctgtacccactagatgccagtagcagccccctagttgtgaaaaccaaaaatgtgttcagacattgtcacatgtcttctggggagcataattgtccttgattgagaaccactggaataaagaaaactaatatattagactttagtttctaaagatttttctgatcttcttaggtgtacttgctgttgcttccttgatatttgtcaaaaagcgtagagataaggagaagtcctccctgctatgtggttcccaaccatcccaccctcaaagcattcttcaagtctttttcaatatcaggtgttggaagtttaagagaaaaaaaaaaaaaaaaggaattcctaactatattcctctttctcccttgaccccttaaattgctaaagattggaaattaaacctagtgcttcagagatgaaacaagacatgggtggatgagaaggaaagaactatttgtatacatacactaattgttggactgcaactagaaatataagctgctgatgtcttccaatattgttgtaaatatctcacatattgctcaacatgataattgctaaagtatactatacataaggtttgacttacaatggtttgacttagatttttcagctttacagtgatatgaaagggatatgaattcagtagaaaccatacttcaaattttgaattttcatctttttctgggcttgtgatgtactctttcatgatgctgagcgatgtcagtgagctacagctcccagtagctcccagtcagccacgtgatcatgaggttaaaccaatactgtacagtgtattgtacagttgattgccatatgatgttgcccaaccataggctaatgtcagtgttctgagcacatttgaggtaggcttggctgaggtaaaatatttgctaggttaagtatatcgcatgcatttttgacttaatggtattttgaatttacagtgggtttatcaggatgtaacaccatcgttagttgaggaacatctgtatttcaagatacattatattttaaataggcttttggcattaccctagttttgtagccaatttaggaaaaaaacatttgaaagacaatacttacatgaattagaaattgcctgttttccaacctgataataaactctgttataaacctaagttatgttcttattcaatacaatgatgacttcaatgtattacattcaatacaatgtaagtaggtttttgcttcttccttcactcattggtgttagacttcttacagtttgtttttctttcgtgaaacttctagttcaatctcttgttaaactattactgtcagtttgccctgctggtattaatctaatactttattttacacacagttttctatatgatacactgaagatatctgtgattctgttatcagaatgagccttgtgaatctatttgaaatgcacagtttttctttatgtttctatgtccacaattagatacagtgttattccatatgattgtatttctgttcacaatcagtaagattttctaagtgttttctttattattttaagtcaggccacatattagataatattctcaaaagttgtccttggatttttatttactttcagaaataaaattgtaaaactccagattttattttagcattgtcaattaagtgttcagaaaataattctcatttagttaggtttcttggtaaactagcatgctatatttttatgattttgtatctcataaggtggctgtcagttaacttctcatttgtatatgtcatttgattaccagtaagataattagattaaaattacttgtttcctttttgttcagaagtgagggttctttattaccaaaaaatgacagcacacatgacaaaaatacataaagccaaaacttaaaactctcccctttctactctcctgccactccccatatagacgttctccctcccaaaaagaggcctttgttaatatggtgtgttccattccagacatcttgtatgcaaatacgtagttatggttgcccctctttttttcccctaacacaaatgcagttatgctgctttgaaaatgactttataagaccaatggtttttacagttggtttagactgaagttgaaaagttggaatgtcttggttttaaatgatgtacacatatttctaaatataaggaagttaaaacttctatattttgtttttttaaggcgggtttcttccctaatgtgggaagttcgatatatagagcataatacacgaacatttaatgagcctggaagccctattgtgaaatctgctaaattcgtgactgatcttcttctacattttataaagtaagttatcttttcatatgtaattatgatagttttaataattaatggcactacagttcctcttaaaatagaaaacatactgtttgtaaagaagaaacatttaagagtagccaaaatagctcagctgggagagcattagactgaagatctaaaggtctctggtttgatcctgggtttcagaagaaacagttcattggctaataataagaatgaaatcataaaaatcgtattaccattgtgttttaaggttaaaattgtttaaaaaaagattattttctcaatctaactgttagacaataataatagtaaaaatttatttaacatgtaatgttttacctacattaactcttctaacccaaataacaatcttacaggtggaagagataattatctggaattttgaattgaggaaactgtggcctatagtggttaacatgtataaggttgcacggtaaatgagtgtctgccatctgatttgagaatttttcatcctaacagtatattgcccctgtcaagtatattaacaagtatttaaatagtccagcaggagactgaatttaactttcatgattcttcttcaaagtcaagtttacttcctcattgatcccataacaagagtgataagcactatttattaaggcagaagtactttcccttccttatcttaaggattttatgcaatcatataatgaattgagcctcctaagttgaaggcagagaaatgaattctatattttgtacaaagactgctgtgattccatgtcattatttgtattgaatttttttttcctggttgagaagtagtaacagcattttaaaagcttacttagctgttatcagctttcatttttacataatgaaaaattcaggattattcagttttttttttttttttaataaacatccctgcttcattctcattaatggacataatctttgagctcctttttgattaatgtgttttaactgaggaatgcaggtcatttcagtataagaaaacaccaaacataattaataaggtaaagtttgtgccctttctctaccttattctctaagaaatctattgctttccaatggtttgggggctattattgctatgcttttgttttccttattgaaattagtaattaagcattatcctctgaagactcttatgtctgtaattttttataagtgttataactatgtgtatgtgtatctgttttttcaaagagaatcagtagaactacatgagatagtatattcatatttcagtgaaataatttagatgaaaattgttaatacatttgaaagtctgtgaaatgtatttctttcaaacggctactttgaaatgtctttgaagtcagattcatgaaatacttaaagcatttgtattgtatatttctaaaaataaatccatggcatttttttagatgtgtatagtttgtttaaaaatactttttttagcagatagtcttcaagatattaagtattccttgaaattttgattattatactttttaattgagttatttggcaaaatttcaagcaattgatacatacaaagtaatgtgatggatccttttaatacagtttcataggatagataaatctgtaagaaagtataatataaggaaaactgtgacaagtagtaagagaattgtataaaagtttagataagaataggattacttttagctgtgatgatggggattcagatacaggggaggagaagtctgttaagacattgtatacaaggtgtagatactgatagattctggaaatgttagatgaaaagaaagccattccagggataaggagcagcgtgaggtaggaagttttaattaagggtaatataaattggagaagtcacagtagagatacagtgctataaatctgagttggggccacatcgtgggtggctttgaatatgaatctaaaatataagctctgagagcaaggggctgttgaaagtatattaacatgagaataacatgttttacactatgccttaaaaatgttaatttgactgcagtgtctaatttaattgacagagaagagacaaaaagcataaatagcagattgactgctacagttgaccaactaagagttgatgagggcttaatttatgaaagtgagaaagggacagatgcccagacatacttgaggatagagttcacagcccttgaccactaattgggtttataaggaggaggaaagggaaagaataatgaaaaaccaagattcccattactgtaagaatgatatgttgttaaagaaaaatagaaaaaggaacaaggatatctggggagtcactattaagtttagacgtggaattttaggtgttggtagaatgctcaggagaagggttaggacaaattgcaggaattttggaattcatttacacagataaaccacaagtatgtgaagcaaaaggtacagtgtcaagatgcgttaagaacaaaattttaggaaatacatttaaaaataaaagaaagagttggaagacaagaaatgtattagctgtttgaagactgaggaatggaagaagcacagcagatacctaagcaaagactgggagatgagacttgggtttggtgtaatgatatagtgaggccacactgaaagctctacatagagtggaggggtcagggttaaatgtgtgtgcataaattattggagtaagccaatggagaagaaagcaagccttcagcttcaacttttaccttaaaatatggttttacgaacaaatttttaaggaaaaaaattatcatggttctaatcttacatgttaacatttccttgttatgtagggatcagacttgttataacataattccactttataattcaatgaagaagaaagttttgtctgattctgaggtatgtaatatttcattattattaccatattgatattctctatataaaaaaatttacatattgtagttttcaggtaaaagctgttgtgaacattattttttgtctagtgtagttaatttaaaaaaaaaaaacaactggcagtagaatattcttcttatagttaaaaaaaattttttaatggggggaacctttttaatgagtcaataaagtgccccagcatatacattacaggtgttccagtgtgtaaaggaaataattgctagttaatcaactataaagtataatgacatctaaaatgttttatgaatctggtaaattcttgcctcaaaggatgaagagaaagatgctgatgtgccaggaacttctactcgaaaaaggaaggtaatatagttttagttttagcagaagtactttatatgaattaaaatgtattaagagctttctttatttttttactcttagcattttaaaagacattaagtatttcaaattagtttcaattacaaaatgtatgttttttagaacttggtgaatattttcttttgtttgaattattgtatattacccaattatctaagactacattaaaagtacaaattttattgtgtaagtgctcttttattaatatatttgaacacttttatcacaggaccatcagcctagaagaagattacgtaatagagcccagtcttacgatattcaagcatggaagaaacagtgtgaagaattgttaaatctcatatttcaatgtgaagattcagagcctttccgtcagccggtagatctccttgaatatccagtaagtatttttgaaaatattttctttacctgtcagtttgctacataccttttacatgacagttggctattttagttactttttatgattatagtttatttctttattattttatacatgttctcattatccatttggctacatagagaatccaaattttgaatagtgtggtaaaatggaaggagccctgaatgaagacactgtaagaattgggttattctgttcctactagttgtgcagctttgagcaagtcccttagttttctcatctgaattttttttttctttttttgtcttccctacactgattccatgaatttattatggcaattatctagaaaaaaagaataatgcctacgtccttacaggattatggagatgtaaagagaatatataaaaatagtttttaggctgggcacggtggctcatgccagtaatcccagcactttgggaggccgaggcaggcagatcacgaggtcaggagatcgagaccatcctggctaacacggtgaaatcccgtctctacttaaaatacaaaaaaaaaaattagccaggcgtgtgggcgggtgcctgtagtcccagctacttgggaggctgaggcaggagaatggcgtgaacccgggaggtggagcttgcagtgagccgagatcccactgccgtactccagcctgggcgacagagcaagagactgagtctcaaaagcaaacaaaaaatagttttaaatgtgaagcactgtgtacaaatgtaaggtgacattattgtatatatttaggttatgccaatgtgtaatctttatggaatacatggtaaaaagagctaagtaaacatgatttaagtatttattttggcgtgcaatcatagcaatctaaatttaatgtagagtgatttctcatccctttaaaaagtaattaaatgttatatagttcaggggtcgggcgtggtgtcttacacctgtaatcccagcactttgggaagccaaggcaggcggatcacctgaggccaggagttcaagaccagcctgaccaacatggtaaaatcccgtctctactaaaagtacaaaaattagccgggctaatttttgttctaacttacaatgatgaaaataaagaaatttcatcaacagataaatatcagaaaatgacttttttcatgtcatttaaaatgccagaaacatagaatgttaaatgtggaagagaaatctttaattccagcggcgtgataaattgagctaattagaacctcacctttcaaaaaacctgggactgagctgatgctataatgttctggctttcatttctctctgtgttttcacaacttatggatttcgctttggttcttgcaagctcagctatgtattcttttgaaaaataagttgcagtttattcattgttttaagcgtttgtattctgggctttgcacagaaaagaagagatagaaataagacaatgtttttattctagaggcacaagtattaacactatacagagataattgagtccttgggcagtggtgaaaaaggaatttggagctgaaacccaaccctaaaccagaaatattgaagaactgtaccctgaatgaaaagaagaattgaataattcctttcacaacaaagaatgtttcccccagtggttctagtgtcagagaggagaaattttcctaaacttacactatacatggacttagaatccaaatttgcaccacccttgtgtagaaatccattatggaaaaaatgttaatccttaactgaatgaactaatgatttaaaaaaaaaacaacacctatataagggagtgatctaccataataagataacagaaacaacaaatgaaaatattagtaccctctccctgaaaatttgagtaatagattattctgaagtactgtacttcattaaaaaaaagagagatgacattccttgtaagattactgttgttatatgtaccaccaagaaaaaagaaatgctgttgagttaaactttgatagatagttttggttaaaggaataaatgaaagaataagaaacatagggaaaaaatatgttatttgagaggaaaagagtgaagacatattaaaatcaactaaatagaatcctggaaatgaaatgtataattactgaaattaaacttactggacagttttaaaaacagactaaagagatagtaagtgtaaagatctaagcaaattctatagaatcccacacagaatgataaagagattaagagtgtgaaggagagggtaagaggtagtggtagatagaaagagaaggtccaaaatatgtctaacaaaaatttcagtggaagagaatagacagaatgggagaggagcaatatttgaggagctaatggccaagaatttttttttcctttttttttttcttttttttttttcttttttttttgggacagagtctcactctgtctccaggctggagtgcagtggctcaatctcggctcactacaaactctgcctcctggattcaagcgattctcctgcctcggcctcacgagtagctgggattataggcgcatgccaccacgcccaactaatttgtatatttttagtagaggtggggtttcaccatgttggccaggatggtctcaatctcttgaccttgtgatctgcctgcctcagcctccccaaagtgctgggattacaggcataagccaccatgcccgatggccaagaattttctagaattggtaaacatttgagaagctatgattcctgatcgggatatacaagaaaagaatctttacctagaataagagtgaaaaaaactcaacaattgaaagtcaaaaagaaaatattgaaacagtcatacagaaaaaatggaggagacctgagactgctccaagccactcactttactgatgttgaactgatactcatcactctaagatttatagacaaaactatttatagctatttatttgtagctatttatagacaaagccacaatgggaacccgtatcccttatgtccagtgcaccagttatatgtgatattctgagacaataggcaaatgaatttttatataaagaaagtagttcacttttcaaacttaaaagggaaatatataagaatgcagtcatcttttacatgttcaaaataatgagaacaatatttagcaaatacttagaacctgtcccaccagctaaacgtgttaggcaacaaaaagtagtagaaataagaaaaatgtaatctcttgcctcttgcagtactccatgatggaggtttgttgttttgttaattactgtcacagaagagaaaaagaaaacttcggataatctactttttataattcagaattaactgtatcatcctttagtggaatatttaaggcagatcatgcacttcatatttcatcttttatatgaaaatattggttcacattttgagtttaatcaaatattcaaagcaagatgttaaaaattaaatttttaatggaaatgaatgggcttaatattctacaacgttcatttattcaacagaattgtattgagcagctattatgtactatactacatggtgtaagcccaagatatgagccaagaaaaattaagtattctacataaataaaatttacattaaccaaaatattgcaaaacattttaaaaaatcatttttatattttgcccaaatccacaaagtttgtaaagccaataatgtaaaatctccaaggacttgttaacgaaagagcccacctctaaataatctatggatcaaacaaatctcatgggaatttaaaaaatgcattgaactcaattaaaacaaattcagtatatcaaaatttgtaggatgcagttaaagcaatgcagggaaatttctaatactaaaatgtactaaagagtacagcagaaaaggagaagtttcaaatcaataatcgaagctcactcctcaagaacctagagaaagaagagtaaaatcacccaaaggtaagccaacaaaaggaaataataaagagctgaaatcagtgaaactcaaaaaagaaaaacaataaagaaaatctatgaaattgaaagctgggttctttgagaagatcaacagaattgacaagcctctgttaagatttacacagcttataaagagagaagacagattgttaatatcagagatgatatagagcatatcactgtagaccctgcaaataccagaaggataataaagtaaaaataagtaatactacaaagaattctacatatataaatttgataacttacatgaaatagaccgatattcaaagtataaactaccacaactcacccagtatgaagcaaataactcagtaaccctattaaggaaatttaatttgtgattataagttttcaaaaaacaaatgtctagtcctggatggcttcactggagaattcaaccaaacatttaaagaattaacacaaattctacacaatctcttccagaagatagaaaaactcaaaattcccaactcactctatgaagaccagtgttcccctgatctcaaaaccagacaataagagtagaaaagaagaaaaacctttaacgaaataacagcaaatagaattcaggaatatattaaaaaatgattttataccatgaccaagtgctgtttattccagagatacaaggatggttcagtattcaaaaaccagtcagtgtaatccatcatattaataggctaatgaagaaaaatctcatgattatatcaagtaatccagaaagaacatttgacaaaatttcacacccatttgttctgtgaatgtgtattttggttatggtaaagtcagtgcggtcacaggaggaaatgaaaacaaggcttggaaggaaaaagtttattatactcgagtcccaggccaagcatggtggctcacacctgtaatcccagcactttgggaggccaaggtaggtggatcacttgagctcaggagtttgagaccagtctgagcaatatggcaaaaccccgtctttacaaaaaatacaaaaattaaccgggtgcagtggcgtgcacctatagttcaagctactaatgaggctgaggtgggaggattgcttgagcctgggaggtagacattgcagtgagccaagatgataccactgaactctagcctggcaatagagtgagaccctatctcattaacaacaaaaaaaaaaaagtaaaaaagagtcccagaaaagtcggggcttaccacatgccacacagggccacaggggaagtgctaggttttggttgggtaggagagaccaaaaccagggaaaagcttagaaatcctttattggggtttctgcaggaaatgcaaggcaggtcacagttaacagcttattattggctagtttgaataattccagtgggccctggggcacaggtatctagttgtctggtcccagctctgagatgatttaaaacagggaaatactggcttagtgtgtgagagtgagaaaaggagctgattgtggctttatacttgggattcattggtatgtatatgaaagacatgttctcagtggaacccctcactaagactgcccaggaaggggcaatcttttcccaggtctgtaaggccccccaggtaccagatcatcattggcctgtgaatagatggcaaatagacaaatgcagaatctgaagaagtgagacattaattcattataaaacccctaagaaaaccgggaacaaaggaaaacttccacaacttgataaagtacatctataaaaagtacatctacagctaatatcatgtttactggtgacagactgagtgctttccccctaagaattaggaatgactgaaaaatgttcactctcaccactgctattcaatgtagtactggaaattctagccaatgcaataaggcaagaaaaggaaataaaaggcataactattggaaaggaagaaataaaaatttgtaaaggcagtaatcctaccttataggacaccttcttcatggtcagacgtatttgatcataatttaaacttgatggtgcaaagtcaagttgttacacttcaaaaaatatagtcgcatactgagggactgttgaaaagctgcaggccaggtgtggtggctaatgcctataatcccagcactttgggaggtcaagacgggcaggtcatttaagcccaggagtttgagaccagtctcagcaacatgctaagacccgtctctacaaaaattataaaaattagctgggtgtggtagcgcctgcctgtagtccccgctacttgagaggctgaggtgggaaggttgcttgagtctgggaggccaaggatgcagtgagctttgatggcaccaactgcactctaacctgggcagcagcaagactgtgtctcccaaaaagggggggtacaatactctttgtccctgctaaaaaaaaatagcatattctgataattttagaattatataccatattatgataattttctgttgctcttcctgtttcacgtgtgtgtttttattggttccaatctgttacctaattttatactctattgactaggtagacatctcaggtgacatgcatagactttgtgacaactatttacattcgtaattctttattactgcaaagcaaaactcccaattttagtactgaagttgagattactagaacttaggtgtctgcataaatgggatgctggcatacttccattgtttttatttccttaaagttacagttgtgttttaatgagtcagacctttagaatttataagtagtataaagttttgagtttttctttcaaactttcggttgtagagctgggctttttccttactcttttactttcccttttccaatacaactccatctcccaagtctccagtgaaaaattaataacttctgtgttcttgaataagaaaattattgttgattgcagtataaggggggggagtatgaaataaataagaattattagaaaaaagggactcttgataaggggaacaggagtgattattaagagttctaggacttgcagtcatcataaaaatcctgtgcgaatccctgcactgagaagtgatgctttgtgtagtaataatcataacaccacctgttttccctctcctaggactacagagacatcattgacactccaatggattttgctaccgttagagaaactttagaggctgggaattatgagtcaccaatggagttatgtaaagatgtcagacttattttcagtaattccaaagcatatacaccaagcaaaagatcaagggtatataattacattattttcttttatgactagattaagttagaggagtgtgttaaatgactaaatgttgctttacttaaaatttaggtcaaagttaactttctgttacattcttaatgttgtcctactggaaaaagaaattatacctttctactcagctccttgtatgaaataacattgatgttatctttgatgtctgggaatggttacttttcttgaagtagtgcggttgatgcaaattgtcctggtatgcaccagagccatctggccttgagttttctagctattatctgccttgtggagcatttttgcgaagatttttacaaataagtaaaaatataatttaacactctgaccagacatgacatcacaaaggagtgctatatagcaacgaaatgagaaagaatagatgctagtcctagatagaaaacaggttcaaacatttgggatagtttcagtgtaataaagttgacaataagttttgtctgtatgagccatttgaatttagagtcggacttttctttaagaatctcaaaacttggaagatttctgttctaaacacaaaaatacataattgttaaaatgcttcagtttaccttttcatcaaaagattaggaaaaagggatgtaaaaaacaataattaaattctaaatattttttactggaaaaatatttacattacagtatttactgaacaaaggtattttcctccaaggaatggttgaacacttttttttttccctcacagatttacagcatgagtttgcgcctgtctgctttctttgaagaacacattagttcagttttatcagattataaatctgctcttcgttttcataaaagaaataccataaccaaaaggaggaagaaaagaaacagaagcagctctgtttccagtagtgctgcatcaaggtatttaatttcttttaaataccactagctgatctataactttcatctaaatgatagaacttggtgttttttaatacttcctttactattccctatattgcagaatgataatttgacatgcaagttcctatgatgtggaggatttttaatcttttaactaaagctatactagtgtaagtgcttaaattcaaactgctaaatctatacagtaaaacagcatttttaggatagtgatggcatcatgctttccgtcatataactttagcatttacagcgatccttcactaaatattcactttcaaaggctcacaatcatatgaaatccactataaatctcatattttaacatatttcataatagtacaaatattgaatccaaaaatattataaactgttttattcgttcacatagagctataagagtgactgacaatactattttatttacagccctgaaaggaaaaaaaggatcttaaaaccccagctaaaatcagaaagctctacctctgcattctctacacctacacgatcaataccgccaagacacaatgctgctcagataaacggtaaaacagaatctagttctgtggttcgaaccagaagcaaccgagtggttgtagatccagttgtcactgagcaaccatctacttcttcagctgcaaagacttttattacaaaagctaatgcatctgcaataccagggaaaacaagtaagactcactttctaaattgaaatgtatatgatgattttcttaatttaagcttttactttgccaaatgcaatatactgagttttagtaattcctagcagacactgttgtttttgccttctttattaatttcctattttaaaaaaaagtttttaataagaagaattaaattgaggtttaggtcattatagactaatgcttaaaaatggcaactctgatctaaactacctgaatttaaatcacagtacccacactagctagcaatttgccaagacttgccttcgaaattttgaaatgatgggacagcttctaaaagggctgaaactattaagagtttctagttggatacgcctttttcctattagaaagtagctttcctaagaaagctttaagcatccttcaggttttgttgtcttggctaattcaaataattcaggttaaccttttttcttatctttaggttcaataactcttaatttttaaattttgtttgtatgttttagtactagagaattctgtgaaacattccaaagctttgaatactctttccagtcctggtcaatccagttttagtcatggcactaggaataattctgcaaaagaaaacatggaaaaggaaaagccagtcaaacgtaaaatgaagtcatctgtactcccaaaggcgtccactctttcaaagtcatcagctgtcattgagcaaggtaaggtattacttgtatctttcaagatacagtagatccttattataaataatgcttttgttggaatcagcatttatatccacttttcaggttgcccacttaaaagtcacacatctgtattagttgataaatttactgcaatgagctattattgtgccactgcactccatcctggtaaacagaatgagaccctgtctcaaaagaaaaaaaaaaaaaagaaataaatcttcaaagtttaaattaaatagggtatagttaacccaatgtagcattcttcacttttctagaaaaatgtttaagtaattattggatcacaaaactacttatttttctagactgacttttactaaataataattcattggggttgtctacaactgtagaatacaacttttgacaccttactgttacacttcaagttctttcacctccaggtttttattgtaaagtctttatgtttgaaagatgaagaaggtaaataataaaatatgccatgtgacatgttaaaataatatggcttcttggcagcagacagcccaataataagcattttgcatcatgactttcaaagtgtgtctaagacatttcttcattgttgtcttcagaaacctctctcaaaatttaatttctctactgaaatcttatttttgttaatggcattgggatctacccgtgtaaacttaaaatcagggagtcatcctagccttctcttcagcaactgaatctgttcaacagctttttgtttttgttgtttctccctccataacatctcttaaatctattctctcctctccattctcacctccatgacattatttcaaggcctacataatttcttgcttggactattttcctagtttcctaactggtcttcctgcctcagttctcaactctttcttaaccagtccctctctcatattgcagccaaagcgatctttttatttctctctagcacatgtggatgtgtgtacgtacacacagagaaaacacttccttgccttcaaatcatatatggaaacattttaaattttttgcctgttacacaagactctttattacttggctcttgcctaacattccaggttcttttaccttctttctctcttttgtatcccagtcatcctaaacacttagaagttcctgagtctttgttcttttctccttttgggggaatgtctttttttccctcctagtttaattgtctgttgcctttcacgtctcacttaaaatatcgtcttctttaggaagtcttctcctggcttctttagatagaatcaagcacttcctttttttttttttttttttttttaaaaaaagacaggatcttgctctgtcaccgaggctgaagtgcaatggcctgatttccattcactgcaacctctgccttgccggttcaagcaattcttctgcctcagcctcccaagtagctggaattataggtgtgtgccatcacacctggctgattttgtatttttgatatagacaggatttcaccattttgaccaggctggtctcaaacttctggcctctggtgatccaccccactcggcctcccaaagtattgtgatttcagatgtgagccaccacgcccagccaagcacttccatctttatgtcccagaaaaccctgttcattacctttggtggtacttataatgttgatcatattattcattttcacatgtctgtatctccaaagaatctgagcttaatgtgtttactcatttttaatgcccattatacagaagataataagtgtttgaataaccaaaatgctttgcatatgagtgtttcacatttttaataatctgccccctttctgaactgaatttagagtttgaatagtatattggccttgcttctttttattcatgaatcctagagaaatcaaatatatatgatgaattggttctagcattatgatagcggaggtctggtatcataacctgtttgatttttaatttttgagtattacaaatactcgagtactggtactgtatagaaatatcattatctattagttaacattaacagtgttttattacaaataatgacacctaaattaactatttagaatcaagcagtgttgtctagtggaatataatacaagccatatatgtaattttaaattttgtactcaccatgttcagtaaaaacaggtaaagttaattttaataatattttatttatcccattatatccaaaatgttatttcagtatttaatcagtattttaaaatgtatactgctttggtatttaaattgaaatttaaataaaaatttagttcctaagctgcataacttttcatgtgcccaggacaacaccgttcaatatcagtggtttttaaacttttttaaaaaacttctgaattcctttttaagaaagctgcattcattcttaacctttttacattatatattgctttataaagttgatgagtcctatagtccctgtctcagaaaaatgtacataaacatatccatacaaaatttgggaccattacaagggattcatgtagtccttaaagcctatcttctgtagtaggtacatgtactggatatttagaacccttgtagtcataatgtaaaacaaaagagattgacagaatccctgggcagtttaaatgtcagttgctccataggaaatacttaactactttatgtccacacccacattttttcccaaccactaatctaaagagagtcatttcactatgggggtcaggaattaaaagctttgcattttctgtctgtacttggctctgacctttggtaagtctcttaaccatgttgttttgttttgttttgttttgtttttgagatggagtctccctcttgtcgcccaggctggagtgcagtggcgtgatctcagctcactgcaacctccgcctcctgggttcaaaggattctcctgcctcagccccctgagtagctgggattacaggcacccaccaccacgcccagctactttttgtacttttagtagagacggggtttcgccatgttggccaggctggtctcgaactcctgacctcaggtgatccgcccactttggcctcccagagtgctgggattataatcgtgagccactgcacccgaccaaccatgattttcttatttacaaagtacagttccaacctgtgaccaaaaatgctgttatgaagatgtattctctttaaaagcagtttccatctatgaatttcctcaaaaatagagcattgcaaatcacctaccatatcaaataaaaacatgtcagaaatttggagggaaagagagttagttactggtagttcctgtggcatatttttcctttatctaagttatagcatttttttctacaacagaaaggtaattcatcatctccagatccttcaggtttagaatttgattgccagattttctctcctgttttatgaagcataatggccttttatcatggagtttgttacagtgggatcttaatcatgataatggtggcacacatagtgattttgattgatatatatatataatcagtatttcctaggattatagtcatttgccatttattacacaaggagataggacttttcaaagcctgtgaaaactgccatttagttgtttttaaatcctctttaaacagttctaagaacaaagtttttgaccaagagtaacttatttctaagttaggaataagaaacctttagtcatcccagctttatactcaatcacaatgttaacttagataagtcacttattttctatgagctttacctccctcatgagatcgttaagaatctgtctcttttaattagaaaataaataagatcattgaactattagtgttttctaggttcttttttctcattttaataaaataatttggattatgtcattacatataagtataccaatggggccttactgacaagtttgagagtcaaacaaaaaaagaaactccatatattaagtgtactgttgttcccttttaggagattgtaagaacaacgctcttgtaccaggaaccattcaagtaaatggccatggaggacagccatcaaaacttgtgaagaggggacctggaaggaaacctaaagtagaagttaataccaatagtggtgaaattatacacaagaaaaggggtagaaagcccaaaaagctacagtatgcaaagccagaagatttagagcaaaataatgtgcatcccatcagagatgaagtacttccttcttcaacatgcaattttctttctgaaactaataatgtaaaggaagatttgttacagaaaaagaatcgtggaggtaggaagcccaaaaggaagatgaagacacaaaaattagatgcagatctcctagtccctgcaagtgtcaaagtgttaaggagaagtaaccgaaaaaagatagatgatcctatagatgaggaagaagagtttgaagaactcaaaggctctgaaccccacatgagaactagaaatcaaggtcgaaggacagctttctataatgaggatgactctgaagaggagcaaaggcagctgttgttcgaagacacctctttaacttttggaacttctagtagaggacgagtccgaaagttgactgaaaaagcaaaagctaatttaattggttggtaacttgtaccaaaatattttacttcaaaatctataaagcaggtacagttaaggaataagtagaactaaggcttctgcttccttgctgctgtggtggagtagggaatgttatgatttgatttgcaaaaaaaaaaaaaaaaaaaaaaacaaacttacgagacacttcacttctttaaatgaatatatatatatatatataaatatatacatatatattttaaatgtttgctatttattgcccttagataggttatgcattttcacattcatttcattcactgtttgaaacgaaagaaattgatctataatgaattcctgatttatttatagaaaagacaattctgctacactctttaggagcataatattcctagtgatagagcatttcatgttaaaatgaattatttttgaccaagtgaggtacatttctattgatacaagtcatgcccctaaaagatagatgttatacagagtagcagtgaattaagaaatgtttcctctaaaagataaatataaaatttccaccatttgataccctaaacagtttaattttaattatagaatgttcatctacaaagtatctagaggaggcttttgctttattccatttctgccaaacaagagaaaatgtactgatgaagggaaacatatccacattggaaaacatttgactgtctaatttttcagaccttgattcttatatcactcaatctctgtttattgtgccaaagactgagaatcagtgcagtggaaagcctgtttttgacgtcaggacagcatacacttttcaatattggaaaggctatatattctaaagagcaagttattaaagagttatgctgagatatatctttttttggtactaatagaaaataatgcactggtgggtcctttgacagagatgttctagaggaaatgtttaagtggttaaaggattcaaggaaaatacaggaaaaaaggtatgggatttgatgtttacttgttgatctggattaatgtcatttctaaactttagacatatctaatagcctaaaatgacttacaaagaaacgtgtctgtgtgtgtatgtgtatatattgataaatgggtataattaaatatatatacaaacacatacttatatactattacccaggtataaattcttttttcaggattttttagatagcagtagaataaaaataattttaaaatgtcatactttatagtttaattttaagggggaaattggttattttcaattattaaaggttaaaataggccaaatcactttttatgcaatcatgttttatacagtggtctcattgcacattgtgttttttctgcactttttacggtatccttatcatgctggtatgtcaatatacaccctaaagaaaaactccatttaatgattgtgccttgtattctattattttttgcatcattagtaaaattaagttgtctattgtaaatgataactatatgacttaggagaatgtattgctatatgtactgctatggaaaaggaaagcagttatttatttgtttatggtacagttagttattttataccttaaaaaccaacatacttaccatttctattgtttaaaaattactgtccatttttttaaaaatttaaagaatgtagtattttctgaggactggtatggtacaaaaatgtaatcaaaattttcagatactacatttttaaaaagtgaggatggggaaaatatgtatcagcatgaccctgatgtagaaaaataaagtctgcaattgaaccttggcaaaatacattacataattatttctgataaggataaatattagctgtcatttaactgtcgtgaagaatgaaaaagtgatacatttgggctttttagatcatttgtaactaaatttgtacagaaattctcctgtgaaattaagtaaaccattttccagatcttgactgcattgctgagattttgcagttgaaaaataatttcaaatttttcaggttttttgtatatttatttttttctaacaaatatatttaactataaaattaaaattcagtagttaaactcttcataatgcagaaatggatggcttgattgtatagaaaaatgagtctttcagttacaccattcttgaaaataatagtgtatgtttgaaatttgtcattaattagttgcttttgaaggcaatctatttgaaatatgtaatttcctgatgttatctgcattgtgttaagaaaaaaataataggctataaaatcaatttttgtaaaaaaaatgtacagttttaagttgtgtacagaaaaaggaagagcgtacacagtcttagagtggaatatacctctagtacagtgtatgtgttaattccaactcaagcaaagaaatgagtatttaccatctttcctataacatgtaaatgtattattcctgttctacagacactgtatatttatgaccttatttggacattattagacactggtttcttagtaaagcacagtaacttggatcagattctttgtaaccgaaacacttaaacatgtattagtaataccaatatatttagattttatatttttataatttagcttctatgcattacagaagttgaattaagaaatttaagtcatcaccatatttttgttttctttaccacttgggaaaataatcctctttgaggcagtaacactatgtatgtactgttaataggccagaaagaaatattggcagttgtcttaggtaagcaataaaaacatctccaatttactgaaagttcagaaatataactcttacatgcatggaaagtgtaaaccaatttaggtatttctaagtacccaaccctagtattctctccctcaactccaacattctcaagtcccagatgaaaagtttcagtggagcattagtttaaatatttagtctaatcagagactcacagccctgggcaatagatacatagctttttaaaattttatcctttgtgacctgctatagtggcattggactattttctgcatgtattataaactaaagattggccttatttcaattgtttttattgcacaacttcactacaaatttaaagactgctggcattattataaaactgtcttcctccaatccaagtaaataagtatgtggaatatttgtgagctggattaggtaacatctgagacttgggattgactaaatttagttatcacttggaaatgaattacataagatacagtaatcagagaaaatatctttatgcagcatgtatgttaacatctagaactgtccctcatctgcagtgcagtagaggaatcagacattttaagaaatgtattcaaatatttttatcttatatgttagcagcatttacatgttttcagatatgatttgtaatataaattaatttctccagttaacaatgtttacttcaattctgacattccattttatacatatatacctactgtatatctctctcaaatgtcagctagaagcaactttttcagttttctatctaatgaaaacggatatattctgaatgtttttgttacctgaaatttatttttgagtaggttgaaaggaccaagatacttgagtgattaaaggactgtaagtgttctgcttatatgcaggagttacatgattttaaaagtcataataggggaagtagttttatcatctgattactcctattaacctaaaaagtatttctaacataacagattgagcagtggttttgttacacttaggcaagaatttattttcccattaaaatttgaaaaatacacttttctttgcttctacccttgcagtttcccttagttcccctaatttccaagtgatctgaactaaaattaatattacatttactaagaaactatttttgttaattatactgagcaaagtttgctccaaatgaaaagtcattacgaactatgtatagtacccaagaaaaggtcaaattgagtcagaagactgtaagtaccacttttggcgctccagagataaaccttgtacacactgcagtagaacagtgagtagaaaatatgagctataaatagatattatttttagttttgatagctctgtgttgaaatacttttttaatgcagataaaaattatcaggaaattaaccttatggcacagaccaaatacttagtgcatggctgagtttctacaattcaaggagccaagaaaataggttcaagatggaatatagtacatgcatataaggcagatgcacacatgctggttactttaagattgtgtgtgttgattagtcacaatgatagactataacatgagacaatacaaagttacatttttggaccatattaaaactgcaagaagacaggggtcttactgaagatcttttagaaaacttaaatcctgtcacaggatatttagacatgtgtagaatgtagctcaatttttaaaaagtaactgacctagagggtgaaagttgaaactgacacattttcaaattaagattatgcttattttgtacagaaaacaatgtttaaacacaagcagatctgttgtatgtaataagtaacacagagttttaaaacaaattaattatttagctttattgaagtttgtttttttccttccgaacctggagtatcatattataaacagcagtttcacaccagaatagcagtgccctttctttttgtacatactgattggacctttctttactgttacgtggacactttctatgttagttttgatgcataattctttgaatccttttatacaaactagaatgtatgtgtaagaatacctgtccctgcaatgtagtaactacaaacttatttttaaataaatagaaaacttgtttttctaagctattttgtagagcctcatgatttattatttgctttttaaagttttttggcatcacataaagcacttagtacataatctcaacaagctgcttaccaaagtggtcatatggcagataccattatgtaaatgctggttcattagagtttccagtgccctgtgtgttttgcaaagtgtgaaaactgtcagttaaaagatatttgggtgccgaaatgtaatttaattccaaaacaactgaaggtcaaaaacttatatgcctttttatgtgtacatttaataaaacaattttattgatttcttaccgtaagttactgtgatgagtgataaatacttcactattcagatactttcgtaagagatacatttcagtggaacactttgcataaatattttctcaaaaatgtgcaatttctgggaaaaaaggaatgatggaaagaaggttattgcagttttcctagaaattttgtcagattggcatgcatttttattgactaagaatcccaattttagcatgaagaccattagatatgaatacataaggccataacatttcaaattaagcacatggagtgatttgtaattttgtgttaatttctccctaagatgttttgttaaaatgattttgtatataataaatttctaagttgaggaaggaaggtaaaaaaaattcctgataaccttttctttatgaagtctgctaataacaatacctagtatatacttagaagaaccagccaagaaaaattacctttcagcaaccactctttacttatttctcttttgtaataataccaattttatgacccaggattccccagtatttaacggaagtaagattaaagaccaaagcacaaaacctctgttccttgcaatatagtacttcgtgtttgtaatcaatgtgtggtaaatttaacacatttaccacttactgattagtctttgggaaaagaatggtggaagtcattcaatgaaatacctttctacctaattggttagacatactggcagcacagtgtaatgaactggcctggagttgggaaatgggaatttgaccttcattcctgacaggaagtagaggaaccccagcaaagtcgtatctcagagcttcattttcctcatctgtaaagttttaaaggttgattaaattatctgtgaggtctaattctgttcaaatagccagtggtgataatctcatttgtatactgtcctctgtattcatggctcaactttggcttttatatttagtccctaaatagaatacatcatttaatgtgtagcttccagacaagctgtgtcttgcggccatgacctgattgttgttaagtgtgaggttttctgaacccttagcagaaggacttttaatgtttttacaacacactattgtattgctataaattgtgaattgctttaaaagtacatgtttactaataaaaagaagtacaggcacacttta

SEQ ID NO: 2 - DNA, 11223 bp, Homo sapiens
  source 1..11223: mol_type = unassigned DNA
gtaattatcattctcttcattttatacctactaaagatttgggagctttttgacaaaggtactctgcatgtcacaaaggaaatccatttaaaaattggttcattattttgactttaaattcccatcaataaagaatgatttattccacacttttattgacatgctgaatttaaaagaaaattaattgtgcatattacagtgcctgtacattgtgataactgatacagttttatcagttttagcagtgaaactttggaaattcacaagtatgaagatacaatatagagaagaaaaaatacttggaaaaccatgtgaagaaattataaataaatactcgtattattgtacttgtttaatagtcttctagcacaatagaattttactcaagtacactgattttaaaattaaggccaaagaatcaagcatttcttgcttttaatcaattggtagtatattaaatatttgagtttatactcttctcttcacattttgtttcttagatgttaacataattggccaagtatttgaattacaatgttttaatatatcacatacaacttaccgagcagtgtttctatttactgtatgaagtattaaggtctcctcaactactgcagtttgtgtagtcatattttttaaatgtctactttctgtatattagagatacctttcaacaaagaacaaaagaaaaaacagtttccactatttcagcatgtttaaatttttttaaaaaaccaattatagaaatctttagtaatgaaccataaaacaatacaattagtacatgtggtcagaattattttgccaaataaattatttcacaaaacagttgtgaaacatggtttgtgaaaaaattacacgaatgtgatgctatgcttgattgataatcagagtaaagttatttctatgtcgtgagtaagcagatataatagcagattttttagtgagggaaatagctttagctatctttaaggctgtgccttagaatacattaggtaagaaataaggtttgtaaaaccctctatttctagaaaacagtgttaattatttggtgtgcacttgttagtgatggatggatttcctttgtgattgtattgagatttttttaaaaagccacctgtaaatatgctagtaaaaaatattttctgcttttctatccttgtgtttctgatttgctcctttttggaatatctgttgaataaggaaattagtcatttctcaatctgtctttatagcttaaaaaaaataatatgtgagctatatatatactagtggcttcatttagtagttttgtatttccaaaggacattattatattggtagagtgtttggagggaaacttttctcccataattgaataccgctttaacttttcagttatataaaggtgaattttcccttatcaggtaagttggtcacactaatattctaccttttcagtagataacaaaaactgcagcaccttgagagttaagtgatttgctcttagaccattaaaaattagtaatgtaaccaggagtagaagtcagccataatatattgtatatttttaaaaattagctgagggattggttacattactgccttaaaaattgtttattgttaaataagagtgttagatgaggatatttttctggttctttttatggataagagataggaaaactctaaagagcagaagaaacaaatacatgtaataagttgaattggatagtattcatagaattgcagacagtgagcaaaacataaaccaaaaaagctaaaaaacagaaagtgagccacccttcatctcattaaaataaagatactacctttaattttttgtatgatgtttaatggtttccaaaatgctcccacatattttacctcctttgggtctcatgaaaaccttgtaagatgatcaagatagattttattattcttggtcttaggaatggagaaactgtacattacagagataaagtgctttgccccaatttactgagttcacacagctattatgcgggcacttggagcataaacccatgactctggttgtactcaaatcttgtcatggttggctttttgagcttttgtcaatgttactgcatagctaacttttaacaatactcagttagtgatatgtctatgaagaaatgttttcataatggtaataaatctgagtggatcttttgacatcttactctttaacttgattttttttcctgtctgtcttttatgagttatctaattgccctaattctttttgctcttcactcttttgttcttttcattgcttgttttatttatgggcaaatggaagataaagcatccagcattgctattaagaaaataaagagaaacaattcgagggagggcctgcgttctcactgcttgctatcatgaatgttgagttttggctataattatagctatttggctctctgtagctaatcaataagtttttcagttactaaaataccacgatacttgattgcatacaaatttaatccctaatgttaagttgtagtagtaatcacaagttcagtttagtaaatattttttgagcaccatgtgaagggtctagtcatatatagtgctttggtctattagaggatgaagaattctggtggaatactccttttttgactagaaatggtgaaagttatgtgaagtatttttaagattaaataatatttcatgtgactgttaaaactgaagtttataaaacagataagtaaactcaccattccacctacagctagaaaataaggaatgtagactatacctttagaataacagttttagtacaaatttcttaaatactggtttttatcaagtatattttaagaatactttaatatttctcctgctttttgaataataattgaattttccattggaaatttatacctttgcaaaagataatggaaatcatgtaacatataatttagtattttgtattcatcagcattttactgcattttttgtgtaattatggaaatgtaaattctttttttgaaattttagaaatagccaaaaatcctgttatgttaaaaagcctttcaagtgaaaaggtgaactttcaaatgcctgtttgatacatagtaattacattttaataatttttaaatataagattataaaacattaatattttaatattgcactgatagtcatcattgtaataagtattctaatttttttctgtatcaagccatgacttatacttttaaaaatttcttgtgctttattttgggaccatctaaatttgtcagcaaattaaagagtttgagattgggaattgagataaagctatttagttcttttatgtttaaataatttacttcattctgaaatcttataaatggattctcaactttcaagtagtattctccagagaaactgaggggtttatttttcttgtttttactgctaactttcctcctaatagaggatctcaatgataaaagcagttggggagtgggtgctgggtttgtttttgttacttataagaagtatatgacacgtgagatgatgttttaaaagaggctaacatagggcatgaaatggaatagacagtatatgtagcttgtactccccctaccctttctgttgtctcatctttaactaaaaacttttaaaagcacttattgggaaagcctttgtggttctttttgttgttgttgtttttcttttttaagtgaacaggagaagaaaccaaagagaattttgtagaaaatattatgagacaactttattctttaaaaacaaacaaactacattctaggccttcagtacttttatttctttacgaatcaactgaacagtccttacataacttgagtattcagaggaaatgtagtatatatttggatattttactgaagcaagtagtgtatattaagaatattccaaactataatagttgggtataacttagtaagcccacatttgtttggcattggcaggaggtaaacatcagaatagctttgttttgatgagagttgttctaaactctaaaggctctatactcccattaaatgctaaaaggcagataaataattcccagaaatagcttgaaaatgtgtgcaactctcaggactgaagctgagggccttctgtgaaagaggtcgccaggtttagaagttaattataaccttaaatgttcctaaatataaattgttttatggtcttttattccccattaaattagcaccggacagctaagttttaactacgagtttaactaaatgaatattaaattaatttgaaaagatagagacttttcacatttcttagtactaggccatttgaatattaaatagtaaataagagatgcttggagttgaatttgtagaccccatgtttttagtagctttcctcatgtttctggcggttttgctttatttctcttactctttcttttctctatagccctagctgtgtctctatctctctttccatatctttctatttctgtcattactcaaagctgtagtttgttgcctcctgtctggttttcattttaatcaaagagtatagtatgtctcttaatagaaagcagttttactgaatttgatttttgctttactttgttgtttcatgggtaaattccaaataagtttttatttgattaaagttttccttatttttataattgttctcttaacctttcttttacttaagagatttttttaaaaaaaaaaagttccagtgtccactaatccagaacaatatatttctccttgcaacgttccaaacgagcagaggctgccaatctaccacccagaatttttgtacatgcatacttcttctgatgttaacatgttctcagtgtgacctgtactttagttttagataattgtagctgtatgtatctaggaaagggagaaagagaagctcaaaataggtacagctcatattgtgttatctcttattaatcaggagaagagtctctctctttctctttcttttcttttctccctcacccaaccctccctttctccctccctcccttcctcccttctttccttccttctttcttcctatcatcatctaacaagataatgtcctatatttatactctctgtgttaaaaggactctctgatgcttccagtctatgtttcagcttctttgggtaaggaagtactttcggtcttcttaccatacactttagtaattatcactttcaggttttaagattctttagattatatctctcttatatatatatatcttggtacaaaaggcatgttaatctgttgccatgaatgtgagtttgtctctccagcttctcacactgcattatatgtggtaaattacttttatgtagtaattctttgacatagtccatgacttgctatcacaagggtatttgagatttgatagttatatgagaaggtatatgaaattatacagaatttaaacatgcagatgaaaacattttaacaaatatttgagtgttttcttttgtgttaagcacaaaatatgaggaccacaagggagaataagacgtggtctttttagtcagcctacagttaagttggggagacaaaatatacaaaatataccttaagggataaatattgataaataatttcaaacagtagcatgacaaatgagaaaagacagttgctgtatcttaagatgtagagatcattgtcagggtggacgattggagacgttttgttgaggagaaggaattgaaactatgccttgaaagataaataggaatcgagtcagtaagaaagagaatgaattttataaacaagaggaacattataaaaagctaagtgtcaagacatgttcatagaaatttagatatttaggaagcttgattgcagtagattttggaaataatgacgaaaaacaaattgagatgaactgtcagtgacttcaaatgatctcataggcataaatacctatgaaccttgtgggggctttagtaaattattttataggatggtcgagaagctaagagaaaaatagtttggttgccagtagtacaatttcaggtatgaaattgaataagtgtgaggacaactactttaattagtcatgaaatattataggggtagttggtaggaaatttgtaaagaaaaaaatacataaatgacttggtgttttgtgactatattaggatttgggctataggatgacagggaggaaagatttaataaatgggtttatagagtgttaagcccgggtgaccttgagaacaatggttttattagtggaaatatgaaagacataagggagaactgacttctttattcagttcatttgtaataatgtttattgcgttgaactacctatgtggaaatggccagctggcacttggagatagactacttagaggggctaggagtaaaaagaagaaagtgttgaagtgtttcttatagtatatagtttactttctcgtggagacaggagaaggaagtgcagaaggcttagaagtaaactttgggaaatactcatatatttagcagaatggaaaaggggtcacaatccttaagagggtgtgtcaaggaagacacaccatgctaatatgccaaaaagaccaaaagaaaataagcctcaaggaaggcctaatcaatagtattgctctccccagagaaatcataatatgggtacaaaatgatgttaaatttgaagatatttattttggtaatatttttcttttgatgaaaatctagagctgatttttcagaatcacttttatataataatttttttttcattttatgtaataatctgatctactgatttttatcaagtcaggtccagcagtttccttttttatgttggcataagttaaagaattttcaagattaaaacagccatgcaataactattttaaagtaatttgataacacctgaactgttggaatttggatttttttttttttttagacggagtcttgctctgtcaccaggctggagtgcagtggcacaatctcagctcactgcaacctccgcctcctggcacaatctcatctcactgcaacctccgcctcccgggttcaggtgatttcctgcctgcctcagcctcccaagtagctgggactacatgcgagcgccaccacacccagctaatttttgtgtttttagtagagacagggcttcaccatgttgaccaggatccaacacttatttcaaatatacatctgagtcttttttgtatctagtttacatatttttccctaggttggcaagctctaatctaaaagtttttttaatacataaggaaggacttcttaagggttattcttagcataaattccatttatcacctcttttcattcttggtatatgagtccatttcataagattattttagggtattttaaaagttgcatacataatttaaaattttggagtatttcctaggtaagatgctttcttctaatgaacggtgtgcttaaaaaaaaaaaaaaaaaaaaaagaacaagacatactgaccctgctaatagaattgaaataactctagaagaggatggttctgcctaagatagcccatattttttaaccttcctactttgtgttaaggttttctagaagcatatacttctcatattcactctctgtatattcttcatgattgatataattgagatcctgaattttagtatgcattctgcttttaatatacttcatctccagatttgtccagtttttttcaacatacctttcagaacctcagatggtaggattttagaactggattctagtgatcatttatctccatcattccacagagattaggtgatttacctattactgatggagagatgtcttgggtcttcgacctcattcctctgtctaatcattcatctttctgcttcaggaaaattatgttagttgtgtatagtaccttccctcaaaaaaggtattagtattctttccttaatgagtcctcatcagagctcttagctcataagtttttgtatcatttaatttctttaaagtttagtataaatctctaaatgcctaacaagtttatactactcatttattggtagtatttatagaattctttagtcagtatgagaattttcaaataagggagtcattattttaacacatctgatcctcccatctcctccttttaaaaataaaacaaaatacaataattgctgaatatacttttattttctttttactcctcttattctttttgaagttttcacagttactggtaaatactgtctcatagattatctcagctaaaccttattgatatttaatgtctatgagctttatgtggtctttatttctatatatccaaaagaatgcataacaagcatacttgattttattagaggaatagttcatctaaaatgcacttattttgataatttctcattacttattagtttcactctatttctaattattttactacaactgcagtaagtgctagctagcataagctatatgtagtattaaattttatgttgacacattttcttgatacgcagtgcatttaattttatgataggcttatatacctttcatttggtgagagagggtattttcttttcgtttttgaagataaaccttactgctttctagtagatgtgaatattcaaatagtgccttcatttaaaattttgtctttgagaattcttactcataaatactttgacccttgtttaaaaagataaaattcttttcttttgattggcttacttaatcgtaccatacattttaaaaccagttaggctatttttagttaatatagagaaagtaaaatattttcctgttttatatcatcaactgtttggaagcctggtctggaaaacctagtctgcagtttatagatatgggtgtttattgtctgaggatctattaataaatagagagttggtcttttaattgatgaagttagaggcaccttttctaactggttcatgagtaaagcagattcttttaatgcctcctttagtaaacaaatggttaatccaaataatatccttttcttttttctgatatgattgaaatatactttcattttctccatataaaagtcactttggagttaaacagatgccttataccaagaaaattaatactgtcttgccatattcacaatgaaatgtgttatataggacacacaggcatcttaaatatgattgctattttttctttgctcttttagagctttataagcttcaactgtccccaagattatttcagagatctactaactgtaaatgtggcaacattgataaataatagggtcaagggttgatcagtcctacttgccccgcaaccctcacccccaaaaagaaaaaatgtttgtttaaacaacacatgaaggataccatgcttaggaacatttatcagatttcctttgaaattgctaaattggactactttcaagtctccactctacttcaaattgtaatcttagacttgtaattatacttctggaaacatttattagagtagatttataaagtagttgcttttttgttctctctcttggcttctgtgaaattttgagtgaactgagttaaaaaaaaatcatttaacaacacatggctaaggcagtttcaatcccctaatttttagtttttgatataaaagtctatttagaggtgtactttgttcatgtggttgcttatttataacagttcatgtaacttttttttttatttgcctaggaaaacactgaaaaataaagacttgaatcctgaatttgtacttatagtctttcatttttggcaaatgcctagctgtatggctcctggtgcccttatggtaatctggagaaatagttatattctttcttgtttttgaattgttttaaagtagacttggaatcagttaccttcttctcctatcttatcatcctccaactttcttcacactcgagatataatcctgtttcacttatgattcatttcctttcttttctaagtttttgtgtatattaaacattagataattttcctagttttatctaatatggttacattttttaaaaagttattctttcttttctattattgtaaccatccaaaagagcattaataagcactgttgcacacatttcttctttttagcaagccaaaggaaaaagctagctttctttttgaggcagtcccatttgtggtattgtattaacttcttaggtaaagtcctttcttttatagaaatattttctctttttaccaaaatgcaaaccttccttgagaaatcagttattcttcttctgaattttaacttctgcatttggtgaactgtacatccatccttatgccacatttttaatactaaagaaagaaagttactacctttctcttttccaagcatttcctgggatattactaataaagctcaatttgttctagatcagtggataccggaacagaaaggaggtatttcttacctgataatgtgtgtgtgttccgattctgctaatccagaaatccaacagcatttgttgaaaatcatccttagttgttatttgttagggagtcaactccctattttagttgtttctgaaattcagtccctagatgtgctttagatagggtagaatttttacctctttcagaacactgctaaagaagttttcaactggaacatagtctgaagaagcactgagagcaaaaaattcagagtggtgattggccacctctgtttgattgacaggttgcagggaggaaacccattcgttctggattaatggaactggaaaacagaaaacaattatcaggtaaaaaaaaccttttctttaacctttgaaattcaccatttaaaagacgagtcagactaccagggaatttattaccaaaaataataaaatctagtgtcatttgagcaatgatctctggctactacttccaagaattaccaaaaaatccgtacagttgctacatgtcagcttttgcagaaagggcatgattgaacggagatagatggagtgtaagacataattatgtgttaaatctgagtttaaaacatgaagcagaattctatgggtgggggagttggtattcttttatttcctgaattgtgtttggtctgtggatagaatactttatttttcaaggctattaatctggcacttttcatgaatattcataaattttattactgtacaatgcttgttttacttaatcttcacttcattaggatgtatgcttttatcttaaaattattgtaataatagtgaccaattcttttcattttaatggtagttttcttactgttggttaaatatatttcag

SEQ ID NO: 3 - DNA, 64 bp, Homo sapiens
  source 1..64: mol_type = unassigned DNA
gttgcagggaggaaacccattcgttctggattaatggaactggaaaacagaaaacaattatcag

SEQ ID NO: 4 - DNA, 18 bp, synthetic construct
  misc_feature 1..18: note = Description of Artificial Sequence: Synthetic oligonucleotide
  source 1..18: mol_type = other DNA
ctttagcagtgttctgaa

SEQ ID NO: 5 - DNA, 18 bp, synthetic construct
  misc_feature 1..18: note = Description of Artificial Sequence: Synthetic oligonucleotide
  source 1..18: mol_type = other DNA
aacttctttagcagtgtt

SEQ ID NO: 6 - DNA, 18 bp, synthetic construct
  misc_feature 1..18: note = Description of Artificial Sequence: Synthetic oligonucleotide
  source 1..18: mol_type = other DNA
ttgaaaacttctttagca

SEQ ID NO: 7 - DNA, 18 bp, synthetic construct
  misc_feature 1..18: note = Description of Artificial Sequence: Synthetic oligonucleotide
  source 1..18: mol_type = other DNA
tccagttgaaaacttctt

SEQ ID NO: 8 - DNA, 18 bp, synthetic construct
  misc_feature 1..18: note = Description of Artificial Sequence: Synthetic oligonucleotide
  source 1..18: mol_type = other DNA
tatgttccagttgaaaac

SEQ ID NO: 9 - DNA, 18 bp, synthetic construct
  misc_feature 1..18: note = Description of Artificial Sequence: Synthetic oligonucleotide
  source 1..18: mol_type = other DNA
cagactatgttccagttg

SEQ ID NO: 10 - DNA, 18 bp, synthetic construct
  misc_feature 1..18: note = Description of Artificial Sequence: Synthetic oligonucleotide
  source 1..18: mol_type = other DNA
ttcttcagactatgttcc

SEQ ID NO: 11 - DNA, 18 bp, synthetic construct
  misc_feature 1..18: note = Description of Artificial Sequence: Synthetic oligonucleotide
  source 1..18: mol_type = other DNA
agtgcttcttcagactat

SEQ ID NO: 12 - DNA, 18 bp, synthetic construct
  misc_feature 1..18: note = Description of Artificial Sequence: Synthetic oligonucleotide
  source 1..18: mol_type = other DNA
ctctcagtgcttcttcag

SEQ ID NO: 13 - DNA, 18 bp, synthetic construct
  misc_feature 1..18: note = Description of Artificial Sequence: Synthetic oligonucleotide
  source 1..18: mol_type = other DNA
ttttgctctcagtgcttc

SEQ ID NO: 14 - DNA, 18 bp, synthetic construct
  misc_feature 1..18: note = Description of Artificial Sequence: Synthetic oligonucleotide
  source 1..18: mol_type = other DNA
gaattttttgctctcagt

SEQ ID NO: 15 - DNA, 18 bp, synthetic construct
  misc_feature 1..18: note = Description of Artificial Sequence: Synthetic oligonucleotide
  source 1..18: mol_type = other DNA
actctgaattttttgctc

SEQ ID NO: 16 - DNA, 18 bp, synthetic construct
  misc_feature 1..18: note = Description of Artificial Sequence: Synthetic oligonucleotide
  source 1..18: mol_type = other DNA
tcaccactctgaattttt

SEQ ID NO: 17 - DNA, 18 bp, synthetic construct
  misc_feature 1..18: note = Description of Artificial Sequence: Synthetic oligonucleotide
  source 1..18: mol_type = other DNA
gccaatcaccactctgaa

SEQ ID NO: 18 - DNA, 18 bp, synthetic construct
  misc_feature 1..18: note = Description of Artificial Sequence: Synthetic oligonucleotide
  source 1..18: mol_type = other DNA
aggtggccaatcaccact

SEQ ID NO: 19 - DNA, 18 bp, synthetic construct
  misc_feature 1..18: note = Description of Artificial Sequence: Synthetic oligonucleotide
  source 1..18: mol_type = other DNA
aacagaggtggccaatca

SEQ ID NO: 20 - DNA, 18 bp, synthetic construct
  misc_feature 1..18: note = Description of Artificial Sequence: Synthetic oligonucleotide
  source 1..18: mol_type = other DNA
aatcaaacagaggtggcc

SEQ ID NO: 21 - DNA, 18 bp, synthetic construct
  misc_feature 1..18: note = Description of Artificial Sequence: Synthetic oligonucleotide
  source 1..18: mol_type = other DNA
ctgtcaatcaaacagagg

SEQ ID NO: 22 - DNA, 18 bp, synthetic construct
  misc_feature 1..18: note = Description of Artificial Sequence: Synthetic oligonucleotide
  source 1..18: mol_type = other DNA
gcaacctgtcaatcaaac

SEQ ID NO: 23 - DNA, 18 bp, synthetic construct
  misc_feature 1..18: note = Description of Artificial Sequence: Synthetic oligonucleotide
  source 1..18: mol_type = other DNA
tccctgcaacctgtcaat

SEQ ID NO: 24 - DNA, 18 bp, synthetic construct
  misc_feature 1..18: note = Description of Artificial Sequence: Synthetic oligonucleotide
  source 1..18: mol_type = other DNA
tttcctccctgcaacctg

SEQ ID NO: 25 - DNA, 18 bp, synthetic construct
  misc_feature 1..18: note = Description of Artificial Sequence: Synthetic oligonucleotide
  source 1..18: mol_type = other DNA
gggtttcctccctgcaac

SEQ ID NO: 26 - DNA, 18 bp, synthetic construct
  misc_feature 1..18: note = Description of Artificial Sequence: Synthetic oligonucleotide
  source 1..18: mol_type = other DNA
cgaatgggtttcctccct

SEQ ID NO: 27 - DNA, 18 bp, synthetic construct
  misc_feature 1..18: note = Description of Artificial Sequence: Synthetic oligonucleotide
  source 1..18: mol_type = other DNA
cagaacgaatgggtttcc

SEQ ID NO: 28 - DNA, 18 bp, synthetic construct
  misc_feature 1..18: note = Description of Artificial Sequence: Synthetic oligonucleotide
  source 1..18: mol_type = other DNA
taatccagaacgaatggg

SEQ ID NO: 29 - DNA, 18 bp, synthetic construct
  misc_feature 1..18: note = Description of Artificial Sequence: Synthetic oligonucleotide
  source 1..18: mol_type = other DNA
tccattaatccagaacga

SEQ ID NO: 30 - DNA, 18 bp, synthetic construct
  misc_feature 1..18: note = Description of Artificial Sequence: Synthetic oligonucleotide
  source 1..18: mol_type = other DNA
gttttccagttccattaa

SEQ ID NO: 31 - DNA, 18 bp, synthetic construct
  misc_feature 1..18: note = Description of Artificial Sequence: Synthetic oligonucleotide
  source 1..18: mol_type = other DNA
ttttctgttttccagttc

SEQ ID NO: 32 - DNA, 18 bp, synthetic construct
  misc_feature 1..18: note = Description of Artificial Sequence: Synthetic oligonucleotide
  source 1..18: mol_type = other DNA
aattgttttctgttttcc

SEQ ID NO: 33 - DNA, 18 bp, synthetic construct
  misc_feature 1..18: note = Description of Artificial Sequence: Synthetic oligonucleotide
  source 1..18: mol_type = other DNA
ctgataattgttttctgt

SEQ ID NO: 34 - DNA, 18 bp, synthetic construct
  misc_feature 1..18: note = Description of Artificial Sequence: Synthetic oligonucleotide
  source 1..18: mol_type = other DNA
tacctgataattgttttc

SEQ ID NO: 35 - DNA, 18 bp, synthetic construct
  misc_feature 1..18: note = Description of Artificial Sequence: Synthetic oligonucleotide
  source 1..18: mol_type = other DNA
ttttttacctgataattg

SEQ ID NO: 36 - DNA, 18 bp, synthetic construct
  misc_feature 1..18: note = Description of Artificial Sequence: Synthetic oligonucleotide
  source 1..18: mol_type = other DNA
aggttttttttacctgat

SEQ ID NO: 37 - DNA, 18 bp, synthetic construct
  misc_feature 1..18: note = Description of Artificial Sequence: Synthetic oligonucleotide
  source 1..18: mol_type = other DNA
agaaaaggttttttttac

SEQ ID NO: 38 - DNA, 18 bp, synthetic construct
  misc_feature 1..18: note = Description of Artificial Sequence: Synthetic oligonucleotide
  source 1..18: mol_type = other DNA
gttaaagaaaaggttttt

SEQ ID NO: 39 - DNA, 18 bp, synthetic construct
  misc_feature 1..18: note = Description of Artificial Sequence: Synthetic oligonucleotide
  source 1..18: mol_type = other DNA
caaaggttaaagaaaagg

SEQ ID NO: 40 - DNA, 18 bp, synthetic construct
  misc_feature 1..18: note = Description of Artificial Sequence: Synthetic oligonucleotide
  source 1..18: mol_type = other DNA
aatttcaaaggttaaaga

SEQ ID NO: 41 - DNA, 18 bp, synthetic construct
  misc_feature 1..18: note = Description of Artificial Sequence: Synthetic oligonucleotide
  source 1..18: mol_type = other DNA
tggtgaatttcaaaggtt

SEQ ID NO: 42 - DNA, 18 bp, synthetic construct
  misc_feature 1..18: note = Description of Artificial Sequence: Synthetic oligonucleotide
  source 1..18: mol_type = other DNA
ttaaatggtgaatttcaa

SEQ ID NO: 43 - DNA, 18 bp, synthetic construct
  misc_feature 1..18: note = Description of Artificial Sequence: Synthetic oligonucleotide
  source 1..18: mol_type = other DNA
gtcttttaaatggtgaat

SEQ ID NO: 44 - DNA, 18 bp, synthetic construct
  misc_feature 1..18: note = Description of Artificial Sequence: Synthetic oligonucleotide
  source 1..18: mol_type = other DNA
gactcgtcttttaaatgg

SEQ ID NO: 45 - DNA, 18 bp, synthetic construct
  misc_feature 1..18: note = Description of Artificial Sequence: Synthetic oligonucleotide
  source 1..18: mol_type = other DNA
agtctgactcgtctttta

SEQ ID NO: 46 - DNA, 18 bp, synthetic construct
  misc_feature 1..18: note = Description of Artificial Sequence: Synthetic oligonucleotide
  source 1..18: mol_type = other DNA
ctggtagtctgactcgtc

SEQ ID NO: 47 - DNA, 18 bp, synthetic construct
  misc_feature 1..18: note = Description of Artificial Sequence: Synthetic oligonucleotide
  source 1..18: mol_type = other DNA
attccctggtagtctgac

SEQ ID NO: 48 - DNA, 18 bp, synthetic construct
  misc_feature 1..18: note = Description of Artificial Sequence: Synthetic oligonucleotide
  source 1..18: mol_type = other DNA
aataaattccctggtagt

SEQ ID NO: 49 - DNA, 18 bp, synthetic construct
  misc_feature 1..18: note = Description of Artificial Sequence: Synthetic oligonucleotide
  source 1..18: mol_type = other DNA
ttggtaataaattccctg

SEQ ID NO: 50 - DNA, 18 bp, synthetic construct
  misc_feature 1..18: note = Description of Artificial Sequence: Synthetic oligonucleotide
  source 1..18: mol_type = other DNA
tatttttggtaataaatt

SEQ ID NO: 51 - DNA, 18 bp, synthetic construct
  misc_feature 1..18: note = Description of Artificial Sequence: Synthetic oligonucleotide
  source 1..18: mol_type = other DNA
tttattatttttggtaat

SEQ ID NO: 52 - DNA, 18 bp, synthetic construct
  misc_feature 1..18: note = Description of Artificial Sequence: Synthetic oligonucleotide
  source 1..18: mol_type = other DNA
tagattttattatttttg

SEQ ID NO: 53 - DNA, 18 bp, synthetic construct
  misc_feature 1..18: note = Description of Artificial Sequence: Synthetic oligonucleotide
  source 1..18: mol_type = other DNA
gacactagattttattat

SEQ ID NO: 54 - DNA, 18 bp, synthetic construct
  misc_feature 1..18: note = Description of Artificial Sequence: Synthetic oligonucleotide
  source 1..18: mol_type = other DNA
caaatgacactagatttt

SEQ ID NO: 55 - DNA, 18 bp, synthetic construct
  misc_feature 1..18: note = Description of Artificial Sequence: Synthetic oligonucleotide
  source 1..18: mol_type = other DNA
aatcaaacagaggtggcc

SEQ ID NO: 56 - DNA, 18 bp, synthetic construct
  misc_feature 1..18: note = Description of Artificial Sequence: Synthetic oligonucleotide
  source 1..18: mol_type = other DNA
caatcaaacagaggtggc

SEQ ID NO: 57 - DNA, 18 bp, synthetic construct
  misc_feature 1..18: note = Description of Artificial Sequence: Synthetic oligonucleotide
  source 1..18: mol_type = other DNA
tcaatcaaacagaggtgg

SEQ ID NO: 58 - DNA, 18 bp, synthetic construct
  misc_feature 1..18: note = Description of Artificial Sequence: Synthetic oligonucleotide
  source 1..18: mol_type = other DNA
gtcaatcaaacagaggtg

SEQ ID NO: 59 - DNA, 18 bp, synthetic construct
  misc_feature 1..18: note = Description of Artificial Sequence: Synthetic oligonucleotide
  source 1..18: mol_type = other DNA
tgtcaatcaaacagaggt

SEQ ID NO: 60 - DNA, 18 bp, synthetic construct
  misc_feature 1..18: note = Description of Artificial Sequence: Synthetic oligonucleotide
  source 1..18: mol_type = other DNA
ctgtcaatcaaacagagg

SEQ ID NO: 61 - DNA, 18 bp, synthetic construct
  misc_feature 1..18: note = Description of Artificial Sequence: Synthetic oligonucleotide
  source 1..18: mol_type = other DNA
cctgtcaatcaaacagag

SEQ ID NO: 62 - DNA, 18 bp, synthetic construct
  misc_feature 1..18: note = Description of Artificial Sequence: Synthetic oligonucleotide
  source 1..18: mol_type = other DNA
acctgtcaatcaaacaga

SEQ ID NO: 63 - DNA, 18 bp, synthetic construct
  misc_feature 1..18: note = Description of Artificial Sequence: Synthetic oligonucleotide
  source 1..18: mol_type = other DNA
aacctgtcaatcaaacag

SEQ ID NO: 64 - DNA, 18 bp, synthetic construct
  misc_feature 1..18: note = Description of Artificial Sequence: Synthetic oligonucleotide
  source 1..18: mol_type = other DNA
caacctgtcaatcaaaca

SEQ ID NO: 65 - DNA, 18 bp, synthetic construct
  misc_feature 1..18: note = Description of Artificial Sequence: Synthetic oligonucleotide
  source 1..18: mol_type = other DNA
gcaacctgtcaatcaaac

SEQ ID NO: 66 - DNA, 18 bp, synthetic construct
  misc_feature 1..18: note = Description of Artificial Sequence: Synthetic oligonucleotide
  source 1..18: mol_type = other DNA
tgcaacctgtcaatcaaa

SEQ ID NO: 67 - DNA, 18 bp, synthetic construct
  misc_feature 1..18: note = Description of Artificial Sequence: Synthetic oligonucleotide
  source 1..18: mol_type = other DNA
ctgcaacctgtcaatcaa

SEQ ID NO: 68 - DNA, 18 bp, synthetic construct
  misc_feature 1..18: note = Description of Artificial Sequence: Synthetic oligonucleotide
  source 1..18: mol_type = other DNA
cctgcaacctgtcaatca

SEQ ID NO: 69 - DNA, 18 bp, synthetic construct
  misc_feature 1..18: note = Description of Artificial Sequence: Synthetic oligonucleotide
  source 1..18: mol_type = other DNA
ccctgcaacctgtcaatc

SEQ ID NO: 70 - DNA, 18 bp, synthetic construct
  misc_feature 1..18: note = Description of Artificial Sequence: Synthetic oligonucleotide
  source 1..18: mol_type = other DNA
tccctgcaacctgtcaat

SEQ ID NO: 71 - DNA, 18 bp, synthetic construct
  misc_feature 1..18: note = Description of Artificial Sequence: Synthetic oligonucleotide
  source 1..18: mol_type = other DNA
ctccctgcaacctgtcaa

SEQ ID NO: 72 - DNA, 18 bp, synthetic construct
  misc_feature 1..18: note = Description of Artificial Sequence: Synthetic oligonucleotide
  source 1..18: mol_type = other DNA
cctccctgcaacctgtca

SEQ ID NO: 73 - DNA, 18 bp, synthetic construct
  misc_feature 1..18: note = Description of Artificial Sequence: Synthetic oligonucleotide
  source 1..18: mol_type = other DNA
tcctccctgcaacctgtc

SEQ ID NO: 74 - DNA, 18 bp, synthetic construct
  misc_feature 1..18: note = Description of Artificial Sequence: Synthetic oligonucleotide
  source 1..18: mol_type = other DNA
ttcctccctgcaacctgt

SEQ ID NO: 75 - DNA, 18 bp, synthetic construct
  misc_feature 1..18: note = Description of Artificial Sequence: Synthetic oligonucleotide
  source 1..18: mol_type = other DNA
tttcctccctgcaacctg

SEQ ID NO: 76 - DNA, 18 bp, synthetic construct
  misc_feature 1..18: note = Description of Artificial Sequence: Synthetic oligonucleotide
  source 1..18: mol_type = other DNA
gtttcctccctgcaacct

SEQ ID NO: 77 - DNA, 18 bp, synthetic construct
  misc_feature 1..18: note = Description of Artificial Sequence: Synthetic oligonucleotide
  source 1..18: mol_type = other DNA
ggtttcctccctgcaacc

SEQ ID NO: 78 - DNA, 18 bp, synthetic construct
  misc_feature 1..18: note = Description of Artificial Sequence: Synthetic oligonucleotide
  source 1..18: mol_type = other DNA
gggtttcctccctgcaac

SEQ ID NO: 79 - DNA, 18 bp, synthetic construct
  misc_feature 1..18: note = Description of Artificial Sequence: Synthetic oligonucleotide
  source 1..18: mol_type = other DNA
tgggtttcctccctgcaa

SEQ ID NO: 80 - DNA, 18 bp, synthetic construct
  misc_feature 1..18: note = Description of Artificial Sequence: Synthetic oligonucleotide
  source 1..18: mol_type = other DNA
atgggtttcctccctgca

SEQ ID NO: 81 - DNA, 18 bp, synthetic construct
  misc_feature 1..18: note = Description of Artificial Sequence: Synthetic oligonucleotide
  source 1..18: mol_type = other DNA
gaatgggtttcctccctg

SEQ ID NO: 82 - DNA, 18 bp, synthetic construct
  misc_feature 1..18: note = Description of Artificial Sequence: Synthetic oligonucleotide
  source 1..18: mol_type = other DNA
cgaatgggtttcctccct

SEQ ID NO: 83 - DNA, 18 bp, synthetic construct
  misc_feature 1..18: note = Description of Artificial Sequence: Synthetic oligonucleotide
  source 1..18: mol_type = other DNA
acgaatgggtttcctccc

SEQ ID NO: 84 - DNA, 18 bp, synthetic construct
  misc_feature 1..18: note = Description of Artificial Sequence: Synthetic oligonucleotide
  source 1..18: mol_type = other DNA
aacgaatgggtttcctcc

SEQ ID NO: 85 - DNA, 18 bp, synthetic construct
  misc_feature 1..18: note = Description of Artificial Sequence: Synthetic oligonucleotide
  source 1..18: mol_type = other DNA
gaacgaatgggtttcctc

SEQ ID NO: 86 - DNA, 18 bp, synthetic construct
  misc_feature 1..18: note = Description of Artificial Sequence: Synthetic oligonucleotide
  source 1..18: mol_type = other DNA
agaacgaatgggtttcct

SEQ ID NO: 87 - DNA, 18 bp, synthetic construct
  misc_feature 1..18: note = Description of Artificial Sequence: Synthetic oligonucleotide
  source 1..18: mol_type = other DNA
cagaacgaatgggtttcc

SEQ ID NO: 88 - DNA, 18 bp, synthetic construct
  misc_feature 1..18: note = Description of Artificial Sequence: Synthetic oligonucleotide
  source 1..18: mol_type = other DNA
ccagaacgaatgggtttc

SEQ ID NO: 89 - DNA, 18 bp, synthetic construct
  misc_feature 1..18: note = Description of Artificial Sequence: Synthetic oligonucleotide
  source 1..18: mol_type = other DNA
tccagaacgaatgggttt

SEQ ID NO: 90 - DNA, 18 bp, synthetic construct
  misc_feature 1..18: note = Description of Artificial Sequence: Synthetic oligonucleotide
  source 1..18: mol_type = other DNA
atccagaacgaatgggtt

SEQ ID NO: 91 - DNA, 18 bp, synthetic construct
  misc_feature 1..18: note = Description of Artificial Sequence: Synthetic oligonucleotide
  source 1..18: mol_type = other DNA
aatccagaacgaatgggt

SEQ ID NO: 92 - DNA, 18 bp, synthetic construct
  misc_feature 1..18: note = Description of Artificial Sequence: Synthetic oligonucleotide
  source 1..18: mol_type = other DNA
taatccagaacgaatggg

SEQ ID NO: 93 - DNA, 18 bp, synthetic construct
  misc_feature 1..18: note = Description of Artificial Sequence: Synthetic oligonucleotide
  source 1..18: mol_type = other DNA
ttaatccagaacgaatgg

SEQ ID NO: 94 - DNA, 18 bp, synthetic construct
  misc_feature 1..18: note = Description of Artificial Sequence: Synthetic oligonucleotide
  source 1..18: mol_type = other DNA
attaatccagaacgaatg

SEQ ID NO: 95 - DNA, 18 bp, synthetic construct
  misc_feature 1..18: note = Description of Artificial Sequence: Synthetic oligonucleotide
  source 1..18: mol_type = other DNA
cattaatccagaacgaat

SEQ ID NO: 96 - DNA, 18 bp, synthetic construct
  misc_feature 1..18: note = Description of Artificial Sequence: Synthetic oligonucleotide
  source 1..18: mol_type = other DNA
ccattaatccagaacgaa

SEQ ID NO: 97 - DNA, 18 bp, synthetic construct
  misc_feature 1..18: note = Description of Artificial Sequence: Synthetic oligonucleotide
  source 1..18: mol_type = other DNA
tccattaatccagaacga

SEQ ID NO: 98 - DNA, 18 bp, synthetic construct
  misc_feature 1..18: note = Description of Artificial Sequence: Synthetic oligonucleotide
  source 1..18: mol_type = other DNA
ttccattaatccagaacg

SEQ ID NO: 99 - DNA, 18 bp, synthetic construct
  misc_feature 1..18: note = Description of Artificial Sequence: Synthetic oligonucleotide
  source 1..18: mol_type = other DNA
gttccattaatccagaac

SEQ ID NO: 100 - DNA, 18 bp, synthetic construct
  misc_feature 1..18: note = Description of Artificial Sequence: Synthetic oligonucleotide
  source 1..18: mol_type = other DNA
agttccattaatccagaa

SEQ ID NO: 101 - DNA, 18 bp, synthetic construct
  misc_feature 1..18: note = Description of Artificial Sequence: Synthetic oligonucleotide
  source 1..18: mol_type = other DNA
cagttccattaatccaga

SEQ ID NO: 102 - DNA, 18 bp, synthetic construct
  misc_feature 1..18: note = Description of Artificial Sequence: Synthetic oligonucleotide
  source 1..18: mol_type = other DNA
ccagttccattaatccag

SEQ ID NO: 103 - DNA, 18 bp, synthetic construct
  misc_feature 1..18: note = Description of Artificial Sequence: Synthetic oligonucleotide
  source 1..18: mol_type = other DNA
tccagttccattaatcca

SEQ ID NO: 104 - DNA, 18 bp, synthetic construct
  misc_feature 1..18: note = Description of Artificial Sequence: Synthetic oligonucleotide
  source 1..18: mol_type = other DNA
ttccagttccattaatcc

SEQ ID NO: 105 - DNA, 18 bp, synthetic construct
  misc_feature 1..18: note = Description of Artificial Sequence: Synthetic oligonucleotide
  source 1..18: mol_type = other DNA
tttccagttccattaatc

SEQ ID NO: 106 - DNA, 18 bp, synthetic construct
  misc_feature 1..18: note = Description of Artificial Sequence: Synthetic oligonucleotide
  source 1..18: mol_type = other DNA
ttttccagttccattaat

SEQ ID NO: 107 - DNA, 18 bp, synthetic construct
  misc_feature 1..18: note = Description of Artificial Sequence: Synthetic oligonucleotide
  source 1..18: mol_type = other DNA
gttttccagttccattaa

SEQ ID NO: 108 - DNA, 18 bp, synthetic construct
  misc_feature 1..18: note = Description of Artificial Sequence: Synthetic oligonucleotide
  source 1..18: mol_type = other DNA
aatcaaacagaggtggcc

SEQ ID NO: 109 - DNA, 18 bp, synthetic construct
  misc_feature 1..18: note = Description of Artificial Sequence: Synthetic oligonucleotide
  source 1..18: mol_type = other DNA
caatcaaacagaggtggc

SEQ ID NO: 110 - DNA, 18 bp, synthetic construct
  misc_feature 1..18: note = Description of Artificial Sequence: Synthetic oligonucleotide
  source 1..18: mol_type = other DNA
tcaatcaaacagaggtgg

SEQ ID NO: 111 - DNA, 18 bp, synthetic construct
  misc_feature 1..18: note = Description of Artificial Sequence: Synthetic oligonucleotide
  source 1..18: mol_type = other DNA
gtcaatcaaacagaggtg

SEQ ID NO: 112 - DNA, 18 bp, synthetic construct
  misc_feature 1..18: note = Description of Artificial Sequence: Synthetic oligonucleotide
  source 1..18: mol_type = other DNA
tgtcaatcaaacagaggt

SEQ ID NO: 113 - DNA, 18 bp, synthetic construct
  misc_feature 1..18: note = Description of Artificial Sequence: Synthetic oligonucleotide
  source 1..18: mol_type = other DNA
ctgtcaatcaaacagagg

SEQ ID NO: 114 - DNA, 18 bp, synthetic construct
  misc_feature 1..18: note = Description of Artificial Sequence: Synthetic oligonucleotide
  source 1..18: mol_type = other DNA
cctgtcaatcaaacagag

SEQ ID NO: 115 - DNA, 18 bp, synthetic construct
  misc_feature 1..18: note = Description of Artificial Sequence: Synthetic oligonucleotide
  source 1..18: mol_type = other DNA
acctgtcaatcaaacaga

SEQ ID NO: 116 - DNA, 18 bp, synthetic construct
  misc_feature 1..18: note = Description of Artificial Sequence: Synthetic oligonucleotide
  source 1..18: mol_type = other DNA
aacctgtcaatcaaacag

SEQ ID NO: 117 - DNA, 18 bp, synthetic construct
  misc_feature 1..18: note = Description of Artificial Sequence: Synthetic oligonucleotide
  source 1..18: mol_type = other DNA
caacctgtcaatcaaaca

SEQ ID NO: 118 - DNA, 18 bp, synthetic construct
  misc_feature 1..18: note = Description of Artificial Sequence: Synthetic oligonucleotide
  source 1..18: mol_type = other DNA
gcaacctgtcaatcaaac

SEQ ID NO: 119 - DNA, 18 bp, synthetic construct
  misc_feature 1..18: note = Description of Artificial Sequence: Synthetic oligonucleotide
  source 1..18: mol_type = other DNA
tgcaacctgtcaatcaaa

SEQ ID NO: 120 - DNA, 18 bp, synthetic construct
  misc_feature 1..18: note = Description of Artificial Sequence: Synthetic oligonucleotide
  source 1..18: mol_type = other DNA
ctgcaacctgtcaatcaa

SEQ ID NO: 121 - DNA, 18 bp, synthetic construct
  misc_feature 1..18: note = Description of Artificial Sequence: Synthetic oligonucleotide
  source 1..18: mol_type = other DNA
cctgcaacctgtcaatca

SEQ ID NO: 122 - DNA, 18 bp, synthetic construct
  misc_feature 1..18: note = Description of Artificial Sequence: Synthetic oligonucleotide
  source 1..18: mol_type = other DNA
ccctgcaacctgtcaatc

SEQ ID NO: 123 - DNA, 18 bp, synthetic construct
  misc_feature 1..18: note = Description of Artificial Sequence: Synthetic oligonucleotide
  source 1..18: mol_type = other DNA
tccctgcaacctgtcaat

SEQ ID NO: 124 - DNA, 18 bp, synthetic construct
  misc_feature 1..18: note = Description of Artificial Sequence: Synthetic oligonucleotide
  source 1..18: mol_type = other DNA
ctccctgcaacctgtcaa

SEQ ID NO: 125 - DNA, 18 bp, synthetic construct
  misc_feature 1..18: note = Description of Artificial Sequence: Synthetic oligonucleotide
  source 1..18: mol_type = other DNA
cctccctgcaacctgtca

SEQ ID NO: 126 - DNA, 18 bp, synthetic construct
  misc_feature 1..18: note = Description of Artificial Sequence: Synthetic oligonucleotide
  source 1..18: mol_type = other DNA
tcctccctgcaacctgtc

SEQ ID NO: 127 - DNA, 18 bp, synthetic construct
  misc_feature 1..18: note = Description of Artificial Sequence: Synthetic oligonucleotide
  source 1..18: mol_type = other DNA
ttcctccctgcaacctgt

SEQ ID NO: 128 - DNA, 18 bp, synthetic construct
  misc_feature 1..18: note = Description of Artificial Sequence: Synthetic oligonucleotide
  source 1..18: mol_type = other DNA
tttcctccctgcaacctg

SEQ ID NO: 129 - DNA, 18 bp, synthetic construct
  misc_feature 1..18: note = Description of Artificial Sequence: Synthetic oligonucleotide
  source 1..18: mol_type = other DNA
gtttcctccctgcaacct

SEQ ID NO: 130 - DNA, 18 bp, synthetic construct
  misc_feature 1..18: note = Description of Artificial Sequence: Synthetic oligonucleotide
  source 1..18: mol_type = other DNA
ggtttcctccctgcaacc

SEQ ID NO: 131 - DNA, 18 bp, synthetic construct
  misc_feature 1..18: note = Description of Artificial Sequence: Synthetic oligonucleotide
  source 1..18: mol_type = other DNA
gggtttcctccctgcaac

SEQ ID NO: 132 - DNA, 18 bp, synthetic construct
  misc_feature 1..18: note = Description of Artificial Sequence: Synthetic oligonucleotide
  source 1..18: mol_type = other DNA
tgggtttcctccctgcaa

SEQ ID NO: 133 - DNA, 18 bp, synthetic construct
  misc_feature 1..18: note = Description of Artificial Sequence: Synthetic oligonucleotide
  source 1..18: mol_type = other DNA
atgggtttcctccctgca

SEQ ID NO: 134 - DNA, 18 bp, synthetic construct
  misc_feature 1..18: note = Description of Artificial Sequence: Synthetic oligonucleotide
  source 1..18: mol_type = other DNA
aatgggtttcctccctgc

SEQ ID NO: 135 - DNA, 18 bp, synthetic construct
  misc_feature 1..18: note = Description of Artificial Sequence: Synthetic oligonucleotide
  source 1..18: mol_type = other DNA
gaatgggtttcctccctg

SEQ ID NO: 136 - DNA, 18 bp, synthetic construct
  misc_feature 1..18: note = Description of Artificial Sequence: Synthetic oligonucleotide
  source 1..18: mol_type = other DNA
cgaatgggtttcctccct

SEQ ID NO: 137 - DNA, 18 bp, synthetic construct
  misc_feature 1..18: note = Description of Artificial Sequence: Synthetic oligonucleotide
  source 1..18: mol_type = other DNA
acgaatgggtttcctccc

SEQ ID NO: 138 - DNA, 18 bp, synthetic construct
  misc_feature 1..18: note = Description of Artificial Sequence: Synthetic oligonucleotide
  source 1..18: mol_type = other DNA
aacgaatgggtttcctcc

SEQ ID NO: 139 - DNA, 18 bp, synthetic construct
  misc_feature 1..18: note = Description of Artificial Sequence: Synthetic oligonucleotide
  source 1..18: mol_type = other DNA
gaacgaatgggtttcctc

SEQ ID NO: 140 - DNA, 18 bp, synthetic construct
  misc_feature 1..18: note = Description of Artificial Sequence: Synthetic oligonucleotide
  source 1..18: mol_type = other DNA
agaacgaatgggtttcct

SEQ ID NO: 141 - DNA, 18 bp, synthetic construct
  misc_feature 1..18: note = Description of Artificial Sequence: Synthetic oligonucleotide
  source 1..18: mol_type = other DNA
cagaacgaatgggtttcc

SEQ ID NO: 142 - DNA, 18 bp, synthetic construct
  misc_feature 1..18: note = Description of Artificial Sequence: Synthetic oligonucleotide
  source 1..18: mol_type = other DNA
ccagaacgaatgggtttc

SEQ ID NO: 143 - DNA, 18 bp, synthetic construct
  misc_feature 1..18: note = Description of Artificial Sequence: Synthetic oligonucleotide
  source 1..18: mol_type = other DNA
tccagaacgaatgggttt

SEQ ID NO: 144 - DNA, 18 bp, synthetic construct
  misc_feature 1..18: note = Description of Artificial Sequence: Synthetic oligonucleotide
  source 1..18: mol_type = other DNA
atccagaacgaatgggtt

SEQ ID NO: 145 - DNA, 18 bp, synthetic construct
  misc_feature 1..18: note = Description of Artificial Sequence: Synthetic oligonucleotide
  source 1..18: mol_type = other DNA
aatccagaacgaatgggt

SEQ ID NO: 146 - DNA, 18 bp, synthetic construct
  misc_feature 1..18: note = Description of Artificial Sequence: Synthetic oligonucleotide
  source 1..18: mol_type = other DNA
taatccagaacgaatggg

SEQ ID NO: 147 - DNA, 18 bp, synthetic construct
  misc_feature 1..18: note = Description of Artificial Sequence: Synthetic oligonucleotide
  source 1..18: mol_type = other DNA
ttaatccagaacgaatgg

SEQ ID NO: 148 - DNA, 18 bp, synthetic construct
  misc_feature 1..18: note = Description of Artificial Sequence: Synthetic oligonucleotide
  source 1..18: mol_type = other DNA
attaatccagaacgaatg

SEQ ID NO: 149 - DNA, 18 bp, synthetic construct
  misc_feature 1..18: note = Description of Artificial Sequence: Synthetic oligonucleotide
  source 1..18: mol_type = other DNA
cattaatccagaacgaat

SEQ ID NO: 150 - DNA, 18 bp, synthetic construct
  misc_feature 1..18: note = Description of Artificial Sequence: Synthetic oligonucleotide
  source 1..18: mol_type = other DNA
ccattaatccagaacgaa

SEQ ID NO: 151 - DNA, 18 bp, synthetic construct
  misc_feature 1..18: note = Description of Artificial Sequence: Synthetic oligonucleotide
  source 1..18: mol_type = other DNA
tccattaatccagaacga

SEQ ID NO: 152 - DNA, 18 bp, synthetic construct
  misc_feature 1..18: note = Description of Artificial Sequence: Synthetic oligonucleotide
  source 1..18: mol_type = other DNA
ttccattaatccagaacg

SEQ ID NO: 153 - DNA, 18 bp, synthetic construct
  misc_feature 1..18: note = Description of Artificial Sequence: Synthetic oligonucleotide
  source 1..18: mol_type = other DNA
gttccattaatccagaac

SEQ ID NO: 154 - DNA, 18 bp, synthetic construct
  misc_feature 1..18: note = Description of Artificial Sequence: Synthetic oligonucleotide
  source 1..18: mol_type = other DNA
agttccattaatccagaa

SEQ ID NO: 155 - DNA, 18 bp, synthetic construct
  misc_feature 1..18: note = Description of Artificial Sequence: Synthetic oligonucleotide
  source 1..18: mol_type = other DNA
cagttccattaatccaga

SEQ ID NO: 156 - DNA, 18 bp, synthetic construct
  misc_feature 1..18: note = Description of Artificial Sequence: Synthetic oligonucleotide
  source 1..18: mol_type = other DNA
ccagttccattaatccag

SEQ ID NO: 157 - DNA, 18 bp, synthetic construct
  misc_feature 1..18: note = Description of Artificial Sequence: Synthetic oligonucleotide
  source 1..18: mol_type = other DNA
tccagttccattaatcca

SEQ ID NO: 158 - DNA, 18 bp, synthetic construct
  misc_feature 1..18: note = Description of Artificial Sequence: Synthetic oligonucleotide
  source 1..18: mol_type = other DNA
ttccagttccattaatcc

SEQ ID NO: 159 - DNA, 18 bp, synthetic construct
  misc_feature 1..18: note = Description of Artificial Sequence: Synthetic oligonucleotide
  source 1..18: mol_type = other DNA
tttccagttccattaatc

SEQ ID NO: 160 - DNA, 18 bp, synthetic construct
  misc_feature 1..18: note = Description of Artificial Sequence: Synthetic oligonucleotide
  source 1..18: mol_type = other DNA
ttttccagttccattaat

SEQ ID NO: 161 - DNA, 18 bp, synthetic construct
  misc_feature 1..18: note = Description of Artificial Sequence: Synthetic oligonucleotide
  source 1..18: mol_type = other DNA
gttttccagttccattaa

SEQ ID NO: 162 - protein, 1821 aa, Homo sapiens
MSCERKGLSELRSELYFLIARFLEDGPCQQAAQVLIREVAEKELLPRRTDWTGKEHPRTYQNLVKYYRHLAPDHLLQICHRLGPLLEQEIPQSVPGVQTLLGAGRQSLLRTNKSCKHVVWKGSALAALHCGRPPESPVNYGSPPSIADTLFSRKLNGKYRLERLVPTAVYQHMKMHKRILGHLSSVYCVTFDRTGRRIFTGSDDCLVKIWATDDGRLLATLRGHAAEISDMAVNYENTMIAAGSCDKMIRVWCLRTCAPLAVLQGHSASITSLQFSPLCSGSKRYLSSTGADGTICFWLWDAGTLKINPRPAKFTERPRPGVQMICSSFSAGGMFLATGSTDHIIRVYFFGSGQPEKISELEFHTDKVDSIQFSNTSNRFVSGSRDGTARIWQFKRREWKSILLDMATRPAGQNLQGIEDKITKMKVTMVAWDRHDNTVITAVNNMTLKVWNSYTGQLIHVLMGHEDEVFVLEPHPFDPRVLFSAGHDGNVIVWDLARGVKIRSYFNMIEGQGHGAVFDCKCSPDGQHFACTDSHGHLLIFGFGSSSKYDKIADQMFFHSDYRPLIRDANNFVLDEQTQQAPHLMPPPFLVDVDGNPHPSRYQRLVPGRENCREEQLIPQMGVTSSGLNQVLSQQANQEISPLDSMIQRLQQEQDLRRSGEAVISNTSRLSRGSISSTSEVHSPPNVGLRRSGQIEGVRQMHSNAPRSEIATERDLVAWSRRVVVPELSAGVASRQEEWRTAKGEEEIKTYRSEEKRKHLTVPKENKIPTVSKNHAHEHFLDLGESKKQQTNQHNYRTRSALEETPRPSEEIENGSSSSDEGEVVAVSGGTSEEEERAWHSDGSSSDYSSDYSDWTADAGINLQPPKKVPKNKTKKAESSSDEEEESEKQKQKQIKKEKKKVNEEKDGPISPKKKKPKERKQKRLAVGELTENGLTLEEWLPSTWITDTIPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQLEYPDSLFQCYNVCWDNGDTEKMSPWDMELIPNNAVFPEELGTSVPLTDGECRSLIYKPLDGEWGTNPRDEECERIVAGINQLMTLDIASAFVAPVDLQAYPMYCTVVAYPTDLSTIKQRLENRFYRRVSSLMWEVRYIEHNTRTFNEPGSPIVKSAKFVTDLLLHFIKDQTCYNIIPLYNSMKKKVLSDSEDEEKDADVPGTSTRKRKDHQPRRRLRNRAQSYDIQAWKKQCEELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTITKRRKKRNRSSSVSSSAASSPERKKRILKPQLKSESSTSAFSTPTRSIPPRHNAAQINGKTESSSVVRTRSNRVVVDPVVTEQPSTSSAAKTFITKANASAIPGKTILENSVKHSKALNTLSSPGQSSFSHGTRNNSAKENMEKEKPVKRKMKSSVLPKASTLSKSSAVIEQGDCKNNALVPGTIQVNGHGGQPSKLVKRGPGRKPKVEVNTNSGEIIHKKRGRKPKKLQYAKPEDLEQNNVHPIRDEVLPSSTCNFLSETNNVKEDLLQKKNRGGRKPKRKMKTQKLDADLLVPASVKVLRRSNRKKIDDPIDEEEEFEELKGSEPHMRTRNQGRRTAFYNEDDSEEEQRQLLFEDTSLTFGTSSRGRVRKLTEKAKANLIGW

SEQ ID NO: 163 - DNA, 16 bp, synthetic construct
  misc_feature 1..16: note = Description of Artificial Sequence: Synthetic oligonucleotide
  source 1..16: mol_type = other DNA
caatcaaacagaggtg

SEQ ID NO: 164 - DNA, 16 bp, synthetic construct
  misc_feature 1..16: note = Description of Artificial Sequence: Synthetic oligonucleotide
  source 1..16: mol_type = other DNA
gtcaatcaaacagagg

SEQ ID NO: 165 - DNA, 16 bp, synthetic construct
  misc_feature 1..16: note = Description of Artificial Sequence: Synthetic oligonucleotide
  source 1..16: mol_type = other DNA
tcaatcaaacagaggt

SEQ ID NO: 166 - DNA, 17 bp, synthetic construct
  misc_feature 1..17: note = Description of Artificial Sequence: Synthetic oligonucleotide
  source 1..17: mol_type = other DNA
tgcaacctgtcaatcaa

SEQ ID NO: 167 - DNA, 17 bp, synthetic construct
  misc_feature 1..17: note = Description of Artificial Sequence: Synthetic oligonucleotide
  source 1..17: mol_type = other DNA
ctgcaacctgtcaatca

SEQ ID NO: 168 - DNA, 16 bp, synthetic construct
  misc_feature 1..16: note = Description of Artificial Sequence: Synthetic oligonucleotide
  source 1..16: mol_type = other DNA
gcaacctgtcaatcaa

SEQ ID NO: 169 - DNA, 16 bp, synthetic construct
  misc_feature 1..16: note = Description of Artificial Sequence: Synthetic oligonucleotide
  source 1..16: mol_type = other DNA
ctgcaacctgtcaatc

SEQ ID NO: 170 - DNA, 16 bp, synthetic construct
  misc_feature 1..16: note = Description of Artificial Sequence: Synthetic oligonucleotide
  source 1..16: mol_type = other DNA
tgcaacctgtcaatca

SEQ ID NO: 171 - DNA, 17 bp, synthetic construct
  misc_feature 1..17: note = Description of Artificial Sequence: Synthetic oligonucleotide
  source 1..17: mol_type = other DNA
attaatccagaacgaat

SEQ ID NO: 172 - DNA, 17 bp, synthetic construct
  misc_feature 1..17: note = Description of Artificial Sequence: Synthetic oligonucleotide
  source 1..17: mol_type = other DNA
cattaatccagaacgaa

SEQ ID NO: 173 - DNA, 16 bp, synthetic construct
  misc_feature 1..16: note = Description of Artificial Sequence: Synthetic oligonucleotide
  source 1..16: mol_type = other DNA
ttaatccagaacgaat

SEQ ID NO: 174 - DNA, 16 bp, synthetic construct
  misc_feature 1..16: note = Description of Artificial Sequence: Synthetic oligonucleotide
  source 1..16: mol_type = other DNA
cattaatccagaacga

SEQ ID NO: 175 - DNA, 16 bp, synthetic construct
  misc_feature 1..16: note = Description of Artificial Sequence: Synthetic oligonucleotide
  source 1..16: mol_type = other DNA
attaatccagaacgaa

SEQ ID NO: 176 - DNA, 17 bp, synthetic construct
  misc_feature 1..17: note = Description of Artificial Sequence: Synthetic oligonucleotide
  source 1..17: mol_type = other DNA
ttccattaatccagaac

SEQ ID NO: 177 - DNA, 17 bp, synthetic construct
  misc_feature 1..17: note = Description of Artificial Sequence: Synthetic oligonucleotide
  source 1..17: mol_type = other DNA
gttccattaatccagaa

SEQ ID NO: 178 - DNA, 16 bp, synthetic construct
  misc_feature 1..16: note = Description of Artificial Sequence: Synthetic oligonucleotide
  source 1..16: mol_type = other DNA
tccattaatccagaac

SEQ ID NO: 179 - DNA, 16 bp, synthetic construct
  misc_feature 1..16: note = Description of Artificial Sequence: Synthetic oligonucleotide
  source 1..16: mol_type = other DNA
gttccattaatccaga

SEQ ID NO: 180 - DNA, 16 bp, synthetic construct
  misc_feature 1..16: note = Description of Artificial Sequence: Synthetic oligonucleotide
  source 1..16: mol_type = other DNA
ttccattaatccagaa

SEQ ID NO: 181 - DNA, 17 bp, synthetic construct
  misc_feature 1..17: note = Description of Artificial Sequence: Synthetic oligonucleotide
  source 1..17: mol_type = other DNA
tcaatcaaacagaggtg

SEQ ID NO: 182 - DNA, 17 bp, synthetic construct
  misc_feature 1..17: note = Description of Artificial Sequence: Synthetic oligonucleotide
  source 1..17: mol_type = other DNA
gtcaatcaaacagaggt